(19) United States
(12) Patent Application Publication
(10) Pub. No.: US 2026/0160759 A1
(43) Pub. Date: Jun. 11, 2026

(54) SYSTEMS AND METHODS FOR MULTIPLEX DETECTION OF BIOMARKERS

(71) Applicant: Veravas, Inc., Oakdale, MN (US)

(72) Inventors: Joshua Caine Soldo, Prior Lake, MN (US); Scott Douglass Bergmann, River Falls, WI (US)

(21) Appl. No.: 19/270,869

(22) Filed: Jul. 16, 2025

Related U.S. Application Data
(63) Continuation of application No. PCT/US2024/012072, filed on Jan. 18, 2024.
(60) Provisional application No. 63/480,437, filed on Jan. 18, 2023.
(60) Provisional application No. 63/481,360, filed on Jan. 24, 2023.

(51) Int. Cl. G01N 33/543; G01N 1/34

(52) U.S. Cl. CPC G01N 33/54313; G01N 1/34; G01N 33/54326; G01N 33/54346

(57) ABSTRACT

Provided herein are methods and systems for multiplex detection and/or measurement of biomarkers of a sample. The methods and systems can be used for rapid disease detection and/or monitoring, vaccine efficacy and immune response monitoring, therapeutic drug monitoring, and/or therapeutic safety and efficacy monitoring.

### CROSS REFERENCE

This application is a continuation application of International Application No. PCT/US2024/012072, filed Jan. 18, 2024, which claims the benefit of U.S. Provisional Application No. 63/480,437, filed Jan. 18, 2023, and U.S. Provisional Application No. 63/481,360, filed Jan. 24, 2023, each of which is entirely incorporated herein by reference.

### BACKGROUND

While biomarkers found in the body can be used to detect, predict, or manage diseases, many exist in too low of an abundance to be detected using commercially available tests. In addition, interference, a substance present in a patient specimen, can alter the correct value of the result of a diagnostic test, e.g., by interfering with antibody binding, or can increase or decrease assay signal by bridging, steric hindrance, or autoantibody mechanisms.

There is a clinical need for simple, inexpensive, automatable, and effective solutions for new diagnostic technology that can prepare clinical samples to improve testing accuracy, measure hard to find biomarkers, reduce costs, and ultimately save lives.

### SUMMARY

Disclosed herein, in some embodiments, are assay methods, comprising: Disclosed herein, in some embodiments, are assay methods, comprising: (i) providing a cleaned substance that has been subjected to cleaning by removing an interference from the cleaned substance with an interference capture composition, wherein the interference capture composition comprises an interference capture moiety, and wherein the cleaned substance is selected from the group consisting of a sample from a subject, a biomarker capture particle, a detection antibody, and any combination thereof; and (ii) capturing a biomarker from the sample by contacting the sample with the biomarker capture particle, and optionally detecting an antigen with the detection antibody, wherein at least one of the sample, the biomarker capture particle, or the detection antibody comprises the cleaned substance. In some embodiments, the biomarker capture particle comprises a biomarker capture moiety. In some embodiments, the biomarker capture particle comprises an antibody and the biomarker comprises an antigen that binds to an epitope of the antibody. In some embodiments, the biomarker comprises an antibody and the biomarker capture particle comprises an antigen epitope that binds to the antibody. In some embodiments, the method comprises the detecting an antigen with the detection antibody, and wherein the contacting with an interference capture composition to remove an interference comprises removing an interference or a conjugate non-specific binding (NSB) from the detection antibody. In some embodiments, the interference capture composition comprises an interference capture particle, a biomarker capture particle, or a combination thereof. In some embodiments, the method comprises contacting the detection antibody with the sample to bind a detection biomarker in the sample with the detection antibody; and detecting or measuring the detection antibody bound to the detection biomarker. In some embodiments, the capture moiety is in a liquid reagent. In some embodiments, the liquid reagent further comprises a sample preservative reagent or stabilization agent. In some embodiments, the liquid reagent further comprises a sample conditioning reagent or agent. In some embodiments, the capture moiety is in a solid reagent. In some embodiments, wherein the method comprises adding the capture moiety into the sample. In some embodiments, capture moiety is present in a sample collection device prior to contacting with the sample. In some embodiments, the capture moiety is biotinylated. In some embodiments, the biomarker capture particle is coated with streptavidin. In some embodiments, the biomarker capture moiety comprises an antibody or an antibody binding fragment. In some embodiments, the biomarker comprises one or more biomarkers selected from the group consisting of an antigen, an antibody, a protein, a small molecule, a therapeutic, a hormone, a peptide, a signaling peptide, an exosome, a cell, or any combination thereof. In some embodiments, the biomarker comprises an antibody and the biomarker capture moiety comprises an antigen of the antibody. In some embodiments, the antibody is selected from the group consisting of autoantibodies, therapeutic antibodies, immunoglobulin classes, immunoglobulin subclasses, circulating antibodies, secretory antibodies, alpaca derived nanobodies, and animal derived antibodies. In some embodiments, the biomarker comprises an antigen. In some embodiments, the biomarker capture moiety comprises an antibody of the antigen. In some embodiments, the antigen comprises a therapeutic, a drug, a small molecule, a peptide, a protein, a vaccine, or an immunogen. In some embodiments, the antigen comprises a repeating epitope, wherein the antigen binds to more than one biomarker capture particle. In some embodiments, the antigen comprises at least a first type of epitope and a second type of epitope. In some embodiments, the biomarker capture particles are coated with at least a first type of antibody specific to the first type of epitope and a second type of antibody specific to the second type of epitope. In some embodiments, the biomarker comprises a disease state-specific biomarker. In some embodiments, the biomarker capture particles comprise agglutination particles, wherein the agglutination particles aggregate upon binding to the biomarker to form aggregated biomarker capture particles. In some embodiments, the aggregation is visually detectable or detectable via a detection technique. In some embodiments, the aggregated biomarker capture particles exhibit a color. In some embodiments, the method further comprises eluting the biomarker from the biomarker capture particles. In some embodiments, the method further comprises quantifying the biomarker. In some embodiments, quantifying the biomarker comprises determining a biomarker mass per sample volume. In some embodiments, the sample comprises a biofluid. In some embodiments, the biofluid comprises a blood, a plasma, a serum, a saliva, or a saline oral rinse. In some embodiments, the interference comprises a lipid, a triglyceride, a bilirubin, a hemolysis product, a cholesterol, a human anti-mouse antibody (HAMA), a rheumatoid interference (RF), a manufacture assay specific interference (MASI), a human anti-animal antibody (HAAA) interference, a free biotin interference, an anti-streptavidin interference, an anti-biotin interference, a human anti-polyethylene glycol (PEG) interference, an anti-albumin, a non-specific binding, an anti-polyvinylpyrrolidone (PVP), an anti-polymer, an anti-alkaline phosphatase (ALP), an anti-ruthenium, an anti-fluorescein, an anti-acridinium ester, an autoantibody, an anti-horse radish peroxidase (HRP), an anti-conjugation linker, an anti-amino acid tag, an anti-histidine tag, an anti-polyhistidine-tag, an over-the-counter (OTC) supplement, a herbal remedy and/or therapeutic, or any combination thereof. In some embodiments, the hemolysis product comprises a hemoglobin, a lactate dehydrogenase, a potassium, or a combination thereof. In some embodiments, the human anti-animal antibody (HAAA) interference comprises a mouse immunoglobulin, a goat immunoglobulin, a sheep immunoglobulin, a rabbit immunoglobulin, a bovine immunoglobulin, or a combination thereof. In some embodiments, the anti-acridinium ester comprises an ABEI, a luminol, an isoluminol, or any combination thereof. In some embodiments, the anti-conjugation linker comprises an LC, an LC-LC, a PEOn, a chromogen, or any combination thereof. In some embodiments, the method further comprises contacting the sample with a conjugate. In some embodiments, the method further comprises cleaning the conjugate with the interference capture particles or the biomarker capture particles prior to contacting the sample with the conjugate. In some embodiments, the sample of the subject has been subjected to one or more adjustments to chemical or physical properties of the sample to eliminate one or more interferences. In some embodiments, the chemical or physical properties are selected from temperature, color, pH, salinity, conductivity, density, viscosity, surface tension, and protein content. In some embodiments, the one or more adjustments comprise (i) adding surfactants, detergents, cell lysis agents, anti-protease agents, protein-based or polymeric-based blocking reagents, displacing agents, agents, or any combination thereof to the sample to release analytes from a matrix or to remove interfering elements; performing a dilution of the sample; or any combination thereof. In some embodiments, the interference capture moiety interacts with the interference. In some embodiments, subject is suspected of having a disease, or has been administered a vaccine against the disease. In some embodiments, the disease comprises an infection. In some embodiments, the infection comprises a viral infection, a bacterial infection, or a protozoan infection. In some embodiments, the disease comprises a tick-borne illness. In some embodiments, the disease comprises Lyme disease. In some embodiments, the disease comprises a Celiac disease. In some embodiments, the disease comprises a severe acute respiratory syndrome. In some embodiments, the disease comprises a coronavirus infection. In some embodiments, the coronavirus infection comprises Coronavirus disease 2019 (COVID-19). In some embodiments, the cleaning comprises cleaning the sample by removing the interference. In some embodiments, the vaccine is configured to generate a component of a pathogen or disease. In some embodiments, the method further comprises determining a vaccine efficacy based on the level of eluted biomarker. In some embodiments, the method further comprises readministering the vaccine to the subject based on the vaccine efficacy. In some embodiments, the eluted biomarker comprises antibodies against a pathogen or disease. In some embodiments, the method further comprises identifying a likelihood of the subject having the disease based on the eluted biomarker. In some embodiments, the method further comprises identifying a likelihood of the disease being active or acute. In some embodiments, the method further comprises administering the disease treatment to the subject when the subject is identified as having the active or acute disease. In some embodiments, the method further comprises not administering or discontinuing the treatment when the subject is identified as not having the active or acute disease. In some embodiments, administering the treatment comprises adjusting a dose amount or timing. In some embodiments, the biomarker is secreted. In some embodiments, the biomarker comprises IgA, IgG, or IgM, or a combination thereof. In some embodiments, the subject has been administered a therapeutic compound. In some embodiments, therapeutic compound comprises a therapeutic drug. In some embodiments, the therapeutic compound, or a fragment thereof is comprised in an antigen. In some embodiments, the captured biomarker comprises autoantibodies against the therapeutic compound. In some embodiments, the method further comprises determining a level of safety of the therapeutic compound based on the quantified biomarker. In some embodiments, the method further comprises administering or adjusting a dose of the therapeutic compound to the subject based on the quantified biomarker. In some embodiments, the therapeutic drug comprises a therapeutic antibody. In some embodiments, the therapeutic antibody comprises an antibody binding fragment. In some embodiments, the captured biomarker comprises the therapeutic antibody. In some embodiments, the antigen comprises an antigen or biomarker of the therapeutic antibody. In some embodiments, the method further comprises determining a pharmacokinetic profile of the therapeutic antibody based on the quantified biomarker. In some embodiments, the method further comprises administering or adjusting a dose of the therapeutic antibody to the subject based on the quantified biomarker. In some embodiments, the subject is a human. In some embodiments, the method further comprises binding the captured biomarker with a detection antibody. In some embodiments, the method further comprises measuring the detection antibody. In some embodiments, the method further comprises comparing the detection antibody to a standard curve. In some embodiments, the standard curve is generated from biomarker capture particles bound to known amounts of the biomarker. In some embodiments, the method further comprises cleaning the detection antibody with the interference capture particles prior to the detection of the biomarker with the detection antibody. In some embodiments, the detection antibody comprises an anti-human antibody. In some embodiments, the detection antibody comprises an antibody against the antigen. In some embodiments, the detection antibody is conjugated to a detection reagent. In some embodiments, the detection reagent comprises an enzyme or label. In some embodiments, the label comprises a fluorescent tag. In some embodiments, the method further comprises multiplexing the biomarker capture particles with additional biomarker capture particles comprising a second antigen, and wherein the biomarker comprises an antibody that binds to the second antigen. In some embodiments, the quantifying the biomarker comprises multiplexing the detection antibody with a second detection antibody that recognizes the second antigen. In some embodiments, the method further comprises multiplexing the detection antibody with an additional detection antibody that recognizes a biomarker in the sample. In some embodiments, the method further comprises monitoring the biomarker over time in a first and second sample to determine an increase or decrease in an amount of biomarker in the second sample of the subject, relative to the quantified biomarker in the first sample. In some embodiments, the biomarker capture particle comprises a microparticle. In some embodiments, the biomarker capture particle comprises a bead. In some embodiments, the biomarker capture particle comprises a metal. In some embodiments, the biomarker capture particle is magnetic or ferromagnetic. In some embodiments, the biomarker capture particle comprises a plurality of biomarker capture particles comprising (i) a plurality of magnetic beads and (ii) a plurality of non-magnetic beads, wherein the plurality of magnetic beads can be different in size than the plurality of non-magnetic beads, and wherein one or more of the plurality of magnetic beads and one or more of the non-magnetic beads form a complex with the biomarker. In some embodiments, a concentration of the plurality of non-magnetic beads decreases upon removal of the complex. In some embodiments, the cleaning comprises cleaning the biomarker capture particle by removing the interference. In some embodiments, the cleaning comprises cleaning the detection antibody by removing the interference. In some embodiments, the assay sensitivity is at least 76%. In some embodiments, the assay sensitivity is at least 99%. In some embodiments, the assay sensitivity is 100%. In some embodiments, the assay specificity is at least 76%. In some embodiments, the assay specificity is at least 99%. In some embodiments, the assay specificity is 100%.

Disclosed herein, in some embodiments, are assay methods, comprising: (i) cleaning a substance by contacting the substance with an interference capture composition to remove an interference from the substance, wherein the interference capture composition comprises an interference capture moiety, and wherein the substance is selected from the group consisting of a sample from a subject, a biomarker capture particle, a detection antibody, and any combination thereof; and (ii) capturing a biomarker from the sample by contacting the sample with the biomarker capture particle, and optionally detecting an antigen with the detection antibody, wherein at least one of the sample, the biomarker capture particle, or the detection antibody comprises the cleaned substance. In some embodiments, the biomarker capture particle comprises a biomarker capture moiety. In some embodiments, the biomarker capture particle comprises an antibody and the biomarker comprises an antigen that binds to an epitope of the antibody. In some embodiments, the biomarker comprises an antibody and the biomarker capture particle comprises an antigen epitope that binds to the antibody. In some embodiments, the method comprises the detecting an antigen with the detection antibody, and wherein the contacting with an interference capture composition to remove an interference comprises removing an interference or a conjugate non-specific binding (NSB) from the detection antibody. In some embodiments, the interference capture composition comprises an interference capture particle, a biomarker capture particle, or a combination thereof. In some embodiments, the method comprises contacting the detection antibody with the sample to bind a detection biomarker in the sample with the detection antibody; and detecting or measuring the detection antibody bound to the detection biomarker. In some embodiments, the capture moiety is in a liquid reagent. In some embodiments, the liquid reagent further comprises a sample preservative reagent or stabilization agent. In some embodiments, the liquid reagent further comprises a sample conditioning reagent or agent. In some embodiments, the capture moiety is in a solid reagent. In some embodiments, wherein the method comprises adding the capture moiety into the sample. In some embodiments, capture moiety is present in a sample collection device prior to contacting with the sample. In some embodiments, the capture moiety is biotinylated. In some embodiments, the biomarker capture particle is coated with streptavidin. In some embodiments, the biomarker capture moiety comprises an antibody or an antibody binding fragment. In some embodiments, the biomarker comprises one or more biomarkers selected from the group consisting of an antigen, an antibody, a protein, a small molecule, a therapeutic, a hormone, a peptide, a signaling peptide, an exosome, a cell, or any combination thereof. In some embodiments, the biomarker comprises an antibody and the biomarker capture moiety comprises an antigen of the antibody. In some embodiments, the antibody is selected from the group consisting of autoantibodies, therapeutic antibodies, immunoglobulin classes, immunoglobulin subclasses, circulating antibodies, secretory antibodies, alpaca derived nanobodies, and animal derived antibodies. In some embodiments, the biomarker comprises an antigen. In some embodiments, the biomarker capture moiety comprises an antibody of the antigen. In some embodiments, the antigen comprises a therapeutic, a drug, a small molecule, a peptide, a protein, a vaccine, or an immunogen. In some embodiments, the antigen comprises a repeating epitope, wherein the antigen binds to more than one biomarker capture particle. In some embodiments, the antigen comprises at least a first type of epitope and a second type of epitope. In some embodiments, the biomarker capture particles are coated with at least a first type of antibody specific to the first type of epitope and a second type of antibody specific to the second type of epitope. In some embodiments, the biomarker comprises a disease state-specific biomarker. In some embodiments, the biomarker capture particles comprise agglutination particles, wherein the agglutination particles aggregate upon binding to the biomarker to form aggregated biomarker capture particles. In some embodiments, the aggregation is visually detectable or detectable via a detection technique. In some embodiments, the aggregated biomarker capture particles exhibit a color. In some embodiments, the method further comprises eluting the biomarker from the biomarker capture particles. In some embodiments, the method further comprises quantifying the biomarker. In some embodiments, quantifying the biomarker comprises determining a biomarker mass per sample volume. In some embodiments, the sample comprises a biofluid. In some embodiments, the biofluid comprises a blood, a plasma, a serum, a saliva, or a saline oral rinse. In some embodiments, the interference comprises a lipid, a triglyceride, a bilirubin, a hemolysis product, a cholesterol, a human anti-mouse antibody (HAMA), a rheumatoid interference (RF), a manufacture assay specific interference (MASI), a human anti-animal antibody (HAAA) interference, a free biotin interference, an anti-streptavidin interference, an anti-biotin interference, a human anti-polyethylene glycol (PEG) interference, an anti-albumin, a non-specific binding, an anti-polyvinylpyrrolidone (PVP), an anti-polymer, an anti-alkaline phosphatase (ALP), an anti-ruthenium, an anti-fluorescein, an anti-acridinium ester, an autoantibody, an anti-horse radish peroxidase (HRP), an anti-conjugation linker, an anti-amino acid tag, an anti-histidine tag, an anti-polyhistidine-tag, an over-the-counter (OTC) supplement, a herbal remedy and/or therapeutic, or any combination thereof. In some embodiments, the hemolysis product comprises a hemoglobin, a lactate dehydrogenase, a potassium, or a combination thereof. In some embodiments, the human anti-animal antibody (HAAA) interference comprises a mouse immunoglobulin, a goat immunoglobulin, a sheep immunoglobulin, a rabbit immunoglobulin, a bovine immunoglobulin, or a combination thereof. In some embodiments, the anti-acridinium ester comprises an ABEI, a luminol, an isoluminol, or any combination thereof. In some embodiments, the anti-conjugation linker comprises an LC, an LC-LC, a PEOn, a chromogen, or any combination thereof. In some embodiments, the method further comprises contacting the sample with a conjugate. In some embodiments, the method further comprises cleaning the conjugate with the interference capture particles or the biomarker capture particles prior to contacting the sample with the conjugate. In some embodiments, the sample of the subject has been subjected to one or more adjustments to chemical or physical properties of the sample to eliminate one or more interferences. In some embodiments, the chemical or physical properties are selected from temperature, color, pH, salinity, conductivity, density, viscosity, surface tension, and protein content. In some embodiments, the one or more adjustments comprise (i) adding surfactants, detergents, cell lysis agents, anti-protease agents, protein-based or polymeric-based blocking reagents, displacing agents, agents, or any combination thereof to the sample to release analytes from a matrix or to remove interfering elements; performing a dilution of the sample; or any combination thereof. In some embodiments, the interference capture moiety interacts with the interference. In some embodiments, subject is suspected of having a disease, or has been administered a vaccine against the disease. In some embodiments, the disease comprises an infection. In some embodiments, the infection comprises a viral infection, a bacterial infection, or a protozoan infection. In some embodiments, the disease comprises a tick-borne illness. In some embodiments, the disease comprises Lyme disease. In some embodiments, the disease comprises a Celiac disease. In some embodiments, the disease comprises a severe acute respiratory syndrome. In some embodiments, the disease comprises a coronavirus infection. In some embodiments, the coronavirus infection comprises Coronavirus disease 2019 (COVID-19). In some embodiments, the cleaning comprises cleaning the sample by removing the interference. In some embodiments, the vaccine is configured to generate a component of a pathogen or disease. In some embodiments, the method further comprises determining a vaccine efficacy based on the level of eluted biomarker. In some embodiments, the method further comprises readministering the vaccine to the subject based on the vaccine efficacy. In some embodiments, the eluted biomarker comprises antibodies against a pathogen or disease. In some embodiments, the method further comprises identifying a likelihood of the subject having the disease based on the eluted biomarker. In some embodiments, the method further comprises identifying a likelihood of the disease being active or acute. In some embodiments, the method further comprises administering the disease treatment to the subject when the subject is identified as having the active or acute disease. In some embodiments, the method further comprises not administering or discontinuing the treatment when the subject is identified as not having the active or acute disease. In some embodiments, administering the treatment comprises adjusting a dose amount or timing. In some embodiments, the biomarker is secreted. In some embodiments, the biomarker comprises IgA, IgG, or IgM, or a combination thereof. In some embodiments, the subject has been administered a therapeutic compound. In some embodiments, therapeutic compound comprises a therapeutic drug. In some embodiments, the therapeutic compound, or a fragment thereof is comprised in an antigen. In some embodiments, the captured biomarker comprises autoantibodies against the therapeutic compound. In some embodiments, the method further comprises determining a level of safety of the therapeutic compound based on the quantified biomarker. In some embodiments, the method further comprises administering or adjusting a dose of the therapeutic compound to the subject based on the quantified biomarker. In some embodiments, the therapeutic drug comprises a therapeutic antibody. In some embodiments, the therapeutic antibody comprises an antibody binding fragment. In some embodiments, the captured biomarker comprises the therapeutic antibody. In some embodiments, the antigen comprises an antigen or biomarker of the therapeutic antibody. In some embodiments, the method further comprises determining a pharmacokinetic profile of the therapeutic antibody based on the quantified biomarker. In some embodiments, the method further comprises administering or adjusting a dose of the therapeutic antibody to the subject based on the quantified biomarker. In some embodiments, the subject is a human. In some embodiments, the method further comprises binding the captured biomarker with a detection antibody. In some embodiments, the method further comprises measuring the detection antibody. In some embodiments, the method further comprises comparing the detection antibody to a standard curve. In some embodiments, the standard curve is generated from biomarker capture particles bound to known amounts of the biomarker. In some embodiments, the method further comprises cleaning the detection antibody with the interference capture particles prior to the detection of the biomarker with the detection antibody. In some embodiments, the detection antibody comprises an anti-human antibody. In some embodiments, the detection antibody comprises an antibody against the antigen. In some embodiments, the detection antibody is conjugated to a detection reagent. In some embodiments, the detection reagent comprises an enzyme or label. In some embodiments, the label comprises a fluorescent tag. In some embodiments, the method further comprises multiplexing the biomarker capture particles with additional biomarker capture particles comprising a second antigen, and wherein the biomarker comprises an antibody that binds to the second antigen. In some embodiments, the quantifying the biomarker comprises multiplexing the detection antibody with a second detection antibody that recognizes the second antigen. In some embodiments, the method further comprises multiplexing the detection antibody with an additional detection antibody that recognizes a biomarker in the sample. In some embodiments, the method further comprises monitoring the biomarker over time in a first and second sample to determine an increase or decrease in an amount of biomarker in the second sample of the subject, relative to the quantified biomarker in the first sample. In some embodiments, the biomarker capture particle comprises a microparticle. In some embodiments, the biomarker capture particle comprises a bead. In some embodiments, the biomarker capture particle comprises a metal. In some embodiments, the biomarker capture particle is magnetic or ferromagnetic. In some embodiments, the biomarker capture particle comprises a plurality of biomarker capture particles comprising (i) a plurality of magnetic beads and (ii) a plurality of non-magnetic beads, wherein the plurality of magnetic beads can be different in size than the plurality of non-magnetic beads, and wherein one or more of the plurality of magnetic beads and one or more of the non-magnetic beads form a complex with the biomarker. In some embodiments, a concentration of the plurality of non-magnetic beads decreases upon removal of the complex. In some embodiments, the cleaning comprises cleaning the biomarker capture particle by removing the interference. In some embodiments, the cleaning comprises cleaning the detection antibody by removing the interference. In some embodiments, the assay sensitivity is at least 76%. In some embodiments, the assay sensitivity is at least 99%. In some embodiments, the assay sensitivity is 100%. In some embodiments, the assay specificity is at least 76%. In some embodiments, the assay specificity is at least 99%. In some embodiments, the assay specificity is 100%.

Disclosed herein, in some embodiments, are assay methods to determine a medicament efficacy, the method comprising: (i) providing a sample of a subject wherein the subject has been administered a medicament; (ii) capturing a biomarker from the sample by contacting the sample with a biomarker capture particle comprising a biomarker capture moiety; (iii) quantifying the eluted biomarker; and (iv) determining the medicament efficacy. In some embodiments, the quantifying the eluted biomarker comprises detecting an antigen with a detection antibody. In some embodiments, the method further comprises cleaning a substance by contacting the substance with an interference capture composition to remove an interference from the substance, wherein the interference capture composition comprises an interference capture moiety, and wherein the substance is selected from the group consisting of the sample, the biomarker capture particle, the detection antibody, and any combination thereof. In some embodiments, the medicament comprises a vaccine, a therapeutic, or a combination thereof. In some embodiments, the sample, the biomarker capture particle, or a combination thereof are subjected to a cleaning prior to capturing the biomarker from the sample, wherein the cleaning comprises contacting with an interference capture composition to remove an interference. In some embodiments, the biomarker capture particle comprises a biomarker capture moiety. In some embodiments, the biomarker capture particle comprises an antibody and the biomarker comprises an antigen that binds to an epitope of the antibody. In some embodiments, the biomarker comprises an antibody and the biomarker capture particle comprises an antigen epitope that binds to the antibody. In some embodiments, the method comprises the detecting an antigen with the detection antibody, and wherein the contacting with an interference capture composition to remove an interference comprises removing an interference or a conjugate non-specific binding (NSB) from the detection antibody. In some embodiments, the interference capture composition comprises an interference capture particle, a biomarker capture particle, or a combination thereof. In some embodiments, the method comprises contacting the detection antibody with the sample to bind a detection biomarker in the sample with the detection antibody; and detecting or measuring the detection antibody bound to the detection biomarker. In some embodiments, the capture moiety is in a liquid reagent. In some embodiments, the liquid reagent further comprises a sample preservative reagent or stabilization agent. In some embodiments, the liquid reagent further comprises a sample conditioning reagent or agent. In some embodiments, the capture moiety is in a solid reagent. In some embodiments, wherein the method comprises adding the capture moiety into the sample. In some embodiments, capture moiety is present in a sample collection device prior to contacting with the sample. In some embodiments, the capture moiety is biotinylated. In some embodiments, the biomarker capture particle is coated with streptavidin. In some embodiments, the biomarker capture moiety comprises an antibody or an antibody binding fragment. In some embodiments, the biomarker comprises one or more biomarkers selected from the group consisting of an antigen, an antibody, a protein, a small molecule, a therapeutic, a hormone, a peptide, a signaling peptide, an exosome, a cell, or any combination thereof. In some embodiments, the biomarker comprises an antibody and the biomarker capture moiety comprises an antigen of the antibody. In some embodiments, the antibody is selected from the group consisting of autoantibodies, therapeutic antibodies, immunoglobulin classes, immunoglobulin subclasses, circulating antibodies, secretory antibodies, alpaca derived nanobodies, and animal derived antibodies. In some embodiments, the biomarker comprises an antigen. In some embodiments, the biomarker capture moiety comprises an antibody of the antigen. In some embodiments, the antigen comprises a therapeutic, a drug, a small molecule, a peptide, a protein, a vaccine, or an immunogen. In some embodiments, the antigen comprises a repeating epitope, wherein the antigen binds to more than one biomarker capture particle. In some embodiments, the antigen comprises at least a first type of epitope and a second type of epitope. In some embodiments, the biomarker capture particles are coated with at least a first type of antibody specific to the first type of epitope and a second type of antibody specific to the second type of epitope. In some embodiments, the biomarker comprises a disease state-specific biomarker. In some embodiments, the biomarker capture particles comprise agglutination particles, wherein the agglutination particles aggregate upon binding to the biomarker to form aggregated biomarker capture particles. In some embodiments, the aggregation is visually detectable or detectable via a detection technique. In some embodiments, the aggregated biomarker capture particles exhibit a color. In some embodiments, the method further comprises eluting the biomarker from the biomarker capture particles. In some embodiments, the method further comprises quantifying the biomarker. In some embodiments, quantifying the biomarker comprises determining a biomarker mass per sample volume. In some embodiments, the sample comprises a biofluid. In some embodiments, the biofluid comprises a blood, a plasma, a serum, a saliva, or a saline oral rinse. In some embodiments, the interference comprises a lipid, a triglyceride, a bilirubin, a hemolysis product, a cholesterol, a human anti-mouse antibody (HAMA), a rheumatoid interference (RF), a manufacture assay specific interference (MASI), a human anti-animal antibody (HAAA) interference, a free biotin interference, an anti-streptavidin interference, an anti-biotin interference, a human anti-polyethylene glycol (PEG) interference, an anti-albumin, a non-specific binding, an anti-polyvinylpyrrolidone (PVP), an anti-polymer, an anti-alkaline phosphatase (ALP), an anti-ruthenium, an anti-fluorescein, an anti-acridinium ester, an autoantibody, an anti-horse radish peroxidase (HRP), an anti-conjugation linker, an anti-amino acid tag, an anti-histidine tag, an anti-polyhistidine-tag, an over-the-counter (OTC) supplement, a herbal remedy and/or therapeutic, or any combination thereof. In some embodiments, the hemolysis product comprises a hemoglobin, a lactate dehydrogenase, a potassium, or a combination thereof. In some embodiments, the human anti-animal antibody (HAAA) interference comprises a mouse immunoglobulin, a goat immunoglobulin, a sheep immunoglobulin, a rabbit immunoglobulin, a bovine immunoglobulin, or a combination thereof. In some embodiments, the anti-acridinium ester comprises an ABEI, a luminol, an isoluminol, or any combination thereof. In some embodiments, the anti-conjugation linker comprises an LC, an LC-LC, a PEOn, a chromogen, or any combination thereof. In some embodiments, the method further comprises contacting the sample with a conjugate. In some embodiments, the method further comprises cleaning the conjugate with the interference capture particles or the biomarker capture particles prior to contacting the sample with the conjugate. In some embodiments, the sample of the subject has been subjected to one or more adjustments to chemical or physical properties of the sample to eliminate one or more interferences. In some embodiments, the chemical or physical properties are selected from temperature, color, pH, salinity, conductivity, density, viscosity, surface tension, and protein content. In some embodiments, the one or more adjustments comprise (i) adding surfactants, detergents, cell lysis agents, anti-protease agents, protein-based or polymeric-based blocking reagents, displacing agents, agents, or any combination thereof to the sample to release analytes from a matrix or to remove interfering elements; performing a dilution of the sample; or any combination thereof. In some embodiments, the interference capture moiety interacts with the interference. In some embodiments, subject is suspected of having a disease, or has been administered a vaccine against the disease. In some embodiments, the disease comprises an infection. In some embodiments, the infection comprises a viral infection, a bacterial infection, or a protozoan infection. In some embodiments, the disease comprises a tick-borne illness. In some embodiments, the disease comprises Lyme disease. In some embodiments, the disease comprises a Celiac disease. In some embodiments, the disease comprises a severe acute respiratory syndrome. In some embodiments, the disease comprises a coronavirus infection. In some embodiments, the coronavirus infection comprises Coronavirus disease 2019 (COVID-19). In some embodiments, the cleaning comprises cleaning the sample by removing the interference. In some embodiments, the vaccine is configured to generate a component of a pathogen or disease. In some embodiments, the method further comprises determining a vaccine efficacy based on the level of eluted biomarker. In some embodiments, the method further comprises readministering the vaccine to the subject based on the vaccine efficacy. In some embodiments, the eluted biomarker comprises antibodies against a pathogen or disease. In some embodiments, the method further comprises identifying a likelihood of the subject having the disease based on the eluted biomarker. In some embodiments, the method further comprises identifying a likelihood of the disease being active or acute. In some embodiments, the method further comprises administering the disease treatment to the subject when the subject is identified as having the active or acute disease. In some embodiments, the method further comprises not administering or discontinuing the treatment when the subject is identified as not having the active or acute disease. In some embodiments, administering the treatment comprises adjusting a dose amount or timing. In some embodiments, the biomarker is secreted. In some embodiments, the biomarker comprises IgA, IgG, or IgM, or a combination thereof. In some embodiments, the subject has been administered a therapeutic compound. In some embodiments, therapeutic compound comprises a therapeutic drug. In some embodiments, the therapeutic compound, or a fragment thereof is comprised in an antigen. In some embodiments, the captured biomarker comprises autoantibodies against the therapeutic compound. In some embodiments, the method further comprises determining a level of safety of the therapeutic compound based on the quantified biomarker. In some embodiments, the method further comprises administering or adjusting a dose of the therapeutic compound to the subject based on the quantified biomarker. In some embodiments, the therapeutic drug comprises a therapeutic antibody. In some embodiments, the therapeutic antibody comprises an antibody binding fragment. In some embodiments, the captured biomarker comprises the therapeutic antibody. In some embodiments, the antigen comprises an antigen or biomarker of the therapeutic antibody. In some embodiments, the method further comprises determining a pharmacokinetic profile of the therapeutic antibody based on the quantified biomarker. In some embodiments, the method further comprises administering or adjusting a dose of the therapeutic antibody to the subject based on the quantified biomarker. In some embodiments, the subject is a human. In some embodiments, the method further comprises binding the captured biomarker with a detection antibody. In some embodiments, the method further comprises measuring the detection antibody. In some embodiments, the method further comprises comparing the detection antibody to a standard curve. In some embodiments, the standard curve is generated from biomarker capture particles bound to known amounts of the biomarker. In some embodiments, the method further comprises cleaning the detection antibody with the interference capture particles prior to the detection of the biomarker with the detection antibody. In some embodiments, the detection antibody comprises an anti-human antibody. In some embodiments, the detection antibody comprises an antibody against the antigen. In some embodiments, the detection antibody is conjugated to a detection reagent. In some embodiments, the detection reagent comprises an enzyme or label. In some embodiments, the label comprises a fluorescent tag. In some embodiments, the method further comprises multiplexing the biomarker capture particles with additional biomarker capture particles comprising a second antigen, and wherein the biomarker comprises an antibody that binds to the second antigen. In some embodiments, the quantifying the biomarker comprises multiplexing the detection antibody with a second detection antibody that recognizes the second antigen. In some embodiments, the method further comprises multiplexing the detection antibody with an additional detection antibody that recognizes a biomarker in the sample. In some embodiments, the method further comprises monitoring the biomarker over time in a first and second sample to determine an increase or decrease in an amount of biomarker in the second sample of the subject, relative to the quantified biomarker in the first sample. In some embodiments, the biomarker capture particle comprises a microparticle. In some embodiments, the biomarker capture particle comprises a bead. In some embodiments, the biomarker capture particle comprises a metal. In some embodiments, the biomarker capture particle is magnetic or ferromagnetic. In some embodiments, the biomarker capture particle comprises a plurality of biomarker capture particles comprising (i) a plurality of magnetic beads and (ii) a plurality of non-magnetic beads, wherein the plurality of magnetic beads can be different in size than the plurality of non-magnetic beads, and wherein one or more of the plurality of magnetic beads and one or more of the non-magnetic beads form a complex with the biomarker. In some embodiments, a concentration of the plurality of non-magnetic beads decreases upon removal of the complex. In some embodiments, the cleaning comprises cleaning the biomarker capture particle by removing the interference. In some embodiments, the cleaning comprises cleaning the detection antibody by removing the interference. In some embodiments, the assay sensitivity is at least 76%. In some embodiments, the assay sensitivity is at least 99%. In some embodiments, the assay sensitivity is 100%. In some embodiments, the assay specificity is at least 76%. In some embodiments, the assay specificity is at least 99%. In some embodiments, the assay specificity is 100%.

Disclosed herein, in some embodiments, are kits, comprising: (a) an interference capture particle comprising an interference capture moiety; and (b) a biomarker capture particle comprising a biomarker capture moiety. In some embodiments, the kit further comprises a detection antibody. In some embodiments, the interference capture moiety comprises a human immunoglobulin, an antibody, an animal antibody, an antibody fragment, a polymerized antibody, a mouse antibody fragment, an aptamer, a protein, an enzyme, a small molecule, a streptavidin, an avidin, a neutravidin, an MIP, a polymer, a conjugation linker, or any combination thereof. In some embodiments, the biomarker capture moiety comprises an antibody, an antibody fragment, a polypeptide binder, a monobody, a non-immunoglobulin binder, a DARPin, an affibody, an anticalin, a molecular imprinted polymer (MIP), an aptamer, a chimeric antibody, a therapeutic antibody, an antigen, a protein, a small molecule, a therapeutic, a hormone, a peptide, a signaling peptide, an exosome, a cell, a disease state-specific antigen, an antibody, a biomarker, or any combination thereof. In some embodiments, the kit is for use in a method disclosed herein. In some embodiments, the kit further comprises a sample receptacle. In some embodiments, the kit further comprises instructions for use.

### BRIEF DESCRIPTION OF THE DRAWINGS

FIG. 1 shows an example sample preparation aspects.

FIGS. 2A-2E show example detection/measurement processes.

FIGS. 3A-3B show protocols for Lyme infection detection.

FIGS. 4A-4J show images of sample wells.

FIG. 5 shows exemplary agglutination of biomarker capture particles.

FIG. 6 shows graphical data from a comparison of capture beads signal with and without BSA beads.

FIG. 7 is a graph that shows cleaning effectiveness of capture beads.

FIG. 8 and FIG. 9 are images showing exemplary custom Catalyst 96 slotted magnets.

FIG. 10 is an image showing an example pipetting technique.

FIG. 11 shows an example calibration curve for IgG.

FIG. 12A-B shows data from a COVID-19 Antibody Test (IU/mL) vs. GenScript® cPass™ Neutralization Antibody Detection Kit (IU/mL). FIG. 12A shows the NIBSC IgG IU/mL over the cPass SemiQuant IU/mL with a fit line. FIG. 12B shows a summary of the data for different parameters including 95% CI, SE, t, and p-value.

FIGS. 13A-13C show exemplary calibration curves for IgG, IgM, and IgA.

FIGS. 14A-14C show longitudinal test study data for an antibody.

FIG. 15 shows interference signal with cleaning by clean beads.

FIG. 16 shows the gel picture of SDS PAGE.

FIGS. 17A-17C show the dose response curves for AB40, AB42, and pTau181 respectively.

FIGS. 18A-18C show the levels of Amyloid beta 1-40 (AB40 or AB1-40, FIG. 18A), phosphorylated Tau (pTau181, FIG. 18B), and AB40+pTau181 (FIG. 18C) were lower in Moderate Alzheimer's patients' SOR samples (N=2) as compared to Healthy Controls' SOR samples (N=6).

### DETAILED DESCRIPTION

Provided herein are methods and systems for multiplex detection and/or measurement of biomarkers of a sample. The methods and systems can, for example, be used for rapid disease detection and/or monitoring, vaccine efficacy and immune response monitoring, therapeutic drug monitoring, and/or therapeutic safety and efficacy monitoring.

### Definitions

As used in the specification and claims, the following terms have the meanings indicated:

“About” with reference to a number refers to that number plus or minus 15% of that number. The term “about” a range refers to that range minus 15% of its lowest value and plus 15% of its greatest value.

“Affinity assays” generally refer to assays that determine the presence or absence of an analyte in a sample, and/or quantitate the amount of analyte in a sample, directly or indirectly, based on a specific or relatively specific interaction between the analyte and a molecule that preferentially binds the analyte. Affinity assays include assays that rely in at least some respect on a specific or relatively specific binding affinity of one entity for another. Affinity assays include, but are not limited to, assays that rely on a binding interaction between a receptor and a ligand, an enzyme and its substrate, a polynucleotide and its complement or substantial complement, a small molecule and a binding protein that binds the small molecule with specificity, etc. Immunoassays include assays that rely on the interaction between, for example, an antigen and an antibody that recognizes the antigen. Immunoassays also include, for example, assays that employ an antibody or fragment thereof to bind an antigen of interest in a sample. Affinity assays also include, for example, competitive assays and sandwich assays. Such assays include those which rely on an interaction of a surface-bound antigen to detect an antibody of interest in a sample, and those that interaction of a surface-bound antibody or fragment thereof to detect an antigen of interest in a sample. As used herein, antigens are not limited to polypeptides or proteins, but can also include small molecules (such as, for example, haptens) and antibodies (for example, antibodies can be used as antigens to generate other antibodies that recognize them). In general, antigen as used herein includes any analyte of interest in a sample immunoassayed with an antibody or fragment thereof using the compositions or methods of the disclosure.

“Analyte,” “target,” or “biomarker,” as used interchangeably herein, generally refers to a molecule or complex to be detected and/or quantitated. Non-limiting examples of biomarkers comprise, but are not limited to, polypeptides (e.g., proteins, phosphorylated or other post-translationally modified forms of a protein, antibodies, etc.), antigens, small molecules, and nucleic acids (e.g., DNAs, RNAs, mRNAs, ribosomal RNAs, microRNAs, transcription factor binding sites, genomic DNAs or RNAs, etc.). A biomarker can comprise antibodies, autoantibodies, therapeutic antibodies, immunoglobulin classes such as IgG, IgM, IgA, IgE, immunoglobulin subclasses such as IgA1, IgA2, IgG1, IgG2, IgG3, IgG4, circulating antibodies, secretory antibodies, animal derived antibodies, antigens, proteins, small molecules, therapeutics, hormones, peptides, signaling peptides, exosomes, cells, or any combination thereof. In some embodiments, a biomarker can comprise a disease state biomarker. In some embodiments a disease state can comprise a cardiac disease, Alzheimer's disease, Parkinson's disease, dementia, a traumatic brain injury (TBI), a neurodegenerative disease, a cancer, a renal disease, an autoimmune disease, an infectious disease, a sexually transmitted disease (STD) or infection (STI), infertility, a women's health condition, anemia, a bone disease, an endocrine disease, inflammation, a metabolic disease, a therapeutic drug monitoring, a sleep apnea, a hepatic disease, a respiratory disease, or any combination thereof. In some embodiments, animal derived antibodies can be derived from an alpaca, a mouse, a pig, a monkey, a rat, a sheep, a goat, a cow, or any combination thereof.

“Biomarker capture particles,” “target capture particles,” or “capture particles,” as used interchangeably herein, generally refers to particles that may interact with a biomarker in a sample. In some embodiments, the biomarker capture particles can comprise capture moieties that may interact with a biomarker in a sample. In some embodiments, the biomarker capture particles can comprise a coating, for example, a streptavidin coating, that may interact with a capture moiety added to or present in a sample. In some embodiments, the biomarker capture particles can comprise capture beads.

“Blocker” generally refers to a protein, polymer, surfactant, detergent, or combinations thereof. In some embodiments, the binding of a capture moiety on a particle described herein (e.g., nanoparticle, microparticle) is blocked with a blocker such as a protein, polymer, surfactant, detergent, or combinations thereof. The blocker is selected from the group consisting of a protein such as albumin, bovine serum albumin, human serum albumin, ovalbumin, gelatin, casein, acid hydrolyzed casein, gamma globulin, purified IgG, animal serum, polyclonal antibody, and monoclonal antibody, a polymer such as polyvinyl alcohol (PVA) and polyvinylpyrrolidone (PVP), a combination of a protein and polymer, a peptide, a PEGylation reagent such as (PEO)n-NHS or (PEO)n-maleimide, a triblock copolymer such as Pluronic F108, F127, and F68, a non-ionic detergent such as Triton X-100, polysorbate 20 (Tween-20), and Tween 80 (non-ionic), a zwitterionic detergent such as CHAPS, a ionic detergent such as sodium dodecyl sulfate (SDS), deoxycholate, cholate, and sarkosyl, a surfactant, a sugar such as sucrose, and a commercial blocker such as Heterophilic Blocking Reagent (Scantibodies), MAK33 (Roche® Diagnostics), Immunoglobulin Inhibiting Reagent (IIR) (Bioreclamation), Heteroblock (Omega Biologicals), Blockmaster (JSR), TRU Block (Meridian Life Sciences), and StabilCoat® & StabilGuard® (Surmodics). In some embodiments, the blocker is bound to a particle described herein (e.g., covalently bound, noncovalently bound). In some embodiments, the blocker is not bound (e.g., covalently bound, non-covalently bound) to a particle described herein.

“Capture molecule,” “biomarker capture moiety,” or “target binding element,” as used interchangeably herein, generally refers to a molecule that is configured to capture a particular biomarker of interest (whether through direct or indirect binding to the biomarker) and that is bound (e.g., covalently or noncovalently, directly or through a linker, e.g., streptavidin-biotin or the like) to a solid support such as a particle (e.g., a microsphere, microbead, or the like). The capture moiety can bind directly to the biomarker and can be specific for that biomarker. The capture moiety can bind to one or more molecules that bind in turn to the biomarker to specifically capture it. Non-limiting examples of capture moiety comprise, but are not limited to, interference, polypeptides (e.g., antibodies, SH2 and other polypeptide binding domains, short synthetic peptides, and antigens), polynucleotides (e.g., polynucleotide capture probes, transcription factor binding sites, aptamers), antigens, biomarkers, polysaccharides, lipids, small molecules, molecular imprinted polymers (MIP), chimeric antibodies, therapeutic antibodies, recombinant antibodies, monoclonal antibodies, polyclonal antibodies, and antibody fragments thereof such as Fab, F(ab′)2, Fc, scFv, alpaca derived nanobodies, phage display VHH constructs, and engineered variants such as diabodies, triabodies, minibodies and single-domain antibodies. The capture moiety particles can be combined, mixed, or pooled to multiplex capture biomarkers using a plurality or pool of different biomarker capture particles (e.g., capture beads), each capture bead coated with a different target binding element or capture moiety, or beads coated with a mixture of different target binding elements or capture moieties, where all particles are magnetic, or where some particles are magnetic and others are non-magnetic.

“Specificity” as used herein refers to an ability to accurately not detect (not measure) the level, concentration, or presence of a biomarker for a true negative result that otherwise may be reported as a false positive. The specificity of a clinical test refers to the ability of the test to correctly identify those patients without the disease. Therefore, a test with 100% specificity correctly identifies all patients without the disease. In a test this typically means that a negative result has no signal above background noise. For example, a pre-analytical cleaning of the sample with interference capture particles can mitigate sample interferences that otherwise can cause bridging (such as HAMA or RF) or heterophilic, autoantibody, or NSB of human immunoglobulins (e.g., hIgG, hIgA, hIgM, hIgE classes, or hIgA1, hIgA2, hIgG1, hIgG2, hIgG3, hIgH4 subclasses) to the solid phase biomarker capture moiety causing a false high signal or a false positive result (HAMA- or RF-like bridging of the conjugate to the solid phase causes false high signal in sandwich immunoassay, and heterophilic, autoantibody, or NSB of immunoglobulins cause false high signal in serology, antibody, or autoantibody assays). If such interference is cleaned, mitigated, or reduced within the assay blocking threshold or assay design, then the biomarker will not be detected by the conjugate for a true negative result. Disclosed herein in some embodiments are methods that can provide a specificity of at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

“Sensitivity” as used herein refers to the ability to accurately detect (measure) the level, concentration, or presence of a biomarker for a true positive result that otherwise may be missed and reported as a false negative. The sensitivity of a clinical test refers to the ability of the test to correctly identify those patients with the disease. A test with 100% sensitivity correctly identifies all patients with the disease. In a test this typically means that a positive result has signal above background noise. For example, the pre-analytical cleaning of a sample with interference capture particles mitigates sample interferences that otherwise can sterically block the solid phase biomarker capture moiety, or compete for binding to the solid phase capture reagent (e.g., free biotin interference competing with the biotin labelled antibody or antigen for binding to streptavidin solid phase or conjugate) causing a false low signal or a false negative result. If such interference is cleaned, mitigated, or reduced within an assay blocking threshold or assay design, then the biomarker will be accurately captured by the solid phase and subsequently detected by the conjugate for a true positive result. Disclosed herein are methods that can improve sensitivity or likelihood of biomarker detection by the capture and purification of biomarker from the sample with antibody or antigen coated biomarker capture particles into a matrix free buffer for subsequent biomarker detection, and in the case of low abundance biomarkers or biomarkers at a concentration below the detection limit of a current assay (<limit of detection (LoD) or <limit of quantitation (LoQ). In some embodiments, the methods disclosed herein can capture, purify, and enrich or concentrate the biomarkers into a smaller volume such that their level is increased 6-fold or greater and they can subsequently be detected above the LoD or LoQ of the assay. In other words, cleaning the sample of interference allows for the subsequent biomarker capture particles to accurately detect and capture the targeted biomarker in the cleaned sample matrix for subsequent purification and enrich for high sensitivity detection. Disclosed herein in some embodiments are methods that can provide a sensitivity of at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

“Accuracy” as used herein can refer to either specificity, sensitivity, or a combination thereof.

“Coupled with,” “bound with,” or grammatical equivalents, generally refer to a covalent (e.g., through one or more carbon-carbon bonds, carbon-nitrogen bonds, carbon-oxygen bonds, etc., either directly or indirectly) or non-covalent binding (either indirectly or directly) or interaction between two moieties or entities. The term coupled with is not intended to connote an orientation or direction of the coupling. Entities that are known to specifically interact with one another can be covalently coupled. One non-limiting example of entities that are known to specifically interact and that can be covalently coupled is an antigen and its specific antibody, which can be made to covalently attach through, for example, coupling chemistry. Non-covalent binding can comprise affinity, ionic, van der Waals (e.g., dipole/dipole or London forces), hydrogen bonding (e.g., between polynucleotide duplexes), and hydrophobic interactions. Where coupling is non-covalent, the association between the entities is preferably specific. Non-limiting examples of specific non-covalent associations include the binding interaction between biotin and a biotin-binding protein such as avidin, SA, neutravidin, a fragment of SA, a fragment of avidin, a fragment of neutravidin, or mixtures thereof; the binding of a biotinylated Fab, a biotinylated immunoglobulin or fragment thereof, a biotinylated small molecule (such as, for example, a hormone or a ligand of a receptor), a biotinylated polynucleotide, a biotinylated macromolecule (e.g., a protein or a natural or synthetic polymer) to a biotin-binding protein such as avidin, SA, neutravidin, a fragment of SA, a fragment of avidin, a fragment of neutravidin, or mixtures thereof; the binding of a substrate to its enzyme; the binding of a glycoprotein to a lectin specific for the glycoprotein; the binding of a ligand to a receptor specific for the ligand; the binding of an antibody to an antigen against which the antibody is raised; and duplex formation between a polynucleotide and a complementary or substantially complementary polynucleotide, etc.

“Determining,” “measuring,” “evaluating,” “assessing,” “assaying,” or “analyzing” as used interchangeably herein, generally refers to forms of measurement. The terms include determining if an element is present or not (for example, detection). These terms can include quantitative, semi-quantitative, qualitative or quantitative and qualitative determinations. Assessing can be relative or absolute. “Detecting the presence of” can include determining the amount of something present in addition to determining whether it is present or absent depending on the context.

“Diagnostic” or “test” generally refers to any antibody-based diagnostic test, non-antibody based diagnostic test, a sample pre-treatment method or device for subsequent analysis by chromatographic, spectrophotometric, and mass spectrometry methods (i.e. HPLC, MS, LCMS, LC-MS/MS, MALDI-TOF) such as immunoextraction (IE) and solid phase extraction (SPE), radioimmunoassay (RIA), enzyme-linked immunoassay (ELISA), chemiluminescence immunoassay (CLIA), fluorescence immunoassay (FIA), chemistry such as turbidimetric or particle-enhanced turbidimetric immunoassay (PETIA), flow cytometry, molecular diagnostics (PCR synthesis, RT-PCR, PCR-ELISA, Fluorescence in situ hybridization, next-generation sequencing (NGS)), lateral flow (LF), microarray, multiplex test, point-of-care (PoC), direct to consumer (DTC), CLIA and CLIA waived tests and devices, Research Use Only (RUO) test, In Vitro Diagnostics (IVD) test, Laboratory Developed Test (LDT), companion diagnostic, and any test for diagnosis, prognosis, screening, risk assessment, risk stratification, and monitoring such as therapeutic drug monitoring.

“Interference” generally refers to a substance present in, or a condition of, a sample, e.g., a biological sample, that can alter the correct value of the result by interfering with a capture moiety or a particle, or that can increase or decrease assay signal by bridging, steric hindrance, or autoantibody mechanisms. Erroneous results can occur unexpectedly with any specimen without the practical means to identify upfront such specimens likely to cause problems. The consequence of such interference is that erroneous results can impact patient care, and can lead to unnecessary invasive, diagnostic or therapeutic procedures, or failure to treat a patient with a false negative test result. Examples of interference include heterophile or heterophile-like interferences such autoantibodies, rheumatoid factor (RF), human anti-mouse antibodies (HAMA), human anti-animal antibodies (HAAA) such as goat, rabbit, sheep, bovine, mouse, horse, pig, and donkey polyclonal and/or monoclonal antibodies, and manufacture assay-specific interference used in the test design or assay formulation such as the chemiluminescent substrate (isoluminol, luminol, ABEI, ruthenium, acridenium ester), fluorescent label (fluorescein or other fluorophores and dyes), anti-alkaline phosphatase (ALP), anti-horse radish peroxidae (HRP)), anti-histidine, capture moieties (streptavidin, neutravidin, avidin, polyA, polyDT, aptamers, antibodies, Fab, F(ab′)2, antibody fragments, recombinant proteins, enzymes, proteins, biomolecules, polymers) and their binding partners (i.e. biotin, fluorescein, PolyDT, PolyA, antigen, etc.), conjugation linkers (LC, LC-LC, PEO, PEON), and solid phase blocking proteins (bovine serum albumin, human serum albumin, ovalbumin, gelatin, purified poly- and monoclonal IgG such as mouse, goat, sheep and rabbit, polyvinyl alcohol or PAA, polyvinylpyrrolidone or PVP, Tween-20, Tween-80, Triton X-100, triblock copolymers such as Pluronic and Tetronic, and other commercially available blockers, blocking proteins and polymer-based blocking reagents such as those from Surmodics and Scantibodies), anti-amino acid tags, recombinant tags or affinity tags on proteins, peptides, antigens, antibodies such as poly(His) tag (6-his tag, 8-his tag), Strep-tag, chitin binding protein (CBP), maltose binding protein (MBP), glutathione-S-transferase (GST), FLAG-tag, epitope tags including ALFA-tag, V5-tag, Myc-tag, HA-tag, Spot-tag, T7-tag and NE-tag, green fluorescent protein (GFP), lipids, triglycerides, bilirubin, hemolysis products (e.g., hemoglobin, enzyme, potassium), cholesterol, free biotin interference, anti-biotin interference, human anti-polyethylene glycol (PEG) interference, anti-albumin, non-specific binding (NSB), anti-polymer antibodies, anti-polyvinylpyrrolidone (PVP) antibodies, anti-polyvinyl alcohol (PVA), autoantibodies, anti-conjugation linkers such as LC, LC-LC, and PEOn, over-the-counter (OTC) supplements, herbal remedies, or therapeutics that can cause problems that negatively impact test performance or accuracy, or cause erroneous results, of non-antibody based diagnostic tests such as molecular diagnostics or mass spectrometry (i.e. HPLC, MS, LCMS, LC-MS/MS), or antibody based tests such as radioimmunoassay (RIA), enzyme-linked immunoassay (ELISA), chemiluminescence immunoassay (CLIA), fluorescence immunoassay (FIA), chemistry such as turbidimetric or particle-enhanced turbidimetric immunoassay (PETIA), Antibody-Oligo Conjugates in Immuno-PCR, lateral flow, flow cytometry, point-of-care (PoC), CLIA and CLIA waived tests and devices.

“Interference capture composition(s)”, generally refers to compositions comprising one or more interference capture moieties that will interact with a sample interference such that the interference, when exposed to the interference capture composition, will interact with and bind to the interference capture composition. In some embodiments, an interference capture composition can comprise “Interference capture particle(s),” or “Clean Bead(s),” as used interchangeably herein. In some embodiments, an interference capture particle can comprise a particle as described herein comprising an interference capture moiety. In some embodiments, when the interference capture particles are isolated or removed from the sample via magnetic binding, magnetic centrifugation, centrifugation, or filtration, the essentially particle-free sample will have a significantly lower level, concentration, threshold, or titer of the interference such that it will no longer interfere with a test or the measurement or characterization of the sample. Examples of interference capture moieties can comprise human immunoglobulins (IgA, IgG, IgM, IgE) which will target autoantibody interference, animal antibodies (mouse, goat, sheep, rabbit, cow (bovine), lama, alpaca) which target heterophilic antibodies such as HAMA, RF, and HAAA, polymerized antibodies or mouse antibody fragments (Fc, Fab, F(ab′)2) which target HAMA and RF interference, proteins, enzymes, or small molecules (ALP, HRP, fluorescein/fluorophores, luminol, isoluminol, acridinium ester, ABEI, ruthenium, luciferin) that target anti-signal generating interference, streptavidin, avidin, or neutravidin which targets biotin interference or biotin metabolite interference (bisnorbiotin, biotin sulfoxide), antibodies, aptamers, antibody fragments, MIPs, or polymers that target specific interferences via epitope binding such as bilirubin, albumin, lipids, triglycerides, cholesterol, icterus (bile pigments), hemoglobin, herbal (for example, hemolysis products, lipemia, icterus, tube additives, administration of radioactive or fluorescent compounds, drugs, herbal medicines, and nutritional supplements are all exogenous interferences that can adversely affect immunoassays), conjugation linkers such as LC, LC-LC, PEOn, PEG, polyhistidines (His-tags), the beads themselves as some sample interferences interact with the bead polymer or co-polymers, iron oxide or exposed iron crystals on the surface, PVP used as a wetting agent for the polymerization, functional groups, or any NSB interactions with the bead. A sample can be pre-analytically cleaned of sample-specific interferences by adding interference capture particles to the sample, incubating the sample with the interference capture particles with or without mixing at ambient temperature, a cool temperature such as 2-8 degree Celsius, or a warm temperature such as 37 degree Celsius, and removing the interference capture particles from the sample using magnetic separation, magnetic isolation or removal, filtration, or centrifugation, prior to the detection, measurement, or quantitation of a biomarker, and a sample can be pre-analytically cleaned of non-specific binding (NSB) to the particle or solid phase using interference capture particles comprising the same or similar particle composition, or using the same lot of particles, as the “target capture particles” whereby any bead-based or particle composition-specific interferences and NSB is captured by the interference capture particles prior to adding the target capture particles to the sample.

“Magnetic particles” or “magnetic beads” generally refer to particles or beads, respectively, which can be attracted or attractable by a magnetic field. The magnetic particles or beads can comprise a magnetic core, e.g., a magnetic metal oxide core. Magnetic particles or beads can comprise paramagnetic particles that are slightly attracted by a magnetic field and do not retain the magnetic properties when the external field is removed.

“Non-specific binding” generally refers to binding of different molecules (e.g., different type, different size, different sequence of nucleotides, etc.) with approximately the same affinity to particles.

“Particles” generally refer to substances that are in a solid form such as beads, e.g., magnetic beads, latex beads, fluorescent beads, streptavidin beads, multiplex immunoassay beads, nanobeads, microbeads, bead surface chemistry, antibody coated beads, or antigen coated beads. The particles can have a variety of shapes, which can be regular or irregular. The particles can have a size from about 10 nanometers to about 100 micrometers. The particles can comprise a coating on its surface, for example, an adsorptively or covalently bound silane coat to which a wide variety of bioaffinity adsorbents can be covalently bound through selected coupling chemistries, thereby coating the surface of the particles with functional groups. Suitable silanes, e.g., amino silanes, useful to coat the particle surfaces comprise p-aminopropyltrimethoxysilane, N-2-aminoethyl-3-aminopropyltrimethoxysilane, triaminofunctional silane, n-dodecyltriethoxysilane and n-hexyltrimethoxysilane. Alternatively, or in addition to, the particles can comprise no coating on its surface.

“Saliva Test” generally refers to the use of at home saliva or saline oral rinse collection, or physician office, retail (Walgreens, CVS, Walmart, etc.), or specialist clinic (neurologist) saliva collection, for age-based risk screening by detecting disease-state specific biomarkers in saliva such as tumor specific antigens suggestive or symptomatic of cancer onset, cancer stage or progression, or cancer recurrence such as autoantibodies against p53, free PSA, total PSA, alphafetoprotein (AFP), carcinoembryonic antigen (CEA), Beta-2-microglobulin (B2M), Beta-human chorionic gonadotropin (Beta-hCG), Bladder Tumor Antigen (BTA), CA125, CA15-3, CA19-9, CA50, CA72-4, and CA242, infectious disease (ID) such as Campylobacter Infection, Hepatitis, Hepatitis B, Hepatitis C, Novel Coronavirus (COVID-19), Influenza (Flu), Measles, Meningococcal Disease, HPV, HSV, HIV, and RSV, sexually transmitted disease or infection (STD or STI) such as Syphilis, Hepatitis, Trichomoniasis, Gonorrhea, Chlamydia, and Human Immunodeficiency Virus (HIV), autoimmune disease such as Celiac disease, vector borne disease such as Lyme disease, Zika, Dengue, and Malaria, or neuromarkers suggestive or symptomatic of neurodegenerative disease (Alzheimer's or Parkinson's) such as amyloid beta or β-amyloid (1-40, 1-42), total tau, phosphorylated tau 181, 205, 212, 217, or 231, neurofilament light (NfL), glial acidic fibrillary protein (GFAP), NSE (Neuron-specific enolase), sTREM2 (Triggering receptor expressed on myeloid cells 2), α-synuclein, Neurogranin, NPTX2 (Neuronal pentraxin-2), and BACE-1 (Beta-secretase 1) to help diagnose disease or detect the early onset of disease. Saliva-based testing can also include the testing of athletes suspected of traumatic brain injury (TBI) or concussion, or to rule-in/rule-out TBI or concussion in the emergency room (ER) or emergency department (ED) for victims of a car accident, a fall, an explosion or impact, or Shaken Baby Syndrome, such as S100B, y-enolase (NSE), alpha-II spectrin, astroglial protein, NfL, ubiquitin carboxy terminal hydrolase-L1 (UCH-L1), and tau where there is a need for the accurate and sensitive detection of neuromarkers in their saliva-based samples. If the target capture beads are added to the saliva or saline oral rinse in the collection device the target capture beads can maximize recovery of biomarkers during their collection or in the collection device whereby the entire or whole sample is treated, exposed, or incubated with the target capture beads. This is key for any biomarker that may otherwise hydrophobically, or via NSB, interact with the collection device or sample tube surface and be lost or no longer detectable in the sample if the target capture beads are not present to maximize their capture and recovery from the sample. The target capture beads can comprise different antibodies to bind one or more different biomarkers, or a panel of biomarkers, during the pre-analytical capture in the collection device or sample tube. Furthermore, if these biomarkers are also inside cells, extracellular particles, exosomes, virions, or bacterium they can also be lysed in the presence of the target capture beads to maximize the capture efficiency and yield of total biomarkers from the sample. Since the entire sample is exposed to the antibody coated target capture beads with or without lysis buffer or agent, this may also improve low abundance biomarker capture for their subsequent concentration or enrichment (e.g. instead of the sample volume being limited to be compatible with test system specific sample volume requirement such as 5-300 μL, the entire 1-5 mL saliva sample is the sample for neuromarkers capture). This novel approach to capture neuromarkers during or after saliva-based sample collection in the collection kit will maximize subsequent sensitivity of their detection.

“Sample” or “biofluid” generally refers to any human or animal serum, plasma (i.e. EDTA, lithium heparin, sodium citrate), blood, whole blood, processed blood, semen or seminal fluid, cells, tissues, biopsy material, DNA, RNA, or any fluid, dissolved solid, processed solid material, oral fluids such as oral mucosal transudates (OMT), saliva (passive drool or swab), buccal samples (collected by swabs or brushes), saline oral rinse (SOR) or oral rinses, gingival crevicular fluid (GCF), sputum, sweat, tears, mucus, urine, stool (liquid and/or solid), vaginal fluid, milk, cerebrospinal fluid, peritoneal fluid, pleural fluid, or digestive fluid to be tested for diagnosis, prognosis, screening, risk assessment, risk stratification, and monitoring such as therapeutic drug monitoring.

“Subject,” “individual,” or “patient” are often used interchangeably herein. A “subject” can be a biological entity. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

“Treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof. Similarly, the plural form of a word e.g., “biomarkers” includes both singular and plural (i.e., one or more), including mixtures thereof unless the context clearly dictates otherwise.

Methods and Systems for Multiplex Detection and/or Measurement

This disclosure provides methods and systems for multiplex detection and/or measurement of one or more biomarkers in a biological sample, with high accuracy and efficiency.

In an aspect, the present disclosure provides a method for detecting and/or measuring one or more biomarkers in a biological sample, comprising step (a) providing a sample of a subject; and step (b) capturing a biomarker from the sample by contacting the sample with biomarker capture particles (a “Capture” or “capturing” process).

In some embodiments, the biological sample is subjected to cleaning by removing assay interferences from the sample with interference capture particles or cleaning and conditioning prior to (b).

### Samples

In some embodiments, the sample can comprise any human or animal serum, plasma (i.e., EDTA, lithium heparin, sodium citrate), blood, whole blood, processed blood, semen or seminal fluid, cells, tissues, biopsy material, DNA, RNA, or any fluid, dissolved solid, processed solid material, oral fluids such as oral mucosal transudates (OMT), saliva (passive drool or swab), buccal samples (collected by swabs or brushes), saline oral rinse (SOR) or oral rinses, gingival crevicular fluid (GCF), sputum, sweat, tears, mucus, urine, stool (liquid and/or solid), vaginal fluid, milk, cerebrospinal fluid, peritoneal fluid, pleural fluid and digestive fluid. In some embodiments, the sample can comprise a sample from a human. In some embodiments, the sample can comprise a sample from an animal.

In some embodiments, the sample comprises a volume of about 10 μL to about 100 mL. In some embodiments, the volume of the sample can be about 10 μL, about 20 μL, about 30 μL, about 40 μL, about 50 μL, about 60 μL, about 70 μL, about 80 μL, about 90 μL, about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL, about 1 mL, about 1.5 mL, about 2 mL, about 2.5 mL, about 3 mL, about 3.5 mL, about 4 mL, about 4.5 mL, about 5 mL, about 5.5 mL, about 6 mL, about 6.5 mL, about 7 mL, about 7.5 mL, about 8 mL, about 8.5 mL, about 9 mL, about 9.5 mL, about 10 mL, about 20 mL, about 40 mL, about 50 mL, about 75 mL, about 100 mL, about 150 mL, or about 200 mL, or a range defined by any of the aforementioned amounts. The sample volume may include at least 10 μL, at least 20 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 350 μL, at least 400 μL, at least 450 μL, at least 500 μL, at least 550 μL, at least 600 μL, at least 650 μL, at least 700 μL, at least 750 μL, at least 800 μL, at least 850 μL, at least 900 μL, at least 950 μL, at least 1 mL, at least 1.5 mL, at least 2 mL, at least 2.5 mL, at least 3 mL, at least 3.5 mL, at least 4 mL, at least 4.5 mL, at least 5 mL, at least 5.5 mL, at least 6 mL, at least 6.5 mL, at least 7 mL, at least 7.5 mL, at least 8 mL, at least 8.5 mL, at least 9 mL, at least 9.5 mL, at least 10 mL, at least 20 mL, at least 40 mL, at least 50 mL, at least 75 mL, at least 100 mL, at least 150 mL, or at least 200 mL. In some embodiments, the sample volume is less than 10 μL, less than 20 μL, less than 30 μL, less than 40 μL, less than 50 μL, less than 60 μL, less than 70 μL, less than 80 μL, less than 90 μL, less than 100 μL, less than 150 μL, less than 200 μL, less than 250 μL, less than 300 μL, less than 350 μL, less than 400 μL, less than 450 μL, less than 500 μL, less than 550 μL, less than 600 μL, less than 650 μL, less than 700 μL, less than 750 μL, less than 800 μL, less than 850 μL, less than 900 μL, less than 950 μL, less than 1 mL, less than 1.5 mL, less than 2 mL, less than 2.5 mL, less than 3 mL, less than 3.5 mL, less than 4 mL, less than 4.5 mL, less than 5 mL, less than 5.5 mL, less than 6 mL, less than 6.5 mL, less than 7 mL, less than 7.5 mL, less than 8 mL, less than 8.5 mL, less than 9 mL, less than 9.5 mL, less than 10 mL, less than 20 mL, less than 40 mL, less than 50 mL, less than 75 mL, less than 100 mL, less than 150 mL, or less than 200 mL. In some embodiments, the sample can comprise a volume less than 200 μL. In some cases, the sample is a point-of-care (POC) finger prick collection sample, pediatric sample, geriatric sample, or IV drug user sample, where blood collection is challenging. In some embodiments, the sample can comprise a volume between about 200 μL and about 5 mL. In some embodiments, the sample can comprise a volume larger than about 5 mL where larger volumes may be needed for biomarker enrichment.

In some embodiments, the sample comprises a biofluid. In some embodiments, the biofluid comprises whole blood, plasma, serum, oral fluid (saliva, drool, oral mucosal transudates (OMT), or oral rinse e.g., saline oral rinse), mucus, urine, semen, vaginal fluid, milk, cerebrospinal fluid, peritoneal fluid, pleural fluid, cerebrospinal fluid (CSF), tissues, sweat, tears, or digestive fluid.

### Subjects

In some embodiments, the subject is suspected of having a disease, or has been administered a vaccine against the disease, or is being screened for a disease. In some embodiments, the subject has a disease. In some embodiments, the disease can comprise an infection, a viral infection, a bacterial infection, a protozoan infection, a tick-borne illness, a Lyme disease, a severe acute respiratory syndrome, or a coronavirus infection, e.g., Coronavirus disease 2019 (COVID-19).

In some embodiments, the disease comprises an infection. In some embodiments, the disease comprises a viral infection, a bacterial infection, or a protozoan infection. In some embodiments, the disease comprises a tick-borne illness. In some embodiments, the disease comprises Lyme disease. In some embodiments, the disease comprises a severe acute respiratory syndrome. the disease comprises a coronavirus infection. the coronavirus infection comprises Coronavirus disease 2019 (COVID-19).

In some embodiments, the subject is an animal. In some embodiments, the subject is a mammal. In some embodiments, the subject is a primate. In some embodiments, the subject is a human.

### Biomarkers

In some embodiments, the biomarker can comprise antibodies, autoantibodies, therapeutic antibodies, immunoglobulin classes such as IgG, IgM, IgA, IgE, immunoglobulin subclasses such as IgAQ1, IgA2, IgG1, IgG2, IgG3, IgG4, circulating antibodies, secretory antibodies, and alpaca derived nanobodies, and animal derived antibodies such as from a mouse, pig, monkey, rat, sheep, goat, or cow.

In some embodiments, the therapeutic antibodies can comprise the FDA approved monoclonal antibody drugs including Infliximab for Crohn's disease, Rituximab for lymphoma, Ustekinumab for Psoriasis, and Tocilizumab for treatment of rheumatoid arthritis and the FDA authorized antibody drugs for the emergency use including Bebtelovimab, a monoclonal antibody directed against the spike protein of SARS-CoV-2.

In some embodiments, the biomarker can comprise antigens, pathogens, proteins, small molecules, therapeutics, hormones, peptides, signaling peptides, exosomes, or cells.

In some embodiments, the biomarker can comprise disease state-specific antigens, antibodies, or biomarkers such as for diseases such as cardiac, Alzheimer's disease, traumatic brain injury (TBI), neurodegenerative disease, cancer, renal, autoimmune, infectious diseases, bacterial infection, viral infection, fungal infection, sexually transmitted disease (STD) or infection (STI), fertility, anemia, endocrinology, inflammation, metabolic disease, sleep apnea, hepatic disease, or respiratory disease. The detection of the biomarkers can be used for diagnosis or prognosis of a disease.

In some embodiments, the biomarker can comprise neuromarkers for Alzheimer's disease or neurodegenerative disease. In some embodiments, the neuromarkers can comprise amyloid beta or β-amyloid (1-38, 1-40, 1-42) also known as Abeta38, Abeta40, or Abeta42, total tau, aggregates of hyperphosphorylated Tau protein or paired helical filaments (PHF-Tau) such as PHF-Tau (Ser202/Thr205), PHF-Tau (Thr181), PHF-Tau (Thr217), PHF-Tau (Thr231), also known as phosphorylated tau 181 (pTau181) or 231 (pTau231), neurofilament light (NfL), glial acidic fibrillary protein (GFAP), NSE (Neuron-specific enolase), sTREM2 (Triggering receptor expressed on myeloid cells 2), α-synuclein, Neurogranin, NPTX2 (Neuronal pentraxin-2), and BACE-1 (Beta-secretase 1). Neuromarkers for traumatic Brian injury (TBI) or concussion can comprise S100B, y-enolase (NSE), alpha-II spectrin, astroglial protein, NfL, ubiquitin carboxy terminal hydrolase-L1 (UCH-L1), and tau.

In some embodiments, the biomarker can comprise one or more biomarkers, or a combination thereof, such as antibodies, antigens, and therapeutics, in a multiplex detection and/or measurement. A multiplex detection and/or measurement of different biomarkers can be used, in particular, in the diagnostic of a stage of a disease, the monitoring of vaccine efficiency, and the monitoring of therapeutic efficiency.

In some embodiments, the biomarker capture particles can comprise beads, e.g., biomarker capture beads. In some embodiments, the biomarker capture particles can comprise magnetic particles or paramagnetic particles. In some embodiments, the biomarker capture particles can comprise non-magnetic particles. In some embodiments, the biomarker capture particles described herein can have a mean diameter from about 0.010 micrometers (μm) to about 3.00 micrometers, or preferably from 0.05 micrometers to 2.8 micrometers in diameter, or still more preferably 0.05 micrometers to 1.6 micrometers, or preferably from about 0.05 micrometers to about 0.55 micrometers in diameter.

### Particles

In some embodiments, the particles can comprise microparticles. In some embodiments, the particles can comprise nanoparticles. In some embodiments, the particles comprise beads. In some embodiments, the particles comprise a metal. In some embodiments, the particles are magnetic.

In some embodiments, the particles described herein (e.g., microparticle, nanoparticle) can comprise a core or support. In some embodiments, the core or support can be a paramagnetic or superparamagnetic material which can experience a force in a magnetic field gradient, but do not become permanently magnetized. Non-limiting examples of paramagnetic or superparamagnetic material can comprise iron oxide, ferromagnetic iron oxide, Fe₂O₃ or Fe₃O₄, maghemite, or combinations thereof.

In some embodiments, the core or support can comprise ceramic, glass, latex, silica, metal, alloy, colloidal metal such as gold, silver or alloy, or polymers.

In some embodiments, the particles can comprise an organic polymer or copolymer. In some embodiments, the organic polymer or copolymer is hydrophobic. In some embodiments, the organic polymer or copolymer can comprise a material selected from the group consisting of, but not limited to polystyrene, derivatized polystyrene, poly(divinylbenzene), styrene-acylate copolymer, styrene-butadiene copolymer, styrene-divinylbenzene copolymer, poly(styrene-oxyethylene), polymethyl methacrylate, polymethacrylate, polyurethane, polyglutaraldehyde, polyethylene imine, polyvinylpyrrolidone, polyvinyl alcohol, polyacrylic acid, N,N′-methylene bis-acrylamide, polyolefeins, polyethylene, polypropylene, polyvinylchloride, polyacrylonitrile, polysulfone, poly(ether sulfone), pyrolized materials, block copolymers, and copolymers of the foregoing, silicones, or silica, methylol melamine, a biodegradable polymer such as dextran or poly(ethylene glycol)-dextran (PEG-DEX), or combinations thereof.

In some embodiments, the surface of the particle can comprise functional groups or a plurality of functional groups for covalent attachment (coupling, conjugation or binding) of a binder, binding partner, capture moiety or combinations thereof such as carboxyl, tosyl, epoxy, amine, sulfhydryl, hydroxyl, ester, and maleimide, click chemistry functionality [Copper(I)-Catalyzed Azide-Alkyne Cycloaddition (CuAAC), Strain-promoted Azide-Alkyne Cycloaddition (SPAAC), Strain-promoted Alkyne-Nitrone Cycloaddition (SPANC), and Reactions of Strained Alkenes such as Alkene and Azide [3+2] cycloaddition, Alkene and Tetrazine inverse-demand Diels-Alder, and Alkene and Tetrazole photoclick reaction], hydrazone-based coupling functionality such as S-HyNic (succinimidyl-6-hydrazino-nicotinamide) and S-4FB (N-succinimidyl-4-formylbenzamide) heterobifunctional crosslinkers, and photoreactive chemistries.

In some embodiments, the biomarker capture particles can comprise any suitable particles described herein or any combinations thereof.

In some embodiments, the biomarker capture particles can comprise a capture moiety. In some embodiments, the capture moiety may be coated on a surface of the biomarker capture particles. In some embodiments, the capture moiety can be covalently or non-covalently bound to the biomarker capture particles. The covalent binding can comprise any suitable binding chemistries, for example, attachment through one or more functional groups selected from the group consisting of carboxyl, hydroxyl, tosyl, epoxy, aldehyde, amine, amide, amino, hydrazide, isothiocyanate, maleimide, and sulfhydryl. In some embodiments, the capture moiety can be bound to the biomarker capture particles with an amine reactive reagent such as sulfo-NHS-LC-biotin.

In some embodiments, the capture moiety (i.e., antibody or antibody fragment such as SH-Fab) can be bound/coated to the biomarker capture particle by a cleavable bond. In some embodiments, the cleavable bond can be a disulfide bond (R—S—S—R). After washing or isolating the particles from the sample matrix, the particles can subsequently be treated with a solution containing a reducing agent such as TCEP or DTT to cleave the disulfide bond and release the capture moiety-biomarker complex into a solution for subsequent treatment or measurement.

In some embodiments, depending on the application for which an affinity assay is to be designed, substances may function as one, or alternatively as the other, member of a binding pair consisting of capture moiety and biomarker. Such substances can be used, for example, as capture moieties (analyte binders) or can be used to generate capture moieties (e.g., by employing them as haptens/antigens to generate specific antibodies) that can be used herein. In some embodiments, such substances can by the biomarkers. Affinity assays, including immunoassays, can be designed to detect the presence and/or level of such biomarkers in a sample.

In some embodiments, the substances can be associated with the particles, and used to capture molecules or biomarkers that interact with them (such as, for example, antibodies or fragments thereof specific for the listed substances, binding proteins, or enzymes).

A nonlimiting list of substances that may function as one, or alternatively as the other, member of a binding pair consisting of capture moiety and biomarker can comprise: inducible nitric oxide synthase (iNOS), CA19-9, IL-1α, IL-1β, IL-2, IL-3, IL-4, ILt, IL-5, IL-7, IL-10, IL-12, IL-13, sIL-2R, sIL-4R, sIL-6R, SIV core antigen, IL-1RA, TNF-α, IFN gamma, GM-CSF; isoforms of PSA (prostate-specific antigen) such as PSA, pPSA, BPSA, in PSA, non α1 antichymotrypsin-complexed PSA, α1-antichymotrypsin-complexed PSA, prostate kallikreins such as hK2, hK4, and hK15, ek-rhK2, Ala-rhK2, TWT-rhK2, XarhK2, HWT-rhK2, and other kallikreins; HIV-1 p24; ferritin, L ferritin, troponin I, BNP, leptin, digoxin, myoglobin, B-type natriuretic peptide or brain natriuretic peptide (BNP), NTproBNP, CNP, NT-proCNP(1-50), NT-CNP-53(51-81), CNP-22(82-103), CNP-53(51-103), atrial natriuretic peptide (ANP); human growth hormone, bone alkaline phosphatase, human follicle stimulating hormone, human leutinizing hormone, prolactin; human chorionic gonadotrophin (e.g., CGα, CGβ); soluble ST2, thyroglobulin; anti-thyroglobulin; IgE, IgG, IgG1, IgG2, IgG3, IgG4, B. anthracis protective antigen, B. anthracis lethal factor, B. anthracis spore antigen, F. tularensis LPS, S. aureas enterotoxin B, Y. pestis capsular F1 antigen, insulin, alpha fetoprotein (e.g., AFP 300), carcinoembryonic antigen (CEA), CA 15.3 antigen, CA 19.9 antigen, CA 125 antigen, HAV Ab, HAV Igm, HBc Ab, HBc Igm, HIV1/2, HBsAg, HBsAb, HCV Ab, anti-p53, histamine; neopterin; s-VCAM-1, serotonin, sFas, sFas ligand, sGM-CSFR, s1CAM-1, thymidine kinase, IgE, EPO, intrinsic factor Ab, haptoglobulin, anti-cardiolipin, anti-dsDNA, anti-Ro, Ro, anti-La, anti-SM, SM, anti-nRNP, antihistone, anti-Scl-70, Scl-70, anti-nuclear antibodies, anti-centromere antibodies, SS-A, SS-B, Sm, U1-RNP, Jo-1, CK, CK-MB, CRP, ischemia modified albumin, HDL, LDL, oxLDL, VLDL, troponin T, troponin I, troponin C, microalbumin, amylase, ALP, ALT, AST, GGT, IgA, IgG, prealbumin, anti-streptolysin, chlamydia, CMV IgG, toxo IgG, toxo IgM, apolipoprotein A, apolipoprotein B, C3, C4, properdin factor B, albumin, α1-acid glycoprotein, α1-antitrypsin, α1 microglobulin, α2-macroglobulin, anti-streptolysin 0, antithrombin-III, apolipoprotein A1, apolipoprotein B, p2-microglobulin, ceruloplasmin, complement C3, complement C4, C-reactive protein, DNase B, ferritin, free kappa light chain, free lambda light chain, haptoglobin, immunoglobulin A, immunoglobulin A (CSF), immunoglobulin E, immunoglobulin G, immunoglobulin G (CSF), immunoglobulin G (urine), immunoglobulin G subclasses, immunoglobulin M, immunoglobulin M (CSF), kappa light chain, lambda light chain, lipoprotein (a), microalbumin, prealbumin, properdin factor B, rheumatoid factor, ferritin, transferrin, transferrin (urine), rubella IgG, thyroglobulin antibody, toxoplasma IgM, toxoplasma IgG, IGF-I, IGF-binding protein (IGFBP)-3, hepsin, pim-1 kinase, E-cadherein, EZH2, and a-methylacyl-CoA racemase, TGF-beta, IL6SR, GAD, IA-2, CD-64, neutrophils CD-64, CD-20, CD-33, CD-52, isoforms of cytochrome P450, s-VCAM-1, sFas, sICAM, hepatitis B surface antigen, thromboplastin, HIV p24, HIV gp41/120, HCV C22, HCV C33, hemoglobin A1c, and GAD65, IA2, vitamin D, 25-OH vitamin D, 1,25(OH)2 vitamin D, 24,25(OH)2 vitamin D, 25,26(OH)2 vitamin D, 3-epimer of vitamin D, FGF-23, sclerostin, procalcitonin, calcitonin, C. dificille toxin A&B, H. pylori, HSV-1, HSV2.

In some embodiments, substances that may function as one, or alternatively as the other, member of a binding pair consisting of capture moiety and biomarker can comprise moieties, for example antibodies or fragments thereof, specific for any of the WHO International Biological Reference Preparations held and, characterized, and/or distributed by the WHO International Laboratories for Biological Standards (available at http://www.who.int/bloodproducts/re_materials, updated as of Jun. 30, 2005, the list is herein incorporated by reference).

A partial list of such suitable international reference standards, identified by WHO code in parentheses following the substance, includes: human recombinant thromboplastin (rTF/95), rabbit thromboplastin (RBT/90), thyroid-stimulating antibody (90/672), recombinant human tissue plasminogen activator (98/714), high molecular weight urokinase (87/594), prostate specific antigen (96/668), prostate specific antigen 90:10 (96/700); human plasma protein C (86/622), human plasma protein S (93/590), rheumatoid arthritis serum (W1066), serum amyloid A protein (92/680), streptokinase (00/464), human thrombin (01/580), bovine combined thromboplastin (OBT/79), anti-D positive control intravenous immunoglobulin (02/228), islet cell antibodies (97/550), lipoprotein a (IFCC SRM 2B), human parvovirus B19 DNA (99/800), human plasmin (97/536), human plasminogen-activator inhibitor 1 (92/654), platelet factor 4 (83/505), prekallikrein activator (82/530), human brain CJD control and human brain sporadic CJD preparation 1 and human brain sporadic CJD preparation 2 and human brain variant CJD (none; each cited in WHO TRS ECBS Report No. 926, 53.sup.rd Report, brain homogenate), human serum complement components C1q, C4, C5, factor B, and whole functional complement CH50 (W1032), human serum immunoglobulin E (75/502), human serum immunoglobulins G, A, and M (67/86), human serum proteins albumin, alpha-1-antitrypsin, alpha-2-macroglobulin, ceruloplasmin, complement C3, transferrin (W1031), anti-D negative control intravenous immunoglobulin (02/226), hepatitis A RNA (00/560), hepatitis B surface antigen subtype adw2 genotype A (03/262 and 00/588), hepatitis B viral DNA (97/746), hepatitis C viral RNA (96/798), HIV-1 p24 antigen (90/636), HIV-1 RNA (97/656), HIV-1 RNA genotypes (set of 10 101/466), human fibrinogen concentrate (98/614), human plasma fibrinogen (98/612), raised A2 hemoglobin (89/666), raised F hemoglobin (85/616), hemoglobincyanide (98/708), low molecular weight heparin (85/600 and 90/686), unfractionated heparin (97/578), blood coagulation factor VIII and von Willebrand factor (02/150), human blood coagulation factor VIII concentrate (99/678), human blood coagulation factor XIII plasma (02/206), human blood coagulation factors II, VII, IX, X (99/826), human blood coagulation factors II and X concentrate (98/590), human carcinoembryonic antigen (73/601), human Creactive protein (85/506), recombinant human ferritin (94/572), apolipoprotein B (SP3-07), beta-2-microglobulin (B2M), human beta-thromboglobulin (83/501), human blood coagulation factor IX concentrate (96/854), human blood coagulation factor IXa concentrate (97/562), human blood coagulation factor V Leiden, human gDNA samples FV wild type, FVL homozygote, FVL heterozygote (03/254, 03/260, 03/248), human blood coagulation factor VII concentrate (97/592), human blood coagulation factor VIIa concentrate (89/688), human anti-syphilitic serum (HS), human anti-tetanus immunoglobulin (TE-3), human antithrombin concentrate (96/520), human plasma antithrombin (93/768), human antithyroglobulin serum (65/93), anti-toxoplasma serum (TOXM), human anti-toxoplasma serum (IgG) (01/600), human anti-varicella zoster immunoglobulin (W1044), apolipoprotein A-1 (SP1-01), human anti-interferon beta serum (G038-501-572), human anti-measles serum (66/202), anti-nuclear ribonucleoprotein serum (W1063), anti-nuclear-factor (homogeneous) serum (66/233), anti-parvovirus B19 (IgG) serum (91/602), anti-poliovirus serum Types 1,2,3 (66/202), human anti-rabies immunoglobulin (RAI), human anti-rubella immunoglobulin (RUBI-1-94), anti-smooth muscle serum (W1062), human anti-double-stranded DNA serum (Wo/80), human anti-E complete blood-typing serum (W1005), human anti-echinococcus serum (ECHS), human anti-hepatitis A immunoglobulin (97/646), human anti-hepatitis B immunoglobulin (W1042), human anti-hepatitis E serum (95/584), anti-human platelet antigen-la (93/710), anti-human platelet antigen-5b (99/666), human anti-interferon alpha serum (B037-501-572), human alphafetoprotein (AFP), ancrod (74/581), human anti-A blood typing serum (W1001), human anti-B blood typing serum (W1002), human anti-C complete blood typing serum (W1004), anti-D (anti-Rh0) complete blood-typing reagent (99/836), human anti-D (anti-Rh0) incomplete blood-typing serum (W1006), and human anti-D immunoglobulin (01/572).

Other examples of substances that may function as one, or alternatively as the other, member of a binding pair consisting of capture moiety and biomarker, depending on the application for which an affinity assay is to be designed include compounds that can be used as haptens to generate antibodies capable of recognizing the compounds, and include but are not limited to, any salts, esters, or ethers, of the following: hormones, including but not limited to progesterone, estrogen, and testosterone, progestins, corticosteroids, and dehydroepiandrosterone, and any non protein/non-polypeptide antigens that are listed as international reference standards by the WHO. A partial list of such suitable international reference standards, identified by WHO code in parentheses following the substance, includes vitamin B12 (WHO 81.563), folate (WHO 95/528), homocystein, transcobalamins, T4/T3, and other substances disclosed in the WHO catalog of International Biological Reference Preparations (available at the WHO website, for example at page http://www.who.int/bloodproducts/ref_materials/, updated Jun. 30, 2005), which is incorporated herein by reference.

The methods and systems described herein can comprise an aforementioned WHO reference standards or a mixture containing a reference standard. Other examples of substances that may function as one, or alternatively as the other, member of a binding pair consisting of analyte binder (capture moiety) and analyte, depending on the application for which an affinity assay is to be designed include drugs of abuse. Drugs of abuse can comprise, for example, drugs and their metabolites (e.g., metabolites present in blood, in urine, and other biological materials), as well any salts, esters, or ethers, thereof: heroin, morphine, hydromorphone, codeine, oxycodone, hydrocodone, fentanyl, demerol, methadone, darvon, stadol, talwin, paregoric, buprenex; stimulants such as, for example, amphetamines, methamphetamine; methylamphetamine, ethylamphetamine, methylphenidate, ephedrine, pseudoephedrine, ephedra, ma huang, methylenedioxyamphetamine (MDS), phentermine, phenylpropanolamine; amiphenazole, bemigride, benzphetamine, bromatan, chlorphentermine, cropropamide, crothetamide, diethylpropion, dimethylamphetamine, doxapram, ethamivan, fencamfamine, meclofenoxate, methylphenidate, nikethamide, pemoline, pentetrazol, phendimetrazine, phenmetrazine, phentermine, phenylpropanolamine, picrotoxine, pipradol, prolintane, strychnine, synephrine, phencyclidine and analogs such as angel dust, PCP, ketamine; depressants such as, for example, barbiturates, gluthethimide, methaqualone, and meprobamate, methohexital, thiamyl, thiopental, amobarbital, pentobarbital, secobarbital, butalbital, butabarbital, talbutal, and aprobarbital, phenobarbital, mephobarbital; benzodiazapenes such as, for example, estazolam, flurazepam, temazepam, triazolam, midazolam, alprazolam, chlordiazepoxide, clorazepate, diazepam, halazepam, lorazepam, oxazepam, prazepam, quazepam, clonazepam, flunitrazepam; GBH drugs such as gamma hydroxyl butyric acid and gamma butyrolactone; glutethimide, methaqualone, meprobamate, carisoprodol, zolpidem, zaleplon; cannabinoid drugs such as tetrahydracannabinol and analogs; cocaine, 3-4 methylenedioxymethamphetamine (MDMA); hallucinogens such as, for example, mescaline and LSD.

In some embodiments, a capture moiety can comprise antibodies, antibody fragments, polypeptide binders, a polymer of antibody, a polymer of antibody fragments, a polymer of antibody and antibody fragments, a receptor, a ligand of a receptor, a ligand binder, a ligand of a ligand binder, an enzyme, an irreversibly inactivated enzyme, alkaline phosphatase, horse radish peroxidase, a peptide, a protein, a polymer, a fluorophore, a fluorescent dye, a quantum dot (Qdot), a fluorescent protein label, a DNA stain, a chemical, and a chemiluminescence chemical such as luminol, isoluminol, derivatives of isoluminol, acridinium ester, ruthenium and N-(4-aminobutyl)-N-ethyl-isoluminol (ABEI), amine-reactive labeling reagents such as, for example, sulfo-NHS-biotin, sulfo-NHS-LC-biotin, sulfo-NHS-LC-LC-biotin, sulfo-NHS-SS-biotin, NHS-PEO4-biotin, NHS-biotin, NHS-LC-biotin, NHS-LC-LC-biotin, PFP-biotin, TFP-PEO-biotin, or NHS-iminobiotin trifluoroacetamide, sulfhydryl-reactive biotin labeling reagents such as, for example, maleimide-PEO2-biotin, biotin-BMCC, PEO-Iodoacetyl biotin, iodoacetyl-LC-biotin, or biotin-HPDP, carboxyl-reactive biotin labeling reagents such as, for example, biotin PEO-amine or biotin PEO-LC-amine, carbohydrate-reactive biotin labeling reagents such as, for example, biocytin hydrazide, biotin hydrazide, or biotin-LC-hydrazide, and photoreactive biotin labeling reagents such as, for example, psoralen-PEO-biotin, monobodies, non-immunoglobulin binders, DARPins, affibodies, anticalins, MIP, aptamers, chimeric antibodies, therapeutic antibodies, alpaca derived nanobodies, phase display VHH constructs, or nucleic acids. In some embodiments, the surface of the biomarker capture particles can be co-coated with animal antibodies or animal serum to block an interference, e.g., a human anti-animal antibody (HAAA) or rheumatoid factor (RF) interference, or co-coated with Manufacturer assay specific reagents such as streptavidin, ALP, HRP, BSA-fluorescein, BSA-ABEI, BSA-acridinium, or BSA-ruthenium to block manufacture assay specific interference (MASI).

In some embodiments, antibody fragments can comprise Fab, F(ab′)2, Fc, scFv, or engineered variants such as diabodies, triabodies, minibodies, VHH constructs and single-domain antibodies. In some embodiments, the antibody fragments can be recombinant, monoclonal, or polyclonal.

In some embodiments, the antibody can comprise autoantibodies, therapeutic antibodies, immunoglobulin classes such as IgG, IgM, IgA, and IgE, immunoglobulin subclasses such as IgA1, IgA2, IgG1, IgG2, IgG3, and IgG4, circulating antibodies, secretory antibodies, alpaca derived nanobodies, and animal derived antibodies. In some embodiments, therapeutic antibodies can comprise the FDA approved monoclonal antibody drugs including Infliximab for Crohn's disease, Rituximab for lymphoma, Ustekinumab for Psoriasis, and Tocilizumab for treatment of rheumatoid arthritis and the FDA authorized antibody drugs for the emergency use including Bebtelovimab, a monoclonal antibody directed against the spike protein of SARS-CoV-2.

In some embodiments, the target capture particles, or biomarker capture particles, can comprise a single antigen coated bead, two or more antigen coated beads, or a pool of different antigen coated beads, a bead coated with more than one recombinant antigen, or a bead coated with viral or bacterial lysate to improve the likelihood and sensitivity of detecting human immunoglobulins such as IgA, IgG, IgM, and IgE, immunoglobulin subclasses, or a combination thereof against the antigen or antigens. The biomarker capture particles can be used in a serology testing.

In some embodiments, the biomarker capture particles can comprise a single antibody coated bead, two or more antibody coated beads, or a pool of different antibody coated beads, a bead coated with more than one antibody, a bead coated with a capture moiety (e.g., antibody fragment, aptamer, MIP, alpaca-derived nanobody), or a pool or combination of such beads to improve the likelihood and sensitivity of detecting an antigen or biomarker, or for multiplex detection of more than one antigen or biomarker. This is antigen testing and is typically performed by a sandwich assay, competitive assay, piggyback assay, delayed addition, or a delayed capture assay. The biomarker capture particles can be used in an antigen assay.

In some embodiments, the biomarker capture particles can comprise a pool of different biomarker capture particles coated with antibodies and antigens to perform a combination or “combo” assay or detect both antibodies and antigens in the same test, or by combining different assay formats as may be needed to detect antigens or biomarkers. By cleaving or eluting the captured antibodies, antigens, or biomarkers from the biomarker capture particles, the eluate can be measured, or the eluate can be neutralized and measured, by mass spectrometry (LC-MS, LC-MS/MS, MALDI-TOF, etc.), ELISA, CLIA, FIA, chemistry, molecular (PCR, NGS), or any measurement method or system.

In some embodiments, the biomarker capture particles can comprise latex beads. In some embodiments, the latex beads can comprise polymeric beads. In some embodiments the latex beads can be different colors such as white, blue, red, black, green, yellow, orange, or brown.

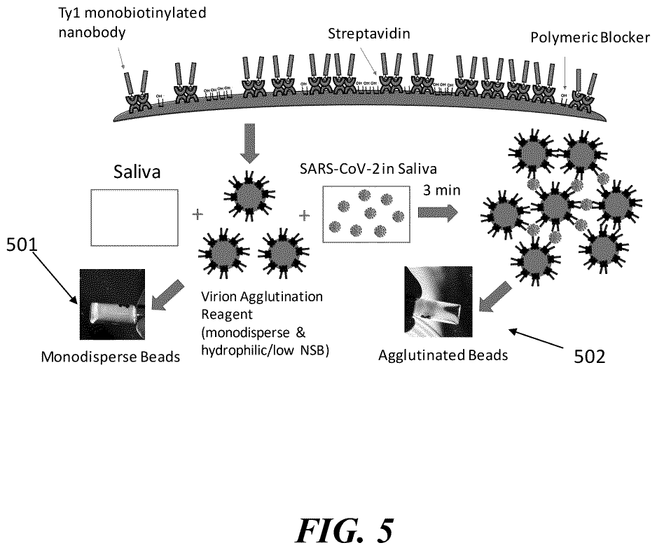

In some embodiments, the biomarker capture particles can comprise agglutination beads. In some embodiments, the biomarker can comprise a plurality of epitopes that each epitope can bind to a biomarker capture moiety. In some embodiments, the plurality of epitopes can comprise two or more repeating epitopes. In some embodiments, the plurality of epitopes can comprise two or more different epitopes. In some embodiments, the plurality of epitopes can comprise any combination of epitopes. In some embodiments, the agglutination beads can comprise a plurality of biomarker capture moieties such that an agglutination bead can capture/bind to a plurality of biomarkers. In some embodiments, the agglutination beads can aggregate or agglutinate when more than one, more than two, more than three, more than four, more than five, more than six, more than seven, more than eight, more than nine, more than ten, or more agglutination beads are bound by one biomarker. In some embodiments, a biomarker can bind to a plurality of agglutination beads, and an agglutination bead of the plurality of agglutination beads can bind to a plurality of biomarkers, thereby forming a network comprising a plurality of biomarkers and a plurality of agglutination beads. In some embodiments, the agglutinated, aggregated, or precipitated agglutination beads can be visually detectable, thereby allowing for a visual rapid test or point of care test where visual detection of bead agglutinates indicates the presence of a biomarker, e.g., a pathogen, disease, or antibody. In some embodiments, the agglutinated, aggregated, or precipitated agglutination beads can be detectable via a detection, for example, turbidimetric detection, UV-vis detection, infrared detection, light scattering, or microscopy detection. Referring to FIG. 5, in the absence of virions, the biomarker capture particles stay monodisperse or a clear brownish liquid (501). However, in the presence of virions, the biomarker capture particles agglutinate, aggregate, or precipitate (502).

In some embodiments, the agglutination beads can comprise different colors (e.g., red, yellow, blue, or any color). In some embodiments, 2 or more different biomarkers could be visually detected in a single test if each different color bead (such as latex beads) comprises different antibody or antibodies against different targets. For example, beads with color A can comprise biomarker capture moieties that are specific for biomarker X indicative of a disease, e.g., SARS-CoV-2; beads with color B can comprise biomarker capture moieties that are specific for biomarker Y indicative of another disease, e.g., Influenza, and beads with color C can comprise biomarker capture moieties that are specific for biomarker Z indicative of a disease, e.g., RSV. When two or more types of beads are used, a precipitation of beads with the specific color can be indicative of a presence of the corresponding biomarker. In some embodiments, the precipitated beads can comprise blended color from two or more types of beads. For example, if beads with color A precipitate, it could be indicative that biomarker X is present in the sample.

In some embodiments, the biomarker capture particles can comprise a first plurality of magnetic beads and a second plurality of latex beads or non-magnetic beads. In some embodiments, the second plurality of latex beads can exhibit a color. In some embodiments, the first plurality of magnetic beads and the second plurality of latex beads can comprise different sizes or dimensions. In some embodiments, a magnetic bead can comprise a first capture moiety and a colored latex bead can comprise a second capture moiety, wherein the first capture moiety and the second capture moiety can capture a first epitope and a second epitope on a single biomarker respectively. If the biomarker is absent, the magnetic beads may be removed leaving 100% of the colored latex beads or non-magnetic beads in solution for a control color, control color intensity or darkness, control density, etc. If the biomarker is present in the sample, it can be captured by the magnetic bead and the colored latex bead such that the magnetic bead will capture both the antigen and the colored latex bead, forming a complex comprising the magnetic bead and the colored latex bead bridged by the biomarker. When a magnet or a magnetic field is applied, the complex can be pulled out of solution, resulting in a color change, color intensity reduction, or density of colored latex beads reduction in the solution or the supernatant. This change in the solution or the supernatant is indicative of the presence of the biomarker. In some embodiments, the change in the solution or the supernatant can easily be detected visually, or with a density meter, a flow cytometer, a turbidimetric detector, A600 absorbance, etc. This is similar or analogous to coating ping pong balls onto a basketball where the ping pong balls are the small, latex or polystyrene non-magnetic beads (e.g., in the 10-300 nm range in a variety of colors), and the basketball is a larger, magnetic bead (e.g., in the 200 nm to 3,000 nm size range). In some cases, only the large magnetic beads will be attracted by a magnet. The non-magnetic small latex or polystyrene beads will not be attracted by a magnet and are very colloidally stable since they are so small (e.g., they will remain homogeneously dispersed with good colloidal stability and very slow settling time, or they will not settle out of solution). In some cases, the main way for the “non-magnetic” ping pong balls to move to the magnet as well is for them to interact and bind to the larger “magnetic” basketball such that they will move to the magnet together. This may require the basketball is coated with a capture moiety or anti-target antibody or binding partner, and also the ping pong balls to be coated with a capture moiety or anti-target antibody or binding partner against the same target, biomarker, antigen, or analyte as the Basketball, but ideally targeting distal epitopes (i.e., the Basketball binds target epitope 1 and the ping pong balls bind target epitope 2) such as antibody pairs commonly used today for sandwich immunoassays.

In some embodiment, the surface of the biomarker capture particles can be attenuated to decrease the density of capture moieties per biomarker capture particle or per unit mass such that less biomarkers are captured per biomarker capture particle to increase test sensitivity to biomarker concentration differences. To enhance assay test sensitivity of the POC, it may be important to control the number of ping pong balls that can bind per basketball. In this way small changes in target or biomarker concentrations can be detected by large changes in residual concentration of ping pong balls in solution that have not bound to target or biomarker. This may require one to maximize the number of ping pong balls that can bind to each basketball while minimizing or attenuating the number of targets or biomarkers that can bind per ping pong ball, or both the basketballs and ping pong balls may have an attenuated surface or less antibody per unit surface area, or per bead (ping pong ball and basketball), or per mass of beads such as micrograms of antibody bound per mg of beads. Attenuation can be accomplished at the primary coating step, or via a secondary coating step, by diluting or co-coating the antibody on the beads with a non-specific antibody, a smaller molecular weight protein such as Streptavidin, BSA, or a lysine rich peptide, or a polymer with amines such as a NH2-PEGx compound such as JSR Blockmaster CE210 and/or CE510, or coating streptavidin beads with a mixture of biotinylated capture moiety with a small molecule such as biotin or biotin-fluorescein. For primary covalent coupling or hydrophobic coating or streptavidin coating this is accomplished by adding a protein solution of the capture moiety or binding partner such as 5, 10, 20, 30, 40, 50, 60, 70, 80, or 90% mixture with a non-specific protein, peptide, polymer, or small molecule at 95, 90, 80, 70, 60, 50, 40, 30, 20, or 10% of the solution such that they co-coat on the beads thereby decreasing or controlling the density of the specific capture moiety, binding partner, or antibody on the bead surface, and if the non-specific protein, peptide, polymer, or small molecule is also covalently labelled or recombinantly tagged with a signal generating molecule, protein, or moiety like fluorescein, a fluoropore, ALP, HRP, chemiluminescence or luminol based substrate (ABEI, luminol, isoluminol, acridinium ester), or Ruthenium, the this can also control or attenuate the signal response per bead. For a secondary coating, this exact same approach can be used but the specific capture moiety, binding partner, or antibody, as well as the non-specific protein or polymer is tagged or labelled such as with biotin, or such as biotin-fluorescein or biotin-signal generating molecule, protein, or moiety, and they are co-coated onto a streptavidin bead surface.

Conjugation reagents for attaching biotin to an antibody or protein can comprise amine-reactive labeling reagents such as, for example, sulfo-NHS-biotin, sulfo-NHS-LC-biotin, sulfo-NHS-LC-LC-biotin, sulfo-NHS-SS-biotin, NHS-PEO4-biotin, NHS-biotin, NHS-LC-biotin, NHS-LC-LC-biotin, PFP-biotin, TFP-PEO-biotin, or NHS-iminobiotin trifluoroacetamide, sulfhydryl-reactive biotin labeling reagents such as, for example, maleimide-PEO2-biotin, biotin-BMCC, PEO-Iodoacetyl biotin, iodoacetyl-LC-biotin, or biotin-HPDP, carboxyl-reactive biotin labeling reagents such as, for example, biotin PEO-amine or biotin PEO-LC-amine, carbohydrate-reactive biotin labeling reagents such as, for example, biocytin hydrazide, biotin hydrazide, or biotin-LC-hydrazide, or photoreactive biotin labeling reagents such as, for example, psoralen-PEO-biotin. Similar chemistries and linkers can be used to conjugate assay signal detecting molecules such as ALP, HRP, luminol, isoluminol, isoluminol derivatives, ABEI, ABEI derivatives, acridinium ester, acridinium ester derivatives, fluorophores, fluorescein and enzymes to proteins and antibodies to prepare the conjugate used in an immunoassay (RIA, ELISA, CLIA, LF, PoC).

In some embodiments, an antibody pair against an antigen or target, such as an antibody pair against the spike proteins of BEI gamma irradiated and inactivated SARS-CoV-2 virions (e.g., Part No. NR-52287; BEI Resources, 10801 University Boulevard, Manassas, VA 20110-2209), or against the spike proteins of SARS-CoV-2 virions in RT-PCR positive saline oral rinse samples, where one antibody is biotinylated and coated on 1.6 micron streptavidin magnetic target capture beads (biotin-labeled humanized monoclonal anti-SARS-CoV-2 spike protein RBD IgG antibody), and the other antibody is conjugated to a fluorescent tag (e.g., iFluor488 or AlexaFluor488 labelled anti-SARS-CoV-2 spike protein NTD antibody), or 70 nm white latex non-magnetic carboxy beads, EDC coated with streptavidin, are coated with a 10:1 mixture of 10 parts biotin-fluorescein to 1 part biotin-labelled humanized monoclonal anti-SARS-CoV-2 spike protein RBD antibody to make “hot” fluorescent beads with a limiting or attenuated amount of anti-RBD antibody per latex beads but with a high relative fluorescence signal (RFU) per latex bead. In the presence of SARS-CoV-2 virions the magnetic target capture beads will capture the virions via binding to the SARS-CoV-2 spike protein RBD, and this magnetic bead-anti-RBD-virion complex will also bind the anti-NTD-Fluorescent conjugate, or the anti-RBD-[latex beads]-fluorescent conjugate. If the magnetic capture beads are separated on a magnet the [anti-RBD magnetic target capture bead]-Virion-[anti-NTD-fluorescent antibody] complex, or [anti-RBD magnetic target capture bead]-Virion-[anti-NTD-antibody fluorescent latex bead] complex, will separate to the magnet whereby no signal or a significantly decreased signal in the supernatant equals a positive result (SARS-CoV-2 virions detected), and no change in RFU signal or no significant decrease in RFU signal equals a negative results (no SARS-CoV-2 virions detected), whereby the supernatant is aspirated and dispensed into a read plate or vessel for fluorescent detection such as by a Agilent BioTek Synergy H1 Multimode Reader.

In some embodiments, to improve detection sensitivity a limiting amount of fluorescent conjugate can be used such that a low viral count or a low viral load (a high RT-PCR cycle threshold (Ct) count such as Ct >36) will deplete RFU signal, but a high viral load (a low Ct value such as Ct<18) will significantly bind all or the majority of the anti-RBD/NTD conjugate to the magnetic beads-virion complex and separate to the magnet with the magnetic bead-virion complex whereby the supernatant RFU signal will significantly decrease or be zero. However, if there is no virus (virions) or very low viral load (Ct >42) then RFU signal of the supernatant will not change or will minimally change for a negative result. One way to prepare the anti-NTD fluorescent conjugate to be limiting, but still have a very good, strong, reproducible signal above background RFU signal, the anti-NTD-Fluorescent antibody conjugate can be serially diluted and the dilution whereby 200 μL of diluted conjugate reads 500-1000 RFUs max can be used for the test. The same approach can be used for the anti-RBD-antibody-[fluorescent latex bead] whereby 200 μL reads 500-1000 RFU. Using this approach the conjugate will be limiting such that a low SARS-CoV-2 viral load (Ct>36) the SARS-CoV-2 virions will bind the majority, if not all, the conjugate and the RFU signal will significantly decrease from 500-1000 RFU to <400 RFU, or preferably to <100 RFU, or more preferably <50 RFU, or most preferably <10 RFU. The magnetic antibody coated target capture beads, coated with anti-RBD antibody, should be in molar excess to bind 95% to 100% of virions in the sample such as adding 80-100 ug (0.08 to 0.10 mg) of target capture beads per 500 uL (0.5 mL) neat saliva or 1000 uL (1.0 mL) SOR. If the target capture beads are in binding, capture, or molar SARS-CoV-2 virion excess, but the conjugate is limiting, a fluorescent test, a visual test, or a UV-vis O.D. 600 nm test, will show a significant decrease in RFU signal, decrease in latex beads, or decrease in O.D. 600 absorbance with SARS-CoV-2 positive samples.

In some embodiments, the capture moiety can bind to a biomarker by a cleavable bond. The cleavable bond can comprise covalent or non-covalent binding. Non-covalent binding can comprise affinity, ionic, van der Waals (e.g., dipole/dipole or London forces), hydrogen bonding (e.g., between polynucleotide duplexes), or hydrophobic interactions. In some embodiments, the non-covalent binding can be specific. In some embodiments, specific non-covalent binding can comprise binding interaction between biotin and a biotin-binding protein such as avidin, captavidin, SA, neutravidin, a fragment of SA, a fragment of avidin, a fragment of neutravidin, or mixtures thereof; the binding of a biotinylated Fab, a biotinylated immunoglobulin or fragment thereof, a biotinylated small molecule (such as, for example, a hormone or a ligand of a receptor), a biotinylated polynucleotide, a biotinylated macromolecule (e.g., a protein or a natural or synthetic polymer) to a biotin-binding protein such as avidin, SA, neutravidin, a fragment of SA, a fragment of avidin, a fragment of neutravidin, or mixtures thereof; the binding of a substrate to its enzyme; the binding of a glycoprotein to a lectin specific for the glycoprotein; the binding of a ligand to a receptor specific for the ligand; the binding of an antibody to an antigen against which the antibody is raised; and duplex formation between a polynucleotide and a complementary or substantially complementary polynucleotide; etc.

In some embodiments, the biomarker capture particles can comprise a coating that may interact with a capture moiety that is added to or present in a sample. In some embodiments, the capture moiety that is added to or present in a sample can bind to a biomarker in the sample to form a biomarker-capture moiety complex and when the biomarker capture particles are introduced, the biomarker capture particles can interact with the biomarker-capture moiety complex to capture the biomarker-capture moiety. In some embodiments, the coating can comprise a streptavidin coating. In some embodiments, the capture moiety is added to or combined with a sample, without capture particles and without being conjugated to a capture particle. In some embodiments, the capture moiety is added to or combined with the sample with capture particles, where the capture moiety is conjugated to the capture particle.

### Preparation

In some embodiments, step (b) can further comprise incubating the sample comprising the biomarker capture particles for a period of time from 5 min to 24 hours. In some embodiments, the incubation time can be, for example 5 min, 10 min, 15 min, 30 min, 60 min, 2 hours, 4 hours, 8 hours, or overnight. In some embodiments, the incubation can be performed during shipment or transit of the sample. In some embodiments, the incubation can be performed at room temperature or ambient temperature, at 2-8° C. (e.g., in a cooler, or on cold packs), or at heated temperature such as from 30 to 50° C. (e.g., 30, 37, or 42° C.) with or without mixing or agitation such as on a rocker, nutator, bottle roller, shaker, or plate mixer.

In some embodiments, the methods described herein can be performed in less than 1 week, 6 days, 5 days, 4 days, 3 days, 2 days, 1 day, 24 hours, 20 hours, 16 hours, 12 hours, 8 hours, 4 hours, 2 hours, 1 hour, 30 minutes, 15 minutes, 10 minutes, 5 minutes or less. In some embodiments, the methods described herein can be performed in less than 1 day.

In some embodiments, the method can further comprise removing liquid phase from the sample subsequent to step (b). In some embodiments, removing the liquid phase can be performed after separating the liquid phase from the biomarker capture particles. A separation can comprise centrifuge or filtration. In some embodiments, a magnetic field or a magnet can be applied to attract the biomarker capture particles thereby separating the biomarker capture particles from the liquid phase. In some embodiments, a wash solution can be dispensed to the sample or the biomarker capture particles to remove or elute un-specifically bound substances (a “clean” or “cleaning” process). To isolate or separate biomarker capture particles (e.g., within a primary blood collection tube, custom sample collection device, secondary transfer tube or custom sample device, pooled samples, or 96-well plate) a magnet-based device will quickly (less than 2 minutes; preferably less than 30 seconds) isolate the magnetic nanoparticles to the side(s) and/or bottom to form an essentially particle-free supernatant. The particle-free supernatant can be subsequently aspirated, drained, or otherwise removed without disrupting the pellet comprising the biomarker capture particles. In some embodiments, the pellet can be isolated or subjected to diagnostic testing. Another approach to isolate or separate magnetic particles is to use a disposable pipette tip comprising a custom magnet inserted inside the disposable tip to quickly isolate the magnetic nanoparticles to the surface of the pipette tip to form an essentially particle-free sample supernatant. The disposable pipette tip with custom magnet can subsequently be removed from the sample without disrupting the pellet comprising the particles. The disposable tip can be inserted into a new tube for isolation and characterization of the particles in a subsequent diagnostic test (i.e., enrichment). For example, the disposable tip with particles can be inserted into a secondary transfer tube containing a buffer. If the magnet is removed from the tip, or if the magnet is turned off (e.g., electromagnet) the particles are free to disperse into the buffer.

In some embodiments, the method can further comprise eluting or cleaving the biomarker from the biomarker capture particles (a “biomarker cleave process” or “biomarker cleaving process”). In some embodiments, the eluted biomarker can comprise the capture moiety bound/coupled with the biomarker, i.e., the capture moiety and biomarker pair is eluted from the biomarker capture particles. In some embodiments, elution may be accomplished after removing the liquid phase. Eluting the biomarker from the biomarker capture particles may be accomplished, for example, by adding an elution solution or cleavage reagent to the biomarker capture particles. The elution solution or cleavage reagent can cleave or elute the captured biomarkers off the biomarker capture particles. After an incubation time, the biomarker capture particles can be removed from the eluate by centrifugation, magnetic centrifugation, or filtration. The eluate can be measured by mass spectrometry (e.g., LC-MS, LC-MS/MS, MALDI-TOF, etc.), ELISA, CLIA, FIA, PCR, NGS, Antibody-Oligo Conjugates in Immuno-PCR or any measurement method or system for a presence, concentration, and/or level of a biomarker. In some embodiments, a neutralizing buffer can be added to the eluate prior to the measurement.

In some embodiments, the biomarker is eluted, disassociated or freed from the biomarker capture particles (e.g., nanoparticle, microparticle) by the elution solution or cleavage reagent by disrupting the binding interaction using elution strategies such as pH. In some embodiments, elution can be performed by increasing pH with a base such as sodium bicarbonate. In some embodiments, elution can be performed by decreasing pH with an acid such as acetic acid, trichloroacetic acid, sulfosalicylic acid, HCl, or formic acid. In some embodiments, the elution solution can comprise pH elution buffers such as 100 mM glycine·HCl with pH 2.5-3.0, 100 mM citric acid with pH 3.0, 50-100 mM triethylamine or triethanolamine, pH 11.5, 150 mM ammonium hydroxide, pH 10.5. In some embodiments, the elution can be performed with a displacer or displacing agent, competitive elution (e.g. >0.1M counter ligand or analog), ionic strength and/or chaotropic effects (e.g., NaCl, KCl, 3.5-4.0M magnesium chloride pH 7.0 in 10 mM Tris, 5M lithium chloride in 10 mM phosphate buffer pH 7.2, 2.5M sodium iodide pH 7.5, 0.2-3.0M sodium thiocyanate), surfactant, detergent, a concentrated inorganic salt, denaturing (e.g., 2-6 M guanidine·HCl, 2-8M urea, 1% deoxycholate, 1% SDS), an organic solvent (e.g. alcohol, chloroform, ethanol, methanol, acetonitrile, hexane, DMSO, 10% dioxane, 50% ethylene glycol pH 8-11.5 (also chaotropic)), radiation or heat (increased temperature), conformational change, disulfide bond reducers (2-mercaptoethanol, dithiothreitol, tris(2-carboxylethyl)phosphine), enzyme inactivation, chaotropic agents (Urea, Guanidinium chloride, Lithium perchlorate), mechanical agitation, sonication, and protein digestive enzymes (pepsin, trypsin), and combinations thereof.

In some embodiments, a biomarker capture particle may be trapped on or by the filter. A collection device with neutralizing buffer can be attached to the filter. An elution buffer can be used to rinse and elute the capture biomarkers to the collection device for neutralization.

In some embodiments, after the cleaning process, the biomarker is not subjected to a biomarker cleaving process to cleave the biomarker off the biomarker capture particles, and alternatively, the method can further comprise adding a buffer to the biomarker capture particles comprising the biomarkers after the liquid phase is removed, thereby generating a buffer solution comprising the biomarker capture particles (a “concentrate” or “concentrating” process). In some embodiments, a volume of the buffer can be less than a volume of the sample. In some embodiments, a volume of the buffer can be substantially less than a volume of the sample.

The biomarker capture particles can be dispersed, reconstituted or resuspended in a buffer such as phosphate buffered saline (i.e., PBS pH 7.2), or LC-MS/MS compatible buffer, prior to the characterization or measurement step. This means the key characterization or measurement step of the captured and enriched biomarkers by the particles occurs in a buffer system and not in the animal or human matrix.

In some embodiments, the method can further comprise, subsequent to the concentrating process, adding a plurality of conjugates to the buffer solution comprising the biomarker capture particles (a “conjugate” or “conjugating” process). To detect, measure, or quantitate one or more biomarkers on the biomarker capture particles a conjugate can be added which will bind to the captured biomarker on the biomarker capture particles. The conjugate can be specific to a single biomarker, or the conjugate can comprise 2 or more antibodies or antigens specific to one captured biomarker, or specific to two or more captured biomarkers. The conjugate can be labelled with a signal detection moiety, or 2 or more different conjugates can be labelled using different fluorophores (different emissions/excitations) and pooled to make a multiplex conjugate. In some embodiments, a conjugate can react or bind to a biomarker captured on the biomarker capture bead. Non-limiting examples of conjugates can comprise a chemiluminescent substrate (isoluminol, luminol, ABEI, ruthenium, acridenium ester), a fluorescent label (fluorescein or other fluorophores and dyes), anti-alkaline phosphatase (ALP), anti-horseradish peroxidase (HRP), a smaller magnetic bead, a smaller non-magnetic bead, or smaller non-magnetic colored beads.

In some embodiments, the conjugate can be a triplex conjugate comprising rabbit anti-human IgA, rabbit anti-human IgG, and rabbit anti-human IgM (Agilent DAKO, 5301 Stevens Creek Blvd. Santa Clara, CA 95051) each labeled with a different fluorophore such as AlexaFluor® (ThermoFisher Scientific, 168 Third Avenue. Waltham, MA USA 02451) 488, 555, or 647, or iFluor® (AAT Bioquest, Inc., 520 Mercury Drive, Sunnyvale, CA 94085, USA) 488, 546, and 597. In some embodiments, the conjugate can be a 5-plex conjugate using 5 different iFluors with monoclonal anti-human IgA (total), and IgG subclasses IgG1, IgG2, IgG3, and IgG4, and/or IgM (Mabtech AB, Box 1233 SE-131 28, Nacka Strand, Sweden). By detecting 2 or more different human immunoglobulin classes or immunoglobulin subclasses, it can improve sensitivity of detecting a positive immune response against a pathogen (bacteria, virus, or fungus) or antigen, as well as antibody profiling to stage the progression of a disease (acute or early infection vs. chronic or late infection). For example, if IgM is detected, it typically can indicate early or acute infection; if IgG is detected, it typically can indicate after seroconversion and is representative of late or chronic infection; and if secretory IgA in saliva or SOR is detected, it can indicate early or acute respiratory pathogen infection as the first line of defense for protective immunity or prior infection or vaccination.

In some embodiments, biomarker capture particles can be coated with different recombinant or purified antigens or proteins for different pathogens, viruses, bacteria, or fungi species, and the conjugate may comprise the exact same recombinant or purified antigens coated on the biomarker capture particles, but each are labeled or conjugated with a different fluorophore for multiplex detection. Since human IgG (bivalent, or 2 Fabs), IgA (tetravalent, or 4 Fabs), and IgM (decavalent, or 10 Fabs) are multivalent, they can bind to antigens they recognize or detect on the biomarker capture particles but also bind to the exact same antigens labeled with fluorophores. This approach enables multiplex detection of one or more different pathogens, viruses, bacteria, or fungi, such as a tick-borne illness panel, infectious disease panel, or sexually transmitted infection panel (e.g., HIV, Hepatitis A, B, and C, HSV 1 and 2, HPV, etc.) where one or more different antigens or proteins for each pathogen is used for both the antigen coated biomarker capture particles and the fluorescently labelled conjugate.

In some embodiments, the conjugate can be cleaned prior to adding to the buffer solution comprising the biomarker capture particles. In some embodiments, the cleaning can be done with biomarker capture particles. In some embodiments, the cleaning can be done with interference capture particles. The cleaning can be important because some conjugates or polyclonal antibodies may non-specifically bind to or cross-react with the biomarker capture particles, capture moieties, or blockers, especially if human or animal derived or origin, causing high background signal or noise in subsequent detections/measurements. By exposing or pre-incubating the conjugate with the biomarker capture particles and removing the biomarker capture particles from the conjugate via filtration, magnetic separation, or centrifugation, a portion of the conjugate that may react with the biomarker capture particles is removed, therefore, when the conjugates are used, the background signal will be mitigated or significantly reduced thereby increasing assay or test signal-to-noise and sensitivity. Small to large quantities of biomarker capture particles can be added to the conjugate to clean the conjugate of biomarker capture particle-specific interference. In some embodiments, the amount of biomarker capture particles for conjugate cleaning can comprise, for example, 0.01 μg (0.00001 mg), 0.1 μg (0.0001 mg), 1.0 μg (0.001 mg), 10 μg (0.01 mg), or 100 μg (0.1 mg) biomarker capture particles per mL of conjugate, or preferably >1.0 μg (0.001 mg) biomarker capture particles per mL of conjugate, or most preferably >3.0 μg (0.003 mg) biomarker capture particles per mL of conjugate.

In some embodiments, after the conjugates are added to the buffer solution comprising the biomarker capture particles, the solution can be incubated for a period of time, for example, from 5 min to 24 hours. In some embodiments, the incubation time can be, for example 5 min, 10 min, 15 min, 30 min, 60 min, 2 hours, 4 hours, 8 hours, or overnight. In some embodiments, the incubation can be performed during shipment or transit of the sample. In some embodiments, the incubation can be performed at room temperature or ambient temperature, at 2-8° C. (e.g., in a cooler, or on cold packs), or at heated temperature such as from 30 to 50° C. (e.g., 30, 37, or 42° C.) with or without mixing or agitation such as on a rocker, nutator, bottle roller, shaker, or plate mixer.

In some embodiments, the method can further comprise removing excess conjugates. In some embodiments, the removing can comprise separating the biomarker capture particles from the liquid phase comprising the buffer solution and the excess conjugates. The separation can be performed by centrifugation or filtration. In some embodiments, magnetic field can be applied during the separation and/or removing.

In some embodiments, the biomarker capture particles can be subjecting to additional cleaning by adding a cleaning buffer to the biomarker capture particles, incubation, and separation.

In some embodiments, the method can further comprise adding an elution buffer to the biomarker capture particles to elute or cleave conjugates from the biomarker capture particles (a “conjugate cleave process” or “conjugate cleaving process”). In some embodiments, the solution can be incubated for a period of time for 1 min to 2 hours. In some embodiments, the incubation period can be, for example, 1 min, 2 min, 3 min, 4 min, 5 min, 10 min, 15 min, 30 min, 60 min, or 2 hours. In some embodiments, the elution can be performed with an acidic elution and incubation for 5 minutes or less, or preferably 2 minutes or less, prior to the subsequent neutralization of the eluate sample pH with a neutralization buffer such as to add 35 μL Neutralization Buffer (e.g., 300 mM TRIS pH 10.0) to 220 μL Elution Buffer (e.g., 100 mM glycine, 0.05% (w/v) Tween-20, pH 2.5) to neutralize the eluate sample pH to 7.2-7.5. For example, after the biomarker capture particles (e.g., capture beads) are washed, e.g., 2-4 times with the wash buffer, the captured biomarker(s) can be eluted from the biomarker capture particles using an acidic elution buffer (e.g., 220 μL 100 mM Glycine pH 2.5, 0.05% Tween-20, or 180 μL 40 mM Acetic Acid, 0.05% Tween-20, pH 3.05), and neutralized with a neutralization buffer (e.g., 35 μL 300 mM TRIS pH 10.0, or 26 μL 300 mM TRIS pH 10.5, 0.05% Tween-20) such that the biomarkers are purified into a final matrix-free buffer with a neutral pH from 7.0 to 8.0.

In some embodiments, subsequent to the elution or cleavage of conjugates from the biomarker capture particles, the biomarker capture particles can be removed from the eluted solution. The eluted solution comprising the eluted conjugates can be subsequently subject to a detection, measurement, and/or characterization (a “characterize” or “characterizing” process). In some embodiments, the detection can comprise a fluorescent detection or a microscopic detection. In some embodiments, a neutralization buffer can be added to the eluted solution prior to the detection.

In some embodiments, the sample can comprise an unprocessed sample, for example, a sample during sample collection. The sample can be collected in a sample collection device such as a serum/plasma primary blood collection tube, a saliva collection tube, a saline oral rinse collection tube, a stool collection device, or a urine collection device. Subsequent to the sample collection, a plurality of biomarker capture particles can be added to the sample. This allows the entire sample collected to be treated and exposed to the biomarker capture particles to (i) maximize capture efficiency and recovery/yield of biomarkers in the sample, especially if they are low abundance biomarkers, or (ii) to liberate, release, lyse, or cleave the sample in the presence of the biomarker capture particles to maximize the recovery of the biomarkers, especially for low abundance biomarkers or biomarkers that may otherwise be lost to the sample collection tube or device surface such as via hydrophobic, ionic, or other non-specific binding (NSB) mechanisms, or lost to other sample constituents such as other proteins, lipids, triglycerides, or molecules hydrophobically or via other binding mechanisms, or lost via susceptibility to protease or enzymatic digestion or degradation when free in solution and not bound or protected by a binding partner.

In some embodiments, a sample, a biomarker capture particle, a detection antibody, or a combination thereof can be pre-treated to remove one or more interferences from the sample, the biomarker capture particle, the detection antibody, or the combination thereof. In some embodiments, pre-treating can occur before capturing by the biomarker capture particles (a “condition” or “conditioning” process). In some embodiments, the removal of the one or more interferences can be performed prior to step (b).

In some embodiments, interferences can comprise one or more lipids, triglycerides, bilirubin, hemolysis products (e.g., hemoglobin, enzyme, or potassium), cholesterol, human anti-mouse antibodies (HAMA), rheumatoid interference (RF), manufacture assay specific interference (MASI), human anti-animal antibody (HAAA) interferences such as mouse, goat, sheep, rabbit, and bovine immunoglobulins, free biotin interference, anti-streptavidin, anti-biotin interference, human anti-polyethylene glycol (PEG) or anti-polyethylene oxide (PEO) interference, anti-albumin, anti-histidine, anti-polyhistidine tag (e.g., 6-his tag, 8-his tag), non-specific binding, anti-polyvinylpyrrolidone (PVP), anti-polymer, anti-alkaline phosphatase (ALP), anti-ruthenium, anti-fluorescein, anti-acridinium ester (includes ABEI, luminol, isoluminol), autoantibodies, anti-horse radish peroxidase (HRP), anti-conjugation linkers such as LC, LC-LC, and PEOn, anti-amino acid tags, anti-polyhistidine-tags, over-the-counter (OTC) supplements, herbal remedies, and/or therapeutics, or any combination thereof. In some embodiments, hemolysis can comprise a rupturing (lysis) of red blood cells (erythrocytes) and the release of their contents (cytoplasm) into surrounding fluid (e.g., blood plasma). In some embodiments, hemolysis products that are problematic and can cause interference and false results in chemistry and immunoassay tests can comprise hemoglobin, potassium, enzyme, e.g., lactate dehydrogenase (LDH), and/or fluid. Interference due to hemolysis can be caused by the release of intracellular material, which falsely elevates serum/plasma concentrations of certain analytes, such as potassium and lactate dehydrogenase, while diluting others, like sodium. Potassium, LDH, AST, magnesium or phosphorus are the parameters that have the greatest difference between the intracellular medium of the RBC and the extracellular medium showing significant interference already at low hemolysis levels. Human anti-animal antibody (HAAA) interference is primarily attributed to human heterophilic antibodies specific to bovine, goat, mouse, rabbit, or sheep IgG commonly used in immunoassays. In some embodiments, rheumatoid factor (RF) interference can be specific to mouse IgG and Fc portion of antibodies used in immunoassays, and manufacture assay specific interference (MASI) is primarily attributed to patient specific interference directly or indirectly (i.e., antibodies against) from diet, nutritional supplements, medications or medical therapy/treatment that bind or interact with immunoassay critical raw materials. In some embodiments, interferences can comprise small molecules or analogues of such small molecules.

In some embodiments, removing the one or more interferences can comprise contacting the sample with interference capture particles.

In some embodiments, interference capture particles can comprise an interference capture moiety that will interact with a sample interference such that the interference, when exposed to the interference capture particle, will interact and bind to the particle surface. In some embodiments, the capture moiety can interact with the interference via a specific binding. In some embodiments, the capture moiety can interact with the interference via a non-specific binding.

In some embodiments, the interference capture particles can be stored in a storage diluent or buffer. The storage diluent or buffer can comprise constituents or ingredients such as chemicals, salts, buffers to raise or lower sample pH, detergents, surfactants, lysis agents, polymers, proteins, peptides, or blockers. In some embodiments, the interference capture particle can be added to the sample with the storage diluent or buffer.

In some embodiments, the interference capture particles can be isolated or removed from the sample via magnetic centrifugation, centrifugation, or filtration. In some embodiments, the substantially particle-free sample can have a significantly lower level, concentration, threshold, or titer of the interferences such that it will no longer interfere with a test, a measurement or a characterization of the sample. In some embodiments, interference capture moieties on the interference capture particles can comprise human immunoglobulins (e.g., IgA, IgG, IgM, or IgE) which can target autoantibody interferences. In some embodiments, interference capture moieties can comprise animal (e.g., mouse, goat, sheep, rabbit, cow or bovine, lama, or alpaca) antibodies which can target heterophilic interferences such as HAMA, RF, and HAAA. In some embodiments, interference capture moieties can comprise polymerized antibodies or mouse antibody fragments (Fc, Fab, F(ab′)2) which can target HAMA and RF interferences. In some embodiments, interference capture moieties can comprise proteins, enzymes, or small molecules (e.g., ALP, HRP, fluorescein/fluorophores, luminol, isoluminol, acridinium ester, ABEI, ruthenium, or luciferin) that can target anti-signal generating interference. In some embodiments, interference capture moieties can comprise streptavidin, avidin, or neutravidin which can target biotin interference or biotin metabolite interference (e.g., bisnorbiotin or biotin sulfoxide). In some embodiments, interference capture moieties can comprise polyhistidine that can target ant-histidine or anti-polyhis tag interference, or PEG or PEO that can target anti-PEG or anti-PEO interference. In some embodiments, interference capture moieties can comprise antibodies, aptamers, antibody fragments, MIPs, or polymers that can target specific interferences via epitope binding such as bilirubin, albumin, lipids, triglycerides, cholesterol, icterus (e.g., bile pigments), hemoglobin, herbal (e.g., hemolysis products, lipemia, icterus, tube additives, radioactive or fluorescent compounds, drugs, herbal medicines, and nutritional supplements). In some embodiments, the interference capture moieties can comprise one or more interference capture moieties that can target one or more types, classes, or groups of interferences disclosed herein.

In some embodiments, the method can comprise conditioning the sample. In some embodiments, the conditioning can be performed prior to removing the one or more interferences or subsequent to removing the one or more interferences. In some embodiments, the conditioning can comprise modifying a chemical or physical properties of the sample. In some embodiments, the chemical or physical property can comprise temperature, pH, color, salinity, conductivity, density, viscosity, surface tension, or protein content. In some embodiments, the conditioning can comprise introducing to the sample surfactants, detergents, cell lysis agents, anti-protease agents, protein-based or polymeric-based blocking reagents, displacing agents, or agents to release analytes or biomarkers from the matrix or to remove interfering elements.

In some embodiments, the cell lysis agent can comprise RIPA buffer to lyse cells such as exosomes, extracellular particles, virions, or bacterium. The lysis agent is compatible with the interference capture particles and does not damage or impair antibody or capture moiety activity or target binding. A pre-analytical lysis step can liberate or release biomarkers from a cell such as exosomes for subsequent binding, capturing, purification, detection, and/or measurement.

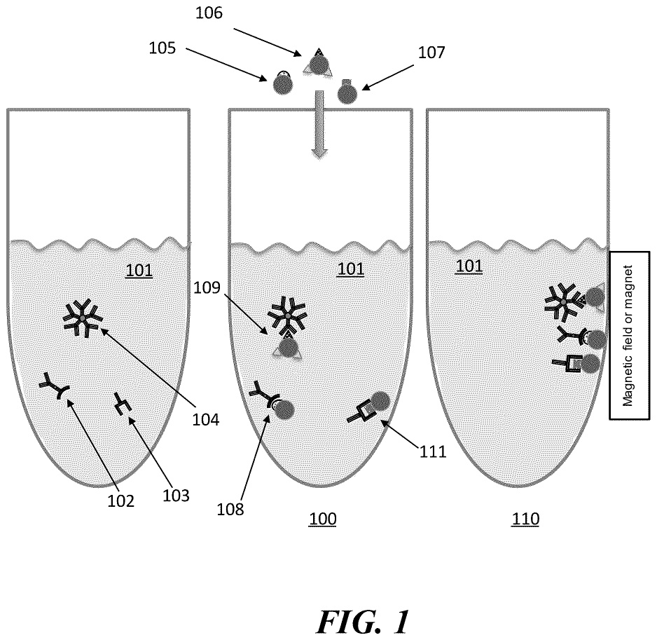

FIG. 1 shows an exemplary sample preparation method. A sample 101 can comprise a plurality of biomarkers, e.g., biomarkers 102, 103, and 104, wherein the plurality of biomarkers is of different types. For example, the plurality of biomarkers can comprise an antigen, an antibody, any other types of biomarkers, or any combination thereof. At operation 100, a plurality of biomarker capture particles, e.g., biomarker capture particles 105, 106, and 107, are introduced to the sample 101. After an incubation time, biomarker capture particles 105, 106, and 107 capture biomarkers 102, 103, and 104 to form a plurality of complexes 108, 109 and 111. At operation 110, a magnetic field or a magnet is applied to attract the complexes 108, 109 and 111.

The liquid phase of the sample 101 can be removed or discarded. An elution solution can be subsequently added to the plurality of complexes to elute or cleave the plurality of biomarkers off the plurality of biomarker capture particles. The eluate comprising the eluted plurality of biomarkers can be subject to detection or measurement. In some embodiments, a neutralizing buffer can be added to the eluate prior to any detection or measurement.

In some embodiments, after removing the liquid phase of the sample 101, a plurality of conjugates can be added to the plurality of complexes to conjugate with the captured biomarkers. An elution solution can be subsequently added to the plurality of complexes comprising the plurality of biomarkers and the plurality of conjugates to elute or cleave the plurality of conjugates off the plurality of biomarker capture particles. The eluate comprising the eluted plurality of conjugates can be subject to detection or measurement. In some embodiments, a neutralizing buffer can be added to the eluate prior to any detection or measurement.

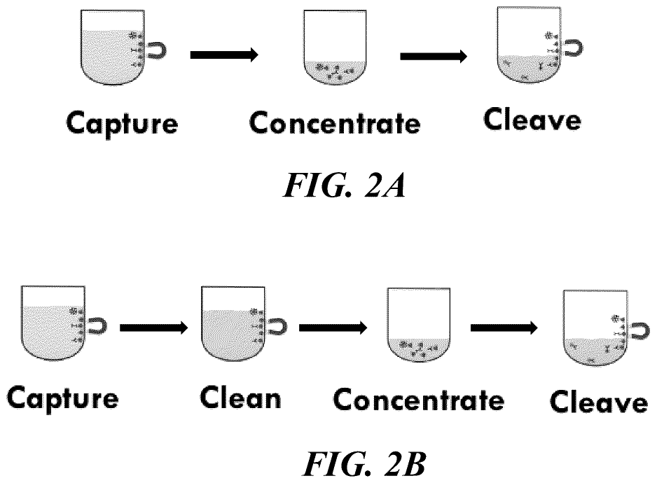

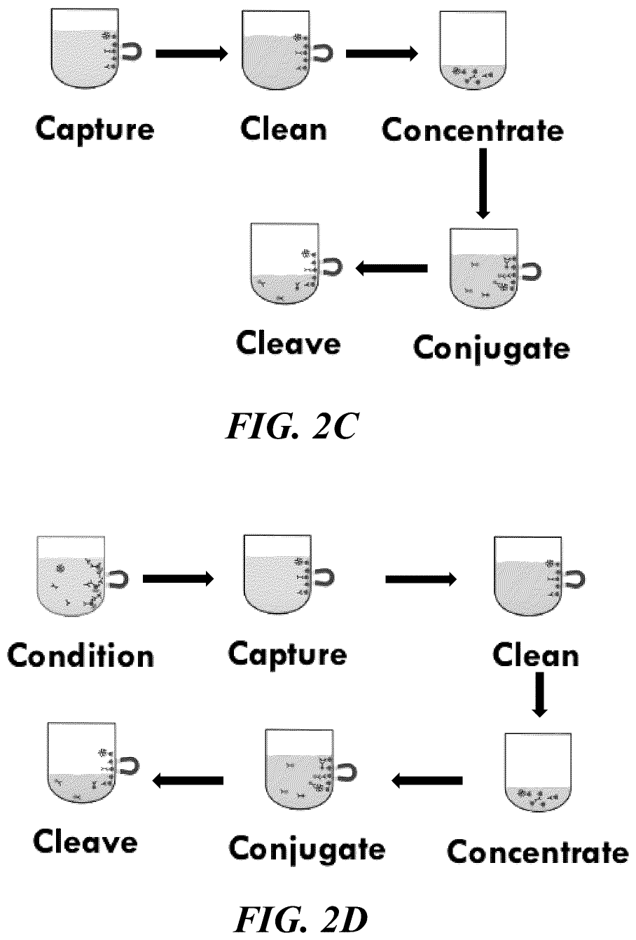

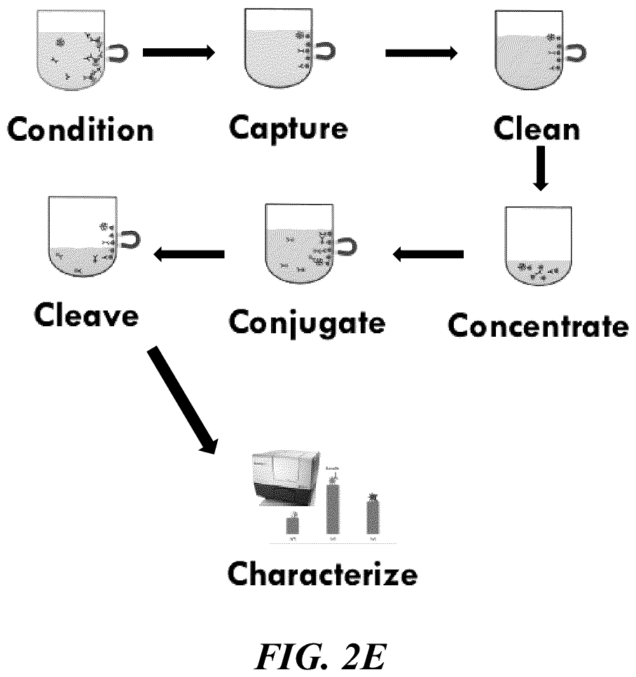

FIG. 2A shows an exemplary method for a biomarker detection and/measurement comprising capturing, concentrating, and biomarker cleaving. FIG. 2B shows an exemplary method for a biomarker detection and/measurement comprising capturing, cleaning, concentrating, and biomarker cleaving. FIG. 2C shows an exemplary method for a biomarker detection and/measurement comprising capturing, cleaning, concentrating, conjugating, and conjugate cleaving. FIG. 2D shows an exemplary method for a biomarker detection and/measurement comprising conditioning, capturing, cleaning, concentrating, conjugating, and conjugate cleaving. The methods disclosed in FIG. 2A-2D can further comprise a characterization process. For example, FIG. 2E shows an exemplary method for a biomarker detection and/measurement comprising conditioning, capturing, cleaning, concentrating, conjugating, conjugate cleaving, and characterizing. Process as shown in FIG. 2E can be referred as “7C” process.

In some embodiments, the method can further comprise binding the captured biomarker with a detection antibody. In some embodiments, the method can further comprise measuring the detection antibody. In some embodiments, the method can further comprise comparing the detection antibody to a standard curve. In some embodiments, the standard curve is generated from biomarker capture particles bound to known amounts of the biomarker.

In some embodiments, the method can further comprise cleaning the detection antibody with the interference capture particles or the biomarker capture particles.

In some embodiments, the detection antibody can comprise an anti-human antibody. In some embodiments, the detection antibody can comprise an antibody against the antigen.

In some embodiments, the detection antibody can be conjugated to a detection reagent. In some embodiments, the detection reagent can comprise an enzyme or label. In some embodiments, the label can comprise a fluorescent tag. Non-limiting examples of the label can comprise an enzyme, protein, or small molecule such as ALP, HRP, isoluminol, luminol, acridinium, ABEI, ruthenium, or Antibody-Oligo Conjugates in Immuno-PCR, or luciferin for ELISA (Enzyme linked immunoassay), CLIA (chemiluminescence immunoassay), or FIA (fluorescence immunoassay).

In some embodiments, the method can further comprise multiplexing the capture particles with additional capture particles comprising a second antigen, and wherein the biomarker comprises an antibody that binds to the second antigen. In some embodiments, quantifying the biomarker can comprise multiplexing the detection antibody with a second detection antibody that recognizes the second antigen. In some embodiments, the method can further comprise multiplexing the capture particles with additional capture particles comprising a third antigen, and wherein the biomarker comprises an antibody that binds to the third antigen. In some embodiments, quantifying the biomarker can comprise multiplexing the detection antibody with a third detection antibody that recognizes the third antigen.

In some embodiments, the method can further comprise multiplexing the detection antibody with an additional detection antibody that recognizes a biomarker in the sample.

In some embodiments, the method can further comprise monitoring the biomarker over time to determine an increase or decrease in an amount of biomarker in a second sample of the subject, relative to the quantified biomarker.

In some embodiments, a biomarker capture particle can comprise a plurality of capture moieties. In some embodiments, the plurality of capture moieties can comprise different types of capture moieties, e.g., antigens, antibodies, any other types of capture moieties, or any combination thereof, thereby allowing for a multiplex or a combo detection.

In some embodiments, a biomarker capture particle can comprise more than one recombinant antigen, or viral or bacterial lysate to improve the likelihood and sensitivity of detecting human immunoglobulins such as IgA, IgG, IgM, and IgE, immunoglobulin subclasses, or a combination thereof against the antigen or antigens.

In some embodiments, a biomarker capture particle can comprise more than one antibody to improve the likelihood and sensitivity of detecting an antigen or biomarker, or for multiplex detection of more than one antigen or biomarker. This is antigen testing and may, for example, be performed by a sandwich assay, completive assay, piggyback assay, or delayed addition or delayed capture assay.

### Detection

In some embodiments, a detection and/or measurement of an eluted biomarker or conjugate can be performed with any suitable detection and/or measurement method, for example, ELISA, CLIA, FIA, lateral flow, POCT, microarray, lab on a chip, HPLC (e.g., reverse-phase, normal-phase, ion-exchange/anion-exchange/cation-exchange, hydrophobic interaction (HIC), hydrophilic interaction, partition, displacement, size-exclusion, and affinity chromatography using either isocratic or gradient elution), mass spectrometry (e.g., LCMS, LC-MS/MS, MALDI-TOF), molecular (e.g., PCR, RT-PCR, nucleic acid amplification test, next generation sequencing (NGS)), and chemistry or bead-based agglutination such as particle-enhanced turbidimetric immunoassay (PETIA). The methods and compositions of the present disclosure can be used in conjunction with any suitable assay, for example, any suitable affinity assay or immunoassay comprising, but not limited to, protein-protein affinity assays, protein-ligand affinity assays, nucleic acid affinity assays, indirect fluorescent antibody assays (IFAS), enzyme-linked immunosorbant assays (ELISAs), radioimmunoassays (RIAs), and enzyme immunoassays (EIAs), direct or indirect assays, competitive assays, sandwich assays, Antibody-Oligo Conjugates in Immuno-PCR, homogeneous or proximity based assays, etc.

In some embodiments, a concentration of the eluted biomarker or conjugate in the sample can be determined by use of a bead-based calibration curve. The calibration curve can be generated using two to ten different calibrator beads with different known amounts of purified biomarker or conjugate captured or conjugated to each calibrator bead. After cleaving/eluting from the calibrator beads, the signal detected is corresponding, e.g., proportional to the known amount of purified biomarker or conjugate from each calibrator bead. When an unknown sample is detected, the concentration can be calculated based on the calibration curve and the detected signal. For example, the calibration curve can be generated using seven different calibrator beads with different amounts of purified human IgG conjugated to each calibrator bead. When testing with fluorescence, the relative fluorescence signal is directly proportional to the amount of antigen-specific IgG immunoglobulins captured by the beads.

In some embodiments, the calibrator beads can comprise more than one type of biomarkers for multiplex calibration. For example, the calibration curve can be generated using seven different triplex calibrator beads with different amounts of purified human IgM, IgG and IgA conjugated to each calibrator bead. When testing with fluorescence, the relative fluorescence signals of each fluorophore are directly proportional to the amount of antigen specific IgG, IgM and/or IgA immunoglobulins captured by the beads.

### Sample Preparation

Samples can be collected into in a primary blood collection tube (PBCT), secondary transfer tube (SST), 24-hour (24-hr) urine collection device, a saliva collection tube, blood spot filter paper, or any collection tube or device such as for stool and seminal fluid, a light green top or green top plasma separator tube (PST) containing sodium heparin, lithium heparin or ammonium heparin, a light blue top tube containing sodium citrate (i.e. 3.2% or 3.8%) or citrate, theophylline, adenosine, dipyridamole (CTAD), a red top tube for Serology or Immunohematology for the collection of serum in a glass (no additives) or plastic tube (contains clot activators), a red top tube for Chemistry for the collection of serum in a glass (no additives) or plastic tube (contains clot activators), a purple lavender top tube containing EDTA K2, EDTA K3, liquid EDTA solution (i.e. 8%), or EDTA K2/gel tubes for testing plasma in molecular diagnostics and viral load detection, a pink top tube for Blood Bank EDTA, a gray top tube containing potassium oxalate and sodium fluoride, sodium fluoride/EDTA, or sodium fluoride (no anticoagulant, will result in a serum sample), a yellow top tube containing ACD solution A or ACD solution B, a royal blue top (serum, no additive or sodium heparin), a white top tube, or any color or tube type, for any application or diagnostic test type, containing no additives or any additive or combinations thereof, for the collection of blood.

A primary blood collection tube (PBCT) and secondary transfer tube (SST) can be any commercially available standard or custom collection tube (with or without gel separators) from companies like Becton Dickinson (BD), Greiner, VWR, and Sigma Aldrich, a glass tube, a plastic tube, a light green top or green top plasma separator tube (PST) containing sodium heparin, lithium heparin or ammonium heparin, light blue top tube containing sodium citrate (i.e. 3.2% or 3.8%) or citrate, theophylline, adenosine, dipyridamole (CTAD), red top tube for Serology or Immunohematology for the collection of serum in a glass (no additives) or plastic tube (contains clot activators), a red top tube for Chemistry for the collection of serum in a glass (no additives) or plastic tube (contains clot activators), a purple lavender top tube containing EDTA K2, EDTA K3, liquid EDTA solution (i.e. 8%), or EDTA K2/gel tubes for testing plasma in molecular diagnostics and viral load detection, a pink top tube for Blood Bank EDTA, a gray top tube containing potassium oxalate and sodium fluoride, sodium fluoride/EDTA, or sodium fluoride (no anticoagulant, will result in a serum sample), a yellow top tube containing ACD solution A or ACD solution B, a royal blue top (serum, no additive or sodium heparin), a white top tube, or any color or tube type, for any application or diagnostic test type, containing no additives or any additive or combinations thereof, for the collection of blood.

A saliva collection tube (neat saliva, drool, spit, sputum) or saline oral rinse (SOR) collection tube (such as 3-5 mL 0.9% sodium chloride, or phosphate buffered saline, in purified water) can be any commercially available standard or custom collection tube (with or without preservatives) from companies such as Abclonal, Canvax Biotech, CD Genomics, DNA Genotek, Eagle Biosciences, IBI Scientific, Oasis Diagnostics, Omni International, Porex Life Sciences Institute & AG Industries, Ray Biotech, Salimetrics, Sarstedt, Stellar Scientific, Spectrum Solution, ThermoFisher, Thomas Scientific, and Zymo Research where the preservative is already in the saliva or SOR collection tube as a liquid, or as a spray dried reagent, or as a lyophilized reagent prior to saliva collection, or the preservative is added to the saliva sample after saliva or SOR collection such as by a collection tube cap, whereby the cap comprises the preservative reagent, comprising a reagent release mechanism whereby a membrane or barrier is broken when the cap is placed onto the collection tube or screwed onto the collection tube, or the preservative is added to the collected saliva or SOR sample by pouring, dispensing, dropping, or mixing with an exogenous preservative reagent. Saliva or SOR samples can be collected in a collection tube and subsequently filtered into a filtrate tube using a filter, or more than one filter, whereby each filter has the same or different porosity or molecular weight cutoff (MWCO) where the preservative is already in the saliva or SOR filtrate tube as a liquid, or as a spray dried reagent, or as a lyophilized reagent prior to saliva or SOR filtration, or the preservative is added to the filtered saliva or filtered SOR sample filtrate tube such as by a filter tube cap, whereby the cap comprises the preservative reagent, comprising a reagent release mechanism whereby a membrane or barrier is broken when the cap is placed onto the filtrate tube or screwed onto the filtrate tube, or added to the filtered saliva or SOR sample by adding, pouring, dispensing, dropping, or mixing with an exogenous preservative reagent.

The collection tube cap or filtrate tube cap, or preservative reagent, or a combination thereof of a collection tube cap or filtrate tube cap also comprising a preservative, can comprise magnetic particles coated with capture molecules or capture moieties, such that when the magnetic particles are added to the collected saliva or SOR sample, or added to the filtered saliva or SOR filtrate, the magnetic particles will capture a particular analyte, target, or biomarker of interest, or capture 2 or more different analytes, targets, or biomarkers of interest from the entire sample, or the magnetic particles will capture an interference of interest, or capture 2 or more different interferences of interest from the entire sample, whereby the concentration of the analyte, target, biomarker, or interference is decreased, removed, or depleted from the sample or sample filtrate when the magnetic particles are subsequently isolated or removed from the sample such as after magnetic separation, filtration, or centrifugation. One or more analyte, target, biomarker, or interference captured by the magnetic particles can subsequently be detected, measured, or quantitated on the particle surface, or one or more analyte, target, biomarker, or interference can be cleaved, eluted, liberated, or dissociated from the particle surface for subsequent detection, measurement, or quantitation. The magnetic particles can be washed with a wash buffer or diluent prior to cleaving, eluting, liberating, or dissociating one or more analyte, target, biomarker, or interference from the magnetic particles for subsequent detection, measurement, or quantitation whereby the magnetic particle washing removes sample matrix, sample constituents, or sample interference that would otherwise obfuscate, interfere, or negatively impact the accuracy or sensitivity of detection, measurement, or quantitation of one or more analyte, target, biomarker, or interference. Cleaved, eluted, liberated, or dissociated analytes, targets, biomarkers, or interference can subsequently be conditioned, quenched, or neutralized with the addition of another buffer or diluent for improved stability prior to their detection, measurement, or quantitation.

The preservative reagent, particle storage buffer or diluent comprising magnetic particles coated with capture molecules or capture moieties, or the preservative reagent with magnetic particles coated with capture molecules or capture moieties, can also comprise a sample conditioning reagent to change, modify, or adjust sample conductivity, density, viscosity, surface tension, or protein content, or to add surfactants, detergents, cell lysis agents, anti-protease agents, anti-phosphatase agents, protein-based or polymeric-based blocking reagents, displacing agents, or agents to release analytes from the matrix or to remove interfering elements such as RIPA Buffer to lyse cells, extracellular particles, exosomes, neuro exosomes, virions, or bacterium, or such as a displacement agent or displacer reagent such as acidic pH, danazol, or 19-nortestosterone derivates, or 8-anilino-1-naphthalenesulfonic acid (ANSA) will liberate one or more bound analyte, target, biomarker, or interference from within cells, extracellular particles, exosomes, virions, or bacterium, or to liberate one or more bound analyte, target, biomarker, or interference from a binding partner such as 25-hydroxyvitamin D (25OHD) from the vitamin D binding protein (VDBP), or testosterone, estradiol, or dihydrotestosterone from sex hormone binding globulin (SHBG).

When the sample conditioning reagent, lysis buffer, displacement agent, or displacer reagent also comprises magnetic particles coated with capture molecules or capture moieties, the liberated analyte, target, biomarker, or interference from cells, extracellular particles, exosomes, neuro exosomes, virions, bacterium, or binding partners can be liberated in the collected sample or sample filtrate in the presence of the magnetic beads to maximize recovery and capture of the targeted one or more analyte, target, biomarker, or interference of interest for improved accuracy or sensitivity of detection, measurement, or quantitation of one or more analyte, target, biomarker, or interference, or for increased sensitivity of detection, measurement, or quantitation of low abundance, dilute, or very low concentration analyte, target, biomarker, or interference of interest.

In some embodiments, the subject can receive a sample collection device or receptacle in the mail, from a testing facility, or at a point of care facility. The sample collection device can comprise a tube and a lid. The subject collects a sample into the tube and caps the tube. In some embodiments, the cap can comprise biomarker capture particles or capture moieties that are released into the sample upon capping. For example, the cap may be a screwcap that releases the biomarker capture particles or capture moieties when the subject screws the cap on the receptacle. The capture particles or capture moieties may comprise capture moieties conjugated to capture particles. The capture particles or capture moieties may comprise capture moieties without capture particles. The tube or cap may also include a solution or buffer. For saliva collection, the subject may be provided an oral rinse. The oral rinse may include the buffer. The collected sample can then be delivered to a laboratory or testing facility. While in transit, the biomarker capture particles or capture moieties are incubated with any biomarkers from the collected sample.

### Methods of Use

In some embodiments, a capture moiety can be any antigens, therapeutics, drugs, small molecules, peptides, proteins, vaccines, or immunogens administered to or given to a subject therapeutically (e.g., orally as a pill or liquid or dissolvable, as a shot, as a supplement or herbal remedy, as a food or liquid, as a lotion, as an IV infusion, as a patch, as an enema, or any way a therapeutic can be administered or introduced into the subject such as by the skin, hair, or any external paths of entry including absorption, ingestion, and injection into the subject) that is immobilized, conjugated, or coated on the surface of the biomarker capture particles whereby such biomarker capture particles can bind immunoglobulins against the capture moiety. In some embodiments, the immunoglobulins can comprise human immunoglobulins classes IgA, IgG, IgM, and IgE, and human immunoglobulin subclasses such as IgAQ1, IgA2, IgG1, IgG2, IgG3, and IgG4, or animal species-specific antibodies such as IgG, where the biomarker capture particles can be used for the detection and monitoring of antibody production against the antigens, therapeutics, drugs, small molecules, peptides, proteins, vaccines, or immunogens.

In some embodiments, the methods provided herein can be used for therapeutic drug monitoring to detect, measure, or quantitate any immune response, or autoantibody response, against a drug or immunogen which can be dangerous to the patient and pose health and/or safety issues, or increase risk for an adverse event or response to the therapeutic including illness, hospitalization, or death, whereas absence of such antibodies indicates the therapeutic is safe and effective without adverse immune response.

In some embodiments, the methods provided herein can be used for vaccine efficacy testing and monitoring to 1) detect, measure, or quantitate any immune response, antibody response, or human immunoglobulin class or subclass response, against a vaccine or immunogen to determine if the vaccine is working as intended, 2) monitor the antibody response (timing, duration, levels, magnitude) by antibody class or subclass such as for antibody profiling to determine antibody response, timing, or how long it takes for antibody production after an initial vaccine shot, after a second vaccine shot, as well as after boosters, and to establish a protective immunity threshold or cutoff. The antibody levels can be monitored in circulation (blood, plasma, or serum), as a secretory response in oral fluids such as saliva or SOR, or in stool (produced in the gut or GI system).

In some embodiments, the antibody can be a therapeutic antibody such as a monoclonal antibody, humanized monoclonal antibody, antibody fragment, aptamer, MIP, nanobody such as Lama or Alpaca derived nanobody, or an animal-produced antibody or farmaceutical antibody, administered or given to a subject (e.g., a human patient or animal) for therapeutic, health improvement, or any health issue reasons, to treat, cure, manage, or mitigate a disease, development of a disease, progression or spread of a disease, or to irradicate, eliminate, or destroy a disease, infection, pathogen, cancer, autoimmune issue. In some embodiments, the therapeutic antibody is immobilized, coated, or conjugated to the biomarker capture particles to monitor any immune response to the therapeutic antibody in a human patient or animal as described above. In some embodiments, the antigen or target for which the therapeutic antibody is developed against, targeted against, recognizes, or binds to in the human patient or animal can be immobilized on the biomarker capture particles and the biomarker capture particles can be used to test human patient or animal samples to monitor and measure the therapeutic antibody level, concentration, or titer for precision medicine and guided therapy. The method can be used to determine a specific therapeutic antibody dose, circulating concentration, or secretory concentration. The method can be used in pharmacokinetics studies to determine the normalization, steady state, equilibration, and metabolization over time in circulation (e.g., blood, serum, or plasma) or secretory (e.g., oral fluids such as saliva and SOR, or stool, GI system) of the therapeutic antibody.

Disclosed herein, in some embodiments, are assay methods, comprising: obtaining a sample of a subject. The sample may have been subjected to cleaning by removing assay interferences from the sample with interference capture particles. Some embodiments include capturing antibodies from the sample. Some embodiments include contacting the sample with biomarker capture particles comprising an antigen of the antibodies. Disclosed herein, in some embodiments, are assay methods, comprising: obtaining a sample of a subject that has been subjected to cleaning by removing assay interferences from the sample with interference capture particles; and capturing antibodies from the sample by contacting the sample with biomarker capture particles comprising an antigen of the antibodies. In some embodiments an antibody can comprise an epitope that binds the antigen. Some embodiments include eluting the antibodies from the biomarker capture particles. Some embodiments include determining an amount of the antibodies in the sample or subject. In some embodiments, determining the amount of the antibodies in the sample or subject comprises determining an antibody mass per sample volume. Some embodiments include cleaning the sample by removing the assay interferences from the sample with the interference capture particles. Some embodiments include cleaning the biomarker capture particles with the interference capture particles. In some embodiments, the interference capture particles are removed to generate a cleaned sample. In some embodiments, the cleaned sample is substantially free of interferences. In some embodiments, biomarker capture particles are added to the cleaned sample for capturing the biomarker of interest (e.g., the antibody).

In some embodiments, the subject is suspected of having a disease, or has been administered a vaccine against the disease. In some embodiments, the antigen comprises a component of the pathogen or disease. In some embodiments, the captured antibodies comprise antibodies against the pathogen or disease. In some embodiments, the vaccine comprises the antigen. Some embodiments include determining a vaccine efficacy based on the amount of the antibodies in the sample or subject. Some embodiments include readministering the vaccine to the subject based on the efficacy of the vaccine efficacy. Some embodiments include identifying a likelihood of the subject having the disease based on the amount of the antibodies in the sample or subject.

Some embodiments include identifying a likelihood of the disease being active or acute. Some embodiments include administering the disease treatment to the subject when the subject is identified as having the active or acute disease. Some embodiments include not administering or discontinuing the treatment when the subject is identified as not having the active or acute disease. In some embodiments, administering the treatment comprises adjusting a dose amount or timing. In some embodiments, the subject has been administered a therapeutic compound. In some embodiments, the therapeutic compound comprises a therapeutic drug. In some embodiments, the antigen comprises the therapeutic compound, or a fragment thereof. In some embodiments, the captured antibodies comprise autoantibodies against the therapeutic compound. Some embodiments include determining a level of safety of the therapeutic compound based on the amount of the antibodies in the sample or subject. Some embodiments include administering or adjusting a dose of the therapeutic compound to the subject based on the amount of the antibodies in the sample or subject. In some embodiments, the therapeutic drug comprises a therapeutic antibody. In some embodiments, the therapeutic antibody comprises an antibody binding fragment. In some embodiments, the captured antibodies comprise the therapeutic antibody. In some embodiments, the antigen comprises an antigen or target of the therapeutic antibody. Some embodiments include determining a pharmacokinetic profile of the therapeutic antibody based on the amount of the antibodies in the sample or subject. Some embodiments include administering or adjusting a dose of the therapeutic antibody to the subject based on the amount of the antibodies in the sample or subject.

In some embodiments, the captured antibodies comprise secretory antibodies. In some embodiments, the captured antibodies comprise IgA, IgG, or IgM, or a combination thereof. Some embodiments include binding the captured antibodies with a detection antibody. Some embodiments include measuring the detection antibody. Some embodiments include comparing the detection antibody to a standard curve. In some embodiments, the standard curve is generated from biomarker capture particles bound to known amounts of the antibodies. Some embodiments include cleaning the detection antibody with the interference capture particles. In some embodiments, the detection antibody comprises an anti-human antibody. In some embodiments, the detection antibody comprises an antibody against the antigen. In some embodiments, the detection antibody is conjugated to a detection reagent. In some embodiments, the detection reagent comprises an enzyme or label. In some embodiments, the label comprises a fluorescent tag. Some embodiments include multiplexing the capture particles with additional capture particles comprising a second antigen, and wherein the antibodies comprise an antibody that binds to the second antigen. In some embodiments, determining the amount of the antibodies in the sample or subject comprising multiplexing the detection antibody with a second detection antibody that recognizes the second antigen. Some embodiments include multiplexing the detection antibody with an additional detection antibody that recognizes a biomarker in the sample. Some embodiments include monitoring antibodies over time to determine an increase or decrease in the amount of antibodies in a second sample of the subject.

Assay Method with Capture Moiety and Biomarker Capture Particles

Disclosed herein, in some embodiments, are assay methods, comprising (i) providing a sample of a subject; (ii) contacting the sample with a capture moiety; and (iii) contacting the sample with biomarker capture particles. In some embodiments, the biomarker capture particles can comprise a coating, e.g., a streptavidin coating. In some embodiments, the capture moiety can interact with the biomarker. In some embodiments, the capture moiety can further comprise a moiety that can interact and/or bind with the biomarker capture particles. In some embodiments, step (ii) comprises adding the capture moiety to the sample in a sample collection device. In some embodiments, the capture moiety can be present in a sample collection device before or during the sample collection. In some embodiments, the capture moiety can be biotinylated capture moiety. In some embodiments, the capture moiety can form a biomarker-capture moiety complex. In some embodiments, a biomarker-capture moiety complex can comprise one or more biomarkers. In some embodiments, a biomarker-capture moiety complex can comprise one or more capture moieties. In some embodiments, a biomarker-capture moiety complex can comprise one or more capture moieties and one or more biomarkers. In some embodiments, step (iii) comprises adding biomarker capture particles in molar excess over total moles of capture moieties. In some embodiments, step (iii) can be performed in a lab, a testing facility, or a point of care facility. The biomarker capture particles can bind the capture moiety and the biomarker-capture moiety complex.

In some embodiments, the sample may have been subjected to removal of interferences by contacting the sample with interference capture compositions. In some embodiments, the interference capture compositions are removed prior to contacting the sample with biomarker capture particles.

In some embodiments, prior to (ii), the method can comprise contacting the sample with an interference capture composition to remove assay interference(s). In some embodiments, after contacting the sample with the interference capture composition, the interference capture composition is removed.

In some embodiments, the capture moiety can be in a reagent, e.g., a liquid reagent or a solid reagent. In some embodiments, the liquid reagent can comprise a sample preservative reagent or stabilization agent. In some embodiments, the liquid reagent can comprise a sample conditioning reagent or agent. In some embodiments, the liquid reagent can comprise a sample preservative reagent or stabilization agent and a sample conditioning reagent or agent. In some embodiments, the liquid reagent can be lyophilized, spray dried, or pelleted to form the solid reagent. In some embodiments, the capture moiety can be in the solid reagent, for example, spray dried, lyophilized, or pellet. In some embodiments, the solid reagent can be derived from lyophilized, spray dried, or pelleted liquid reagent disclosed in the present disclosure. In some embodiments, the sample conditioning reagent or agent can further comprise a lysis agent, cell lysis agent, displacer agent, or binding partner displacement or dissociation agent. When the biomarker is inside a cell, extracellular particle, exosome, neuro exosome, virion, or bacterium or bound to a binding partner (e.g., vitamin D binding protein, sex hormone binding globulin, autoantibody, immune complex), the biomarker can be liberated or released in the presence of the capture moiety for enhanced biomarker capture and recovery by the capture moiety.

In some embodiments, the reagent comprising the capture moiety can already be present in the sample collection device prior to the sample or filtered sample, being added or collected into the sample collection device. In some embodiments, the reagent comprising the capture moiety can be added to the sample collection device after sample collection, or sample collection and filtration. In some embodiments, the reagent comprising the capture moiety can be stored in a screw cap with a screw cap release mechanism whereby the reagent can be added and mixed with the sample after the screw is tightened and the barrier is broken allowing the sample and reagent to mix. In some embodiments, the reagent comprising the capture moiety can be stored in a separate tube, vial, bottle, ampule, or vessel and can be added to the sample, or filtered sample, after the sample is collected in the sample collection device by opening or breaking the storage device and allowing the reagent within it to be added, poured, dumped, dropped, spilled, or mixed into the sample. In some embodiments, the reagent comprising the capture moiety can be added drop by drop from a dropper bottle, poured from a storage bottle after unscrewing or removing the cap, or poured/squeezed from an ampule after removing the tab. In some embodiments, the solid reagent comprising the capture moiety can be added or dropped into the sample where the pellet dissolves and releases the capture moiety into the sample. In some embodiments, the capture moiety can be stored inside a dissolvable capsule, pellet, or pill as a liquid or solid reagent, whereby the dissolvable, or time-delayed dissolvable, capsule, pellet, or pill is added to the sample and when the capsule, pellet, or pill dissolves in the sample, the capture moiety is released and mixed into the sample for biomarker capture.

In some embodiments, the biomarker capture particles can comprise streptavidin coated particles or anti-fluorescein antibody coated particles, which can target or bind a moiety on the capture moiety such as biotin or fluorescein. In some embodiments, the moiety can comprise a tag or fusion protein added to the capture moiety recombinantly, such as a his-tag, 6His-tag, or maltose binding protein (MBP). The moiety on the capture moiety can enable rapid and efficient capture of total capture moiety or biomarker-capture moiety complex from the sample. In some embodiments, the capture moiety can be captured by a binding partner immobilized or coated on the biomarker capture particles, such as an anti-capture moiety antibody. For example, if the capture moiety is an animal (such as mouse, rabbit, goat, sheep, cow, horse, lama, alpaca, camel, or pig) derived antibody, the biomarker capture particles can be coated with an anti-animal antibody such as anti-mouse, rabbit, goat, sheep, cow, horse, lama, alpaca, camel, or pig antibody. If the capture moiety is an aptamer or molecular imprinted polymer (MIP), or tagged, conjugated, or labelled with an oligonucleotide, peptide, polymer, or other tag, it can be capture by biomarker capture particles coated with anti-aptamer, anti-MIP, anti-oligonucleotide (complementary sequence), anti-peptide, or anti-polymer, or anti-other binding partner coated biomarker capture particles.

In some embodiments, the assay method can further comprise a concentrating process as disclosed elsewhere in the present disclosure. In some embodiments, the assay method can further comprise a cleaving process as disclosed elsewhere in the present disclosure. In some embodiments, the assay method can further comprise a cleaning process as disclosed elsewhere in the present disclosure. In some embodiments, the assay method can further comprise a biomarker cleaving process as disclosed elsewhere in the present disclosure. In some embodiments, the assay method can further comprise a conjugating process as disclosed elsewhere in the present disclosure. In some embodiments, the assay method can further comprise a conjugate cleaving process as disclosed elsewhere in the present disclosure. In some embodiments, the assay method can further comprise a characterizing process as disclosed elsewhere in the present disclosure.

In some embodiments, the assay method can further comprise isolating the biomarker capture particles from the sample and removing or aspirating the sample matrix. In some embodiments, the biomarker capture particles can be isolated/separated from the sample via centrifugation, filtration, or magnetic separation. In some embodiments, the captured biomarkers can subsequently be directly detected on the biomarker capture particles with or without washing the biomarker capture particles prior to detection. In some embodiments, the captured biomarkers can be cleaved or eluted from the biomarker capture particles, for subsequent detection and/or measurement via any suitable detection method presented in the disclosure. In some embodiments, the biomarker can be concentrated, enriched, purified, conjugated, and/or neutralized prior to detection/measurement. This assay method may improve biomarker binding kinetics and biomarker capture efficiency, including reduced time, due to the homogeneous reaction between the capture moiety and the biomarker.

In some embodiments, the capture moiety and the biomarker can comprise any biomarker, capture moiety pair that is disclosed in the present disclosure. In some embodiments, the biomarkers can comprise a plurality of biomarkers. In some embodiments, the biomarkers can comprise more than one type of biomarkers and the capture moieties can comprise more than one type of capture moieties for multiplexed capture and detection/measurement.

### Vaccine Efficacy and Immune Response Monitoring

The methods provided herein can be used for vaccine efficacy and immune response monitoring. After vaccination, it is important to know if antibodies generate against the vaccine or immunogen and the level of the antibodies. Singleplex detection of antibodies such as total IgG, total IgM, or total IgA, or antibody profiling to multiplex detect immunoglobulin classes IgG, IgM, and IgA, and/or immunoglobulin subclasses IgAQ1, IgA2, IgG1, IgG2, IgG3, and/or IgG4 can elicit a vaccine efficacy and immune response. In some embodiments, a recombinant protein or peptide comprising the epitope(s) the vaccine is generating an immune response against can be detected by the methods provided in the present disclosure.

The methods provided herein can be used to monitor any existing vaccines. Non-limiting examples vaccines comprise vaccine for Chickenpox (Varicella), Dengue, Diphtheria, Flu (Influenza), Hepatitis A, Hepatitis B, Hib (Haemophilus influenzae type b), HPV (Human Papillomavirus), Measles, Meningococcal, Mumps, Pneumococcal, Polio (Poliomyelitis), Rotavirus, Rubella (German Measles), Shingles (Herpes Zoster), Tetanus (Lockjaw), Whooping Cough (Pertussis), Adenovirus, Anthrax, Cholera, Japanese Encephalitis (JE), Rabies, Smallpox, Tuberculosis, Typhoid Fever, and Yellow Fever. Vaccines can comprise inactivated vaccines, live-attenuated vaccines, messenger RNA (mRNA) vaccines, subunit, recombinant, polysaccharide, and conjugate vaccines, toxoid vaccines, Novavax vaccine, DNA and recombinant vector vaccines (also known as platform-based vaccines), and viral vector vaccines.

In some embodiments, the biomarker capture particles can comprise one or more different types of biomarker capture moieties that can bind with the antibodies. The biomarker capture moieties can comprise an antigen, a vaccine, or a fragment of the vaccine. After the capturing or capturing and conjugating, the biomarkers or the conjugates can be eluted from biomarker capture particles to be detected and measured for the presence of a target antibody or a plurality of target antibodies. In some embodiments, the vaccine efficacy can be monitored in an extended period of time to generate a response profile of the vaccine.

In another aspect, the present disclosure provides a method for determining a vaccine efficacy, the method comprises: (a) providing a sample of a subject wherein the subject has been administered a vaccine; (b) capturing a biomarker from the sample by contacting the sample with biomarker capture particles comprising a biomarker capture moiety; (c) quantifying the eluted biomarker; and (d) determining the vaccine efficacy. In some embodiments, the method can comprise readminister the vaccine to the subject based on the vaccine efficacy. In some embodiments, the sample may have been subjected to removal of interferences by contacting the sample with interference capture compositions. In some embodiments, the interference capture compositions are removed prior to contacting the sample with biomarker capture particles. In some embodiments, prior to (b), the method can comprise contacting the sample with an interference capture composition to remove assay interference(s). In some embodiments, after contacting the sample with the interference capture composition, the interference capture composition is removed.

### Therapeutic Drug Monitoring

The methods provided herein can be used for monitoring an effectiveness of a medicament. In some embodiments, a medicament can comprise a vaccine or therapeutic. In some embodiments, methods disclosed herein can be used for a therapeutic drug monitoring or therapeutic antibody monitoring for companion diagnostics, precision medicine, or guided therapy by monitoring the level, concentration, or dose of the therapeutic in patients, or serially monitoring patients over time. In some embodiments, a therapeutic drug or a binding element of the therapeutic drug can be immobilized, conjugated, or bound on capture particles (e.g., therapeutic capture particles). After elution from the capture particles, the therapeutic drug can be detected and measured. The therapeutic drug can be monitored or characterized in a biofluid. In some embodiments, the biofluid can comprise whole blood, serum, plasma, urine, oral fluid (saliva, drool, oral mucosal transudates (OMT), or oral rinse), peritoneal fluid, pleural fluid, cerebrospinal fluid (CSF), tissues, sweat, or tears. In some embodiments, clinical decisions can be made based on the therapeutic drug level detected in the sample of the patient.

In an aspect, the present disclosure provides a method for determining a therapeutic efficacy, the method comprises: (a) providing a sample of a subject wherein the subject has been administered a therapeutic; (b) capturing a biomarker from the sample by contacting the sample with biomarker capture particles comprising a biomarker capture moiety; (c) quantifying the eluted biomarker; and (d) determining the therapeutic efficacy. In some embodiments, the sample may have been subjected to removal of interferences by contacting the sample with interference capture compositions. In some embodiments, the interference capture compositions are removed prior to contacting the sample with biomarker capture particles. In some embodiments, prior to (b), the method can comprise contacting the sample with an interference capture composition to remove assay interference(s). In some embodiments, after contacting the sample with the interference capture composition, the interference capture composition is removed.

In some embodiments, the subject has been administered a therapeutic compound. In some embodiments, the therapeutic compound can comprise a therapeutic drug or a therapeutic antibody. In some embodiments, the biomarker can comprise the therapeutic compound, or a fragment thereof. In some embodiments, the captured biomarker can comprise autoantibodies against the biomarker. In some embodiments, the method can comprise determining a level of safety of the therapeutic compound based on the quantified biomarker. In some embodiments, the method can further comprise administering or adjusting a dose of the therapeutic compound to the subject based on the quantified biomarker. In some embodiments, the therapeutic antibody can comprise an antibody binding fragment. In some embodiments, the captured biomarker can comprise the therapeutic antibody.

In some embodiments, the method can further comprise determining a pharmacokinetic profile of the therapeutic antibody based on the quantified biomarker. In some embodiments, the method can further comprise administering or adjusting a dose of the therapeutic antibody to the subject based on the quantified biomarker. In some embodiments, the method can comprise adjusting an administration timing or duration based on the quantified biomarker.

### Therapeutic Safety and Efficacy Monitoring

The methods provided herein can be used for therapeutic safety and efficacy monitoring by monitoring patients, or serially monitoring patients over time, to detect a possible immune response or autoantibody response against the therapeutic. In some embodiments, a therapeutic is immobilized, conjugated, or bound on the capture particles. In some embodiments, immune response is monitored or characterized, including antibody profiling to multiplex detect immunoglobulin classes IgG, IgM, and IgA, and/or immunoglobulin subclasses IgAQ1, IgA2, IgG1, IgG2, IgG3, and/or IgG4, against the therapeutic. In some embodiments, the therapeutic can be a drug, pharmaceutical, small molecule, a protein or peptide, an antibody, humanized monoclonal antibody or chimeric antibody, or a therapeutic antibody. The detection of the immune response can aid in adjusting the dose or administration of the therapeutic. In some embodiments, adjusting the dose can comprise reducing the dose for patient safety. In some embodiments, adjusting the dose can comprise increasing the dose to overcome antibody binding complexes so the active dose is effective even in the presence of autoantibodies.

Multiplex Detection and/or Measurement of Biomarker from a Saliva Sample

In an aspect, the present disclosure provides a method of detecting and/or measuring biomarkers from a saliva sample. The method can comprise cleaning the saliva sample by interference capture particles (or cleaning beads) to capture, remove, deplete, reduce, or eliminate saliva specific interferences that would otherwise, if still present in the sample, will negatively impact the accuracy, specificity, and/or sensitivity of the subsequent biomarker capture by biomarker capture particles. In some embodiments, the method can comprise conditioning the saliva sample using a cleaning reagent. In some embodiments, the cleaning reagent can comprise lysing reagent for lysing of a pathogen, exosome, extra cellular particle, or cell thereby liberating or releasing the biomarkers from within the pathogen, exosome, extra cellular particle, or cell. In some embodiments, the method can comprise isolating or removing the cleaning beads from the cleaned sample, or cleaned and conditioned sample, via magnetic separation, filtration, or centrifugation. In some embodiments, the method can comprise adding biomarker capture particles (or capture beads) to the cleaned, or cleaned and conditioned, sample to capture the biomarkers in the saliva sample. In some embodiments, the method can comprise washing the capture beads and captured biomarkers of sample matrix with a wash buffer to remove or reduce non-specific binding to the capture beads. In some embodiments, the method can comprise reducing the wash volume or elution buffer to concentrate the captured biomarker. In some embodiments, the method can comprise cleaving or eluting the captured biomarker into a buffer. In some embodiments, a neutralizing buffer can be added to the buffer. In some embodiments, the eluted biomarker can be detected or measured by any suitable detection method or existing testing methods, test systems, or assays.

In some embodiments, normal abundance or low abundance saliva-based biomarkers of interest can be captured and enriched from the entire collected saliva or SOR sample using magnetic biomarker capture particles for biomarker purification, detection, measurement, or quantitation. The biomarker capture particles, antigen coated biomarker capture particles, or a mixture or pool of 2 or more different antibody, antigen, or antibody and antigen coated biomarker capture particles can be stored in a saliva collection tube cap, in a reagent vial or container, or already in the collection tube with a preservative, conditioning reagent, or a mixture of preservative and conditioning reagent. Biomarker capture particles would be released from the cap, or added to the sample, or mixed with the collected sample such that the preservative and the biomarker capture particles would be introduced into the entire collected saliva or SOR sample as a biomarker capture and enrichment reagent to subsequently purify biomarkers from the saliva or SOR for subsequent testing or measurement by a test, diagnostic test, or assay method. The biomarker capture particles can target a single biomarker, or a pool of different biomarker capture particles can target a panel of biomarkers (multiplex capture). Since the biomarker capture particles are introduced into the entire 0.5-5 mL, (e.g., 0.5 mL, 1 mL, 2 mL, 3 mL, 4 mL, 5 mL), or >5 mL collected saliva or SOR sample, the entire or whole saliva sample volume is the sample volume for biomarker capture by the biomarker capture particles. This approach is of particular importance for the capture of low abundance or dilute biomarkers in saliva or SOR whereby this approach facilitates biomarker concentration or enrichment for subsequent biomarker detection, measurement, or quantitation. For biomarkers that may be liberated or released in saliva from a binding partner (i.e. 25OH Vitamin D from Vitamin D Binding Protein, or Testosterone from Sex Hormone Binding Globulin), or liberated from within a particle or cell such as an exosome or extra cellular particle, virion, or bacterium, the biomarker capture particle storage buffer can also comprise a sample conditioning buffer or cellular lysing agent such as RIPA Buffer, or displacement agent or chemical, as well as the preservative. In this way the biomarkers are liberated or released in the presence of the antibody and antigen coated biomarker capture particles to maximize biomarker recovery and the sensitivity of biomarker detection. Once the saliva or SOR collection tube with magnetic biomarker capture particles reaches the lab, the lab can easily isolate the magnetic beads with a magnet, either to the side of the tube with a strong magnetic such that the tube content or sample can be removed and discarded, or via a magnet in a tip approach whereby the beads are removed from the tube and placed into a new tube, wash the beads, and subsequently elute, neutralize, and test or measure one or more biomarkers purified from the saliva sample by an ELISA, automated CLIA, chemistry, a multiplex test (i.e. Luminex, FIA, mass spec), or method of choice. In some embodiments, since the magnetic biomarker capture particles are isolated to a magnet and the saliva or SOR sample is removed and discarded prior to washing the beads and the captured biomarkers elute from the beads. This cleaning process removes the saliva matrix and the majority of saliva-based interference prior to the test such that the eluted and neutralized biomarkers are in a buffer without all or the majority of such saliva-based interference. This approach significantly simplifies subsequent biomarker testing and increases the sensitivity of biomarker detection. In some embodiments the biomarkers that have been captured by the magnetic biomarker capture particles in the collected saliva sample can be directly measured on the biomarker capture particle with a conjugate after the magnetic biomarker capture particles are isolated to a magnet, the saliva or SOR sample is removed and discarded, and the beads are washed.

In some embodiments, the use of a conditioning agent to lyse cells or displace and liberate biomarkers in the presence of the biomarker capture particles facilitates the capture of total biomarkers in the saliva or SOR sample, or both total free biomarkers as well as liberated or released biomarkers from within cells or from binding partners.

In some embodiments, the biomarker or biomarkers of interest can be captured in the saliva collection device such that the magnetic beads can subsequently be isolated and washed, and the captured biomarkers eluted and neutralized into a buffer for subsequent testing by existing tests or testing platforms such as Luminex, ELISA, automated CLIA, and mass spectrometry. This approach overcomes several key challenges of working with and testing saliva such as; 1) improved sensitivity, or increased likelihood of detection of low abundance biomarkers since the entire saliva sample collected is the sample for the magnetic biomarker capture particles, and the eluted biomarkers can be in a much smaller sample size for their concentration/enrichment, 2) improved accuracy, or the saliva matrix, and all the known challenges saliva testing presents, can be mitigated by simple magnetic isolation and washing of the biomarker capture particles by the lab prior to elution of the biomarkers into a simple, clean (saliva matrix free) easy to test buffer, 3) improved recovery, or since the biomarker capture particles are added to the saliva sample immediately after saliva sample collection this maximizes biomarker capture and recovery from the sample prior to any possible biomarker loss via hydrophobic or non-specific binding mechanisms, and this is particularly true if the biomarker capture particles are stored in a lysis buffer or displacing agent that liberates bound biomarkers or biomarkers within cells as they will get released in the presence of the capture beads thereby maximizing their capture and recovery from the sample, 4) improved stability, or the biomarker capture particles can be stored in a preservative reagent, and biomarkers, proteins, and antigens are generally more stable once they are bound or immobilized to a solid phase and once the biomarker capture particles bind their target biomarkers the captured biomarkers will be very stable until they are eluted from the biomarker capture particles for testing, and 5) improved laboratory workflow, or since the biomarkers have already been captured by the magnetic beads in the saliva collection device prior to the lab receiving the sample tube the biomarker capture step is already complete, and the lab may not need to centrifuge the sample eliminating this time and effort such that upon saliva or SOR sample receipt the lab may use a magnet to isolate the magnetic biomarker capture particles, remove the saliva matrix (which can be saved for other testing), wash the particles, and elute and neutralize the biomarkers for testing. This sample workflow can also be fully automated by any liquid hander, and the buffer-based biomarker sample can easily be tested by any existing testing platform, including fully automated CLIA analyzers or by analyzers on a track system, which would be challenging or not possible for viscous, neat, saliva samples.

Multiplex Detection and/or Measurement of Neuromarker

In an aspect, the present disclosure provides a method for detecting and/or measuring a neuromarker in a sample. In some embodiments, the neuromarkers can be biomarkers for the Alzheimer's disease or amyloid pathology (amyloid plaque), for example, phosphorylated Tau 181 (pTau181), amyloid beta 1-42 (Aβ42 or AB42) or amyloid beta 1-40 (Aβ40 or AB40) in cerebral spinal fluid, or phosphorylated Tau 217 (pTau217) in plasma. In some embodiments, the sample can comprise plasma, saliva, or urine.

The method for detecting/measuring a neuromarker can comprise cleaning the sample by interference capture particles (or cleaning beads or clean beads) to capture, remove, deplete, reduce, or eliminate interferences that may otherwise, if still present in the sample, negatively impact the accuracy, specificity, and/or sensitivity of the subsequent biomarker capture by biomarker capture particles. In some embodiments, the method can comprise conditioning the sample using a cleaning reagent. In some embodiments, the cleaning reagent can comprise lysing reagent for lysing of a pathogen, exosome, extra cellular particle, or cell thereby liberating or releasing the biomarkers from within the pathogen, exosome, extra cellular particle, or cell. In some embodiments, the method can comprise isolating or removing the cleaning beads from the cleaned sample, or cleaned and conditioned sample, via magnetic separation, filtration, or centrifugation. In some embodiments, the method can comprise adding biomarker capture particles (or capture beads) to the cleaned, or cleaned and conditioned, sample to capture the biomarkers in the sample. In some embodiments, the method can comprise washing the capture beads and captured biomarkers of sample matrix with a wash buffer to remove or reduce non-specific binding to the capture beads. In some embodiments, the method can comprise reducing the wash volume or elution buffer to concentrate the captured biomarker. In some embodiments, the method can comprise cleaving or eluting the captured biomarker into a buffer. In some embodiments, a neutralizing buffer can be added to the buffer. In some embodiments, the eluted biomarker can be detected or measured by any suitable detection method or existing testing methods, test systems, or assays.

In some embodiments, an elution buffer can comprise an acidic buffer (low pH) such as 100-125 mM glycine pH 2.5 or 0.15% (v/v) TFA Trifluoroacetic acid (TFA) or 40 mM Acetic Acid, 0.05% Tween-20, pH 3.05. In some embodiments, an elution volume can be small or less than the original sample volume, or less than the final wash buffer volume, to enrich or concentrate the eluted and purified pTau181 and Abeta42. In some embodiments, a neutralization buffer can comprise 300 mM Tris, or 1M Tris, pH 8.0, or 300 mM TRIS pH 10.5, 0.05% Tween-20.

### Sandwich Competitive Assay

In an aspect, the present disclosure provides a sandwich competitive assay (SCA) method of detecting and/or measuring biomarkers. The SCA method can use a 96-well or a 384-well plate to test 1 sample, or two or more samples in batch for increased testing throughout.

The SCA method comprises adding a sample such as serum, plasma, saliva, diluted saliva with a diluent such as 0.9% NaCl or PBS, or saline oral rinse to a well of a 96-well or 384-well plate. The sample can be tested neat or “as is” in the primary collection tube or secondary transfer tube, or the sample can be processed prior to use, or prior to testing, such as after sample centrifugation or filtration to clarify the sample by removing any cells, debris, particulates, lipids or other pre-analytical sample interferences that can be pelleted out with centrifugation or eliminated or removed with filtration. The sample can also be conditioned or pre-incubated with a conditioning agent, interference capture beads, or a conditioning reagent comprising interference capture beads, prior to sample centrifugation or filtration.

The SCA method can comprise different orders of addition, or “assay formats”. In some embodiments, a plurality of larger magnetic biomarker capture beads and a plurality of conjugates (e.g., smaller non-magnetic biomarker capture beads or non-magnetic conjugate capture moiety such as a detection antibody) can be added to the sample to bind to the biomarker and form an immune complex. In some embodiments, a plurality of larger magnetic biomarker capture beads can be added to the sample first followed by a plurality of smaller non-magnetic biomarker capture beads or non-magnetic conjugate capture moiety as a second reagent addition, to the sample to form an immune complex. In some embodiments, a plurality of smaller non-magnetic biomarker capture beads non-magnetic conjugate capture moiety can be added to the sample first followed by a plurality of larger magnetic biomarker capture beads, to form an immune complex. In some embodiments, a labeled capture moiety, such as a biotin labeled capture moiety, and a plurality of smaller non-magnetic biomarker capture beads or non-magnetic conjugate capture moiety can be added to the sample, followed by the addition of a plurality of large magnetic anti-label bead, such as a larger magnetic streptavidin coated capture bead, to bind the label of the labeled capture moiety or the biotin of the immune complex.

The SCA method can further comprise an incubation. In some embodiments, the incubation can be performed with mixing. In some embodiments, the incubation can be performed at controlled temperature such as 2-8° C., room temperature or ambient temperature, 30° C., 37° C., or 42° C. The incubation time can be 30 seconds, 1 min, 2 min, 3 min, 4 min, 5 min, 10 min, 15 min, 30 min, 45 min, 60 min, 90 min, 120 min, 4 hours, 8 hours, or overnight.

The larger magnetic capture beads, or the labeled capture moiety, are in molar excess over total biomarker in the sample. This molar excess can be a 2×, 3×, 4×, 5×, 6×, 7×, 8×, 9×, 10×, 20×, 40×, 100×, 1000×, or greater than 1000× molar excess to capture 100% of the biomarker, or to capture up to 100% of the biomarker in the incubation time.

The smaller non-magnetic capture beads, or the non-magnetic conjugate capture moiety, are in a limiting molar ratio less than the total moles of biomarker in the sample such as a 1:2, 1:3, 1:4, 1:5, 1:6, 1:7, 1:8, 1:9, 1:10, 1:20, 1:40, 1:100, 1:1000, 1:2000, 1:5000, 1:10,000, 1:100,000 molar ratio of the smaller non-magnetic capture beads or the non-magnetic conjugate capture moiety, per moles of biomarker. In some embodiments, the smaller non-magnetic capture beads or the non-magnetic conjugate capture moiety can be of any limiting amount, mass, concentration, moles, or molarity, such that a subsequent decrease of its amount, mass, concentration, moles, or molarity in the sample can be detected by a reader such as a fluorimeter, luminometer, or UV/vis detector. In some embodiments, more than 10%, more than 20%, more than 30%, more than 40%, more than 50%, more than 60%, more than 70%, more than 80%, more than 90%, more than 95%, more than 99%, or more of the smaller non-magnetic capture beads or the non-magnetic conjugate capture moiety can be consumed or complexed with the [larger magnetic capture bead]-biomarker immune complex as a [larger magnetic capture bead]-biomarker-[smaller non-magnetic capture beads] complex or a [larger magnetic capture bead]-biomarker-[non-magnetic conjugate capture moiety] complex.

In some embodiments, the smaller non-magnetic capture beads or the non-magnetic conjugate capture moiety can be labelled with fluorescein or a fluorophore for fluorescent detection by a fluorimeter. In some embodiments, the smaller non-magnetic capture beads or the non-magnetic conjugate capture moiety can be labelled with a chemiluminescence substrate such as ABEI, luminol, isoluminol, and acridinium ester, for luminescence detection by a luminometer. In some embodiments, the smaller non-magnetic capture beads or the non-magnetic conjugate capture moiety can be labelled with an electrochemiluminescence substrate such as ruthenium, for electrochemical luminescence (ECL).

In some embodiments, the detection can be done by a UV/vis detector, in a wavelength of light from 200 nm to 800 nm. In some embodiments, the smaller non-magnetic capture beads or the non-magnetic conjugate capture moiety can be labelled with Alkaline Phosphates (ALP) or horse radish peroxidase (HRP).

Subsequent to the formation of the immune complex, i.e., the [larger magnetic capture bead]-biomarker-[smaller non-magnetic capture beads] complex or the [larger magnetic capture bead]-biomarker-[non-magnetic conjugate capture moiety] complex, the 96-well plate or 384-well plate can be placed on a respective or appropriately strong 96-well or 384-well plate magnet to separate or isolate the larger magnetic capture beads to the side, sides, or bottom of each well. Since biomarker, biomarker-[smaller non-magnetic capture beads] complex, or the biomarker-[non-magnetic conjugate capture moiety] complex are also attached to, bound to, or complexed to the larger magnetic capture beads they can move to the magnet, or be separated, isolated, or removed from the sample supernatant along with the larger magnetic capture beads.

The SCA method can further comprise aspirating or dispensing the sample supernatant into an appropriate 96-well or 384-well detection or read plate for measurement by a fluorimeter, luminometer, or UV/vis detector of any residual signal of the smaller non-magnetic capture beads or the non-magnetic conjugate capture moiety. A 100% conjugate signal or a high level of residual conjugate signal detected in the sample supernatant is indicative of a negative test result for the biomarker, or the biomarker is not detected. A reduced conjugate signal, for example, less than 90%, less than 80%, less than 70%, less than 60%, less than 50%, less than 40%, less than 30%, less than 20%, less than 10%, less than 5%, less than 1%, or less, conjugate signal detected in the sample supernatant, is indicative of a positive result or the biomarker is detected.

In some embodiments, the conjugate can comprise non-magnetic white biomarker capture beads or non-magnetic colored latex biomarker capture beads. In some embodiments, the conjugate can have sufficient size for visual detection such as by the human eye. In some embodiments, the conjugate can comprise a mixture of 2 different color non-magnetic latex beads such as green and red, where one color bead is also a biomarker capture beads (i.e., the “green” beads) and the other color bead is an inert, non-functional, or non-biomarker binding bead (i.e., the “red” beads). If the biomarker is present in the sample, it will form an immune complex with the larger, brown, magnetic capture beads (added to the sample in molar excess of the biomarker) and the smaller non-magnetic white biomarker capture beads or mixture of green non-magnetic capture beads and red non-magnet beads (added to the sample at a limiting molar ratio less than the total moles of biomarker). After magnetic separation, the brown magnetic capture beads are removed from the sample supernatant with (if biomarker is present and the immune complex forms) or without (no biomarker present) the non-magnetic white latex capture beads or the “green” non-magnetic latex capture beads. In the use case of the white non-magnetic latex biomarker capture beads, a visual decrease, reduction, or absence of white beads (i.e., clear solution) indicates a positive result and the biomarker has been detected, and the presence or visual detection of the white beads (i.e., a white solution, “milky” solution, or whitish cloudy solution) indicates a negative test result or the biomarker has not been detected. In the use case of the green non-magnetic latex biomarker capture beads mixed with red non-magnetic latex beads, a “red” color of the solution after magnetic separation of the magnetic biomarker capture beads indicates a positive result or the biomarker has been detected, while a “brown” color of the solution after magnetic separation of the magnetic biomarker capture beads indicates a negative test result or the biomarker has not been detected. The SCA format can be used at the point-of-care (POC).

### Kits

Some embodiments relate to a kit. The kit may include any aspect described herein. In some embodiments, the kit can comprise a sample collection device for collecting a biological sample as disclosed herein. In some embodiments, the kit can comprise a sample receptacle. In some embodiments, the kit can comprise a biomarker capture particle as disclosed herein. In some embodiments, the kit can comprise a capture moiety as disclosed herein. In some embodiments, the kit can comprise an interference capture composition as disclosed herein, e.g., cleaning particle. In some embodiments, the kit can comprise buffer solutions as disclosed herein. In some embodiments, the kit can comprise wash solutions disclosed herein. In some embodiments, the kit can comprise a reagent for measuring a biomarker present in a sample. In some embodiments, the kit can be used in the methods as disclosed herein. In some embodiments, the kit can comprise instructions for use, such as instructions for performing a method disclosed herein.

In some embodiments, the interference capture composition can comprise an interference capture moiety as disclosed herein. In some embodiments, the biomarker capture particle can comprise a biomarker capture moiety as disclosed herein.

In some embodiments, the kit can comprise a detection antibody.

In some embodiments, the interference capture moiety can comprise a human immunoglobulin, an antibody, an animal antibody, an antibody fragment, a polymerized antibody, a mouse antibody fragment, an aptamer, a protein, an enzyme, a small molecule, a streptavidin, an avidin, a neutravidin, an MIP, a polymer, a conjugation linker, or any combination thereof.

In some embodiments, the biomarker capture moiety can comprise an antibody, an antibody fragment, a polypeptide binder, a monobody, a non-immunoglobulin binder, a DARPin, an affibody, an anticalin, a molecular imprinted polymer (MIP), an aptamer, a chimeric antibody, a therapeutic antibody, an antigen, a protein, a small molecule, a therapeutic, a hormone, a peptide, a signaling peptide, an exosome, a cell, a disease state-specific antigen, an antibody, a biomarker, or any combination thereof.

### EXAMPLES

The following examples are included for illustrative purposes only and are not intended to limit the scope of the present disclosure.

### Example 1: Detection of 25OH Vitamin D

25OH Vitamin D or 25OHD can be measured by immunoassay and/or LC-MS/MS tests to assess patient vitamin D sufficiency or deficiency. 25OHD is present in 2 different forms in circulation, 25OHD3 and 25OHD2, depending on diet, supplements, and UV light exposure such as sun light. 25OHD2 is more hydrophobic than 25OHD3, and it is bound to the vitamin D binding protein (VDBP) in circulation to protect it and transport it to its cellular targets or the kidney for additional hydrolysis to 25OH2D. 25OHD may be liberated or released from the VDBP in order to be captured, detected, and measured by immunoassay or LC-MS/MS. This release is accomplished either via a low acidic pH, e.g., a pH of less than 4.3, or with a displacing agent or chemical. The best 25OHD total assays liberate or release the 25OHD2 and 25OHD3 in the presence of the capture antibody to maximize analyte recovery, especially the more hydrophobic 25OHD2 which can otherwise be lost hydrophobically to lipids, trigs, fats, or other proteins in the sample.

Biomarker capture particles, e.g., magnetic capture particles, coated with anti-25OHD2 and anti-25OHD3 antibody are added to a saliva sample in a collection device with a releasing agent, to liberate or release the potentially low abundance total 25OHD in the presence of the capture antibodies to maximize recovery and enrich it. Once the saliva collection device reaches a lab, the biomarker capture particles are isolated such as on a magnet, or via centrifugation. The biomarker capture particles are then washed to remove sample matrix and NSB and to reduce sample volume to concentrate/enrich the biomarker capture particles. The total 25OHD that are captured on the biomarker capture particles are then eluted and neutralized from the biomarker capture particles for subsequent measurement by immunoassay or LC-MS/MS.

### Example 2: Detection of Neuromarkers

Accurate and sensitive detection of neuromarkers in saliva based samples from at home saliva or saline oral rinse collection, or saliva collection at physician office, pharmacy, or specialist clinic (neurologist), for age-based risk screening for neurodegenerative disease such as Alzheimer's, Parkinson's or diagnosis of disease or early onset of disease, or saliva-based testing of athletes suspected of TBI or concussion, or to rule-in/out TBI or concussion in the ER such as car accident, fall, or infant shaking patients or victims.

Biomarker capture particles are added to the saliva or saline oral rinse collection device to maximize recovery of these neuromarkers during collection or in the collection device. This is key for any neuromarkers that may hydrophobically or NSB interact with the collection device and tube and be lost or no longer detectable. The biomarker capture particles can comprise multiple different antibodies to bind 1 or more different neuromarkers or a panel of neuromarkers during the pre-analytical capture. If these neuromarkers are inside neuro exosomes, these exosomes can also be lysed in the presence of the antibody coated biomarker capture particles to maximize the capture efficiency and yield from the sample.

### Example 3: Agglutination Study

Magnetic beads (or biomarker capture particles, 550 nm) coated with streptavidin and co-coated with 2 different biotinylated monoclonal antibodies were tested for agglutination against 200 ng IFN (protein), 200 ng BSA (negative protein control 1), 200 ng Gro (negative protein control 2), a triplex mixture of IFN, BSA, and Gro (200 ng each), and a buffer blank (matrix control—no proteins).

The antibody pair co-coated on the magnetic beads was shown in Table 1. The beads were prepared as follows: in a 5 mL sample tube, the biotinylated antibodies were added first, the streptavidin beads were subsequently added drop-by-drop (slowly) to the biotinylated antibodies while mixing (vortex mixing) the biotinylated antibodies to mitigate bead aggregation or biotin-protein mediated cross-linking of the streptavidin beads. The sample tube was then capped and incubated on the rocker for 2 hours at room temperature. After two hours, the beads were washed 3× with TBS (10 mM TRIS, 150 mM NaCl, pH 7.4) with 0.05% Tween-20 and 0.05% sodium azide. 2 mg total of magnetic beads were coated with 100 μg of total antibodies (50 μg antibody/mg beads), 50 μg of each biotinylated anti-hIFNg antibody.

TABLE 1
Antibody pair co-coated on the magnetic beads

| | | | | | Biotinylate |
|---|---|---|---|---|---|
| | Manufac- | Clone | | Lot | Conc. |
| anti-hIFNg | ture | # | P/n | number | (mg/mL) |
| mAbMsahINFg | Invitrogen | M701 | M701 | XB349230 | 0.84 |
| mAbMsahINFg | Invitrogen | MD-1 | MD-1 | 2428662 | 0.88 |

Analytes containing the corresponding IFN, BSA, and Gro were added to a 30 μL buffer in an assay plate to make solutions with 200 ng IFN (sample A), 200 ng BSA (sample B), 200 ng Gro (sample C), mixture of 200 ng IFN, 200 ng BSA, and 200 ng Gro (sample D). Sample E is a control sample (blank, buffer only). 20 μg magnetic beads were added to samples A-E and mixed 10× by pipetting.

After an incubation time, the magnetic beads were allowed to settle for 30 min and the bottom of the clear, flat bottom wells were imaged by the Cytation 5 Cell Imaging Multimode Reader.

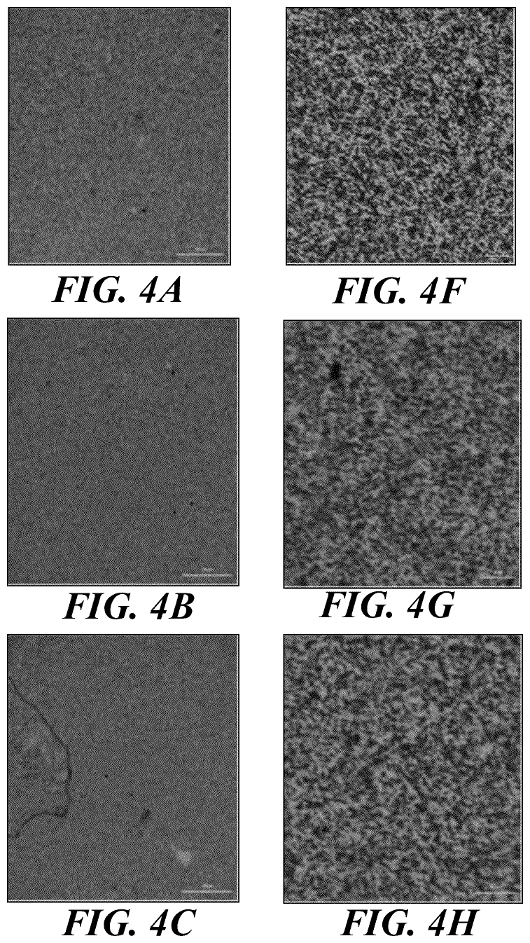

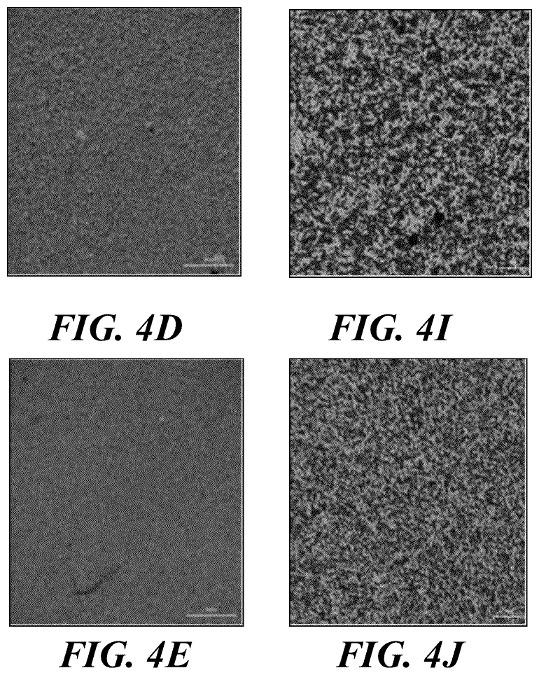

FIG. 4A-4E show images of samples A-E, respectively. FIG. 4F-4J show images of samples A-E, respectively at a higher magnificence.

Table 2 shows the cell count, object sum area, and object sum intensity results of sampled A-E.

TABLE 2
Summary of cell count, object sum area, and object sum intensity results

| | Cell | | OSA | OSA | OSA | OSA | OSA |
|---|---|---|---|---|---|---|---|
| Sample | Count | OSA-S | [S 9-30] | [S 9-12] | [S 12-15] | [S 15-20] | [S 20-30] |
| A | 6451 | 1.00 × 10⁶ | 7.05 × 10⁵ | 3.01 × 10⁵ | 2.37 × 10⁵ | 1.52 × 10⁵ | 1.45 × 10⁴ |
| B | 4252 | 7.48 × 10⁵ | 3.89 × 10⁵ | 2.35 × 10⁵ | 1.16 × 10⁵ | 3.59 × 10⁴ | 2.45 × 10³ |
| C | 4851 | 8.60 × 10⁵ | 4.41 × 10⁵ | 2.70 × 10⁵ | 1.33 × 10⁵ | 3.59 × 10⁴ | 1.93 × 10³ |
| D | 7899 | 1.23 × 10⁶ | 9.74 × 10⁵ | 3.18 × 10⁵ | 3.17 × 10⁵ | 2.90 × 10⁵ | 4.86 × 10⁴ |
| E | 5486 | 9.54 × 10⁵ | 5.11 × 10⁵ | 3.06 × 10⁵ | 1.54 × 10⁵ | 4.97 × 10⁴ | 1.00 × 10³ |

The data shows significantly larger aggregates against IFN or the triplex mixture containing IFN as compared to BSA or Gro.

### Example 4: Multiplex Detection and Quantitation of Total IgA, IgG, and IgM Against a Viral Vaccine in Serum

This example shows the multiplex detection of total IgA, IgG, and IgM immunoglobulins against 50 or more different HSV epitopes. The attenuated HSV virus used as the vaccine immunogen was conjugated to the biomarker capture particles (or capture beads), through a thiol-maleimide based chemistry or biotin-PEG linker chemistry or coated to the biomarker capture particles after digesting the immunogen or virion through a thiol-maleimide based chemistry or biotin-PEG linker chemistry. Using either approach will make sure all vaccine epitopes are represented on the capture beads to detect and quantitate any immunoglobulin response to the vaccine, e.g., IgA, IgG and/or IgM.

### Example 5: Multiplex Assay for Active Lyme Infection

This example illustrates the multiplex assay for detection of active Lyme (Borrelia burgdorferi) infection. Borrelia antibody testing and antigen testing were combined in a single multiplex test for the detection of active Lyme disease in serum or saliva. Borrelia antibody testing uses a pool or mixture of 4 different Borrelia antigen-coated capture beads (DpbA, OspA, OspC, and VIsE) to capture, detect and quantify anti-Borrelia IgG, IgM, and IgA antibodies. Feasibility testing demonstrated 100% sensitivity and 100% specificity using a CDC verification panel (N=32) and Lyme disease Biobank samples sourced from the Bay Area Lyme Foundation (N=19).

Two testing protocols were used to determine if a patient has an active Lyme disease infection. The first protocol used a 5-plex test which combined Borrelia antibody testing with additional tests that measured biomarkers for the inflammatory response to Lyme infection. This could be also used as a Tick-borne Illness Screening Test which can determine if a patient presenting with signs and symptoms of Lyme disease has a different tick-borne infection such as Anaplasmosis, Babesiosis, Ehrlichiosis, Powassan virus disease, Borrelia miyamotoi disease, Borrelia mayonii disease, or Rocky Mountain spotted fever (RMSF). The second protocol quantified (e.g., ng/mL or pg/mL) antibody levels for antibody class profiling or “fingerprinting”. This protocol mimics the testing algorithm for serial cardiac troponin testing to rule-in or rule-out acute myocardial infarction (AMI). Longitudinal Borrelia antibody testing was performed, such as Day 0 versus Day 3, to determine if there was an acute change in IgM and/or IgG antibody levels, or a ratio of IgG and IgM, indicating an active infection. Due to the challenges of obtaining serum samples over multiple days, saliva or saline oral rinse (SOR) was used as a sample type. This would have the added benefit of making the test more accessible to different patient populations and geographies using at-home saliva-based sample collection.

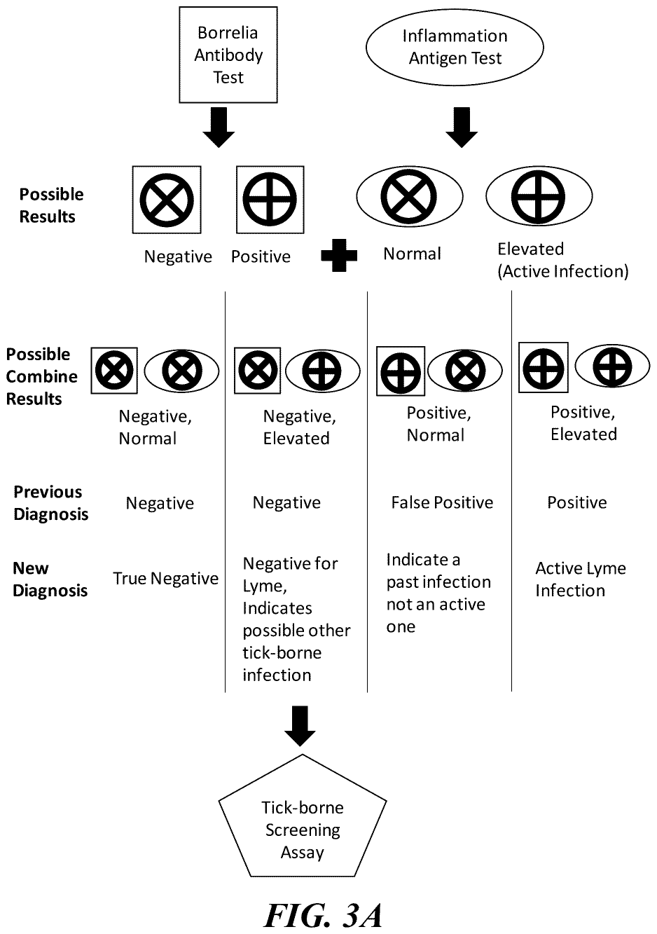

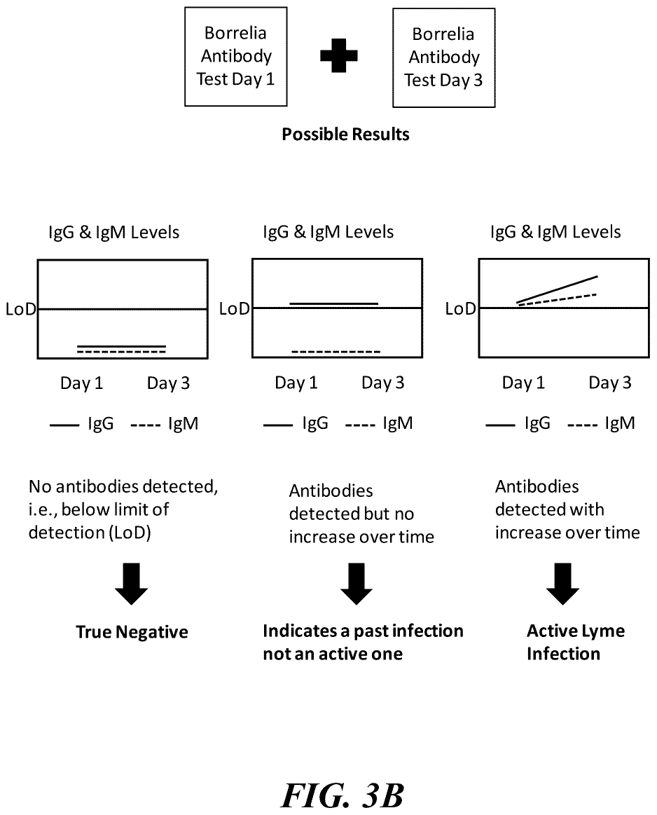

FIG. 3A shows the first protocol, combining the Borrelia antibody testing with an inflammatory antigen testing. Borrelia antibody testing can have 2 possible results, i.e., negative and positive. Inflammatory antigen testing can have 2 possible results, i.e., negative and positive. A combination of Borrelia antibody testing result and inflammatory antigen testing can indicate if the subject was experiencing a Lyme injection. For example, a negative Borrelia antibody testing with a negative inflammatory antigen testing means the subject is not experiencing and has not had a Lyme infection; a negative Borrelia antibody testing with a positive inflammatory antigen testing means the subject is not experiencing and has not had a Lyme infection but could be infected by other tick-borne infection; a positive Borrelia antibody testing with a negative inflammatory antigen testing means the subject has had a Lyme infection (a past infection) but is not experiencing an active Lyme infection; a positive Borrelia antibody testing with a positive inflammatory antigen testing means the subject is experiencing an active Lyme infection. In contrast, antibody testing without other tests may possibly have a false positive result or not be able to capture other tick-bome infections.

FIG. 3B shows the second protocol, Longitudinal Borrelia antibody testing at day 1 and day 3. It quantitates the change in anti-Borrelia IgG and/or IgM levels over time and determines if there is an active infection as opposed to past injection. If no antibodies are detected in both day 1 and day 3, it indicates a true negative (no Lyme infection). If antibodies are detected but do not increase over time, it indicates a past Lyme infection. If antibodies are detected and increase over time, it indicates an active Lyme infection.

A 7C Process (as shown in FIG. 2E) was used for the testing. In the conditioning (C1) step patient samples were pre-analytically conditioned with paramagnetic interference capture particles, e.g., “clean beads” to selectively bind and remove sample-specific heterophilic antibody interference and/or autoantibody interference, as well as free biotin, anti-streptavidin, and anti-biotin interference. During the capture (C2) step specific antibodies and/or antigens were subsequently captured by adding paramagnetic capture beads to the conditioned sample. The capture beads comprise paramagnetic streptavidin beads coated with biotinylated antibodies and/or biotinylated antigens. Once the target antibody or antigen was bound to the beads the samples were cleaned (C3) by washing the beads and removing the sample matrix. The sample was concentrated (C4) by reducing the sample volume. Targeted fluorescent conjugates (C5) were added to samples. Specific human immunoglobulins were detected by adding a rabbit polyclonal anti-human IgG, anti-human IgM, and anti-human IgA multiplex fluorescent conjugates, and specific antigens were detected by adding a fluorescently labeled monoclonal antibody(s). After a sample incubation, the capture beads were washed to remove excess conjugates. The conjugate was cleaved (C6) from the capture beads and characterized (C7) via fluorescent measurement. The amount of neutralizing antibodies and antigens detected in the sample was determined by use of a bead-based calibration curve. The calibration curve was generated using seven different calibrator beads with different amounts of purified human IgG conjugated to each calibrator bead. The relative fluorescence units were directly proportional to the amount of antigen-specific IgG immunoglobulins captured by the Capture Beads.

For Protocol 1, if 2 out of 3 IgG, IgM, or IgA antibodies are positive, then a sample is antibody positive, or if 2 out of 3 IgG, IgM, or IgA antibodies are negative, then the sample is antibody negative. For Protocol 2, if IgG and IgM are both positive then the sample is positive, if not, the sample is negative. In both cases, a positive IgG, IgM, or IgA test result occurs when the respective antibody class test signal is above the cutoff established with Lyme disease negative samples. The results for Protocol 1 and Protocol can be found in Table 3. Samples tested were either true positive Lyme disease serum samples (comprise anti-Borrelia antibodies; patients clinically diagnosed with Lyme disease) or true negative serum samples (do not comprise anti-Borrelia antibodies, the patients were not clinically diagnosed with Lyme disease) as determined and validated by the CDC and the Bay Area Lyme Foundation. Prior to our analysis, the samples were also tested by IgM Western blot, IgG Western blot, and ELISA (IgG/IgM C-6 Peptide ELISA) to confirm positive and negative results.

TABLE 3
Testing results of protocol 1 and protocol
2 in comparison with reference tests

| | Protocol 1 | | |
|---|---|---|---|
| | Disease | Disease | |
| Test IgG | Pos | Neg | n |
| Pos Test | 22 | 0 | 22 |
| Neg Test | 0 | 29 | 29 |
| n | 22 | 29 | 51 |

| | | 95% CI | 95% CI |
|---|---|---|---|
| | Value | Low | High |
| Sensitivity | 100% | 82% | 100% |
| Specificity | 100% | 85% | 100% |
| PPV | 100% | 82% | 100% |
| NPV | 100% | 85% | 100% |

| | Protocol 2 | | |
|---|---|---|---|
| | Disease | Disease | |
| Test IgG | Pos | Neg | n |
| Pos Test | 22 | 0 | 22 |
| Neg Test | 0 | 29 | 29 |
| n | 22 | 29 | 51 |

| | | 95% CI | 95% CI |
|---|---|---|---|
| | Value | Low | High |
| Sensitivity | 100% | 82% | 100% |
| Specificity | 100% | 85% | 100% |
| PPV | 100% | 82% | 100% |
| NPV | 100% | 85% | 100% |

### Example 6: COVID-19 Neutralizing Antibody Test

This example illustrates the development of a COVID-19 neutralizing antibody test.

Eliminate BSA coated beads from the Capture Bead pool: BSA coated beads (or biomarker capture particles), added to the RBD, NTD, and Delta RBD Capture Bead pool as “null beads” to increase the total mass of beads per test, demonstrated false positive human serum results in troubleshooting studies. The mechanism of cross-reactivity is likely human anti-BSA heterophilic interference. To improve assay efficacy and accuracy, BSA coated beads were eliminated from the Capture Bead pool.

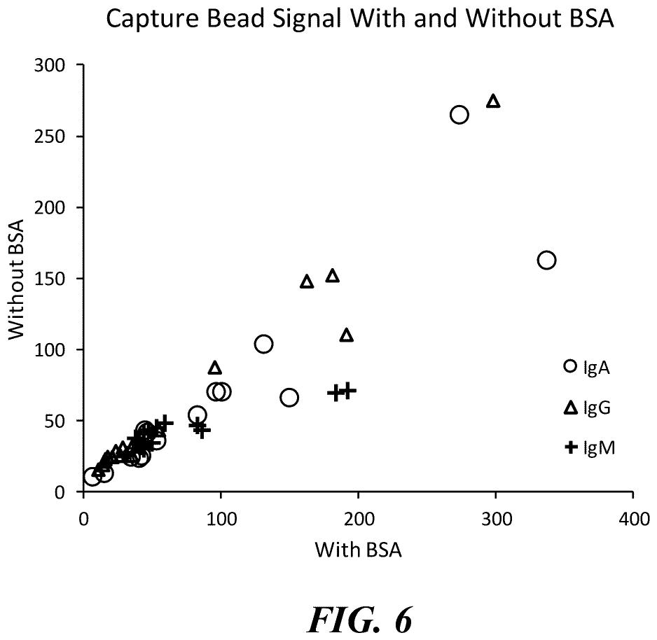

FIG. 6 shows that the main response of capture beads was equivalent with and without BSA coated beads. However, the population of samples located within the red encircled area demonstrates a subpopulation where the capture beads with BSA had a significant increase compared to without BSA beads. Absence or reduced signal specifically associated with the absence of BSA coated beads may be signal that is associated with antibodies directed to Covid-19 RBD or NTD.

The triplex conjugate with RBD/NTD/Delta RBD Capture Beads was cleaned to remove SARS-CoV-2 spike protein RBD and NTD cross-reactivity and decrease background signal. The Agilent DAKO anti-human IgG polyclonal rabbit antibody raw material used for the anti-human IgG conjugate labelled with Alexa Fluor 555 has SARS-CoV-2 spike protein RBD and NTD cross-reactivity. This cross-reactivity resulted in a higher than desired relative fluorescence units (RFU) background signal and assay cutoff (LoQ) in the test as compared to the Agilent DAKO anti-human IgM and anti-human IgA polyclonal rabbit antibody raw materials.

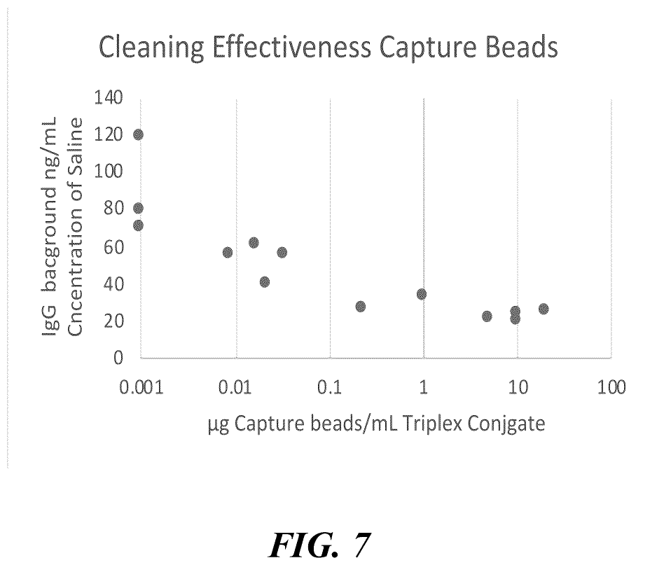

A triplex conjugate was subjected to exposure to increasing the mass of capture beads. Unit of conjugate was 200 μL finished conjugate which is the volume applied per test dose of the Covid19 Test. FIG. 7 shows the reduction in background (measuring Saline as a sample) of IgG with increased mass (pg) of capture beads per mL triplex conjugate.

In the current configuration, reduction in background was sufficiently low at 0.22 μg beads/test dose conjugate. In the above graph to use a logarithmic scale, zero pg capture beads are placed at 0.001 μg

It was determined that the amount of capture beads/test dose of conjugate would be 0.3 μg.

Replace current Triplex Conjugate fluorophores with new fluorophores to enhance assay sensitivity: new fluorophores from other Manufacturers (iFluors from AAT Bioquest, and CFluors from Biotium) may enhance assay sensitivity as compared to the current ThermoFisher Alexa Fluor 488, 555, and 647 fluorophores used to make the Triplex conjugate, or 488-Anti-Human IgM, 555-Anti-Human IgG, and 647-Anti-Human IgA conjugates.

Experiment #1: Molar excess vs S/N for AlexaFluor, CF Biotium and iFluor. The currently used DAKO brand anti-IgG, IgA, and IgM rabbit polyclonal antibodies were fluorinated. Various molar excess of reactions was used and compared to the current ThermoFisher AlexaFluor brand. An example of each comparison is shown in Table 4.

TABLE 4
Comparison of calibration using different fluorophores

| | | Alexa | Alexa | Alexa | CF488A | CF488A | CF488A | iFluor488 | iFluor488 | iFluor488 | |
|---|---|---|---|---|---|---|---|---|---|---|---|
| IgM | ng/mL | 488 5X | 488 10X | 488 25X | 5X | 10X | 25X | 5X | 10X | 25X | |
| Cal 6 | 518 | 28040 | 44247 | 46918 | 31561 | 36538 | 40882 | 34282 | 46199 | 26973 | 495, 530 |
| Cal 5 | 245 | 27859 | 39384 | 44770 | 31268 | 32475 | 39909 | 36814 | 44723 | 25561 | 495, 530 |
| Cal 4 | 178 | 25334 | 36248 | 41236 | 29654 | 32170 | 36495 | 34984 | 41780 | 26059 | 495, 530 |
| Cal 3 | 84 | 20881 | 30481 | 33266 | 23581 | 26818 | 29112 | 26747 | 32125 | 21022 | 495, 530 |
| Cal 2 | 35 | 13942 | 19474 | 20703 | 16044 | 17127 | 18938 | 19178 | 20076 | 15239 | 495, 530 |
| Cal 1 | 17 | 11885 | 14756 | 15159 | 12591 | 13406 | 15048 | 13743 | 15870 | 12122 | 495, 530 |
| Cal 0 | 0 | 7562 | 7768 | 8029 | 7759 | 8008 | 8033 | 7943 | 7667 | 7721 | 495, 530 |
| | S/N | 3.7 | 5.7 | 5.8 | 4.1 | 4.6 | 5.1 | 4.3 | 6.0 | 3.5 | |

| | | Alexa | Alexa | Alexa | CF543 | CF543 | iFluor | iFluor | iFluor | |
|---|---|---|---|---|---|---|---|---|---|---|
| IgG | ng/ml | 555 5X | 555 10X | 555 25X | 5X | 10X | 546 5X | 546 10X | 546 25X | |
| Cal 6 | 739 | 32395 | 71103 | 32307 | 41748 | 35904 | 22224 | 53761 | 66512 | 550, 580 |
| Cal 5 | 459 | 31531 | 66277 | 27858 | 39648 | 35193 | 22957 | 52947 | 60165 | 550, 580 |
| Cal 4 | 276 | 26608 | 56471 | 24305 | 36148 | 31154 | 20307 | 39332 | 53329 | 550, 580 |
| Cal 3 | 119 | 17288 | 34048 | 16899 | 26685 | 24312 | 14310 | 32643 | 37540 | 550, 580 |
| Cal 2 | 45 | 7737 | 17486 | 9515 | 12576 | 12531 | 6895 | 15829 | 17657 | 550, 580 |
| Cal 1 | 21 | 5246 | 10964 | 5150 | 7475 | 7461 | 3649 | 9416 | 10891 | 550, 580 |
| Cal 0 | 0 | 563 | 546 | 548 | 504 | 677 | 1046 | 479 | 574 | 550, 580 |
| | S/N | 57.5 | 130.2 | 59.0 | 82.8 | 53.0 | 21.2 | 112.2 | 115.9 | |

| | | Alexa | Alexa | Alexa | CF597R | CF597R | CF597R | iFluor597 | iFluor597 | iFluor597 | |
|---|---|---|---|---|---|---|---|---|---|---|---|
| IgA | ng/mL | 647 5X | 647 10X | 647 25X | 5X | 10X | 25X | 5X | 10X | 25X | |
| Cal 6 | 371 | 131 | 163 | 83 | 12982 | 29152 | 36654 | 12671 | 17625 | 12512 | 585, 621 |
| Cal 5 | 241 | 137 | 127 | 96 | 12616 | 27873 | 30815 | 13372 | 16948 | 11295 | 585, 621 |
| Cal 4 | 167 | 123 | 113 | 101 | 10812 | 23138 | 24979 | 11643 | 16059 | 9663 | 585, 621 |
| Cal 3 | 79 | 72 | 132 | 125 | 6416 | 14221 | 13860 | 8429 | 11030 | 7353 | 585, 621 |
| Cal 2 | 32 | 100 | 124 | 108 | 3512 | 6049 | 6060 | 3739 | 5010 | 4047 | 585, 621 |
| Cal 1 | 15 | 107 | 107 | 46 | 1870 | 3365 | 3365 | 2396 | 3216 | 2193 | 585, 621 |
| Cal 0 | 0 | 148 | 75 | 101 | 119 | 154 | 109 | 122 | 123 | 123 | 585, 621 |
| | S/N | 0.9 | 2.2 | 0.8 | 109.1 | 189.3 | 336.3 | 103.9 | 143.3 | 101.7 | |

The data shows that iFluors provided superior average signal to noise ratios. AlexaFluor had limited benefit compared the CF or iFluor. Even when AlexaFluor had a clearly superior S/N for IgG at 10×, the responses bracketing that at 5× and 25× suggest that a narrow range of good signal may be subject to manufacturing error if mixtures are not handled well.

Experiment #2. Direct comparison of Cleaned conjugate with AlexaFluor and iFluor using controls and select samples. Using the IFU protocol for PN500081, select samples and controls were run in direct comparison. The following data demonstrate good equivalency between AlexaFluor and iFluor (Table 5).

TABLE 5
Comparison of cleaned conjugates

| | TriCal Capture (μL) | | | | | |
|---|---|---|---|---|---|---|
| | IgM | IgM | IgG | IgG Master | Master | Master |
| | Master Lot A | Master Lot A | Master Lot A | Lot A | Lot A | Lot A |
| Conjugate | Alexa | iFluor | Alexa | iFluor | Alexa | iFluor |
| BR 60 | 61.7 | 47.0 | 416.5 | 458.6 | 17.7 | 24.8 |
| BR 15 | 34.5 | 19.0 | 105.9 | 98.8 | 5.5 | 12.0 |
| Neg Ctl | 19.5 | 6.6 | 14.2 | 13.0 | 5.1 | 7.2 |
| SB 2/10 | 37.0 | 15.3 | 125.0 | 104.8 | 73.7 | 57.8 |
| VK 2/10 | 28.6 | 12.3 | 22.5 | 24.6 | 26.1 | 30.0 |
| DN 3/10 | 63.6 | 46.6 | 100.3 | 105.0 | 126.0 | 160.1 |
| MRS 2/10 | 21.8 | 8.7 | 12.5 | 11.5 | 12.7 | 12.4 |
| KM 2/10 | 28.9 | 13.1 | 44.2 | 44.0 | 45.6 | 45.5 |

The data shows that iFluor combined with cleaned conjugate at 0.3 μg capture beads per dose conjugate is acceptable.

Experiment #3. Capture beads were built with the following mixtures per dose
i. Wild Type 100 μg, Delta 33 μg, NTD 33 μg
ii. Wild Type 100 μg, Delta 33 μg, Omicron 33 μg, NTD 33 μg

The underlying theory is that the presence of Omicron variant RBD on the capture bead will incur a greater RFU signal. Samples collected from an investor meeting early in 2022 were tested and the results are shown in Table 6.

TABLE 6
Summary of antibody detection results

| | Capture Beads Wild + | | | | Capture Beads w/ | | | Omicron/ | | |
|---|---|---|---|---|---|---|---|---|---|---|
| Mar. 29, 2022 | Delta only | | | | Omicron | | | Delta | | |
| Sample ID | IgA | IgG | IgM | Sample ID | IgA | IgG | IgM | IgA | IgG | IgM |
| BR 60 | 22.0 | 517.7 | 45.6 | BR 60 | 21.2 | 548.3 | 46.9 | 96% | 106% | 103% |
| BR 15 | 11.8 | 136.1 | 23.9 | BR 15 | 10.1 | 106.4 | 21.9 | 85% | 78% | 92% |
| Neg Ctl | 6.3 | 15.5 | 7.2 | Neg Ctl | 6.9 | 18.1 | 11.2 | 111% | 117% | 157% |
| BR 60 | 24.7 | 600.6 | 56.6 | BR 60 | 22.6 | 614.2 | 49.2 | 91% | 102% | 87% |
| BR 15 | 10.8 | 111.1 | 20.9 | BR 15 | 12.1 | 113.0 | 22.8 | 113% | 102% | 109% |
| Neg Ctl | 6.2 | 18.8 | 10.8 | Neg Ctl | 7.3 | 22.3 | 12.8 | 116% | 119% | 118% |
| S1 | 11.6 | 13.2 | 15.2 | S1 | 13.7 | 14.4 | 13.8 | 119% | 109% | 91% |
| S2 | 10.0 | 18.8 | 14.3 | S2 | 10.4 | 22.4 | 15.3 | 104% | 119% | 107% |
| S3 | 42.6 | 45.5 | 18.1 | S3 | 40.1 | 51.3 | 19.5 | 94% | 113% | 108% |
| S4 | 46.6 | 22.1 | 12.0 | S4 | 53.2 | 31.3 | 13.1 | 114% | 142% | 109% |
| S5 | 27.6 | 16.3 | 13.3 | S5 | 25.1 | 17.9 | 12.8 | 91% | 110% | 96% |
| S6 | 25.2 | 27.2 | 16.4 | S6 | 22.2 | 25.2 | 15.5 | 88% | 93% | 94% |
| S7 | 19.6 | 20.7 | 15.5 | S7 | 15.8 | 17.8 | 13.1 | 80% | 86% | 85% |
| S8 | 25.7 | 35.6 | 14.8 | S8 | 24.8 | 33.9 | 14.8 | 97% | 95% | 100% |
| S9 | 14.5 | 23.4 | 12.9 | S9 | 11.5 | 22.5 | 14.8 | 80% | 96% | 115% |
| S10 | 12.3 | 12.4 | 10.1 | S10 | 10.9 | 12.1 | 8.5 | 89% | 98% | 84% |
| S11 | 54.1 | 29.1 | 11.5 | S11 | 60.4 | 32.2 | 15.5 | 112% | 111% | 134% |
| S12 | 14.5 | 15.7 | 11.2 | S12 | 16.4 | 17.2 | 10.5 | 113% | 109% | 94% |
| S13 | 36.5 | 14.6 | 10.8 | S13 | 35.8 | 16.7 | 12.0 | 98% | 115% | 111% |
| S14 | 31.1 | 30.4 | 16.3 | S14 | 27.2 | 28.6 | 16.7 | 87% | 94% | 102% |

Experiment #3 Omicron variant RBD capture beads may increase secretory IgA, IgG, or IgM response in some patient saline oral rinse patient samples as compared to the Delta variant RBD capture beads, and by having both Omicron variant RBD and Delta variant RBD capture beads in the capture bead pool with Wild type RBD and NTD capture bead would increase overall sensitivity of detecting anti-SARS-CoV-2 spike protein RBD antibodies.

Experiment #4: The sample capture beads mixtures as in experiment #3 were compared but with positive samples recently collected. The following data (Table 7) compares with and without Omicron.

TABLE 7
Summary of antibody detection results

| | | | | | | | | Omicron/ | | |
|---|---|---|---|---|---|---|---|---|---|---|
| Apr. 21, 2022 | Capture Beads Wild + Delta only | | | | Capture Beads w/Omicron | | | Delta | | |
| Sample ID | IgA | IgG | IgM | BR 60 | IgA | IgG | IgM | IgA | IgG | IgM |
| BR 60 | Neg | | 55.6 | BR 60 | Neg | | 44.5 | Neg | | 80% |
| BR 15 | Neg | 216.9 | Neg | BR 15 | Neg | 211.0 | Neg | Neg | 97% | Neg |
| Neg Ctl | Neg | Neg | Neg | Neg Ctl | Neg | Neg | Neg | Neg | Neg | Neg |
| BR 60 | Neg | | 59.1 | BR 60 | Neg | | 43.0 | Neg | | 73% |
| BR 15 | Neg | 228.9 | Neg | BR 15 | Neg | 353.5 | 46.6 | Neg | 154% | Neg |
| Neg Ctl | Neg | Neg | Neg | Neg Ctl | Neg | Neg | Neg | Neg | Neg | Neg |
| S15 | Neg | Neg | Neg | S15 | Neg | Neg | Neg | Neg | Neg | Neg |
| S16 | 132.6 | 87.2 | Neg | S16 | 134.7 | 125.2 | Neg | 102% | 144% | Neg |
| S17 | 49.0 | Neg | Neg | S17 | 74.3 | 108.5 | Neg | 152% | 201% | Neg |
| S18 | 45.2 | Neg | Neg | S18 | 49.5 | Neg | Neg | 110% | Neg | Neg |
| S19 | Neg | Neg | Neg | S19 | Neg | Neg | Neg | Neg | Neg | Neg |
| S20 | Neg | Neg | Neg | S20 | Neg | Neg | Neg | Neg | Neg | Neg |

The addition of Omicron RBD to the mixture did not significantly affect the controls (with the noted exception of the duplicate IgG BR 15 that increased from 228.9 to 353.5 while its partner duplicate decreased slightly). However, two patient samples significantly increased. S2 IgG, S3 IgA and IgG had 52%, 44% and 101% increases, respectively. In fact, S3 IgG transforming from negative to positive is a significant change. Positive changes in signal show that the addition of Omicron does effectively increase the sensitivity of the assay. Omicron variant RBD capture beads may increase secretory IgA, IgG, or IgM response in some patient saline oral rinse patient samples as compared to the Delta variant RBD capture beads, and by having both Omicron variant RBD and Delta variant RBD capture beads in the capture bead pool with Wild type RBD and NTD capture bead would increase overall sensitivity of detecting anti-SARS-CoV-2 spike protein RBD antibodies.

The current test was validated as a semi-automated method on the laboratory bench. The test protocol comprises many manual pipetting and mixing steps, and timed heated (37° C.) incubations, which due to human error and operator-to-operator differences are not as well controlled as automation and may therefore be limiting in the pursuit of accuracy and precision and continuous quality management. Fully automate the test using a liquid handler such as the Beckman Coulter® Biomek® i7 to improve assay precision, reproducibility, sensitivity, and throughout.

The manual method used pipette mixing to disrupt and re-suspend beads after pellet formation during magnetic washing. The i7 pipettor could not accomplish this function. Several programmable pipetting techniques were attempted. These were followed by visual inspection of the bead re-suspension within the DW plates. In each case large particulates were observable meaning that re-suspension was inadequate. Following this, an attempt to suspend by increasing shaker speed on the QInstrument was proposed and tested. The initial result was that increasing shaker speed to 2,000 RPM accomplished re-suspension. However, the built-in clamping devices on the heater shaker were not sufficiently secure to keep the DW plate on the heater shaker and the DW plates will fall off. Subsequent testing showed that reducing shaking speed to 1,500 RPM was minimally sufficient to re-suspend and various attempts to show that the plates were secure resulted in a 1 in 10 failure to keep the plates on the heater shaker. After placing an adapter, the DW plates at 1,500 RPM were stable during the shake. Visual confirmation showed that the beads were well suspended and disperse.

The manual method of washing used a V&P plate magnet (PN VP 771BT-SF) fitted within the BioTek plate washer. This plate magnet caused problematically differentiated signal on alternate columns of the deep well plate. An example of that data is shown in Table 8.

TABLE 8
Summary of detection results using manual method

| IgG | 1 | 2 | 3 | 4 | 5 | 6 | 7 | 8 | 9 | 10 | 11 | 12 | Ave | SD | CV |
|---|---|---|---|---|---|---|---|---|---|---|---|---|---|---|---|
| A | 1633 | 1752 | 1753 | 1708 | 1780 | 1715 | 1781 | 1602 | 1705 | 1777 | 1683 | 1594 | 1707 | 67 | 4% |
| B | 1503 | 1628 | 1722 | 1577 | 1720 | 1413 | 1769 | 1539 | 1669 | 1510 | 1651 | 1543 | 1604 | 107 | 7% |
| C | 1663 | 1515 | 1916 | 1389 | 1725 | 1498 | 1692 | 1459 | 1694 | 1590 | 1656 | 1577 | 1615 | 141 | 9% |
| D | 1520 | 1511 | 1684 | 1529 | 1689 | 1542 | 1671 | 1551 | 1693 | 1425 | 1588 | 1531 | 1578 | 87 | 6% |
| B | 1591 | 1634 | 1747 | 1490 | 1703 | 1531 | 1703 | 1535 | 1640 | 1455 | 1536 | 1424 | 1582 | 104 | 7% |
| F | 1493 | 1426 | 1757 | 1577 | 1785 | 1478 | 1753 | 1590 | 1667 | 1504 | 1658 | 1546 | 1603 | 120 | 8% |
| G | 1686 | 1512 | 1781 | 1520 | 1748 | 1525 | 1737 | 1489 | 1740 | 1602 | 1696 | 1560 | 1633 | 109 | 7% |
| H | 1615 | 1749 | 1873 | 1663 | 1801 | 1670 | 1828 | 1716 | 1727 | 1681 | 1697 | 1669 | 1724 | 76 | 4% |
| Ave | 1588 | 1591 | 1779 | 1557 | 1744 | 1547 | 1742 | 1560 | 1692 | 1568 | 1646 | 1556 | | | |
| SD | 75 | 119 | 78 | 100 | 41 | 99 | 52 | 79 | 33 | 119 | 56 | 69 | | | |
| CV | 5% | 8% | 4% | 6% | 2% | 6% | 3% | 5% | 2% | 8% | 3% | 4% | | | |

In this configuration, odd numbered columns had pellets located on the right wall of the well while even numbered columns had pellets located on the left side wall of each well. At the same time the dispense nozzle of the washer was directed on the left side of each well. This resulted in even numbered columns having the wash buffer flow directly over them—essentially washing more effectively those pelleted beads than the odd numbered columns.

2 custom magnetic separation plates were ordered from Alpaqua. Each were a custom version of the Magnum FLX and Catalyst 96 magnetic separation plates already offered by Alpaqua altered so that they fit into the plate carrier of the ELx405DW washer. The Magnum FLX offers the strongest magnets which create a circular bead pellet at the bottom of each well. Utilizing this magnet revealed a complete correction of the add-even column issue. However, two issues were shown: A) visual inspection of the washed plates showed the left side of each circular pellet was washed away and b) an unknown effect occurred with columns 8 through 11 had an unusual number of well with low signal. Thus, over all variability was increased. Table 9 shows results with Magnum FLX.

TABLE 9
Antibody detection results

| IgG | 1 | 2 | 3 | 4 | 5 | 6 | 7 | 8 | 9 | 10 | 11 | 12 | Ave | SD | CV |
|---|---|---|---|---|---|---|---|---|---|---|---|---|---|---|---|
| A | 1004 | 1153 | 1073 | 1158 | 1447 | 1162 | 1031 | 1161 | 1165 | 1168 | 1180 | 1055 | 1146 | 113 | 10% |
| B | 1068 | 1220 | 1188 | 1161 | 1164 | 1173 | 1098 | 1174 | 1166 | 1205 | 1148 | 1121 | 1157 | 43 | 4% |
| C | 1303 | 1165 | 1635 | 1169 | 1206 | 742 | 648 | 954 | 691 | 270 | 764 | 1109 | 971 | 366 | 38% |
| D | 1168 | 1152 | 1174 | 1112 | 1135 | 1049 | 995 | 240 | 981 | 524 | 1127 | 1182 | 987 | 296 | 30% |
| E | 1092 | 1124 | 1174 | 1033 | 1048 | 951 | 987 | 584 | 921 | 773 | 923 | 816 | 952 | 166 | 17% |
| F | 1148 | 1185 | 1139 | 1045 | 1063 | 1038 | 849 | 877 | 838 | 771 | 1052 | 998 | 1000 | 136 | 14% |
| G | 1111 | 1207 | 1184 | 971 | 1216 | 934 | 957 | 820 | 521 | 639 | 987 | 1120 | 972 | 222 | 23% |
| H | 1090 | 1192 | 1125 | 1142 | 1108 | 992 | 678 | 986 | 901 | 915 | 882 | 839 | 988 | 151 | 15% |
| Ave | 1123 | 1175 | 1212 | 1099 | 1173 | 1005 | 905 | 850 | 898 | 783 | 1008 | 1030 | | | |
| SD | 88 | 32 | 175 | 74 | 126 | 138 | 165 | 311 | 220 | 315 | 146 | 136 | | | |
| CV | 8% | 3% | 14% | 7% | 11% | 14% | 18% | 37% | 25% | 40% | 14% | 13% | | | |

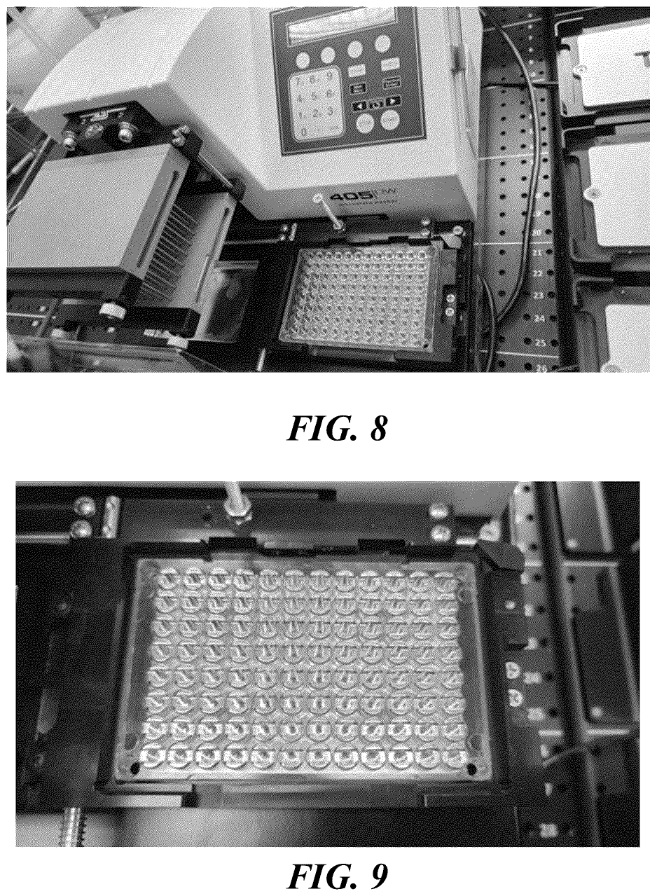

Subsequent to the custom Magnum FLX, a custom Catalyst 96 was trailed. The geometry of these magnets is that each well will have a pair of pellets on opposite sides of the well. The orientation of these is adjustable. We elected to have them on an up-down orientation so that a gap of no beads is present at the side of the well where the washer dispenses liquid. Pictures of the custom Catalyst 96 slotted magnet are shown in FIG. 8 and FIG. 9.

The Custom Catalyst 96 precision data is shown in Table 10.

TABLE 10
Antibody detection results with Custom Catalyst 96

| IgG - | | | | | | | | | | | | | | | |
|---|---|---|---|---|---|---|---|---|---|---|---|---|---|---|---|
| Slotted | 1 | 2 | 3 | 4 | 5 | 6 | 7 | 8 | 9 | 10 | 11 | 12 | Avg | SD | CV |
| A | 1659 | 1803 | 1571 | 1603 | 1579 | 1509 | 1577 | 1668 | 1602 | 1346 | 1662 | 1586 | 1597 | 108 | 7% |
| B | 1564 | 1747 | 1888 | 1509 | 1700 | 1693 | 1674 | 1719 | 1703 | 1514 | 1732 | 1714 | 1680 | 106 | 6% |
| C | 1661 | 1638 | 1489 | 1683 | 1439 | 1134 | 1312 | 1610 | 1439 | 1489 | 1522 | 1647 | 1505 | 162 | 11% |
| D | 1557 | 1685 | 1707 | 1794 | 1670 | 1382 | 1632 | 1548 | 1593 | 1431 | 1609 | 1628 | 1603 | 114 | 7% |
| E | 1663 | 1513 | 1552 | 1630 | 1479 | 1564 | 1562 | 1542 | 1524 | 1350 | 1660 | 1621 | 1555 | 87 | 6% |
| F | 1590 | 1682 | 1581 | 1645 | 1521 | 1575 | 1494 | 1641 | 1487 | 1364 | 1639 | 1587 | 1567 | 88 | 6% |
| G | 1495 | 1667 | 1541 | 1507 | 1503 | 1568 | 1515 | 1433 | 1308 | 1315 | 1617 | 1666 | 1511 | 117 | 8% |
| H | 1553 | 1737 | 1659 | 1585 | 1520 | 1654 | 1516 | 1569 | 1464 | 1481 | 1717 | 1686 | 1595 | 93 | 6% |
| Avg | 1593 | 1684 | 1623 | 1619 | 1551 | 1510 | 1535 | 1591 | 1515 | 1411 | 1644 | 1642 | | | |
| SD | 62 | 87 | 127 | 94 | 92 | 178 | 109 | 88 | 120 | 77 | 66 | 45 | | | |
| CV | 4% | 5% | 8% | 6% | 6% | 12% | 7% | 6% | 8% | 5% | 4% | 3% | | | |

The custom Catalyst 96 plate magnet from Alpaqua gave acceptable precision with whole plate CV at 8%. Having reduced variability from magnets and re-suspension of beads, Pipetting parameter techniques may further be optimized.

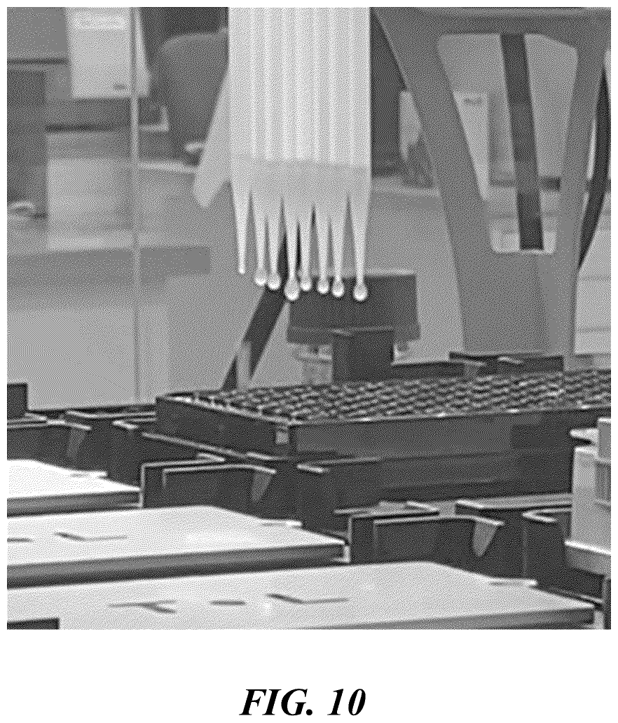

The Span-8 pipetting parameters were determined to be a source of variability because of a) slow speed of delivery and b) height of delivery. Visual evidence revealed that the pipetting technique resulted in large drops being delivered to the deep wells from the top of the wells. FIG. 10 shows an exemplary pipetting technique.

A different technique was programmed where instead of pipetting multiple 100 μL aliquots from a large primary aspiration, single dispenses from small volume tips were programmed. Additionally, the delivery was at the bottom of the well. After this programming was completed, a full plate using a single pooled sample was run using the IFU protocol, running completely on the 17. Table 11 shows the antibody detection results.

TABLE 11
Antibody detection results with Beckman pipettes system

| | | | | | | | | | | | | | Row | | |
|---|---|---|---|---|---|---|---|---|---|---|---|---|---|---|---|
| IgG | 1 | 2 | 3 | 4 | 5 | 6 | 7 | 8 | 9 | 10 | 11 | 12 | Ave | SD | CV % |
| A | 30 | 747 | 770 | 735 | 725 | 733 | 725 | 736 | 700 | 731 | 745 | 708 | 732 | 19 | 2.6% |
| B | 719 | 753 | 725 | 784 | 758 | 735 | 768 | 704 | 767 | 746 | 733 | 738 | 746 | 23 | 3.0% |
| C | 1496 | 750 | 772 | 743 | 780 | 778 | 757 | 743 | 781 | 715 | 779 | 711 | 755 | 26 | 3.4% |
| D | 3644 | 719 | 793 | 761 | 819 | 738 | 779 | 767 | 781 | 714 | 726 | 696 | 754 | 38 | 5.1% |
| E | 6242 | 791 | 719 | 760 | 750 | 801 | 786 | 739 | 741 | 740 | 743 | 746 | 756 | 26 | 3.4% |
| F | 8634 | 742 | 806 | 766 | 760 | 756 | 733 | 747 | 711 | 740 | 733 | 728 | 747 | 25 | 3.3% |
| G | 10508 | | | | | | | | | | | | | | |
| H | 797 | 752 | 734 | 742 | 774 | 745 | 708 | 746 | 733 | 3504 | 6734 | 6023 | 742 | 19 | 2.5% |
| | Column | 751 | 760 | 756 | 767 | 755 | 751 | 740 | 745 | 731 | 743 | 721 | | | |
| | Average | | | | | | | | | | | | | | |
| | SD | 23 | 35 | 17 | 32 | 28 | 25 | 20 | 35 | 14 | 19 | 19 | | | |
| | CV % | 3.1% | 4.7% | 2.3% | 4.1% | 3.7% | 3.3% | 2.8% | 4.7% | 1.9% | 2.5% | 2.7% | | | |

The Biomek® i7 has been optimized to use a custom Alpaqua Catalyst 96 magnet, or a slotted magnet, in the ELx405 deep well washer. Washer programming demonstrated acceptable performance, and pipetting parameters have been optimized to acceptable performance. The Beckman Coulter® Biomek® i7 system has acceptable performance and is ready to be validated and employed in the COVID-19 Neutralizing Antibody Test Laboratory Developed Test per the Validation Plan.

### Example 7: COVID-19 Neutralizing Antibody Test

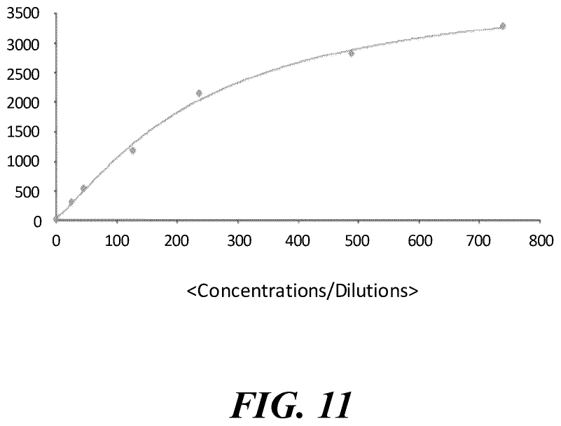

Disclosed herein is a Neutralizing Antibody Test Serum Protocol (“Serum Protocol”) which was used in this antibody test. Serum samples were pre-analytically treated with paramagnetic “clean beads” to selectively bind and remove sample-specific heterophilic antibody interference and/or autoantibody interference. After a sample incubation, the clean beads were isolated with a magnet, and the conditioned sample was aspirated and transferred to the reaction plate (Condition). Neutralizing SARS-CoV-2 antibodies were subsequently captured by adding paramagnetic “capture beads” to the conditioned sample. The capture beads comprised paramagnetic streptavidin beads coated with biotinylated SARS-CoV-2 spike protein RBD-specific recombinant antigens and SARS-CoV-2 spike protein NTD-specific recombinant antigen (Capture). After a sample incubation, the capture beads were washed to remove the sample matrix and non-specific antibodies clean) and the sample volume was decreased (Concentrate). Neutralizing SARS-CoV-2 antibodies were detected by adding a rabbit polyclonal anti-human IgG fluorescent conjugate (Conjugate). After a sample incubation, the capture beads were washed to remove excess conjugate. The conjugate was released from the capture beads (Cleave), aspirated, and dispensed into a neutralizing buffer for subsequent fluorescent detection and measurement (Characterize). The amount of neutralizing SARS-CoV-2 IgG antibodies detected in the serum was determined by use of a bead-based calibration curve. The calibration curve (FIG. 11) was generated using seven different calibrator beads with different amounts of purified human IgG conjugated to each calibrator bead. The relative fluorescence unit is directly proportional to the amount of antigen-specific IgG immunoglobulins captured by the capture beads. The IgG has the following assay limits: limit of blank (LoB) of 3.30 μg/mL or 1553 IU/mL, limit of detection (LoD) of 4.46 μg/mL or 2099 IU/mL, limit of quantitation (LoQ) of 4.46 μg/mL or 2099 IU/mL.

### Clinical Agreement Studies

Frozen serum samples collected from non-vaccinated patients that were SARS-CoV-2 positive by the emergency use authorized Roche® Cobas® SARS-CoV-2 RT-PCR were tested by the Serum Protocol. Table 12 summaries the testing results.

TABLE 12
Testing results for serum samples collected from non-
vaccinated patients that were SARS-CoV-2 positive

| | | n | Positive | Negative | PPA | 95% CI |
|---|---|---|---|---|---|---|
| Days Post- | | | | | | |
| Symptom Onset | | | | | | |
| 0-7 | Days | 4 | 4 | 0 | 100% | 39.6%-100% |
| 8-14 | Days | 16 | 15 | 1 | 93.8% | 66.7%-99.7% |
| ≥15 | Days | 29 | 29 | 0 | 100% | 85.4%-100% |
| All Days | | 49 | 48 | 1 | 98.0% | 87.8%-99.9% |
| Days after PCR | | | | | | |
| Positive Results | | | | | | |
| 0-7 | Days | 15 | 14 | 1 | 93.3% | 66.0%-99.7% |
| 8-14 | Days | 14 | 14 | 0 | 100% | 73.2%-100% |
| ≥15 | Days | 20 | 20 | 0 | 100% | 73.2%-100% |

| | | | | | |
|---|---|---|---|---|---|
| All Days | 49 | 48 | 1 | 98.0% | 87.8%-99.9% |

Frozen serum samples collected from patients prior to November 2019 were tested by the Serum Protocol. Three false positive samples were detected in the clinical remnants. There were no false positives detected in the normal human serum or blood bank samples. The testing results are shown in Table 13.

TABLE 13
Testing results for serum samples collected
from patients prior to November 2019

| Category | | n | Positive | Negative | NPA | 95% CI |
|---|---|---|---|---|---|---|
| Human serum | | 50 | 0 | 50 | 100% | 91.1%-100% |
| before 2019 | | | | | | |
| 8-14 | Days | 40 | 0 | 40 | 100% | 89.1%-100% |
| ≥15 | Days | 47 | 3 | 44 | 93.6% | 81.4%-98.3% |

| | | | | | |
|---|---|---|---|---|---|
| All Days | 137 | 3 | 134 | 97.8% | 93.2%-99.4% |

The NISBC Verification Panel was tested by the Serum Protocol. The testing results are shown in Table 14.

TABLE 14
NISBC Verification Panel testing results

| Serum COVID-19 | Disease | Disease |
|---|---|---|
| Antibody Protocol | Positive | Negative |
| Test Positive | 23 | 1 |
| Test Negative | 0 | 13 |

| | Agreement | 95% Cl |
|---|---|---|
| Positive Percent Agreement (PPA) | 100% | 82.2-100% |
| Negative Percent Agreement (NPA) | 92.8% | 64.2-99.6% |

Finally, the performance of the Serum Protocol was compared to results obtained from the GenScript® cPass™ SARS-CoV-2 Neutralization Antibody Detection Kit (EUA201427). The results are shown in Table 15.

TABLE 15
Comparison of performance

| IgG [CutOff = 4.465 μg/mL] | Disease Positive | Disease Negative |
|---|---|---|
| Test Positive | 48 | 3 |
| Test Negative | 1 | 134 |

| | Agreement | 95% Cl |
|---|---|---|
| Sensitivity | 98.0% | 87.8-99.9% |
| Specificity | 97.8% | 93.2-99.4% |
| Positive Predictive Value (PPV) | 94.1% | 82.8-98.5% |
| Negative Predictive Value (NPV) | 99.3% | 95.3-100% |

These studies indicate that the Serum Protocol has created a sensitive and specific test or detecting neutralizing antibodies against SARS-CoV-2 spike protein RBD and NTD in serum.

### Example 8: COVID-19 Neutralizing Antibody Test for Salivary Sample

Disclosed herein is a COVID-19 Neutralizing Antibody Test SOR Protocol (“SOR Protocol”) which was used in this antibody test to multiplex detect and quantify levels of neutralizing SARS-CoV-2 antibodies (IgM, IgG, and IgA) against spike protein RBD and NTD. The samples were saline oral rinse (SOR) saliva samples.

The clinical performance of the SOR Protocol was evaluated prospectively using fresh saline oral rinse samples from non-vaccinated patients that were SARS-CoV-2 positive by the emergency use authorized OraRisk® COVID-19 RT-PCR (EUA200464). The testing results are shown in Table 16.

TABLE 16
Testing results for positive samples

| Days Post- | | | | | PPA | 95% CI |
|---|---|---|---|---|---|---|
| Symptom Onset | | n | Positive | Negative | (%) | (%) |
| 0 to 7 | Days | 44 | 42 | 2 | 95.5 | 83.3-99.2 |
| 8 to 14 | Days | 21 | 20 | 1 | 95.2 | 74.1-99.3 |
| ≥15 | Days | 5 | 5 | 0 | 100.0 | 46.3-100 |

| | | | | | |
|---|---|---|---|---|---|
| All Days | 70 | 67 | 3 | 95.7 | 87.2-99.9 |

Pre-pandemic saliva samples collected prior to November 2019 (N=37), SOR samples that were SARS-CoV-2 negative by OraRisk® COVID-19 RT-PCR and met the following SOR inclusion criteria (N=11) were tested. SOR Inclusion Criteria include (i) never vaccinated for COVID-19; (ii) never tested positive for COVID-19; (iii) never exposed to someone who was COVID-19 positive; and (iv) never had COVID-19 symptoms in the past 12 months.

The testing results are shown in Table 17.

TABLE 17
Testing results for negative samples

| Category | n | Positive | Negative | NPA | 95% Cl |
|---|---|---|---|---|---|
| SOR | 11 | 0 | 11 | 100% | 67.9-100% |
| Saliva | 37 | 0 | 37 | 100% | 88.3-100% |
| Total | 48 | 0 | 48 | 100% | 90.8-100% |

Concordance between serum and saliva (saline oral rinse) sample was tested by the Serum Protocol and SOR Protocol. Matched serum and SOR samples were collected concurrently from 27 unique subjects. The comparison is shown in Table 18.

TABLE 18
Concordance between serum and saliva samples

| SOR COVID-19 | Serum - | Serum - |
|---|---|---|
| Antibody Test | Test Positive | Test Negative |
| SOR - Test Positive | 26 | 0 |
| SOR - Test Negative | 0 | 1 |

| Agreement | | 95% CI |
|---|---|---|
| Sensitivity/PPA | 100% | 84.0%-100% |
| Specificity/NPA | 100% | 5.46%-100% |

The data shows that the SOR Protocol has high specificity and sensitivity and has the potential to support the development of any low abundance antibody detection platform. The increased sensitivity and selectivity allow novel sample types like SOR to be used for detection of secretory antibodies.

### Example 9: COVID-19 Antibody Test

The clinical performance of the disclosed COVID-19 Neutralizing Antibody Test was further assessed using the serum protocol on serum samples (“Serum Protocol”) and the results were compared with the GenScript® cPass™ SARS-CoV-2 Neutralization Antibody Detection Kit (EUA201427) method. The Serum Protocol comprised collecting positive serum samples from patients who tested positive for SARS-CoV-2 by the Roche® Cobas® SARS-CoV-2 RT-PCR, and who also tested positive by GenScript® cPass™ Neutralization Antibody Detection Kit. The presumptive negative samples were collected pre-November 2019 and independently tested negatives for neutralizing antibody to RBD using the GenScript® cPass™ Neutralization Antibody Detection Kit.

The following human serum samples were used in the serum clinical agreement study:
(i) Retrospective frozen (−20° C.) serum samples from patients who tested positive by Roche® Cobas® SARS-CoV-2 RT-PCR (4 to 28 days after symptoms onset) that were also positive by the GenScript® cPass™ Neutralization Antibody Detection Kit (N=49).
(ii) Retrospective frozen (−20° C.) serum samples collected from patients prior to December 2019 and negative by the GenScript® cPass™ Neutralizing Antibody Detection Kit (N=137). These 137 negative serum samples comprised normal human serum pre-pandemic collected in Texas (N=50), blood bank samples collected in Florida (N=40), and clinical remnant samples collected in Florida (N=47).
(iii) Prospectively collected matched serum and saline oral rinse (SOR) samples (N=28) from vaccinated patients (N=27) and from a non-vaccinated patient who was COVID-19 negative by OraRisk® COVID-19 RT-PCR (EUA200464), never tested positive for COVID-19, never was exposed to someone positive for COVID-19, and never had COVID-19 symptoms (N=1).
(iv) NISBC Verification Panel (WHO Reference Panel, First WHO International Reference Panel for anti-SARS-CoV-2 immunoglobulin, NIBSC code: 20/268) (N=37). The first WHO International Reference Panel of anti-SARS-CoV-2 immunoglobulin consists of the equivalent of 0.25 mL of pooled plasma samples obtained from individuals recovered from Coronavirus Disease 2019 (COVID-19) and a negative control plasma obtained from healthy blood donors before 2019.

GenScript® cPass™ Neutralization Antibody Detection Kit as Comparator Protocol:
(a) The 137 retrospective frozen serum samples collected from patients prior to December 2019 were tested by both GenScript® cPass™ Neutralization Antibody Detection Kit and the Serum Protocol.
(b) The 49 retrospective frozen serum samples that were RT-PCR positive for COVID-19 by the Roche® Cobas® SARS-CoV-2 RT-PCR with known Days After Symptom Onset, were also tested for neutralizing antibody to RBD by the GenScript® cPass™ Neutralization Antibody Detection Kit. These 49 samples were also tested by the Serum Protocol and results compared against the GenScript® cPass™ Neutralization Antibody Detection Kit.
(c) The 28 prospectively collected serum samples from the matched sample study were tested by both GenScript® cPass™ Neutralization Antibody Detection Kit and Serum Protocol.
(d) The 37 NISBC Verification Panel serum samples were tested by both GenScript® cPass™ Neutralization Antibody Detection Kit and Serum Protocol. All serum samples tested using the GenScript® cPass™ Neutralization Antibody Detection Kit and the Serum Protocol were compared to determine the sensitivity, PPA, specificity, and NPA of the Serum Protocol, and the correlation (r and p value) between the methods.

### Negative Agreement

The 137 retrospective frozen serum samples collected from patients prior to December 2019 were tested by the GenScript® cPass™ Neutralization Antibody Detection Kit and the Serum Protocol. The results are summarized in Table 19. The 95% CIs were calculated using an on-line Clinical Calculator (vassarstats.net/clin1.html).

TABLE 19
Negative Agreement
IgG Negative Agreement

| Category | n | Positive | Negative | Specificity | 95% CI | NPA | 95% CI |
|---|---|---|---|---|---|---|---|
| Normal Human | 50 | 0 | 50 | 100.0% | 91.1% -100% | 100.0% | 91.1% -100% |
| Serum Pre-Pandemic | | | | | | | |
| Blood Bank | 40 | 0 | 40 | 100.0% | 89.1% -100% | 100.0% | 89.1% -100% |
| Pre-Pandemic | 47 | 3 | 44 | 93.6% | 81.4% -98.3% | 100.0% | 90.0% -100% |
| Clincal Remants | | | | | | | |
| Total | 137 | 3 | 134 | 97.8% | 93.2% -99.4% | 100.0% | 96.5% -100% |

### Positive Agreement

The 49 retrospective frozen serum samples that were RT-PCR positive for COVID-19 by the Roche® Cobas® SARS-CoV-2 RT-PCR and also tested positive by the GenScript® cPass™ Neutralizing Antibody Detection Kit were tested by the Serum Protocol. The results are summarized in Table 20. The 95% CIs were calculated using an on-line Clinical Calculator (http://vassarstats.net/clin1.html).

TABLE 20
Positive Agreement
IgG Positive Agreement by Days Post Symptom Onset

| Days Post-Symptom | | | | | | | | |
|---|---|---|---|---|---|---|---|---|
| Onset | | n | Positive | Negative | Sensitivity | 95% CI | PPA | 95% CI |
| 4-7 | Days | 4 | 4 | 0 | 100.0% | 39.6%- | 100.0% | 39.6%- |
| | | | | | | 100% | | 100% |
| 8-14 | Days | 16 | 15 | 1 | 93.8% | 66.7%- | 100.0% | 74.7%- |
| | | | | | | 99.7% | | 100% |
| ≥15 to 28 | Days | 29 | 29 | 0 | 100.0% | 85.4%- | 100.0% | 85.4%- |
| | | | | | | 100% | | 100% |

| | | | | | | | |
|---|---|---|---|---|---|---|---|
| All Days | 49 | 48 | 1 | 98.0% | 87.8%- | 100.0% | 90.8%- |

| | |
|---|---|
| 99.9% | 100% |

### Overall Agreement

Per the FDA EUA “Template for Test Developers of Serology Tests that Detect or Correlate to Neutralizing Antibodies” the Serum Protocol was compared against the GenScript® cPass™ Neutralization Antibody Detection Kit using the 137 retrospective frozen serum samples collected from patients prior to December 2019 and the 49 retrospective frozen serum samples that were RT-PCR positive for COVID-19 by the Roche® Cobas® SARS-CoV-2 RT-PCR and also tested positive by the GenScript® cPass™ Neutralizing Antibody Detection Kit. The results are summarized in Table 21. The 95% CIs were calculated using an on-line Clinical Calculator (http://vassarstats.net/clin1.html).

TABLE 21
Overall Agreement

IgG CutOff = 4.465 μg/mL

| IgG | Disease Pos | Disease Neg |
|---|---|---|
| Test Pos | 48 | 3 |
| Test Neg | 1 | 134 |
| | Agreement | 95% CI |
| Sensitivity | 98.0% | 87.8%-99.9% |
| Specificity | 97.8% | 93.2%-99% |
| PPV | 94.1% | 82.8%-98.5% |
| NPV | 99.3% | 95.3%-100% |

### Prospective Study Agreement

The 28 prospectively collected serum samples from the matched sample study were tested by the GenScript® cPass™ Neutralizing Antibody Detection Kit and the Serum Protocol. The results are summarized in Table 22. The 95% CIs were calculated using an on-line Clinical Calculator (http://vassarstats.net/clin1.html).

TABLE 22
Prospective Study Agreement

IgG CutOff = 4.465 μg/mL

| IgG | Disease Pos | Disease Neg |
|---|---|---|
| Test Pos | 27 | 0 |
| Test Neg | 0 | 1 |
| | Agreement | 95% CI |
| Sensitivity | 100.0% | 84.5%-100% |
| Specificity | 100.0% | 5.46%-100% |
| PPV | 100.0% | 84.5%-100% |
| NPV | 100.0% | 5.46%-100% |

### NISBC Verification Panel Agreement

The 37 NISBC Verification Panel serum samples were tested by the GenScript® cPass™ Neutralizing Antibody Detection Kit and the Serum Protocol. The results are summarized in Table 23. The 95% CIs were calculated using an on-line Clinical Calculator (http://vassarstats.net/clin1.html).

TABLE 23
NISBC Verification Panel Agreement

IgG CutOff = 4.465 μg/mL

| IgG | Disease Pos | Disease Neg |
|---|---|---|
| Test Pos | 23 | 1 |
| Test Neg | 0 | 13 |
| | Agreement | 95% CI |
| Sensitivity | 100.0% | 82.2%-100% |
| Specificity | 92.8% | 64.2%-99.6% |
| PPV | 95.8% | 76.9%-99.8% |
| NPV | 100.0% | 71.6%-100% |

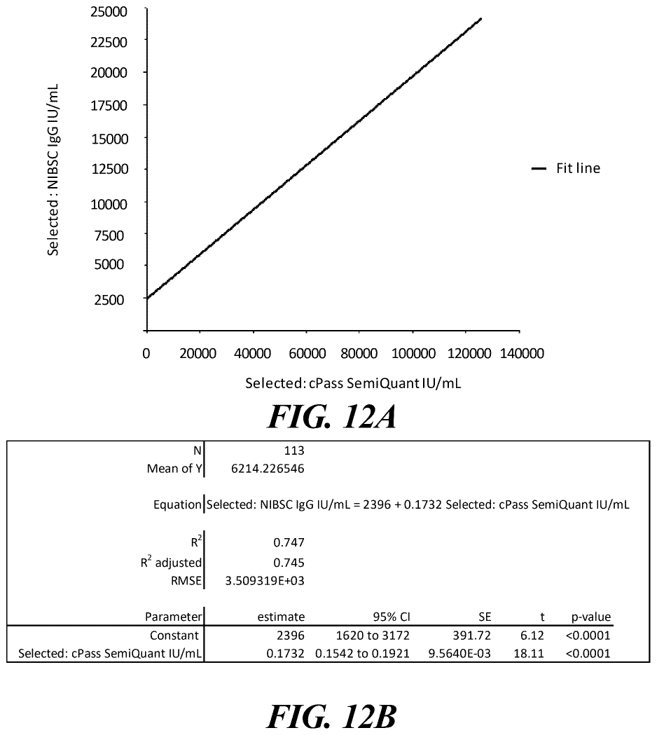

The correlation of the quantitative Serum Protocol IgG values in CRM units (IU/mL) was compared against the semi-quantitative GenScript® cPass™ Neutralization Antibody Detection Kit in CRM units (IU/mL) to assess the correlation between the methods. The linear regression and passing-bablok fits were calculated using Analyze-It for Microsoft Excel (version 5.90, build 7870.29081) using (N=113). Results are summarized in FIG. 12A-B which shows data from a COVID-19 Antibody Test (IU/mL) vs. GenScript® cPass™ Neutralization Antibody Detection Kit (IU/mL). FIG. 12A shows the NIBSC IgG IU/mL over the cPass SemiQuant IU/mL with a fit line. FIG. 12B shows a summary of the data for different parameters including 95% CI, SE, t, and p-value.

The samples that were out of the measuring range high (N=39) were high by both methods and had to be excluded from this analysis (see Table 24).

TABLE 24
Samples Out of Range High by Serum Protocol vs. GenScript ®
cPass ™ Neutralization Antibody Detection Kit

| | | | NIBSC | cPass | |
|---|---|---|---|---|---|
| | | IgG | IgG | SemiQuant | cPass |
| Source | ID | μg/mL | IU/mL | IU/mL | Call |
| Cantor | R18 | >49.3 | >23,167 | 44,263 | POS |
| Cantor | R43 | >49.3 | >23,167 | 132,319 | POS |
| Cantor | R44 | >49.3 | >23,167 | 62,069 | POS |
| Cantor | R534 | >49.3 | >23,167 | 32,535 | POS |
| Cantor | R536 | >49.3 | >23,167 | 48,569 | POS |
| Cantor | R545 | >49.3 | >23,167 | 74,634 | POS |
| Cantor | R546 | >49.3 | >23,167 | nd* | POS |
| Cantor | R555 | >49.3 | >23,167 | 87,685 | POS |
| Cantor | R556 | >49.3 | >23,167 | 59,887 | POS |
| Cantor | R570 | >49.3 | >23,167 | 46,919 | POS |
| Cantor | R578 | >49.3 | >23,167 | 66,244 | POS |
| Cantor | R59 | >49.3 | >23,167 | 37,144 | POS |
| Cantor | R61 | >49.3 | >23,167 | 54,602 | POS |
| Cantor | R625 | >49.3 | >23,167 | 29,568 | POS |
| Cantor | R626 | >49.3 | >23,167 | 36,220 | POS |
| Cantor | R636 | >49.3 | >23,167 | 49,317 | POS |
| Cantor | R677 | >49.3 | >23,167 | 50,393 | POS |
| Cantor | R70 | >49.3 | >23,167 | 113,956 | POS |
| Cantor | R707 | >49.3 | >23,167 | 32,222 | POS |
| Cantor | R723 | >49.3 | >23,167 | 39,845 | POS |
| Cantor | R725 | >49.3 | >23,167 | 41,966 | POS |
| Cantor | R726 | >49.3 | >23,167 | 40,480 | POS |
| Cantor | R731 | >49.3 | >23,167 | 78,739 | POS |
| Cantor | R736 | >49.3 | >23,167 | 32,795 | POS |
| Cantor | R739 | >49.3 | >23,167 | 33,609 | POS |
| Cantor | R741 | >49.3 | >23,167 | 15,897 | POS |
| Cantor | R745 | >49.3 | >23,167 | 38,125 | POS |
| Matched Collection | 3 | >49.3 | >23,167 | 123,958 | POS |
| Matched Collection | 4 | >49.3 | >23,167 | 83,731 | POS |
| Matched Collection | 15 | >49.3 | >23,167 | 126,161 | POS |
| Marched Collection | 27 | >49.3 | >23,167 | nd* | POS |
| Matched Collection | 30 | >49.3 | >23,167 | nd* | POS |
| NIBSC | VP10 | >49.3 | >23,167 | 51,374 | POS |
| NIBSC | VP4 | >49.3 | >23,167 | 57,703 | POS |
| NIBSC | VP5 | >49.3 | >23,167 | 116,155 | POS |
| NIBSC | VP7 | >49.3 | >23,167 | 27,647 | POS |
| NIBSC | VP8 | >49.3 | >23,167 | 57,906 | POS |
| NIBSC | VP9 | >49.3 | >23,167 | 59,888 | POS |
| NIBSC | VP18 | >49.3 | >23,167 | 50,000 | POS |

### Prospectively Collected Matched Serum and SOR Study

To test concordance between serum samples tested by the Serum Protocol and saline oral rinse samples tested by the SOR Protocol, matched serum and saline oral rinse samples were collected concurrently from 28 unique healthy donors.

### Study Population and Size

Matched serum and SOR samples were prospectively collected from healthy donors with an even distribution between male and female patients (N=28). 27 of the healthy donors had been COVID-19 vaccinated, and 1 of the healthy donors was COVID-19 negative by OraRisk® COVID-19 RT-PCR, was never exposed to someone positive for COVID-19, and never tested positive for COVID-19 (N=1).

### Prospectively Collected Matched Serum and SOR Study Protocol

The matched serum/SOR samples were assayed with the same lot of disclosed COVID-19 Antibody Test reagents using the Serum Protocol for the serum samples, and SOR Protocol for the SOR samples, and the results were compared against the GenScript® cPass™ Neutralization Antibody Detection Kit as a comparator method.

GenScript® cPass™ Neutralization Antibody Detection Kit as Comparator Results

The 28 prospectively collected matched serum and SOR samples from vaccinated patients and from a non-vaccinated patient who was COVID-19 negative by OraRisk® COVID-19 RT-PCR, never tested positive for COVID-19, never was exposed to someone positive for COVID-19, and never had COVID-19 symptoms, was tested by the disclosed COVID-19 Antibody Test and the GenScript® cPass™ Neutralization Antibody Detection Kit. The results are summarized in Tables 25 and 26. The 95% CIs were calculated using an on-line Clinical Calculator (http://vassarstats.net/clin1.html).

TABLE 25
Serum IgG and IgA Combined Agreement vs.
GenScript ® cPass ™
Neutralization Antibody Detection Kit

| | |
|---|---|
| | IgG CutOff = 4.465 μg/mL |
| IgG and IgA | IgA CutOff = 1.15 μg/mL |

| Combined | Disease Pos | Disease Neg |
|---|---|---|
| Test Pos | 27 | 0 |
| Test Neg | 0 | 1 |

| | Agreement | 95% CI |
|---|---|---|
| Sensitivity | 100% | 84.5%-100% |
| Specificity | 100% | 5.46%-100% |
| PPV | 100% | 84.5%-100% |
| NPV | 100% | 5.46%-100% |

TABLE 26
SOR IgG, IgM, and IgA Combined Agreement vs. GenScript ®
cPass ™ Neutralization Antibody Detection Kit

| | |
|---|---|
| IgG, IgM, | IgG CutOff = 67.5 ng/mL |
| and IgA | IgM Cutoff = 39.1 ng/mL |
| Combined | IgA CutOff = 34.7 ng/mL |

| All days | Disease Pos | Disease Neg |
|---|---|---|
| Test Pos | 27 | 0 |
| Test Neg | 0 | 1 |
| | Agreement | 95% CI |
| Sensitivity | 100% | 84.5%-100% |
| Specificity | 100% | 5.46%-100% |
| PPV | 100% | 84.5%-100% |
| NPV | 100% | 5.46%-100% |

### Overall Summary

The IgG LoQ cutoff was validated with sensitivity, specificity, PPA, NPA, correlation coefficient (r value), and p value for SARS-CoV-2 binding IgG antibodies to SARS-CoV-2 based on correlation to the semi-quantitative GenScript® cPass™ Neutralization Antibody Detection Kit (EUA201427). The Disclosed COVID-19 Neutralizing Antibody Test demonstrated 94.1% PPA (95% CI 82.8-98.5%) and 99.3% NPA (95% CI 95.3-100%) as compared against the GenScript® cPass™ Neutralization Antibody Detection Kit (EUA201427). The Disclosed COVID-19 Neutralizing Antibody Test IgG results (IU/mL) have also demonstrated a correlation to the GenScript® cPass™ Neutralization Antibody Detection Kit (r=0.87, p<0.001).

### Example 10: Clinical Agreement Study of Salivary Samples

The clinical performance of the Disclosed COVID-19 Neutralizing Antibody Test was further evaluated by testing human saline oral rinse samples that were collected from RT-PCR positive patients, as well as by testing presumptive negative saline oral rinse samples that should not have antibodies to SARS-CoV-2, and frozen saliva samples collected prior to November 2019 (pre-pandemic).

### Study Population and Size

The following patient samples were used in the clinical study:
(i) Prospectively collected saline oral rinse specimens from OraRisk® COVID-19 RT-PCR positive SARS-CoV-2 patients (N=70). All 70 patients indicated on their patient intake form they had never been COVID-19 vaccinated.
(ii) Prospectively collected saline oral rinse specimens from patients who met the following inclusion criteria (N=11): a. tested COVID-19 negative by OraRisk® COVID-19 RT-PCR; b. never been vaccinated; c. never tested positive for COVID-19; d. never been exposed to someone who was COVID-19 positive; e. never had COVID-19 symptoms in the past 12 months including fatigue, flu like symptoms, shortness of breath or difficulty breathing, and loss of taste or smell.

Pre-pandemic frozen saliva samples were collected prior to November 2019 (N=32).

### Saline Oral Rinse Clinical Agreement Study

The clinical sensitivity of the Disclosed COVID-19 Neutralizing Antibody Test was evaluated by testing saline oral rinse samples that were RT-PCR positive by the OraRisk® COVID-19 RT-PCR (EUA200464). Positive saline oral rinse samples were prospectively collected from unvaccinated patients who tested positive for SARS-CoV-2 by OraRisk® COVID-19 RT-PCR with known days after symptom onset. The results are presented in the following tables. All samples were collected and tested by OraRisk® COVID-19 RT-PCR on the same day. The 95% CIs were calculated using an on-line Clinical Calculator (http://vassarstats.net/clin1.html). The positive percent agreement (PPA) of the Disclosed COVID-19 Neutralizing Antibody Test SOR Protocol (SOR Protocol) for all tested saline oral rinse specimens collected from 2 to 16 days post-symptom onset was 95.5% (67/70; 95% CI 83.3-99.2%). Positive agreement results are shown in Tables 27 and 28.

TABLE 27
SOR Protocol Positive Agreement by Days Post-Symptom Onset

| Days | | | | | | |
|---|---|---|---|---|---|---|
| Post-Symptom | | | | | | |
| Onset | | n | Positive | Negative | PPA | 95% CI |
| 0-7 | Days | 44 | 42 | 2 | 95.5% | 83.3%-99.2% |
| 8-14 | Days | 21 | 20 | 1 | 95.2% | 74.1%-93.3% |
| ≥15 | Days | 5 | 5 | 0 | 100% | 46.3%-100% |
| All | Days | 70 | 67 | 3 | 95.7% | 87.2%-98.9% |

TABLE 28
SOR Protocol Positive Agreement
by Days After PCR Positive Result

| Days After PCR | | | | | | |
|---|---|---|---|---|---|---|
| Positive Result | | n | Positive | Negative | PPA | 95% CI |
| 0-7 | Days | 44 | 42 | 2 | 95.5% | 83.3%-99.2% |
| 8-14 | Days | 21 | 20 | 1 | 95.2% | 74.1%-93.3% |
| ≥15 | Days | 5 | 5 | 0 | 100% | 46.3%-100% |
| All | Days | 70 | 67 | 3 | 95.7% | 87.2%-98.9% |

The clinical specificity of the SOR Protocol was evaluated by testing frozen saliva samples collected prior to November 2019 (pre-pandemic saliva samples), and by testing presumptive negative saline oral rinse samples that were prospectively collected from patients that met the study inclusion criteria. The 95% CIs were calculated using an on-line Clinical Calculator (http://vassarstats.net/clin1.html). There were no false positives detected in the pre-pandemic saliva samples nor in the apparently negative saline oral rinse samples. The negative percent agreement (NPA) of the SOR Protocol was 100% (48/48; 95% CI 90.8-100%). Table 29 shows the negative agreement.

TABLE 29
SOR Protocol Negative Agreement
Negative Agreement

| Category | n | Positive | Negative | NPA | 95% CI |
|---|---|---|---|---|---|
| Apparently Negative | 11 | 0 | 11 | 100% | 67.9%-100% |
| Saline Oral Rinse | | | | | |
| Pre-Pandemic Saliva | 69 | 0 | 69 | 100% | 93.4%-100% |
| Samples | | | | | |
| Total | 80 | 0 | 80 | 100% | 94.3%-100% |

### Example 11: Multiplex Secretory Antibody Detection and Quantitation in Saline Oral Rinse for SARS-CoV-2 Vaccine Efficacy Testing

The Disclosed COVID-19 Neutralizing Antibody Test may include a semi-automated, bead-based fluorescent immunoassay intended for the multiplex quantitative detection of neutralizing SARS-CoV-2 IgG, IgA, and IgM secretory antibodies in saline oral rinse. The saline oral rinse sample was pre-analytically treated with paramagnetic clean beads to selectively bind and remove sample-specific heterophilic antibody interference and/or autoantibody interference. After a sample incubation, the clean beads were isolated with a magnet, and the conditioned sample was aspirated and transferred to the reaction plate. Neutralizing SARS-CoV-2 antibodies were subsequently captured by adding paramagnetic Capture Beads to the conditioned sample. The Capture Beads comprised paramagnetic streptavidin beads coated with biotinylated SARS-CoV-2 spike protein RBD-specific recombinant antigens (Wild Type and Delta variant) and SARS-CoV-2 spike protein NTD-specific recombinant antigen (Wild Type). After a sample incubation, the Capture Beads were washed to remove the sample matrix and non-specific antibodies. Neutralizing SARS-CoV-2 antibodies were detected by adding a rabbit polyclonal anti-human IgA, anti-human IgG, and anti-human IgM triplex (Alexa Fluor 488, 555, and 647) fluorescent conjugate. After a sample incubation, the Capture Beads were washed to remove excess conjugate. The conjugate was eluted from the Capture Beads, aspirated, and dispensed into a neutralizing buffer for subsequent fluorescent detection and measurement. The amount of neutralizing SARS-CoV-2 IgG antibodies detected in the serum or lithium heparin plasma sample, or the amount of neutralizing SARS-CoV-2 IgG, IgA, and IgM secretory antibodies detected in saline oral rinse, was determined by use of a bead-based calibration curve. The calibration curve was generated using seven different triplex calibrator beads with different amounts of purified human IgM, IgG and IgA conjugated to each calibrator bead. The relative fluorescence units of each fluorophore are directly proportional to the amount of antigen specific IgG, IgM and/or IgA immunoglobulins captured by the capture beads.

Saline oral rinse samples were tested using the following Disclosed COVID-19 Neutralizing Antibody Test Reagents:
Reagent A—Clean Bead: Streptavidin coated superparamagnetic nanoparticles coupled with biotin labeled animal antibodies (rabbit, goat, and bovine), and biotin labeled human IgM, IgG and IgA, and bovine serum albumin coated superparamagnetic nanoparticles, in TRIS buffer and 0.05% detergent. Preservative: 0.05% sodium azide.
Reagent B—Capture Bead: Streptavidin coated superparamagnetic nanoparticles coupled with biotin labeled SARS-CoV-2 spike protein RBD recombinant antigens (Wild Type and Delta variant) produced in HEK293 cells and purified from culture supernatant, and biotin labeled SARS-CoV-2 spike protein NTD recombinant antigen (Wild Type) produced in HEK293 cells and purified from culture supernatant, in TRIS buffer, 10% goat serum, 0.1% bovine serum albumin, 0.001% polymer, and 0.05% detergent. Preservative: 0.05% sodium azide.
Reagent C—Triplex Conjugate: Rabbit polyclonal anti-human IgM antibody, anti-human IgG antibody, and anti-human IgA antibody, each labeled with a different fluorophore (Alexa Flour 488, 555, or 647), in TRIS buffer, 0.1% bovine serum albumin, and 0.05% detergent. Preservative: 0.05% sodium azide.
Reagent D—25× Wash Buffer: TRIS buffer, 0.05% detergent, and <0.05% polymeric blocker. Preservative: 0.05% sodium azide.
Reagent E—Elution Buffer: Glycine buffer, 0.05% detergent. Preservative: 0.05% sodium azide.
Reagent F—Neutralizing Buffer: TRS buffer. Preservative: 0.05% sodium azide.
Diluent A: Phosphate buffered saline. Preservative: 0.05% sodium azide.
Diluent B: 0.9% saline. Preservative: 0.05% sodium azide.
Reagent G—Calibrator 0: Bovine serum albumin coated superparamagnetic nanoparticles in TRIS buffer, 0.1% bovine serum albumin, and 0.05% detergent. Preservative: 0.05% sodium azide.
Reagent G—Calibrator 1 through Calibrator 6: Streptavidin coated superparamagnetic nanoparticles coupled with biotin labeled human IgM, IgG and IgA, and bovine serum albumin coated superparamagnetic nanoparticles, in TRIS buffer, 0.1% bovine serum albumin, and 0.05% detergent. Preservative: 0.05% sodium azide.

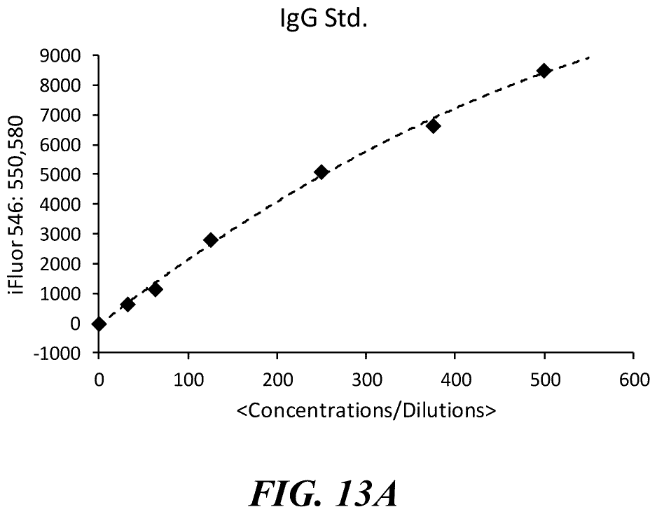

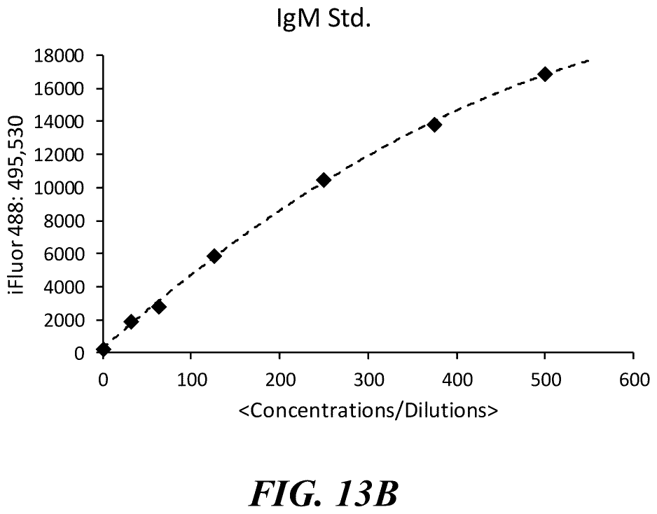

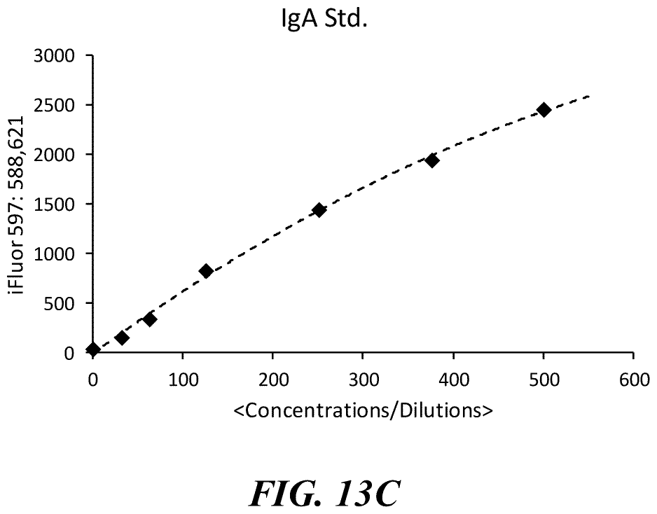

Reagent G triplex human IgA, IgG, and IgM calibrator beads were made as follows to generate dose response calibration curves to quantitate antibody levels detected in a sample:
Preparation and biotinylation of IgA, IgG, and IgM: Human immunoglobulins A, G and M are purified from pools created from sera of multiple donors. Individual immunoglobulins were then biotinylated with either an NHSester-PEG4-biotin reagent or TFPester-PEG4-biotin reagent in a pH 8 bicarbonate or phosphate-bicarbonate buffer at a molar excess of 10 moles of biotinylation reagent to 1 mole of protein. The biotinylation agent was solubilized in DMSO at a concentration of 20 mM. Then a sufficient volume of biotinylation/DMSO solution was introduced into the protein solution to produce the reaction at the 10 to 1 molar excess. The two solutions were mixed well and then incubated at ambient temperate for at least 2 hours with continued mixing. The incubation can be as long as 18 hours. At the end of incubation, the solutions were desalted into a neutral pH 7.4 PBS buffer by means of size exclusion chromatography to remove non-reacted biotinylation.
Preparation of streptavidin coated paramagnetic microparticles (PMP): 1.6 μm PMPs carboxyl-activated were obtained from Japan Synthetic Rubber Company (JSR). These PMPs were washed into a 40 mM MES pH 5.3 buffer at 25 mg PMP/mL. Lyophilized Streptavidin purchased from Agilent was reconstituted to a concentration of 20 mg/mL with purified deionized water (at 18 MOhm resistivity). This solution was desalted by means of size exclusion chromatography into 40 mM MES pH 5.2 buffer to remove NaCl from the streptavidin. This streptavidin MES solution was diluted to 10 mg/mL in 40 mM MES. 4 parts volume of PMP suspension at 25 mg/mL and 5 parts volume streptavidin at 10 mg/mL were mixed together to produce a mixture of 11.1 mg/mL PMP with 5.56 mg/mL Streptavidin in 40 mM MES pH 5.2. This mixture was well mixed and sonicated to ensure bead dispersity. A solution of 10 mg/mL EDC (1-Ethyl-3-(3-dimethylaminopropyl)carbodiimide) in 40 mM MES pH 5.2 was prepared so that when the PMP/streptavidin and EDC solutions were mixed together there would be 1 mg EDC for each 10 mg PMP—or 100 μL EDC solution for each 0.9 mL of PMP/streptavidin. PMP/Streptavidin and EDC both in MES, were introduced together at a constant rate of 18 mL per minute PMP/streptavidin to 2 mL EDC to produce a mixture of 10 mg/mL PMP, 5 mg/mL streptavidin and 1 mg/mL EDC. This mixture was incubated at ambient temperature for at least 2 hours but not more than 20 hours. The reacted PMPs were magnetically separated, and the supernatant streptavidin was removed. The pelleted beads were wash three times in PBS pH 7.4 and then brought to volume with PBS pH 7.4 to a concentration of 10 mg/mL PMPs and sonicated to ensure dispersity. Protein coating the PMPs was measured via MicroBCA assay from ThermoFisher. Typical Streptavidin concentration was 18 μg/mg PMP.
Preparation of Immunoglobulin coated PMPs. Streptavidin coated PMPs were washed into PBS three times then brought to volume to produce a PMP mixture of 20 mg/mL in PBS with pH 7.4. Biotinylated Immunoglobulins were individually prepared by dilution into PBS pH 7.4 to a concentration of 0.6 mg/mL. Each mixture was placed into round bottom vessels with a central mixing blade motivated by an overhead mixer attached to shaft. Both vessels, one with PMPs and the other with biotinylated immunoglobulin, were stirred by means of their mixing blades to approximately 150 RPM. The PMPs were sonicated to ensure dispersity and pumped at 20 mL/minute into the rapidly mixing immunoglobulin mixture. When all PMPs were pumped into the immunoglobulin mixture, the resultant mixture was incubated at ambient temperature for 2 hours. At the end of two hours, the PMPs were magnetically separated, and the remaining supernatant was removed. The pelleted PMPs were washed 3 times with Tris (10 mM) buffered saline (150 mM) with Tween-20 (0.5%) and sodium azide (0.05%) after which the PMPs were brought to volume with the same TBS-tween-azide to a concentration of 10 mg/mL and sonicated to ensure dispersity.
Value assignment of Immunoglobulin per mg PMP: [mass of immunoglobulin at the beginning of the coating step (the inputted solution of 0.6 mg/mL immunoglobulin as determined by A280/1.4)—mass of immunoglobulin at the end of the coating step (the concentration of supernatant immunoglobulin after magnet separation determined by A280/1.4)]/mg of PMPs being coated. The amount was typically 16 μg Immunoglobulin per mg PMP.
Preparation of Triplex calibrators: a mixture of IgA, IgG and IgM coated streptavidin PMPs in Tris buffered saline with Tween 20 and sodium azide was prepared such that a 60 μL volume has 500 ng mass of each immunoglobulin. Each individual immunoglobulin PMP was concentrated to effect an immunoglobulin concentration of 25 μg/mL. Each individual immunoglobulin coated PMP at 25 μg/mL immunoglobulin is then mixed at a ratio of 1:1:1 so that the resultant mixture has a concentration of 8.333 μg/mL of each immunoglobulin. To prepare a calibration curve from a stock of 500 ng/mL immunoglobulin PMPs, the PMPs were mixed with 1.6 μm PMPs coated with BSA to create 7 individual PMP mixtures with concentrations of 500 ng/mL, 375 ng/mL, 250 ng/mL, 125 ng/mL, 62.5 ng/mL, 31.25 ng/mL and 0 ng/mL.
Use of calibrator PMPs in the serology or human immunoglobulin IgG, IgM, and/or IgA assay: 60 μL of each of these triplex calibrator PMPs were placed individually in wells of a 96 well microtiter plate, for example, in a single column such as A1 through G1 of a 96 well microtiter plate. These PMPs then were exposed to the same reagents, and incubations as the remaining steps of protocol as the unknowns. Conjugate eluted from the calibration PMPs was proportionally to the concentration of immunoglobulins. Curve fitting is accomplished by use of a 4 parameter Log Logit calculation. FIGS. 13A-13C show examples of calibration curves for human IgG, IgM, and IgA.

Saline oral rinse samples were collected by having patients swish and gargle with 5 mL of saline (0.9% sodium chloride in water) for 30 seconds and expectorate into a collection tube. The collection tube was subsequently sealed with a screw cap (e.g., Falcon 50 mL Conical Centrifuge Tube). Collection was supervised by a nurse practitioner, nurse, or healthcare provider.

The test includes the use of external human plasma-based controls supplied by Bio-Rad [VIROTROL SARS-CoV-2 Single Level Control (PN 200300A or 200305A). The test uses 1,000 μL of each control (N=1 replicate) per assay:
Positive Control 1: 15 μL Bio-Rad VIROTROL SARS-CoV-2 Single Level Control (PN 200300A or 200305A) diluted into 985 μL Diluent A included in the kit as the positive control for neutralizing IgG and IgM antibodies to SARS-CoV-2.
Positive Control 2: 60 μL Bio-Rad VIROTROL SARS-CoV-2 Single Level Control (PN 200300A or 200305A) diluted into 940 μL Diluent A included in the kit as the positive control for neutralizing IgA antibodies to SARS-CoV-2.
Negative Control: Diluent B included with the kit is used as the negative control for neutralizing IgG, IgA, and IgM antibodies to SARS-CoV-2.

The Limit of Blank (LoB), Limit of Detection (LoD) and Limit of Quantitation (LoQ) was determined using saline oral rinse samples with varying amounts of IgG, IgM, and IgA secretory antibodies to SARS-CoV-2. LoB of IgG, IgM, and IgA were 43.6 ng/mL, 12.0 ng/mL, and 9.8 ng/mL respectively. LoD of IgG, IgM, and IgA were 67.5 ng/mL, 26.6 ng/mL, and 17.2 ng/mL respectively. LoQ of IgG, IgM, and IgA were 67.5 ng/mL, 39.1 ng/mL, and 34.7 ng/mL respectively.

The analytical measuring interval was defined by Cal 6. Numeric values are interpreted as “negative” (<LoQ) and as “positive” (≥LoQ). Values above the measuring range are reported as >Cal 6 (lot specific value assignment for IgG, IgA, and IgM). When sample results exceed the upper limit of the analytical measuring interval (ULMI) they can be diluted with Diluent B and retested. The recommended dilution is 1:4 (3 parts Diluent B and 1 part sample).

The precision was determined by testing four samples over three days with two runs per day. The four SOR samples were contrived based on levels of IgG, IgM, and IgA detected, including samples at the LoQ to samples at the higher end of the clinical range. One lot of Disclosed COVID-19 Neutralizing Antibody Test reagents was used to produce this data. Tables 30-32 shows the saline oral rinse IgG, IgM, and IgA precision.

TABLE 30
Saline Oral Rinse IgG Precision

| | | CV | CV | |
|---|---|---|---|---|
| | Average | Within | Within | Total |
| Sample ID | (ng/mL) | Run | Day | CV |
| Sample 1 | 96.9 | 8.5% | 8.2% | 15.9% |
| Sample 2 | 209.5 | 5.7% | 14.3% | 18.1% |
| Sample 3 | 143.7 | 7.5% | 8.1% | 12.5% |
| Sample 4 | 66.7 | 5.5% | 15.7% | 19.4% |

IgG Within Run CVs were lower than or equal to 8.5%, Within Day CVs were lower than or equal to 14.3%, (a CV of 15.7% was observed at a dose of 66.7 ng/mL, or less than the LoQ of 67.5 ng/mL), and Total CVs were lower than or equal to 18.1%.

TABLE 31
Saline Oral Rinse IgM Precision

| | | CV | CV | |
|---|---|---|---|---|
| | Average | Within | Within | Total |
| Sample ID | (ng/mL) | Run | Day | CV |
| Sample 1 | 52.5 | 8.6% | 7.6% | 10.0% |
| Sample 2 | 47.8 | 6.1% | 14.4% | 17.9% |
| Sample 3 | 14.6 | 14.7% | 27.7% | 40.7% |
| Sample 4 | 26.1 | 5.3% | 15.6% | 15.1% |

IgM Within Run CVs were lower than or equal to 8.6% (a CV of 14.7% was observed at a dose of 14.6 ng/mL, or less than the LoQ of 39.1 ng/mL), Within Day CVs were lower than or equal to 14.4%, (a CV of 27.7% was observed at a dose of 66.7 ng/mL, and a CV of 15.6% at a dose of 26.1 ng/mL, or less than the LoQ of 39.1 ng/mL), and Total CVs were lower than or equal to 17.9% (a CV of 40.7% was observed at a dose of 14.6 ng/mL, or less than the LoQ of 39.1 ng/mL).

TABLE 32
Saline Oral Rinse IgA Precision

| | | CV | CV | |
|---|---|---|---|---|
| | Average | Within | Within | Total |
| Sample ID | (ng/mL) | Run | Day | CV |
| Sample 1 | 64.5 | 9.2% | 7.9% | 12.0% |
| Sample 2 | 64.6 | 4.2% | 15.3% | 18.2% |
| Sample 3 | 121.2 | 4.8% | 8.6% | 12.8% |
| Sample 4 | 105.6 | 5.5% | 8.2% | 10.7% |

IgA Within Run CVs were lower than or equal to 9.2%, Within Day CVs were lower than or equal to 15.3%, and Total CVs were lower than or equal to 18.3%.

The linearity was determined to demonstrate that the assay measures a proportionate concentration of human immunoglobulin to the amount present in the sample. Saline oral rinse samples were also pooled to create samples with high concentrations of IgG, IgM, and IgA secretory antibodies to SARS-CoV-2. These samples were then diluted with Diluent B in 20% increments. The diluted samples were tested in duplicate, and the Linearity results calculated using Analyze-It for Microsoft Excel (version 5.90, build 7870.29081). For saline oral rinse, linearity was demonstrated for the interval of 67.5 ng/mL to 377 ng/mL for IgG, 39.1 ng/mL to 383 ng/mL for IgM, and 34.7 ng/mL to 244 ng/mL for IgA, with all deviations from linearity within 15%. No endogenous saline rinse samples were available for IgG, IgM, or IgA at the upper measuring range or higher than Cal 6 for this study.

The recovery was determined to demonstrate that the assay measures a proportionate concentration of human immunoglobulin to the amount present in the sample.

12 potential cross-reactive antibodies (Table 33) were evaluated. All serum samples were obtained before November 2019.

TABLE 33
Potential Cross-Reactive Antibodies

anti-influenza A (IgG and IgM)
anti-influenza B (IgG and IgM)
anti-HCV (IgG and IgM)
anti-HBV (IgG and IgM)
anti-Haemophilus influenzae (IgG and IgM)
anti-229E (alpha coronavirus)
anti-NL63 (alpha coronavirus)
anti-OC43 (beta coronavirus)
anti-HKU1 (beta coronavirus)
ANA
anti-respiratory syncytial virus (IgG and IgM)
anti-HIV

While cross-reactivity for these viruses would not be expected in saline oral rinse, ELISA antibody positive serum samples for each of these potential 12 cross-reactive antibodies or disease states were tested by diluting each sample in 10% artificial saliva (Pickering, Part No. 1700-0316) in saline (0.9% NaCl in water). BioRad VIROCLEAR SARS-CoV-2 Single Level Control (Part No. 200500 or 200505) was also diluted as the negative control since serum spiked into SOR can over-recover. Results of cross-reactivity PASS if the cross-reactive samples read <LoQ for IgG (<67.5 ng/mL), IgM (<39.1 ng/mL), or IgA (<34.7 mg/mL), or ≤BioRad VIROCLEAR SARS-CoV-2 Single Level Control. The resulting overall specificity was 100%. The testing results are shown in Table 34.

TABLE 34
Cross-reactivity results

| | | IgM Conc | | IgG Conc | | IgA Conc | |
|---|---|---|---|---|---|---|---|
| ID | Disease (antibodies) | (ng/mL) | ≤Viroclear | (ng/mL) | ≤Viroclear | (ng/mL) | ≤Viroclear |
| Viroclear | | <LoQ | PASS | 109 | PASS | <LoQ | PASS |
| INF11228-01 | Influenza A IgG and IgM | <LoQ | PASS | 97 | PASS | <LoQ | PASS |
| INF11228-02 | Influenza B IgG and IgM | <LoQ | PASS | 109 | PASS | <LoQ | PASS |
| HCV211228-01 | Anti-HCV (IgG and IgM) | <LoQ | PASS | 97 | PASS | <LoQ | PASS |
| HBV211228-01 | Anti-HBC (IgG and IgM) | <LoQ | PASS | 101 | PASS | <LoQ | PASS |
| HAI211228-01 | Haemophillus influenzae (IgG and IgM) | <LoQ | PASS | 106 | PASS | <LoQ | PASS |
| COV211228-01 | Anti-229E (alpha corona virus) IgG | <LoQ | PASS | 91 | PASS | <LoQ | PASS |
| COV211228-02 | Anti-NL63 (alpha corona virus) IgG | <LoQ | PASS | 78 | PASS | <LoQ | PASS |
| ANA211228-01 | Anti-Nuclear Antibodies (ANA) | <LoQ | PASS | 78 | PASS | <LoQ | PASS |
| RSV211228-01 | RSV (IgG and IgM) | <LoQ | PASS | 91 | PASS | <LoQ | PASS |
| HIV211228-01 | Anti-HIV IgG | <LoQ | PASS | 76 | PASS | <LoQ | PASS |
| TB211220-01 | Anti-H37Rv Tuberculosis IgG | <LoQ | PASS | 87 | PASS | <LoQ | PASS |
| TB211220-02 | Anti-ErdmanTuberculosis IgG | <LoQ | PASS | 65 | PASS | <LoQ | PASS |

Since most of the samples tested were less than LoQ for IgM and IgA, cross-PR 4T reactivity testing was repeated by spiking 1 μL of each of the 12 antibody reactivity samples into 999 μL Saline Oral Rinse Positive Sample for IgM (108 ng/mL), IgG (300 ng/mL), and IgA (66 ng/mL), as well as a 1 μL of a Positive Serum Control sample and 1 μL Bio-Rad VIROCLEAR negative control. Results of cross-reactivity PASS if, 1) the Positive Serum Control spike is >VIROCLEAR SARS-COV-2 Single Level Control for IgM, IgG, and IgA, and 2) the cross-reactive samples read ≤BioRad VIROCLEAR SARS-CoV-2 Single Level Control, or ≤VIROCLEAR SARS-CoV-2 Single Level Control spike+15%. Testing results are shown in Table 35.

TABLE 35
Cross-reactivity results

| | | IgM | ≤VIROCLEAR | IgG | ≤VIROCLEAR | IgA | ≤VIROCLEAR |
|---|---|---|---|---|---|---|---|
| | Disease | Conc | or ≤VIROCLEAR +15% | Conc | or ≤VIROCLEAR +15% | Conc | or ≤VIROCLEAR +15% |
| ID | (Antibodies) | (ng/mL) | (Pass/Fail) | (ng/mL) | (Pass/Fail) | (ng/mL) | (Pass/Fail) |
| SOR Positive | | 108 | | 300 | | 66 | |
| Sample | | | | | | | |
| Positive | | 132 | | 442 | | 80 | |
| Serum | | | | | | | |
| Control | | | | | | | |
| Spike | | | | | | | |
| Viroclear | | 126 | | 388 | | 76 | |
| Spike | | | | | | | |
| Viroclear | | 145 | | 446 | | 87 | |
| Spike + 15% | | | | | | | |
| INF11228- | Influenza A | 152 | Fail | 357 | Pass | 71 | Pass |
| 01* | IgG and IgM | | | | | | |
| INF11228-02 | Influenza B | 127 | Pass | 401 | Pass | 80 | Pass |
| | IgG and IgM | | | | | | |
| HCV211228- | Anti-HCV | 135 | Pass | 332 | Pass | 75 | Pass |
| 01 | (IgG and | | | | | | |
| | IgM) | | | | | | |
| HBV211228- | Anti-HBC | 122 | Pass | 363 | Pass | 76 | Pass |
| 01 | (IgG and | | | | | | |
| | IgM) | | | | | | |
| HAI211228- | Haemophillus | 108 | Pass | 279 | Pass | 56 | Pass |
| 01 | influenzae | | | | | | |
| | (IgG and | | | | | | |
| | IgM) | | | | | | |
| COV211228- | Anti-229E | 116 | Pass | 336 | Pass | 78 | Pass |
| 01* | (alpha corona | | | | | | |
| | virus) IgG | | | | | | |
| COV211228- | Anti-NL63 | 109 | Pass | 278 | Pass | 65 | Pass |
| 02 | (alpha corona | | | | | | |
| | virus) IgG | | | | | | |
| COV211228- | Anti-OC43 | 111 | Pass | 314 | Pass | 64 | Pass |
| 03 | (beta corona | | | | | | |
| | virus) IgG | | | | | | |
| COV211228- | Anti-HKUI | 100 | Pass | 252 | Pass | 62 | Pass |
| 04 | (beta corona | | | | | | |
| | virus) IgG | | | | | | |
| ANA211228- | Anti-Nuclear | 114 | Pass | 281 | Pass | 67 | Pass |
| 01 | Antibodies | | | | | | |
| | (ANA) | | | | | | |
| RSV211228- | RSV (IgG | 144 | Pass | 264 | Pass | 63 | Pass |
| 01* | and IgM) | | | | | | |
| HIV211228- | Anti-HIV | 110 | Pass | 281 | Pass | 65 | Pass |
| 01 | IgG | | | | | | |

*These samples were grossly lipemic and full of particulates. An attempt was made to clarify them by centrifugation which failed. It was observed that the particulates floated.

The potential for microorganisms to interfere with the assay results was evaluated by spiking 13 different microorganisms into pooled saline oral rinse samples. Table 36 shows the microorganism testing results.

TABLE 36
Concentration of microorganisms

| | | Type/ | | Concen- | |
|---|---|---|---|---|---|
| Virus/Bacteria | Species | strain | # | tration | tested |
| Actinomyces | viscosus | ATCC | 43146 | >10\hat{ }4 | CFU/mL |
| Candida | albicans | ATCC | 18804 | 4.9 × 10\hat{ }6 | CFU/mL |
| Chlamydolphia | pneumoniae | ATCC | 53592 | 7.0 × 10\hat{ }6 | IFU/mL |
| | AR-39 | | | | |
| Herpes | Type 1 | ATCC | VR-260 | 8.0 × 10\hat{ }6 | TCID50/ |
| Simplex | | | | | mL |
| Herpes | Type 2 | ATCC | VR-734 | 8.0 × 10\hat{ }5 | TCID50/ |
| Simplex | | | | | mL |
| Lactobacillus | johnsonii | ATCC | 33200 | >10\hat{ }4 | CFU/mL |
| Mycobacterium | tuberculosis | ATCC | 25177 | >10\hat{ }4 | CFU/mL |
| Mycoplasma | pneumonia | ATCC | 15531 | 8.0 × 10\hat{ }5 | CFU/mL |
| Porphyromonas | gingivalis | ATCC | 49417 | >10\hat{ }4 | CFU/mL |
| Staphylococcus | epidermis | ATCC | 12228 | 1.6 × 10\hat{ }7 | CFU/mL |
| Streptococcus | pyogenes | ATCC | 19615 | 1.8 × 10\hat{ }6 | CFU/mL |
| Streptococcus | salivarius | ATCC | 7073 | >10\hat{ }4 | CFU/mL |
| Streptococcus | mutans | ATCC | 25175 | 4.4 × 10\hat{ }5 | CFU/mL |

Potential microbial interference from bacterial and viral microorganisms that may be present in oral fluid specimens was tested. To prepare the samples, saline oral rinse antibody positive samples were spiked with microbial organisms and then tested with the SOR Protocol. No false positive or false negative results were observed at the microbial concentrations tested Table 37. Testing results of microorganism interferences

TABLE 37
Testing results of microorganism interferences

| Saline oral rinse (Sample with high level of antibody) | | |
|---|---|---|
| IgG | IgM | IgA |

| | | | % of | | % of | | % of |
|---|---|---|---|---|---|---|---|
| Sample/Organism | | ng/mL | control | ng/mL | control | ng/mL | control |
| 1 | Positive Control | 524.7 | 100.0% | 124.9 | 100.0% | 122.0 | 100% |
| 2 | Strept. mutans | 510.3 | 97.3% | 118.9 | 95.2% | 116.7 | 96% |
| 3 | Staph. epidermidis | 446.6 | 85.1% | 116.4 | 93.2% | 120.2 | 99% |
| 4 | Mycoplasma pneumoniae | 516.5 | 98.4% | 109.0 | 87.3% | 120.0 | 98% |
| 5 | Porphyromonas | 498.1 | 94.9% | 110.5 | 88.4% | 119.2 | 98% |
| | gingivalis | | | | | | |
| 6 | Actinomyces viscosus | 496.5 | 94.6% | 121.6 | 97.3% | 101.9 | 84% |
| 7 | Strept. Pyogenes | 486.4 | 92.7% | 106.1 | 84.9% | 110.0 | 90% |
| 8 | Strept. Salivarius | 517.9 | 98.7% | 118.7 | 95.0% | 124.1 | 102% |
| 9 | Mycobacterium | 498.4 | 95.0% | 114.0 | 91.3% | 109.3 | 90% |
| | tuberculosis | | | | | | |
| 10 | Candida albicans | 457.3 | 87.2% | 118.3 | 94.7% | 124.4 | 102% |
| 11 | Lactobacilli johnsonii | 513.4 | 97.8% | 121.3 | 97.1% | 104.7 | 86% |
| 12 | Chlymdophilia | 491.6 | 93.7% | 120.6 | 96.6% | 120.6 | 99% |
| | pnemoniae | | | | | | |
| 13 | Herpes simplex I | 510.1 | 97.2% | 119.1 | 95.3% | 98.7 | 81% |
| 14 | Herpes simplex II | 466.4 | 88.9% | 118.6 | 94.9% | 113.8 | 93% |

| | | Saline oral rinse (Sample with low level of antibody) | | | | | |
|---|---|---|---|---|---|---|---|
| | | IgG (LoQ < 67.5) | | IgM (LoQ < 39.1) | | IgA (LoQ < 34.7) | |
| | | | % of | | % of | | % of |
| Sample/Organism | | ng/mL | control | ng/mL | control | ng/mL | control |
| 1 | Control | 103.1 | 100% | 41.5 | 100% | <LoQ | N/A |
| 2 | Strept. mutans | 104.1 | 101% | <LoQ | N/A | <LoQ | N/A |
| 3 | Staph. epidermidis | 112.3 | 109% | 40.6 | 98% | <LoQ | N/A |
| 4 | Mycoplasma pneumoniae | 98.3 | 95% | <LoQ | N/A | <LoQ | N/A |
| 5 | Porphyromonas | 100.5 | 98% | <LoQ | N/A | <LoQ | N/A |
| | gingivalis | | | | | | |
| 6 | Actinomyces viscosus | 106.3 | 103% | <LoQ | N/A | <LoQ | N/A |
| 7 | Strept. Pyogenes | 121.6 | 118% | 46.1 | 111% | <LoQ | N/A |
| 8 | Strept. Salivarius | 101.8 | 99% | 39.6 | 95% | <LoQ | N/A |
| 9 | Mycobacterium | 93.8 | 91% | <LoQ | N/A | <LoQ | N/A |
| | tuberculosis | | | | | | |
| 10 | Candida albicans | 108.0 | 105% | 41.4 | 100% | <LoQ | N/A |
| 11 | Lactobacilli johnsonii | 94.9 | 92% | 40.4 | 97% | <LoQ | N/A |
| 12 | Chlymdophilia | 90.6 | 88% | <LoQ | N/A | <LoQ | N/A |
| | pnemoniae | | | | | | |
| 13 | Herpes simplex I | 94.5 | 92% | <LoQ | N/A | <LoQ | N/A |
| 14 | Herpes simplex II | 92.2 | 89% | <LoQ | N/A | <LoQ | N/A |

Potential sample interferences unrelated to SARS-CoV-2 infection that could impact the accuracy of test results were also tested (Table 38).

TABLE 38
Testing results of interferences
SOR Hemoglobin Endogenous Interference

| | | IgM | % of | IgG | % of | IgA | % of |
|---|---|---|---|---|---|---|---|
| Compound | Concentration | μg/mL | Control | μg/mL | Control | μg/mL | Control |
| Hemoglobin | 1000 mg/dL | 148.6 | 97% | 437.9 | 114% | 111.9 | 102% |
| Control | — | 152.5 | — | 384.9 | — | 109.7 | — |

Hemoglobin does not significantly interfere with the measurement of saline oral rinse IgG, IgM, or IgA secretory antibodies to SARS-CoV-2 in this assay.

Biotin up to 3,500 ng/mL does not significantly interfere with the measurement of saline oral rinse IgG, IgA, or IgM secretory antibodies to SARS-CoV-2 in the COVID-19 Antibody Test (Table 39).

TABLE 39
Testing results for biotin interferences

| SOR IgG Biotin Interference | | | | SOR IgM Biotin Interference | | | | SOR IgA Biotin Interference | | | |
|---|---|---|---|---|---|---|---|---|---|---|---|
| | | 3500 | | | | 3500 | | | | 3500 | |
| | | ng/mL | | | | ng/mL | | | | ng/mL | |
| Sample | Control | Biotin | % | Sample | Control | Biotin | % | Sample | Control | Biotin | % |
| ID | ng/mL | ng/mL | Difference | ID | ng/mL | ng/mL | Difference | ID | ng/mL | ng/mL | Difference |
| Sample | 67.1 | 61.2 | −10% | Sample | 44.4 | 43.4 | −2% | Sample | 63.9 | 57.2 | −12% |
| 1 | | | | 1 | | | | 1 | | | |
| Sample | 126.1 | 119.7 | −5% | Sample | 79.6 | 73.1 | −9% | Sample | 55 | 54.4 | −1% |
| 2 | | | | 2 | | | | 2 | | | |

Saline oral rinse samples were spiked with a variety of substances that could be present in saline oral rinse samples from eating or drinking, or from bleeding/hemoglobin to assess their impact on IgG, IgM, and IgA results. Some of the exogenous interference substances tested such as toothpaste, mouthwash, tums, tobacco, and coffee interfered higher than 20% with the measurement of saline oral rinse IgG, IgA, or IgM secretory antibodies to SARS-CoV-2 in this assay (Table 40, ctrl.=control). It was required that patients do not eat, drink, smoke, chew gum, or brush their teeth for at least 30 minutes prior to collecting their saline oral rinse sample.

TABLE 40
Testing results for biotin interferences

| Condition: | Sample 1 | | | | | | Sample 2 | | | | | |
|---|---|---|---|---|---|---|---|---|---|---|---|---|
| each substance | IgM | | IgA | | IgG | | IgM | | IgA | | IgG | |
| spiked at 10 | | % of | | % of | | % of | | % of | | % of | | % of |
| μg/mL | ng/mL | ctrl. | ng/mL | ctrl. | ng/mL | ctrl. | ng/mL | ctrl. | ng/mL | ctrl. | ng/mL | ctrl. |
| Control | 136.9 | 110% | 121.3 | 110% | 217.8 | 110% | 150.2 | 110% | 128.6 | 110% | 440.2 | 110% |
| Mouthwash | 142.3 | 114% | 107.6 | 98% | 207.3 | 105% | 166.1 | 122% | 169.0 | 145% | 467.4 | 117% |
| Tobacco | 138.8 | 112% | 106.8 | 97% | 226.5 | 114% | 89.5 | 66% | 43.7 | 37% | 241.9 | 60% |
| Coffee | 149.5 | 120% | 127.8 | 116% | 244.3 | 123% | 120.0 | 88% | 73.7 | 63% | 345.5 | 86% |
| Gum | 129.6 | 104% | 122.3 | 111% | 207.0 | 105% | 126.2 | 92% | 88.0 | 75% | 326.6 | 82% |
| Chocolate | 131.4 | 106% | 118.1 | 107% | 207.9 | 105% | 149.4 | 109% | 119.3 | 102% | 440.0 | 110% |
| Toothpaste | 83.5 | 67% | 42.7 | 39% | 145.4 | 73% | 89.9 | 66% | 43.5 | 37% | 198.9 | 50% |
| Cough Syrup | 131.7 | 106% | 115.3 | 104% | 187.2 | 95% | 149.1 | 109% | 127.8 | 109% | 408.3 | 102% |
| Vodka | 139.2 | 112% | 120.6 | 109% | 208.4 | 105% | 139.0 | 102% | 98.3 | 84% | 356.5 | 89% |
| Tums | 215.3 | 173% | 122.7 | 111% | 281.5 | 142% | 166.5 | 122% | 85.4 | 73% | 369.2 | 92% |

Class specificity studies were conducted to determine the ability of the triplex conjugate to differentiate between human IgG, IgM, and IgA immunoglobulins. Conjugate cross-reactivity was tested by testing the individual calibrator beads coated with either purified human IgG, IgA, or IgM against the individual conjugates and the triplex conjugate. The class specificity of each conjugate (anti-human IgG, anti-human IgM, and anti-human IgA) and the triplex conjugate was evaluated by testing the reactivity of 60 μg of each individual Cal 6 bead (IgG, IgM, and IgA coated beads), or 1.3× more Cal beads than used in the assay to accentuate any potential cross-reactivity against 200 μL of individual anti-IgA or anti-IgG or anti-IgM fluorescent conjugates. The presence of conjugate cross-reactivity was determined by comparing the average triplex signal to the average individual conjugate relative fluorescence units (RFU) signal. The % cross-reactivity was calculated based on the ratio of the average triplex conjugate anti-IgG, anti-IgM, and anti-IgA RFU signal and the average individual anti-IgG, anti-IgM, and anti-IgA conjugate RFU signal when tested against their respective IgG, IgM, or IgA Cal beads. Cross-reactivity of ±10% is acceptable. Each conjugate demonstrated strong fluorescence signal for its specific class with without significant cross-reactivity to the other classes. Cross-reactivity was less than 10% for each conjugate. The triplex conjugate correctly differentiates between human IgG, IgM, and IgA immunoglobulins.

Analytical sensitivity for IgG, IgM, and IgA secretory antibodies to SARS-CoV-2 spike protein RBD and NTD in saline oral rinse was determined based on the Limit of Quantitation (LoQ) of IgG, IgM, and IgA secretory antibodies. Antibody results are reported as Negative, with a Test Result Interpretation “IgG secretory antibodies to SARS-CoV-2 are not detected”, and/or “IgM secretory antibodies to SARS-CoV-2 are not detected”, and/or “IgA secretory antibodies to SARS-CoV-2 are not detected” if IgG, IgM, and IgA antibody levels are less than LoQ. If IgG, and/or IgM, and/or IgA antibody levels are higher than or equal to LoQ, the antibody results are reported as Positive; Numerical result (ng/mL for IgG; ng/mL for IgM, and ng/mL for IgA) is reported outside of the laboratory, with a Test Result Interpretation “IgG secretory antibodies to SARS-CoV-2 are detected”, and/or “IgM secretory antibodies to SARS-CoV-2 are detected”, and/or “IgA secretory antibodies to SARS-CoV-2 are detected”.

Interpretation of multiplex quantitative results in saline oral rinse for IgG, IgM, and IgA secretory antibodies to SARS-Cov-2 spike protein RBD and NTD (Table 41).

TABLE 41
Result interpretation

| Level: Numerical Result | | Test Result |
|---|---|---|
| (with CRM units) | Result | Interpretation |
| IgG < 67.5 ng/mL, and | Negative | Neutralizing secretory |
| IgM < 39.1 ng/mL, and | | antibodies to |
| IgA < 34.7 ng/mL | | SARS-CoV-2 are |
| | | not detected. |
| Cal 6 ≥ IgG ≥ 67.5 | Positive; Numerical | Neutralizing secretory |
| ng/mL, and/or | result is reported | antibodies to |
| Cal 6 ≥ IgM ≥ 39.1 | outside the | SARS-CoV-2 |
| ng/mL, and/or | laboratory | are detected. |
| Cal 6 ≥ IgA ≥ 34.7 | | |
| ng/mL | | |
| IgG > Cal 6*, and/or | Positive; Report | |
| IgM > Cal 6*, and/or | outside of the | |
| IgA > Cal 6* | laboratory indicates | |
| | that the result is | |
| | above ULMI** | |

*Upper limit of measuring interval (ULMI).
**Samples above the ULMI can be diluted 3:1 with Diluent B to generate results within the measuring interval.

### Example 12: Clinical Study to Assess Vaccine Efficacy

Saline oral rinse patient samples were tested for secretory neutralizing IgA, IgG, and IgM against the SARS-CoV-2 spike protein RBD and NTD from June 2021 through April 2022 by the COVID-19 Neutralizing Antibody Test. Some patients were also tested longitudinally over time to follow and track their secretory antibody levels pre-vaccination, post-vaccination (vaccine #1, vaccine #2, and vaccine #3), and post-booster shot (vaccine #1, vaccine #2, and vaccine #3), and to determine which level of secretory IgA was protective by measuring antibody levels pre- and post-breakthrough infection by COVID-19.

From Jun. 30, 2021 to Aug. 17, 2021, 55 non-vaccinated patients with no known prior COVID-19 exposure or symptoms provided saline oral rinse samples which were tested by the SOR Protocol. 24 patients had detectable neutralizing antibodies, with 24/24 (100%) having detectable secretory IgA indicative of prior COVID-19 infection. 3/24 (12.5%) also had detectable secretory IgM indicating a recent infection. 31 patients who wanted to know if they previously had COVID-19 or antibodies (asymptomatic) were negative for secretory antibodies. These results demonstrate asymptomatic patients with a prior COVID-19 infection produce primarily secretory IgA, but produce both secretory IgA and IgM from a current or recent infection.

From Jun. 30, 2021 to Jan. 4, 2022, 286 fully vaccinated patients with no known prior COVID-19 exposure or symptoms provided saline oral rinse samples which were tested by the SOR Protocol. A fully vaccinated patient is defined as a patient who has received both the 1st and 2nd vaccine shots for the vaccine #1 or #2, and it has been at least 14 days since their 2nd vaccine shot, or it is defined as a patient who has received the single vaccine #3 shot and it has been at least 28 days since their shot. This study enrolled a total of 286 fully vaccinated patients (N=155 vaccine #1, N=116 vaccine #2, and N=14 vaccine #3). There were also 2 additional patients tested who had received a vaccine #1 booster shot for a total of 3 vaccine shots.

Table 42 shows the vaccine efficiency test results for vaccine #1. 83 out of 155 patients (53.5%) did not have detectable secretory IgA in their saline oral rinse samples, and 106 out of 155 patients (68.4%) did not have detectable secretory IgG. In comparison, 72 out of 155 patients (46.5%) had detectable secretory IgA, and 49 out of 155 patients (31.63%) had detectable secretory IgG. When looking at Days Post-Vaccination, Positive Predictive Value (PPA) was 61.7% >180 days post-vaccination (66 out of 107 patients were Positive), and for all days tested the PPA was 60.0% (93 out of 155 patients were Positive). Only 3 out of 155 patients (1.9%) also had detectable secretory IgM.

TABLE 42
Vaccine efficiency test results for vaccine #1
Vaccine #1 (n = 155)

| | | | Min | Mean | Max |
|---|---|---|---|---|---|
| Immunoglobulin | Positive | Negative | (ng/mL) | (ng/mL) | (ng/mL) |
| IgA | 72 | 83 | 35 | 85 | 277 |
| IgG | 49 | 106 | 68 | 165 | 365 |

Vaccine #1

| Days post- | | | | | | |
|---|---|---|---|---|---|---|
| vaccination | | n | Positive | Negative | PPA | 95% CI |
| 0-60 | days | 8 | 7 | 1 | 87.5% | 46.7%-99.3% |
| 61-120 | days | 12 | 7 | 5 | 58.3% | 28.6%-83.5% |
| 121-180 | days | 28 | 13 | 15 | 46.4% | 28.0%-65.8% |
| ≥181 | days | 107 | 66 | 41 | 61.7% | 51.7%-70.8% |

| | | | | | |
|---|---|---|---|---|---|
| All days | 155 | 93 | 62 | 60.0% | 51.8%-67.7% |

Table 43 shows the vaccine efficiency test results for vaccine #2. 73 out of 116 patients (62.90%) did not have detectable secretory IgA in their saline oral rinse samples, and 92 out of 116 patients (79.3%) did not have detectable secretory IgG. In comparison, 43 out of 116 patients (37.1%) had detectable secretory IgA, and 24 out of 116 patients (20.7%) had detectable secretory IgG. When looking at Days Post-Vaccination, Positive Predictive Value (PPA) was 55.1% >180 days post-vaccination (38 out of 69 patients were Positive), and for all days tested the PPA was 50.0% (58 out of 116 patients were Positive). Only 6 out of 116 patients (5.2%) also had detectable secretory IgM.

TABLE 43
Vaccine efficiency test results for vaccine #2
Vaccine #2 (n = 116)

| | | | Min | Mean | Max |
|---|---|---|---|---|---|
| Immunoglobulin | Positive | Negative | (ng/mL) | (ng/mL) | (ng/mL) |
| IgA | 43 | 73 | 35 | 86 | 210 |
| IgG | 24 | 92 | 68 | 129 | 265 |

Vaccine #2

| Days post- | | | | | |
|---|---|---|---|---|---|
| vaccination | n | Positive | Negative | PPA | 95% CI |
| 0-60 | 7 | 3 | 4 | 42.9% | 11.8%- |
| days | | | | | 79.8% |
| 61-120 | 12 | 7 | 5 | 58.3% | 28.6%- |
| days | | | | | 83.5% |
| 121-180 | 28 | 10 | 18 | 35.7% | 19.3%- |
| days | | | | | 55.9% |
| ≥181 | 69 | 38 | 31 | 55.1% | 42.7%- |
| days | | | | | 66.9% |
| All days | 116 | 58 | 58 | 50.0% | 40.6%- |
| | | | | | 59.4% |

Table 44 shows vaccine efficiency test results for the vaccine #3. 11 out of 14 patients (78.6%) did not have detectable secretory IgA in their saline oral rinse samples, and 10 out of 14 patients (71.4%) did not have detectable secretory IgG. In comparison, 3 out of 14 patients (21.4%) had detectable secretory IgA, and 1 out of 14 patients (7.1%) had detectable secretory IgG. When looking at Days Post-Vaccination, Positive Predictive Value (PPA) was 83.3% >180 days post-vaccination (5 out of 6 patients were Positive), and for all days tested the PPA was 35.7% (5 out of 14 patients were Positive). 1 out of 11 patients (7.1%) had detectable secretory IgM.

TABLE 44
Vaccine efficiency test results for vaccine #3
Vaccine #3 (n = 14)

| | | | Min | Mean | Max |
|---|---|---|---|---|---|
| Immunoglobulin | Positive | Negative | (ng/mL) | (ng/mL) | (ng/mL) |
| IgA | 3 | 11 | 56 | 111 | 199 |
| IgG | 1 | 10 | 162 | — | 162 |

Vaccine #3

| Days post- | | | | | |
|---|---|---|---|---|---|
| vaccination | n | Positive | Negative | PPA | 95% CI |
| 0-60 | 2 | 0 | 2 | 0% | 0%- |
| days | | | | | 80.2% |
| 61-120 | 3 | 0 | 3 | 0% | 0%- |
| days | | | | | 69.0% |
| 121-180 | 3 | 0 | 3 | 0% | 0%- |
| days | | | | | 69.0% |
| ≥181 | 6 | 5 | 1 | 83.3% | 36.5%- |
| days | | | | | 99.1% |
| All days | 14 | 5 | 9 | 35.7% | 14.0%- |
| | | | | | 64.4% |

Table 45 shows vaccine efficiency test results for all 3 vaccines combined (#1, #2, and #3). 167 out of 286 patients (58.4%) did not have detectable secretory IgA in their saline oral rinse samples, and 211 out of 286 patients (73.8%) did not have detectable secretory IgG. In comparison, 119 out of 286 patients (41.6.47) had detectable secretory IgA, and 75 out of 286 patients (26.2%) had detectable secretory IgG. When looking at Days Post-Vaccination, Positive Predictive Value (PPA) was only 59.8% >180 days post-vaccination (109 out of 286 patients were Positive), and for all days tested the PPA was only 54.9% (157 out of 286 patients were Positive). Only 10 out of 286 patients (3.5%) had detectable secretory IgM.

TABLE 45
Vaccine efficiency test results for all 3 vaccines combined
All Vaccines (n = 286)

| | | | Min | Mean | Max |
|---|---|---|---|---|---|
| Immunoglobulin | Positive | Negative | (ng/mL) | (ng/mL) | (ng/mL) |
| IgA | 119 | 167 | 35 | 123 | 277 |
| IgG | 75 | 211 | 68 | 166 | 365 |

All Vaccines

| Days post- | | | | | |
|---|---|---|---|---|---|
| vaccination | n | Positive | Negative | PPA | 95% CI |
| 0-60 | 17 | 10 | 7 | 58.8% | 33.5%- |
| days | | | | | 80.6% |
| 61-120 | 27 | 14 | 13 | 51.9% | 32.4%- |
| days | | | | | 70.8% |
| 121-180 | 60 | 21 | 36 | 40.0% | 27.8%- |
| days | | | | | 53.5% |
| ≥181 | 182 | 109 | 73 | 59.8% | 52.4%- |
| days | | | | | 67.0% |
| All days | 286 | 157 | 129 | 54.9% | 48.9%- |
| | | | | | 60.7% |

2 out of 2 patients (100%) who were fully vaccinated and boosted with the vaccine #1 booster were both positive for secretory IgA and secretory IgG. They also had high levels of secretory IgA (96.8 ng/mL and 191.4 ng/mL, respectively) and high levels of secretory IgG (205.7 ng/mL and 278.3 ng/mL, respectively). The PPA was 100% for these 2 boosted patients.

The results of this vaccinated patient study indicated only 157 out of 286 (54.9%) of fully vaccinated patients had detectable secretory IgA and/or secretory IgG in their saliva (saline oral rinse), and only 119 out of 286 patients (41.6%) had detectable secretory IgA (min 35.2 ng/mL, max 277.2 ng/mL, mean 87.1 ng/mL) where 17 patients (5.9%) had IgA levels less than 40 ng/mL, 20 patients (7.0%) had IgA levels from 40-50 ng/mL, 17 patients (5.9%) had IgA levels from 50-60 ng/mL, and 67 patients (23.4%) had IgA levels higher than 60 ng/mL. Since secretory neutralizing antibodies such as secretory IgA against the SARS-CoV-2 spike protein RBD and NTD is a patient's first line of defense against a respiratory pathogen infection such as SARS-CoV-2, this data suggests vaccination alone may not confer protective immunity to prevent COVID-19 infection. The SARS-CoV-2 spike protein is a transmembrane protein that assembles into trimers to form spikes on its surface. Each S1 spike monomer comprises the N-terminal domain (NTD) and receptor binding domain (RBD). SARS-CoV-2 uses the transmembrane receptor angiotensin-converting enzyme 2 (ACE-2) to infect epithelial cells in the airways and lungs by spike S1-RBD recognition and binding to ACE-2, and spike S2 viral fusion and entry. If a patient has a sufficient titer or level of secretory neutralizing antibodies they will bind to the SARS-CoV-2 spikes and block or prevent them from binding to ACE-2. Interestingly, the 2 boosted patients tested both had high levels of secretory IgA detected indicating the booster shot increased secretory IgA levels in their saliva which offers additional protective immunity.

From Nov. 18, 2021 to Feb. 18, 2022, 32 fully vaccinated and boosted patients provided saline oral rinse samples which were tested by the SOR Protocol. The fully vaccinated patients (N=20 vaccine #2, N=9 vaccine #1, N=4 vaccine #3) received either the vaccine #1 Booster (N=15) or the vaccine #2 Booster (N=18).

For the vaccine #1 Booster (N=15), 4 out of 15 patients (26.7%) did not have detectable secretory IgA in their saline oral rinse samples, and 2 out of 15 patients (13.3%) did not have detectable secretory IgG. In comparison, 11 out of 15 patients (73.3%) had detectable secretory IgA (min 35 ng/mL, max 222 ng/mL, mean 63 ng/mL), and 13 out of 15 patients (86.7%) had detectable secretory IgG (min 73 ng/mL, max 560 ng/mL, mean 164 ng/mL). When looking at Days Post-Booster, Positive Predictive Value (PPA) was 100% (15 out of 15 patients were Positive) at all Days Post-Booster analyzed. 7 out of 15 patients (46.7%) also had detectable secretory IgM (min 41 ng/mL, max 278 ng/mL, mean 101 ng/mL).

For the vaccine #2 Booster (N=18), 3 out of 18 patients (16.7%) did not have detectable secretory IgA in their saline oral rinse samples, and 4 out of 18 patients (22.2%) did not have detectable secretory IgG. In comparison, 15 out of 18 patients (83.3%) had detectable secretory IgA (min 35 ng/mL, max 135 ng/mL, mean 69 ng/mL), and 14 out of 18 patients (77.8%) had detectable secretory IgG (min 71 ng/mL, max 338 ng/mL, mean 130 ng/mL). When looking at Days Post-Booster, overall Positive Predictive Value (PPA) was 94.4% (17 out of 18 patients were Positive) and was 93.8% for 61-120 days post-booster (9 out of 10 patients were Positive). 2 out of 18 patients (11.1%) also had detectable secretory IgM (46 ng/mL and 48 ng/mL, respectively).

For all Booster patients (N=33), 7 out of 33 patients (21.2%) did not have detectable secretory IgA in their saline oral rinse samples, and 6 out of 33 patients (18.2%) did not have detectable secretory IgG. In comparison, 26 out of 33 patients (78.8%) had detectable secretory IgA, and 27 out of 33 patients (81.8%) had detectable secretory IgG. When looking at Days Post-Booster, overall Positive Predictive Value (PPA) was 97.0% (32 out of 33 patients were Positive). 9 out of 33 patients (27.3%) also had detectable secretory IgM (46 ng/mL and 48 ng/mL, respectively).

The results of this vaccinated and boosted patient study indicated 32 out of 33 (97.0%) of fully vaccinated and boosted patients had detectable secretory IgA and/or secretory IgG in their saliva (saline oral rinse), and 26 out of 33 (78.8%) had detectable secretory IgA where 6 patients (18.2%) had IgA levels <40 ng/mL, 7 patients (21.2%) had IgA levels from 40-50 ng/mL, 3 patients (9.1%) had IgA levels from 50-60 ng/mL, and 10 patients (30.3%) had IgA levels >60 ng/mL. Since secretory neutralizing antibodies such secretory IgA against the SARS-CoV-2 spike protein RBD and NTD is a patient's first line of defense against a respiratory pathogen infection such as SARS-CoV-2, this data suggests the booster shot significantly increased detectable secretory IgA from 41.6% (119 out of 286 patients) in fully vaccinated patients to 78.8% (26 out of 33 patients) in fully vaccinated and boosted patients. This booster study data also agrees with the original 2 boosted patients tested in the fully vaccinated study as both fully vaccinated and boosted patients had detectable IgA as well.

From Mar. 24, 2021 to Apr. 28, 2022, 3 family members (Father age 47-48, Mother age 49-50, and Daughter age 12-13) were longitudinally tested by providing saline oral rinse samples over time for testing by the SOR Protocol. This testing occurred after natural infection for the Father and Daughter, before and after vaccination shots and booster shots for all 3 family members, and before and after breakthrough COVID-19 infection of the Father for all 3 family members.

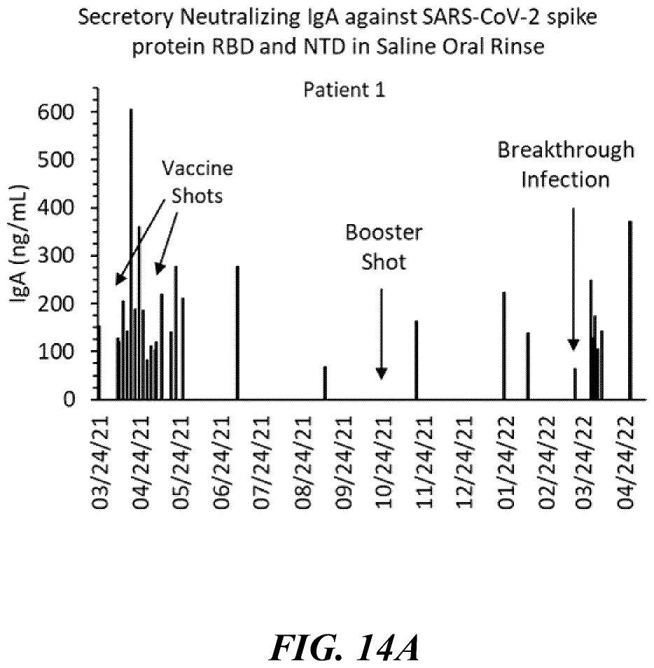

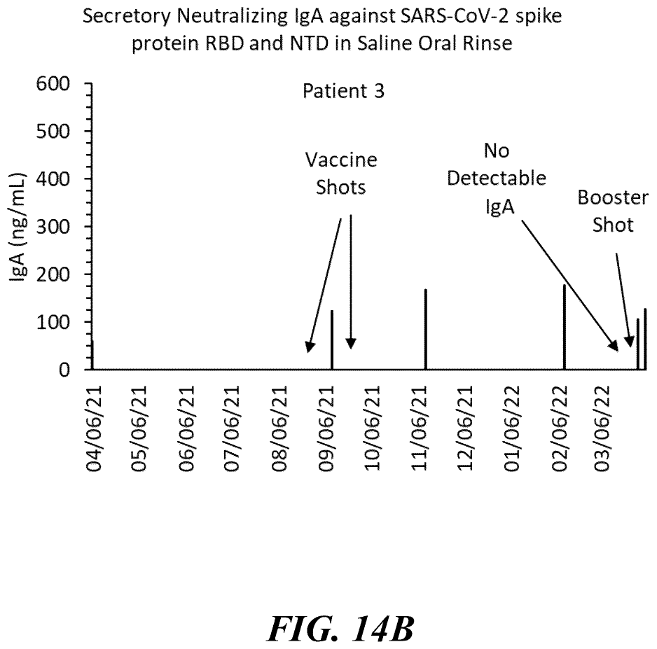

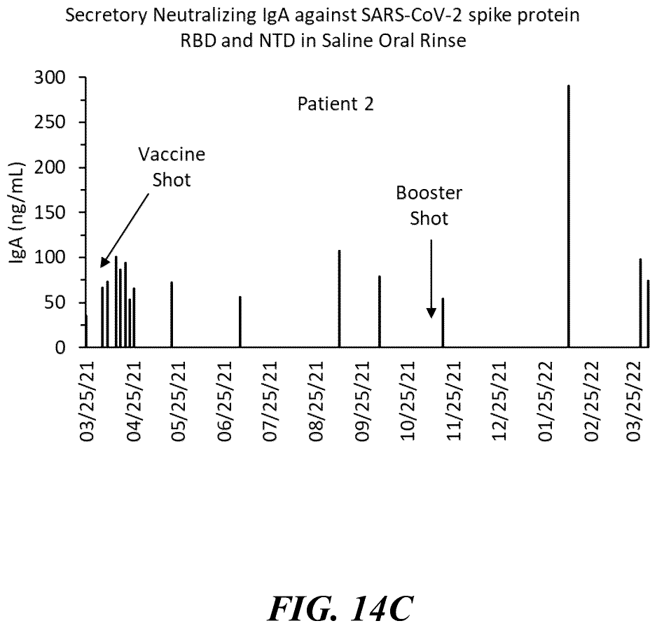

The Father had previously been COVID-19 positive in February 2020 and February 2021 (infected 2 different times by SARS-CoV-2) with his 2nd infection confirmed positive by RT-PCR on Feb. 18, 2021 prior to longitudinal testing for this study. The Father was fully vaccinated by the vaccine #1 vaccine (Shot 1 on Apr. 7, 2021, and Shot 2 on May 5, 2021), and boosted by the vaccine #1 Booster on Nov. 13, 2021. The Father had a breakthrough COVID-19 infection 135 days post-booster shot and tested positive for SARS-CoV-2 by 2 different rapid tests (iHealth rapid test, and Roche® rapid test) and by RT-PCR (Access Genetics, LLC, OraRisk® COVID-19 RT-PCR (EUA200464)) on Mar. 30, 2022. The Father's secretory IgA values decreased from a max value of 222.2 ng/mL on Jan. 23, 2022 (71 days post-booster shot) to 64.5 ng/mL on Mar. 18, 2022 (125 days post-booster shot, and 10 days prior to breakthrough infection during travel between Minnesota and Arizona with his Daughter from Mar. 25-28, 2022). Table 46 and FIG. 14A show the antibody longitudinal test study data of the Father.

TABLE 46
Longitudinal test study of antibody of the Father

| | | Days After Last | | | | |
|---|---|---|---|---|---|---|
| | | Vaccine Shot | | | | |
| | | (vaccine #1) or | | | | |
| Patient 1 | | Booster Shot | Result (ng/mL) | | | |
| (Father) | Date | (vaccine #1) | IgA | IgG | IgM | Interpretation |
| Pre-Shot | Mar. 24, 2021 | 14 days prior to | 152.1 | <LoQ | <LoQ | POS |
| | | Shot 1 (COVID-19 | | | | |
| | | positive by RT- | | | | |
| | | PCR on Feb. 18, 2021) | | | | |
| Shot 1 | Apr. 7, 2021 | 0 | 127.9 | <LoQ | <LoQ | POS |
| | Apr. 8, 2021 | 1 | 119.3 | <LoQ | <LoQ | POS |
| | Apr. 11, 2021 | 4 | 205.5 | <LoQ | <LoQ | POS |
| | Apr. 14, 2021 | 7 | 142.8 | <LoQ | <LoQ | POS |
| | Apr. 17, 2021 | 10 | 603.4 | 168.6 | <LoQ | POS |
| | Apr. 20, 2021 | 13 | 187.8 | 182.5 | <LoQ | POS |
| | Apr. 23, 2021 | 16 | 360.9 | 235.9 | <LoQ | POS |
| | Apr. 26, 2021 | 19 | 186.5 | 209.4 | <LoQ | POS |
| | Apr. 29, 2021 | 22 | 82.2 | 90.5 | <LoQ | POS |
| | May 2, 2021 | 25 | 111.9 | 115.1 | <LoQ | POS |
| Shot 2 (28 | May 5, 2021 | 0 | 103.4 | 89.7 | <LoQ | POS |
| days after | May 6, 2021 | 1 | 119.3 | 93.9 | <LoQ | POS |
| Shot 1) | May 10, 2021 | 5 | 218.5 | 87.4 | <LoQ | POS |
| −71% | May 17, 2021 | 12 | 139.5 | 99.7 | <LoQ | POS |
| | May 21, 2021 | 16 | 277.6 | 179.9 | <LoQ | POS |
| | May 26, 2021 | 21 | 211.4 | 202.5 | <LoQ | POS |
| | Jul. 6, 2021 | 62 | 276.7 | 141.9 | <LoQ | POS |
| | Sep. 10, 2021 | 128 | 68.2 | | <LoQ | POS |
| Booster (192 | Nov. 13, 2021 | — | — | — | — | — |
| days after | Nov. 18, 2021 | 5 | 163.9 | 86.9 | <LoQ | POS |
| Shot 2) | Jan. 23, 2022 | 71 | 222.2 | 194.7 | <LoQ | POS |
| | Feb. 10, 2022 | 89 | 137.5 | 144.8 | <LoQ | POS |
| | Mar. 18, 2022 | 125 | 64.5 | 70.6 | <LoQ | POS |
| COVID-19 | Mar. 28, 2022 | 135 | — | — | — | — |
| Breakthrough | | | | | | |
| Infection | | | | | | |
| Symptoms | | | | | | |
| Onset (135 | | | | | | |
| days after | | | | | | |
| Booster) | | | | | | |
| RT-PCR | Mar. 30, 2022 | 2 | 248.6 | 173.7 | <LoQ | POS |
| Positive (Ct | | | | | | |
| 24.8); | | | | | | |
| iHealth rapid | | | | | | |
| test positive; | | | | | | |
| Roche ® | | | | | | |
| rapid test | | | | | | |
| positive | | | | | | |
| RT-PCR | Mar. 31, 2022 | 3 | 128.3 | 145.9 | <LoQ | POS |
| Positive (Ct | | | | | | |
| 25.6) | | | | | | |
| RT-PCR | Apr. 2, 2022 | 5 | 173.1 | 145.3 | <LoQ | POS |
| Positive (Ct | | | | | | |
| 26.1); | | | | | | |
| Roche ® | | | | | | |
| rapid test | | | | | | |
| positive | | | | | | |
| Roche ® | Apr. 3, 2022 | 6 | — | — | — | — |
| rapid test | | | | | | |
| positive | | | | | | |
| RT-PCR | Apr. 4, 2022 | 7 | 105.2 | 156.6 | <LoQ | POS |
| Positive (Ct | | | | | | |
| 30.4) | | | | | | |
| Roche ® | Apr. 5, 2022 | 8 | — | — | — | — |
| rapid test | | | | | | |
| weak | | | | | | |
| positive | | | | | | |
| iHealth rapid | Apr. 6, 2022 | 9 | — | — | — | — |
| test weak | | | | | | |
| positive | | | | | | |
| RT-PCR | Apr. 7, 2022 | 10 | 143.1 | 124.7 | <LoQ | POS |
| Positive (Ct | | | | | | |
| 36.2); | | | | | | |
| iHealth rapid | Apr. 28, 2022 | 31 | 416.0 | 198.7 | <LoQ | POS |
| test negative | | | | | | |

The Daughter had previously been COVID-19 positive in February 2021 with a positive RT-PCR on Feb. 22, 2021, prior to longitudinal testing for this study. The Daughter was positive resultant to exposure to her Father who was RT-PCR confirmed COVID-19 positive on Feb. 18, 2021, and where the Daughter had been quarantining and home schooling at the time. The Daughter was fully vaccinated by the vaccine #2 authorized for ages 12-15 (Shot 1 on Sep. 1, 2021, and Shot 2 on Sep. 22, 2021), and boosted by the vaccine #2 Booster on Mar. 19, 2022. While the Father had a breakthrough COVID-19 infection 135 days post-booster shot and tested positive for SARS-CoV-2 on Mar. 30, 2022, the Daughter did not get infected with COVID-19 and tested negative multiple times by RT-PCR. The Daughter's secretory IgA values increased from undetectable the day she received her Booster shot on Mar. 19, 2022, to 105.6 ng/mL on Mar. 30, 2022, or 11 days post-booster shot, and 127.5 ng/mL on Apr. 4, 2022, or 16 days post-booster shot. The Daughter did not get infected by her Father even though they travelled together to Arizona and she was exposed to her father when he had COVID-19 symptoms. Unlike the first time where the Father exposed and infected his daughter, this did not happen the second time after the Daughter was boosted. Table 47 and FIG. 14B show the antibody longitudinal test study data of the Daughter.

TABLE 47
Longitudinal test study of antibody of the Daughter

| | | Days After Last | | | | |
|---|---|---|---|---|---|---|
| | | Vaccine Shot | | | | |
| | | (vaccine #2) or | | | | |
| Patient 3 | | Booster Shot | Result (ng/mL) | | | |
| (Daughter) | Date | (vaccine #2) | IgA | IgG | IgM | Interpretation |
| Pre-Shot | Apr. 6, 2021 | 148 days prior to | 59.8 | <LoQ | <LoQ | POS |
| | | Shot 1 (COVID | | | | |
| | | positive by RT- | | | | |
| | | PCR on Feb. 22, 2021) | | | | |
| Shot 1 | Sep. 1, 2021 | — | — | — | — | — |
| | Sep. 10, 2021 | 9 | 124.3 | 512.6 | <LoQ | POS |
| Shot 2 (28 | Sep. 22, 2021 | — | — | — | — | — |
| days after | Nov. 11, 2021 | 50 | 168.3 | 157.3 | <LoQ | POS |
| Shot 1) | Feb. 10, 2022 | 113 | 176.9 | 208.2 | <LoQ | POS |
| | Mar. 18, 2022 | 177 | <LoQ | <LoQ | <LoQ | NEG |
| Booster (178 | Mar. 19, 2022 | — | — | — | — | — |
| days after | | | | | | |
| Shot 2) | | | | | | |
| RT-PCR | Mar. 30, 2022 | 11 | 105.6 | <LoQ | <LoQ | POS |
| Negative; | | | | | | |
| iHealth rapid | | | | | | |
| test negative | | | | | | |
| RT-PCR | Apr. 4, 2022 | 16 | 127.5 | 227.50 | <LoQ | POS |
| Negative | | | | | | |

The Mother had previously been COVID-19 positive in February/March 2020 and developed Long COVID symptoms. The Mother received the vaccine #3 on Apr. 1, 2021, and she was boosted by the vaccine #1 Booster on Nov. 13, 2021. While the Father had a breakthrough COVID-19 infection 135 days post-booster shot and tested positive for SARS-CoV-2 on Mar. 30, 2022, the Mother did not get infected with COVID-19 and tested negative multiple times by RT-PCR. The Mother's secretory IgA values were 97.8 ng/mL on Mar. 30, 2022, and 73.9 ng/mL on Apr. 4, 2022. The Mother did not get infected by the Father even though she was exposed to the father at home when he had COVID-19 symptoms and after he tested RT-PCR positive. Although the Mother was exposed to the Father when he was COVID-19 positive in February 2021 and also in March 2022, the Mother never contracted the virus nor tested positive for COVID-19 by RT-PCR. Table 48 and FIG. 14C show the antibody longitudinal test study data of the Mother.

TABLE 48
Longitudinal test study of antibody of the Mother

| | | Shot | | | | |
|---|---|---|---|---|---|---|
| Patient 2 | | (vaccine #3) or Booster | Result (ng/mL) | | | |
| (Mother) | Date | Shot (vaccine #1) | IgA | IgG | IgM | Interpretation |
| Pre-Shot | Mar. 25, 2021 | 7 days prior to Shot 1 | 35.3 | <LoQ | <LoQ | POS |
| | | (COVID-19 symptoms | | | | |
| | | on Mar. 5, 2020, positive | | | | |
| | | for anti-spike S1 IgG | | | | |
| | | antibodies by | | | | |
| | | Euroimmun ELISA | | | | |
| | | May 5, 2020) | | | | |
| Shot 1 | Apr. 1, 2021 | — | — | — | — | — |
| | Apr. 2, 2021 | 1 | <LoQ | <LoQ | <LoQ | NEG |
| | Apr. 5, 2021 | 4 | 67.0 | <LoQ | <LoQ | POS |
| | Apr. 8, 2021 | 7 | 72.9 | <LoQ | <LoQ | POS |
| | Apr. 14, 2021 | 13 | 100.7 | <LoQ | 43.4 | POS |
| | Apr. 17, 2021 | 16 | 87.0 | <LoQ | 42.4 | POS |
| | Apr. 20, 2021 | 19 | 94.4 | <LoQ | 48.1 | POS |
| | Apr. 23, 2021 | 22 | 53.8 | <LoQ | <LoQ | POS |
| | Apr. 26, 2021 | 25 | 65.4 | <LoQ | <LoQ | POS |
| | May 21, 2021 | 50 | 72.2 | <LoQ | <LoQ | POS |
| | Jul. 6, 2021 | 96 | 56.0 | <LoQ | <LoQ | POS |
| | Sep. 10, 2021 | 162 | 107.8 | <LoQ | <LoQ | POS |
| | Oct. 7, 2021 | 189 | 79.2 | <LoQ | <LoQ | POS |
| Booster (226 | Nov. 13, 2021 | — | — | — | — | — |
| days after | | | | | | |
| Shot 1) | | | | | | |
| RT-PCR | Nov. 18, 2021 | 5 | 53.9 | <LoQ | <LoQ | POS |
| Negative | | | | | | |
| RT-PCR | Feb. 10, 2022 | 89 | 291.4 | 150.1 | <LoQ | POS |
| Negative | | | | | | |
| RT-PCR | Mar. 30, 2022 | 137 | 97.8 | <LoQ | <LoQ | POS |
| Negative | | | | | | |
| RT-PCR | Apr. 4, 2022 | 142 | 73.9 | 100.8 | <LoQ | POS |
| Negative | | | | | | |

The results of this longitudinal test study demonstrated the primary secretory immune response to natural infection is secretory neutralizing IgA antibodies against the SARS-CoV-2 spike protein RBD and NTD. For the Father, Mother, and Daughter, only IgA was detected resultant of their natural infections, and secretory IgG was not also detected until after vaccination and the booster shot. This further demonstrates the role of secretory IgA for protective immunity.

The Father demonstrated a very interesting antibody profile of IgA and IgG after each vaccine shot and the booster shot where both IgA and IgG significantly increased after each shot, but at 128 days after the second vaccine shot, and again at 125 days (4 months) after his booster shot, his IgA values precipitously decreased from values >200 ng/mL to 68.2 ng/mL on Sep. 10, 2021, and to 64.5 ng/mL on Mar. 18, 2022. Only 10 days later, or on Mar. 28, 2022, the Father developed COVID-19 symptoms and tested positive by both rapid test and RT-PCR on Mar. 30, 2022. The Father had a breakthrough infection even though he had previously been infected with COVID-19 in February 2020 and again February 2021, and he was fully vaccinated and boosted. Interestingly, after his 3rd COVID-19 infection both his secretory IgA and IgG levels significantly increased to 416 ng/mL IgA and 199 ng/mL IgG on Apr. 28, 2022, or 31 days after symptoms onset.

Neither the Mother nor Daughter contracted COVID-19 from the Father, and they subsequently tested negative 2 different times by RT-PCR at 2 and 7 days after the Father's symptoms onset when he was contagious. Both the Mother and Daughter had detectable secretory IgA levels of 97.8 ng/mL and 105.6 ng/mL, respectively on Mar. 30, 2022, and 73.9 ng/mL and 127.5 ng/mL, respectively on Apr. 4, 2022. Based on these results, secretory IgA values >65 ng/mL appear to offer protective immunity and they did not get infected with COVID-19.

The following is another COVID-19 breakthrough infection case study with antibody test and PCR test results.

A 24-year-old apparently healthy adult, white, Caucasian, female with no known co-morbidities or prior health issues was fully vaccinated against COVID-19. She received her first vaccine #2 shot on Apr. 3, 2021, second shot on May 6, 2021, and subsequent booster shot on Nov. 21, 2021. She had no prior COVID-19 infections. On Mar. 18, 2022, or 117 days (4 months) after her booster, she was likely exposed to SARS-CoV-2 during an outing in a restaurant without masking. She experienced COVID-19 symptoms of a sore throat, cough, and fever on Mar. 21, 2022, or 3 days post-exposure, and tested negative for SARS-CoV-2 by ThermoFisher TaqPath COVID-19 PCR. However, on Mar. 23, 2022, or 5 days post-exposure, she tested COVID-19 positive twice by Abbott BinaxNOW COVID-19 Antigen Self-Test. On Mar. 24, 2022, Mar. 25, 2022, Mar. 26, 2022, Mar. 28, 2022, and Mar. 31, 2022 the patient provided saline oral rinse (SOR) samples for testing by the FDA emergency use authorized OraRisk® COVID-19 RT-PCR (EUA200464) to confirm SARS-CoV-2 specific infection, as well as by the SOR Protocol to detect and quantitate secretory IgA, IgG, and IgM neutralizing antibodies against SARS-CoV-2 spike protein RBD and NTD. All SOR samples were collected from 9 AM to 10 AM. The Mar. 24, 2022 SOR sample, or 3 days after symptoms onset, was negative for secretory neutralizing antibodies based on the assay's Limit of Quantitation, or the dose as which the total CV less than 20% for IgA (34.7 ng/mL), IgG (67.5 ng/mL), and IgM (39.1 ng/mL), but was RT-PCR positive for SARS-CoV-2 (Ct 31.54). However, her Mar. 25, 2022 SOR sample, or 4 days after symptoms onset, had detectable IgA (58.9 ng/mL) and IgG (79.3 ng/mL), her Mar. 26, 2022 SOR sample had detectable IgA (40.3 ng/mL) and IgM (38.9 ng/mL), her Mar. 28, 2022 SOR sample had detectable IgA (125.7 ng/mL) and IgM (49.6 ng/mL), and her Mar. 31, 2022 SOR sample had detectable IgA (69.5 mg/mL) above the LoQ cutoffs demonstrating a rise in secretory IgA levels beginning 4 days after symptoms onset, an increase in secretory IgM from 5 to 7 days after symptoms onset, and detectable IgG close to the LoQ on 3/25. The Mar. 25, 2022 (Ct 32.72), Mar. 26, 2022 (Ct 31.92), and Mar. 28, 2022 (Ct 36.31) SOR samples were all RT-PCR positive for SARS-CoV-2. Her Mar. 31, 2022 SOR samples was RT-PCR negative. Table 49 shows the antibody testing results.

TABLE 49
Antibody testing results.

| | | | | Abbott | | |
|---|---|---|---|---|---|---|
| | | | | BinaxNOW | | OraRisk ® |
| | IgA | IgG | IgM | COVID-19 | RT-PCR | COVID-19 |
| | (ng/mL) | (ng/mL) | (ng/mL) | Antigen Self-Test | Taqpath | RT-PCR |
| Date | SOR | SOR | SOR | Nasal Swab | Nasal Swab | SOR |
| Mar. 21, 2022 | NA | NA | NA | NA | NEG | NA |
| Mar. 23, 2022 | NA | NA | NA | POS (x2) | NA | NA |
| Mar. 24, 2022 | Undetected | Undetected | Undetected | NA | NA | POS (Ct |
| | | | | | | 31.5) |
| Mar. 25, 2022 | 58.9 | 79.3 | Undetected | NA | NA | POS (Ct |
| | | | | | | 33.2) |
| Mar. 26, 2022 | 40.3 | Undetected | 38.9 | POS | NA | POS (Ct |
| | | | | | | 31.9) |
| Mar. 28, 2022 | 125.7 | Undetected | 49.6 | NEG | NA | POS (Ct |
| | | | | | | 36.3) |
| Mar. 31, 2022 | 69.5 | Undetected | Undetected | NA | NA | Undetected |

This data demonstrates an acute increase in secretory IgA levels beginning 4 days after COVID-19 breakthrough infection of a fully vaccinated and boosted patient, or an increase in secretory neutralizing IgA levels >LoQ from 58.9 ng/mL to 125.7 ng/mL. Her IgA, IgG, and IgM secretory neutralizing antibodies were undetected in her saliva-based SOR sample 3 days after symptoms onset suggesting she did not have a sufficient protective antibody titer or threshold to protect her from breakthrough infection at 3.9 months, or 117 days, after her vaccine #2 booster shot. Secretory neutralizing IgA2 specific to the SARS-CoV-2 spike protein RBD and NTD may play a more important role than IgG in protective immunity and neutralization efficacy. This data suggests IgA2 may be a diagnostic target in SOR or saliva-based testing to help establish a protective immunity threshold or cutoff, and to assess vaccine efficacy at producing neutralizing IgA2.

Secretory IgM was also detected beginning 5 days post symptoms onset and increased from 38.9 to 49.6 ng/mL by 7 days post symptoms onset. Since the disclosed COVID-19 Neutralizing Antibody Assay in the Serum Protocol uses both the SARS-CoV-2 S1 spike protein RBD and S1 spike protein NTD antigens in its capture reagent and test design, the secretory neutralizing IgM detected was likely specific to SARS-CoV-2 spike protein NTD and not to RBD as the vaccine #2 mRNA vaccine produces RBD and not NTD as the immunogen to produce neutralizing antibodies in the patient.

### Example 13: Testing of Total SARS-CoV-2 Spike Protein Anti-RBD and Anti-NTD Neutralizing Antibodies

This example shows the use of biomarker capture particles, i.e., magnetic streptavidin particles coated with biotinylated SARS-CoV-2 spike protein RBD and NTD (capture beads), to capture, purify, and detect/measure biomarkers (total SARS-CoV-2 spike protein anti-RBD and anti-NTD neutralizing antibodies) from saline oral rinse by cPass™ ELISA.

The protocol comprised the following steps:
1) 100 μL Capture Beads (coated with SARS-CoV-2 spike protein RBD and NTD) were added to 1 mL or 2 mL (2× volume) saline oral rinse sample in a 2 mL vial;
2) the vial was incubated for 30 minutes at 37° C. with mixing on a rocker in a 37° C. incubator;
3) the Capture Beads were magnetically separated and aspirated, the supernatant was discarded, and the Capture Beads were washed 3 times with a Wash Buffer;
4) 100 μL Elution Buffer was added, the solution was vortex mixed to resuspend the Capture Beads, and incubated for 1 min in the Elution Buffer to elute total COVID-19 Neutralizing IgA, IgG, and/or IgM total antibodies captured by the Capture Beads;
5) the Capture Beads were separated on a magnet and aspirated and the eluate was dispensed into a new 2 mL vial;
6) 20 μL Neutralization Buffer was immediately added and the solution was vortex mixed to quickly neutralize the eluate pH;
7) the neutralized eluate was tested by cPass™ ELISA testing; and
8) cPass™ ELISA protocol was followed and cPass™ ELISA reagents were used to test all samples and controls.

The testing results are shown in Table 50.

TABLE 50
cPass ™ ELISA Testing results

| | | | | % |
|---|---|---|---|---|
| | | | | Inhibi- |
| Sample | Sample ID | Well | OD450 | tion |
| Negative cPass ™ Control | Neg Ctrl | A1 | 1.821 | 0% |
| Positive cPass ™ Control | Pos Ctrl | B1 | 0.846 | 54% |
| Negative Internal Control | Neg IC | C1 | 2.061 | 0% |
| (saline) | | | | |
| Positive Internal Control - Low | Pos IC Low | D1 | 2.078 | 0% |
| 2x volume Positive Internal | 2x Pos IC | E1 | 1.581 | 13% |
| Control - Low | Low | | | |
| Positive Internal Control - High | Pos IC High | F1 | 1.634 | 10% |
| 2x volume Positive Internal | 2x Pos IC | G1 | 1.086 | 40% |
| Control - High | High | | | |
| COVID-19 Neutralizing | Pos Sample | H1 | 1.452 | 20% |
| Antibody Positive Patient | | | | |
| Sample | | | | |

The Neg Ctrl (0% Inhibition) from cPass, and Neg IC (0% Inhibition) the disclosed method, did not inhibit the cPass™ ELISA and correctly read negative.

The Pos Ctrl (54% Inhibition) from cPass, and 2× Pos IC Low (13% Inhibition), Pos IC High (10% Inhibition), and 2× Pos IC High (40% Inhibition) from the disclosed method, demonstrated inhibition in the cPass™ ELISA. The Pos Sample (20% Inhibition) also demonstrated inhibition in the cPass™ ELISA.

While the Pos IC Low (0% Inhibition) did not inhibit the cPass™ ELISA, when twice the sample volume (2× volume) was tested it did demonstrate 13% inhibition in the cPass™ ELISA.

These results demonstrate neutralizing IgA, IgG, and IgM antibodies against SARS-CoV-2 spike protein RBD and NTD can be captured and purified from saline oral rinse (saliva) using magnetic antigen-coated Capture Beads for subsequent detection by the cPass™ ELISA. The cPass™ ELISA is a competitive immunoassay that detects neutralizing anti-RBD antibodies by competition between RBD-conjugate and the ACE2 human receptor which binds RBD.

### Example 14: Collection Kit

A subject receives a saliva receptacle (e.g., a collection tube) and lid (e.g., cap) in the mail. The subject expectorates into the tube, and caps the tube. The cap includes particles that are released into the sample upon capping. For example, the cap may be a screwcap that releases the particles when the subject screws the cap on the receptacle. The particles include capture beads (i.e., biomarker capture particles) with conjugated antibodies recognizing an antigen. For example, the subject may be suspected of having respiratory disease such as COVID-19, and the antigen is a respiratory disease antigen such as a SARS-CoV-2 spike protein. The tube or cap may also include a solution or buffer. The subject may be provided an oral rinse. The oral rinse may include the buffer. The subject then delivers or mails the collection tube to a laboratory. While in transit, the particles are incubated with any respiratory disease antibodies from the saliva sample. Thus, the particles are incubated with the entire sample in a convenient manner.

### Example 15: Sandwich Competitive Biomolecule Assay

A biomolecule or biomarker screen is performed on a well plate such as a 96-well plate or a 384-well plate. Each well may include a sample from a separate individual (or subject). The samples may include filtered or non-filtered saliva samples. The screening is to detect if the samples contain the biomolecule. Magnetic capture particles are added to each well. The assay may also be performed in a format other than a well plate, such as in a tube. The magnetic capture particles are conjugated to an antibody that recognizes an antigen of the biomolecule being screened. The wells including the sample and magnetic capture particles are then incubated. During incubation, the antibody of the capture particles may bind to the biomolecule.

Next, a non-magnetic conjugate is added to the wells. The conjugate includes an antibody that binds to the antigen, or that binds to a second antigen of the biomolecule being screened. The conjugate is added in a rate-limiting amount compared to the capture particles. For example, the ratio of conjugate to capture antibodies may be a ratio between 1:2 and 1:10,000. The wells including the sample with the capture particles and the conjugate are then incubated. During this second incubation, the antibody of the conjugate may bind to the biomolecule, and may compete with binding of the capture particles.

The conjugate may include a label. The label may be a fluorophore, a color, an enzyme, or a radiolabel. The conjugate may include a particle such as a non-magnetic bead. Any aspect of the conjugate may be labeled. For example, the conjugate may include a labeled antibody or a labeled particle.

The incubations may be between 5 minutes and 2 hours, and may include shaking. The incubations may be at room temperature or at a heightened temperature such as 30° C. or 37° C.

After the second incubation, the magnetic capture particles are removed from the wells with a magnet, and any conjugate signal in the wells is measured. When the sample includes the biomolecule, the biomolecule binds to the magnetic capture particle, and the conjugate binds to the biomolecule that is bound to the magnetic capture particle. This leads to the conjugate being removed from the well with the magnetic capture particle, and so no conjugate signal, a low amount of conjugate signal, or a decrease in conjugate signal in the wells is indicative of the sample comprising the biomolecule. On the other hand, if the sample does not include the biomolecule, then the biomolecule does not bind to the magnetic capture particles and hence no conjugate is pulled out by the magnet when the magnet pulls the magnetic capture particles out of the wells. Thus, a presence, lack of presence, or amount of the biomolecule in the sample is inferred based on a presence, lack of presence, or amount of the conjugate signal in the wells. Alternatively, the assay may be performed by measuring conjugate pulled out by the magnetic capture particles.

In the case of non-magnetic bead as the conjugate, a clearance of the supernatant is indicative of the sample comprising the biomolecule of interest. In the absence of the biomolecule of interest, the concentration of the non-magnetic beads will remain substantially unchanged after the magnetic capture particles are pulled out the sample. In the presence of the biomolecule of interest, the concentration of the non-magnetic beads will reduce or the non-magnetic beads will be completely pulled out of the sample when the magnetic capture particles are pulled out the sample, leading to a clear supernatant.

This may be a quick and easy way for measuring biomolecules, and may be done without any wash steps following the incubations.

### Example 16: Competitive Antibody Assay

A similar type of screen as in Example 15 may be performed as follows, but in which the biomolecule is an antibody that is being screened for, and the capture particles include an antigen of the antibody.

An antibody screen is performed on a well plate such as a 96-well plate or a 384-well plate. The assay may also be performed in a format other than a well plate, such as in a tube. Each well may include a sample from a separate individual or subject. The samples may include filtered or non-filtered saliva samples. Magnetic capture particles are added to each well. The capture particles are conjugated to a biomolecule that includes an antigen recognized by the antibody being screened. The wells including the sample and capture particles are then incubated. During incubation, the antigen of the magnetic capture particles may bind to the antibody.

Next, a non-magnetic conjugate is added to the wells. The conjugate includes an antibody that binds to the antigen, or that binds to a second antigen of the biomolecule that includes the antigen. The wells including the sample with the capture particles and the conjugate are then incubated to bind the conjugate to the capture particles. The conjugate competes with any of the antibody from the sample for binding to the magnetic capture particles.

The magnetic capture particles are then removed from the wells with a magnet, and any conjugate signal in the wells is measured. A presence, lack of presence, or amount of the antibody in the sample is inferred based on a presence, lack of presence, or amount of the conjugate signal in the wells. Alternatively, the assay may be performed by measuring conjugate pulled out by the magnetic capture particles.

### Example 17: Visual Colorimetric Assay

An assay is performed to determine the presence or lack of presence of a biomolecule of interest a sample, using magnetic capture particles and a conjugate that includes non-magnetic colored detection particles (e.g., non-magnetic beads). In the assay, the sample is incubated with magnetic capture particles that include a molecule such as an antibody or antigen that binds to the biomolecule of interest in the sample.

The conjugate is then incubated with the sample, and the conjugate includes an antibody or other molecule that binds to the biomolecule of interest. Thus, if the magnetic capture particles bind to the biomolecule of interest, the colored conjugate is indirectly bound to the magnetic capture particles.

The magnetic capture particles are pulled out of the sample using a magnet, and a color change is indicative of the sample comprising the biomolecule of interest. For example, when the beads of the conjugate are red, and a green color (e.g. in the form of green non-magnetic beads) is added to the sample prior to removal of the capture particles by the magnet, then the sample may turn green indicative of the presence of the biomolecule in the sample when the capture particles are removed by the magnet (along with the red-colored conjugate bound to the magnetic capture particles), or the sample may stay brown indicative of the absence of the biomolecule in the sample when the capture particles are removed by the magnet (because the red-colored conjugate is not bound to the magnetic capture particles when the sample does not include the biomolecule of interest).

Such an assay may be used in point-of-care screening or detection. Such a detection format may be used in assays such as those in Examples 15 and 16.

### Example 18: Reformatting an Old Assay

An assay is developed using a first sample type (e.g., blood samples) to measure a biomolecule of interest. The sample does not generally work with a second sample type (e.g., saliva samples), perhaps because the second sample type is too viscous or includes factors that interfere with the assay. A sample of the second sample type is contacted with capture particles to capture the biomolecule of interest. The biomolecules are then eluted from the capture particles into a buffer compatible with the assay developed for the first sample type. The sample may be cleaned using cleaning beads, or subject to any other treatment prior to eluting the biomolecules from the capture particles, which may remove any interfering factors. After the elution, the biomolecule of interest is assayed using the assay developed for the first sample type, even though the biomolecule came from the second sample type. Thus, methods and protocols described herein may be used for unexpected benefits such as refurbishing existing assays to work with new sample types.

### Example 19: Capture Moiety Without Inclusion of Capture Beads

A different approach may be used, relative to some examples above that may include capture moiety coated magnetic beads in a collection tube or capture moiety coated magnetic beads in a screw cap release agent. Instead, a biotinylated capture moiety may be combined with a collected sample in a collection tube, or may be already be present in the collection tube during sample collection. Then, when the sample arrives a lab, the lab adds streptavidin coated magnetic beads in biotin-binding molar excess over total moles of biotinylated capture moieties to rapidly bind total biotin-capture moieties and [biotin-capture moieties]-[biomarker] complex. The streptavidin beads can subsequently be isolated from the sample, and the sample matrix removed or aspirated from the beads, via centrifugation, filtration, or magnetic separation, and the captured biomarkers can subsequently be directly detected on the magnetic beads with or without washing the beads prior to conjugate detection, or after the biomarkers have been cleaved or eluted from the beads by any detection method of choice.

A reason for including capture moieties without capture beads in this example may be that the biomarker capture beads may be expensive or include pre-coating of the magnetic beads with capture moiety which may add time, cost, or complexity. Thus, as a different approach the capture moiety itself can be added to the sample to avoid these issues.

The approaches in this example may improve biomarker binding kinetics and biomarker capture efficiency, including reduced time, by the capture moiety as the method may include a homogeneous reaction in the absence of any beads. For example, the use of beads may be considered a heterogeneous reaction, and the size of beads may result in steric hindrance and NSB issues making biomarker capture challenging or less effective.

The capture moiety can already be present in the capture tube as a liquid reagent, or as a solid reagent such as spray dried, lyophilized, or pellet (e.g. liposphere), prior to the sample, or filtered sample, being added or collected into the capture tube, or the capture moiety can be added to the collection tube as a liquid reagent or solid reagent after sample collection, or sample collection and filtration, into the collection tube such as a reagent stored in a screw cap with a screw cap release mechanism whereby the reagent (liquid or dry) can be added and mixed with the sample after the screw is tightened and the barrier is broken allowing sample and reagent to mix together. The liquid or solid reagent can also be added to the sample, or filtered sample, after the sample is collected in the collection tube whereby the reagent is stored in a separate tube, vial, bottle, ampule, or vessel, and by opening or breaking this storage vessel it allows the reagent within it to be added, poured, dumped, dropped, spilled, or mixed into the sample. An example is drop by drop from a dropper bottle, or poured as a liquid from a storage bottle after unscrewing or removing the cap so the reagent storage bottle can be emptied or poured into the sample, a breakable ampule or twist off tab where the ampule can be squeezed to force the reagent out and empty it into the sample. As another option the reagent is dry such as a pellet and can be added or dropped into the sample where the pellet dissolves and releases the capture moiety into the sample, or the capture moiety is stored inside a dissolvable capsule, pellet, or pill as a liquid or solid reagent, whereby the dissolvable, or time-delayed dissolvable, capsule, pellet, or pill is added to the sample and when the capsule, pellet, or pill dissolves in the sample whereby the capture moiety is released and mixed into the sample for biomarker capture.

The capture moiety, or capture moiety-biomarkers complex, can subsequently be captured by an anti-capture moiety magnetic bead by the lab or prior to testing the sample. The anti-capture moiety magnetic beads such as streptavidin coated beads or anti-fluorescein antibody coated beads, can target a tag on the capture moiety such as biotin or fluorescein, whereby the tag (biotin or fluorescein) on the capture moiety enables rapid and efficient capture of total capture moiety or capture moiety-biomarker complex from the sample. This tag may be not limited to biotin or fluorescein, and can be or include a tag or fusion protein added to the capture moiety recombinantly such as a his-tag, 6His-tag, or maltose binding protein (MBP). As another option the capture moiety can be captured by a binding partner immobilized or coated on the magnetic capture beads such as an anti-capture moiety antibody. For example, if the capture antibody is an animal derived antibody such as mouse, rabbit, goat, sheep, cow, horse, lama, alpaca, camel, or pig, the magnetic beads can be coated with an anti-animal antibody such as anti-mouse, rabbit, goat, sheep, cow, horse, lama, alpaca, camel, or pig antibody. If the capture moiety is an aptamer or molecular imprinted polymer (MIP), or tagged, conjugated, or labelled with an oligonucleotide, peptide, polymer, or other tag, it can be capture by magnetic beads coated with anti-aptamer, anti-MIP, anti-oligonucleotide (complementary sequence), anti-peptide, or anti-polymer, or anti-other binding partner coated magnetic beads.

After the anti-capture moiety magnetic beads are added to the sample, they may bind some, the majority of, or all of the capture moiety and capture moiety-biomarker complex. The anti-capture moiety magnetic capture beads can then be captured, isolated, or separated from the sample with a magnet, filtration, or centrifugation whereby the sample matrix can be easily removed or aspirated from the capture beads, or the magnetic beads can be removed from the sample matrix with a magnetic, for subsequent biomarker detection on the magnetic beads, or magnetic bead washing followed by biomarker detection on the magnetic beads, or magnetic bead washing and biomarker Elution, Elution and neutralization, cleaving, or cleaving and quenching for subsequent biomarker detection in the absence of the magnetic beads. The biomarker can also be concentrated, enriched, or purified by the washing and eluting process, or washing and cleaving process, of the magnetic beads.

The liquid reagent comprising the capture moiety can be a sample preservative reagent or stabilization agent. It can be a sample conditioning reagent or agent. It can be a sample preservative reagent or stabilization agent also comprising a sample conditioning reagent or agent. The same is true for the lyophilized, spray dried, or pelleted capture moiety whereby the capture moiety is in a liquid reagent as a mixture with a sample preservative or stabilization agent, a sample conditioning reagent or agent, or a sample preservative or stabilization agent and a sample conditioning reagent or agent, prior to lyophilization, spray drying, or pelleting the capture moiety. The same is true where the capture moiety is inside a pellet, pill, or capsule that can be added to a sample to dissolve, or for a time-delayed release, of the capture moiety into the sample whereby the capture moiety and sample preservative or stabilization agent, the capture moiety and sample conditioning agent, or a mixture of the capture moiety, sample preservative or stabilization agent and sample conditioning agent are added to the sample after the pellet, pill, or capsule dissolves in the sample.

The sample conditioning reagent or agent can also comprise a lysis agent, cell lysis agent, displacer agent, or binding partner displacement or dissociation agent whereby the biomarker can be liberated or released in the presence of the capture moiety for maximum biomarker capture and recovery by the capture moiety such as when the biomarker is inside a cell (cell, extracellular particle, exosome, neuro exosome, virion, bacterium) or bound to a binding partner (vitamin D binding protein, sex hormone binding globulin, autoantibody, immune complex) prior to lysis, displacement, dissociation, or liberation of the biomarker from within (inside) a cell or from a binding partner or binding complex.

### Example 20: Biomarker Isolation and Enrichment for Detection

Assay accuracy can be affected by interferences or the matrix. The example demonstrates the efficacy of an assay method disclosed in this disclosure to improve the assay accuracy and sensitivity, by at least 6 times or greater.

The assay method comprises (i) pre-analytically treating, conditioning, or preparing a patient sample with clean beads (e.g., particles for interference removal) to selectively bind or capture one or more different interferences thereby decreasing the concentrations of the interference(s), eliminating the interference(s), or mitigating the interference mechanism; (ii) capturing the biomarker(s) with Capture Beads (e.g., particles for capturing one or more biomarkers); and (iii) detecting the biomarker(s) in the cleaned sample.

After the clean beads capture any interferences, the clean beads can be isolated or removed from the patient sample, e.g., via centrifugation, filtration, or magnetic separation, thereby generating a cleaned sample. In the biomarker capture step (i.e., step (ii)), antigen, antibody, or antigen and antibody coated Capture Beads, or a plurality or a pool of 2 or more different antigen, antibody, or antigen and antibody coated Capture Beads are added to the cleaned sample wherein the interferences are removed, for the accurate binding and high recovery of the biomarker or biomarkers from the cleaned sample. In some embodiments, the method can achieve more than about 70% recovery, more than about 80% recovery, more than about 90% recovery, more than about 95% recovery, or more, of the total biomarker(s) from the cleaned sample (e.g., blood, serum, plasma, saliva, saline oral rinse, urine, cerebral spinal fluid (CSF), etc.).

After the biomarker(s) have been captured by the Capture Beads, the Capture Beads can be washed with a wash buffer comprising surfactant(s) (e.g., 0.05%-0.10% Tween-20) or detergent(s) (e.g., 0.03%-0.5% Pluronic F108 tri-block copolymer) in TBS (10-20 mM TRIS, 150 mM NaCl) having a pH 7.2-8.0, to strip, remove, or wash away the sample matrix and any sample matrix constituents that have passively or non-specifically bound or absorbed to the Capture Beads surface. In some embodiments, the washing can be performed once, twice, trice, four times, or more.

In some embodiments, after the washing, the biomarkers captured by the Capture Beads can be directly detected on the bead surface using an anti-antigen or antibody conjugate or detection reagent, or via a competition or inhibition assay, or a binding interaction to the captured and purified biomarker on the bead surface.

In some embodiments, after the washing, the biomarkers captured by the Capture Beads can be eluted from the beads using an acidic elution buffer (e.g., 220 μL 100 mM Glycine pH 2.5, 0.05% Tween-20, or 180 μL 40 mM Acetic Acid, 0.05% Tween-20, pH 3.05), and neutralized with a neutralization buffer (e.g., 35 μL 300 mM TRIS pH 10.0, or 26 μL 300 mM TRIS pH 10.5, 0.05% Tween-20) such that the biomarkers are purified into a final matrix-free buffer with a pH from about 7.0 to about 8.0. The purified, or purified and enriched/concentrated, biomarker(s) are now ready for highly accurate, precise, reproducible, sensitive, and specific detection such as by ELISA (HRP or ALP), chemiluminescence (luminol, isoluminol, acridinium ester, ABEI, etc.), electrochemiluminescence (ruthenium), fluorescence, mass spec (LCMS, LC-LCMS, MALDI-TOF, etc.), molecular diagnostics (RT-PCR, LAMP, etc.), turbidimetric, bead-based agglutination, UV-Vis, biosensor, etc.

The use of a plurality of different clean beads can maximize cleaning efficiency and efficacy and increase subsequent test specificity and sensitivity. The use of a plurality of different antigen, antibody, or antigen and antibody coated Capture Beads can maximize biomarker capture efficiency, recovery, and subsequent test sensitivity.

In some embodiments, by selectively targeting, capturing, and removing or decreasing an interference mechanism(s) or immunoglobulin interference (e.g., heterophilic, autoantibody), serology or autoantibody test accuracy, sensitivity, and specificity can be improved. In some embodiments, a test accuracy, sensitivity, or specificity can be at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%. In some embodiments, false negatives can be reduced. In some embodiments, false positives can be reduced. In some embodiments, signal to noise ratio can be increased.

In some embodiments, the clean beads can comprise polyhistidine or polyhis (e.g., 8-histidine peptide or 6-histidine peptide) coated magnetic beads for the removal of human anti-histidine interference. In some cases, recombinantly produced antigens and/or peptides may comprise 6-histidine, 8-histidine, or polyhistidine recombinant tags used to aid in the purification from cell culture supernatant or cell culture medium. When the polyhistidine tagged recombinant antigens or peptides are coated onto the surface of an assay or test solid phase (e.g., ELISA plate wells, biosensor surface, non-magnetic or latex beads, magnetic beads, colloidal gold, etc.) passively, hydrophobically, ionically, via affinity or antibody capture, covalently, or via streptavidin/avidin/neutravidin if the antigens or peptides are biotinylated, the polyhistidine may subsequently be detected by sample-specific anti-polyhis or anti-histidine interference, immunoglobulins, or autoantibodies, causing false positive results if autoantibodies or immunoglobulins (IgG, IgM, and/or IgA) bind to the solid phase, or false negative results if anti-histidine interference binds to the solid phase and sterically blocks the antigen itself from being recognized by anti-antigen human immunoglobulins if present.

In some embodiments, the clean beads can comprise polyethylene oxide or PEO coated magnetic beads for the removal of human anti-PEO interference and/or human anti-PEG interference. In some embodiments, the clean beads can comprise PEO4 linkers for the binding of TFP-PEO4-biotin or NHS-PEO4-biotin labeled capture moieties to streptavidin coated magnetic beads. In some embodiments, the coated beads can be blocked with Pluronic F108 (a triblock copolymer). In some cases, therapeutics, drugs, disease modifying therapeutics (DMT), implantable medical devices (e.g., catheters, pacemakers, stents), or artificial organs may be pegylated or coated or modified with PEO or PEG to make them inert, avoid or mitigate clotting or adherence of blood cells or proteins, or avoid or mitigate immune rejection. In some cases, patients may develop autoantibodies against the PEO or PEG and may have these interfering antibodies present or in circulation (e.g., in blood, serum, plasma, saliva, oral rinse, etc.), causing false positive results if anti-PEO/PEG autoantibodies or immunoglobulins (IgG, IgM, and/or IgA) bind to the solid phase, or false negative results if anti-PEO/PEG interference binds to the solid phase and sterically blocks the antigen itself from being recognized by anti-antigen human immunoglobulins if present.

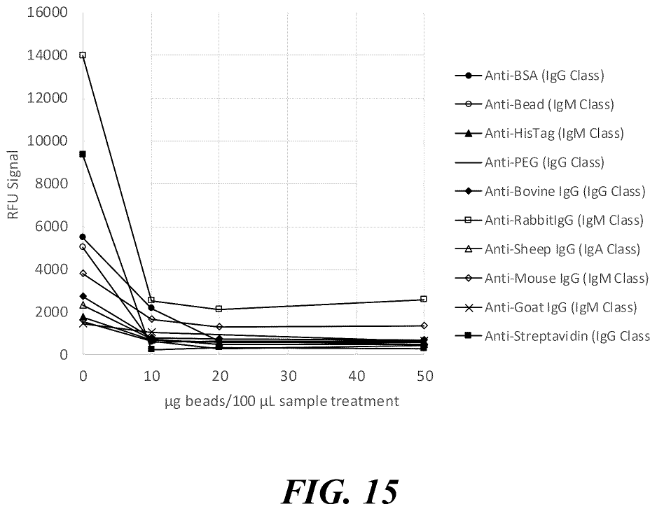

In some embodiments, multiple interferences, e.g., multiple immunoglobulin interferences, can be removed from the sample in a single reaction or cleaning process. In some embodiments, the clean beads can comprise a plurality or a pool of clean beads wherein the plurality of clean beads can comprise different interference capture moieties. FIG. 15 shows an antibody testing with sample cleaned by clean beads had reduced sample interference signal of 10 different immunoglobulin interferences by an average of about 73%.

Table 51 demonstrates using clean beads pre-analytically to clean a serum sample of heterophilic or autoantibody IgM immunoglobulin interference improves the specificity, sensitivity, and signal-to-noise (S/N) of antibody assays by removing immunoglobulin interferences that can contribute to high assay background (false positive signal due to NSB, heterophilic binding, or autoantibody binding of IgM immunoglobulin) or decreased analyte signal (false negative signal due to steric hindrance or blocking the antigen by the IgM on the bead surface. A problematic negative sample read 4,932 RFUs without cleaning (“Without”) and 2,923 RFUs after cleaning with the clean beads (“With”). A positive sample read 7,730 RFU without cleaning (“Without”) and 11,506 RFUs after cleaning with the clean beads (“With”). The S/N was increased from 1.6 (without cleaning) to 3.9 (after cleaning with the clean beads).

TABLE 51
IgM RFU signal with and without cleaning

| Clean beads | Problematic negative | Positive sample | S/N |
|---|---|---|---|
| With | 2923 | 11506 | 3.9 |
| Without | 4932 | 7730 | 1.6 |

In some embodiments, the reaction or the cleaning process can be fully automated, e.g., by a liquid handler or integrated into the primary sample collection tube for “time-of-collection” sample cleaning.

In some embodiments, the cleaning process can be performed for antibody detection and/or purification. In some embodiments, the cleaning process can be performed for antigen detection and/or purification.

Table 52 shows another example that the cleaning process (e.g., using the clean beads) can increase the signal to noise ratio of the assay. For Celiac biomarkers assay with BioRad serum as controls for IgA and IgG against human IgG, the BioRad controls were cleaned with the clean beads prior to testing the BioRad controls in the assay. Both IgG and IgA anti-hTtG detection had lowered background and improved signal-to-noise ratio (S/N) in comparison to the testing without cleaning the controls with clean beads.

TABLE 52
RFU signals and S/N for BioRad controls with
and without cleaning with clean beads

| | RFU signal | | RFU signal Without | |
|---|---|---|---|---|
| | With clean | | cleaning the controls | |
| Controls | beads | S/N | with clean beads | S/N |
| IgG++ | 1156 | 1.20 | 1587 | 0.99 |
| IgG+ | 1633 | 1.70 | 2024 | 1.26 |
| IgG− | 960 | 1.00 | 1602 | 1.00 |
| IgA++ | 1308 | 4.72 | 1195 | 3.16 |
| IgA+ | 834 | 3.01 | 878 | 2.32 |
| IgA− | 277 | 1.00 | 378 | 1.00 |

### Example 21: Antibody Detection for COVID-19

The serology antibody test of detecting anti-SARS-CoV-2 RBD IgG was performed and compared with comparators 1-4.

Table 53 shows the sensitivity compared to comparators 1-4. The data show that the method disclosed in this application has higher sensitivity of detecting anti-SARS-CoV-2 RBD IgG than the comparators 1-4.

TABLE 53
Sensitivity comparison

| SARS-CoV-2 | Days After | | | | | |
|---|---|---|---|---|---|---|
| Anti-RBD | Positive RT- | | | | | |
| Antibody Test | PCR Result | n | Positive | Negative | PPA | 95% CI |
| Comparator 1 | 0-7 Days | 75 | 37 | 38 | 49.3% | 38.3%-60.4% |
| Comparator 2 | | 33 | 25 | 8 | 75.8% | 59.0%-87.2% |
| Comparator 3 | | 32 | 29 | 3 | 90.6% | 47.9%-98.0% |
| Comparator 4 | | 324 | 165 | 159 | 50.9% | 45.6%-56.0% |
| This | | 15 | 14 | 1 | 93.3% | 66.0%-99.7% |
| application | | | | | | |
| Comparator 1 | 8-14 Days | 92 | 74 | 18 | 80.4% | 71.2%-87.3% |
| Comparator 2 | | 64 | 61 | 3 | 95.3% | 87.1%-98.4% |
| Comparator 3 | | 77 | 67 | 10 | 87.0% | 77.4%-93.6% |
| Comparator 4 | | 138 | 108 | 30 | 78.3% | 76.4%-87.6% |
| This | | 14 | 14 | 0 | 100% | 73.2%-100% |
| application | | | | | | |
| Comparator 1 | ≥15 Days | 52 | 51 | 1 | 98.1% | 89.9%-99.9% |
| Comparator 2 | | 95 | 92 | 1 | 96.8% | 91.1%-98.9% |
| Comparator 3 | | 233 | 225 | 8 | 96.6% | 93.4%-98.5% |
| Comparator 4 | | 249 | 238 | 11 | 95.6% | 92.2%-97.8% |
| This | | 20 | 20 | 0 | 100% | 80.%-100% |
| application | | | | | | |

### Example 22: Purification of COVID-19 Neutralizing Antibodies in Saline Oral Rinse

The Saline Oral Rinse (SOR) purification protocol was performed as follows. The protocol can be fully automated.

Clean Step: 100 μL clean beads (1.6 μm, Low NSB, superparamagnetic) were added to 1,000 μL or 2,000 μL SOR and incubated for 30 min to remove sample interference(s), thereby generating a cleaned SOR sample.

Capture Step: 100 μL Capture Beads (166 μg SARS-CoV-2 spike protein coated Capture Beads comprising 100 μg RBD (wild type) beads, 33 μg RBD Delta beads, and 33 μg NTD beads) were added to 1,000 μL or 2,000 μL cleaned SOR sample and incubated for 30 min to capture total anti-COVID-19 neutralizing immunoglobulins.

Wash Step: the Capture Beads were washed 3 times with Wash Buffer to remove SOR sample matrix.

Purification Step: the captured total anti-COVID-19 neutralizing immunoglobulins were eluted with an elution buffer (e.g., 220 μL of 100 mM glycine pH 2.5, 0.05% Tween-20), and neutralized with neutralization buffer (e.g., 35 μL of 300 mM TRIS pH 10.0, 0.05% Tween-20).

Enrichment Step: the purified SOR sample was enriched/concentrated, e.g., in 235 μL buffer.

The enriched SOR sample was tested by the GenScript® cPass™ SARS-CoV-2 Neutralization Antibody Detection Kit (EUA201427).

The anti-RBD immunoglobulins as summarized in Table 54. Inhibition of signal implies the presence of SARS-COV2 antibodies. Neg=negative. Pos=positive.

Neg SOR Pool: SOR collected from healthy adults who have never been infected by COVID-19 nor vaccinated against COVID-19. They presumably don't have any neutralizing antibodies against COVID-19.

Mid SOR Pool: SOR collected from adults who have been infected by COVID-19 and/or vaccinated against COVID-19 with levels of anti-RBD IgA, IgG, and/or IgM from 100 to 250 ng/mL by the COVID-19 Antibody Test disclosed in this application. They have neutralizing antibodies against COVID-19.

High SOR Pool: SOR collected from adults who have been infected by COVID-19 and/or vaccinated against COVID-19 with levels of anti-RBD IgA, IgG, and/or IgM from >500 ng/mL by the COVID-19 Antibody Test disclosed in this application. They have neutralizing antibodies against COVID-19.

Patient 1: A non-pooled SOR sample collected from a patient currently positive for COVID-19 by RT-PCR more than 5 days after symptoms onset. This patient may have neutralizing antibodies against COVID-19.

TABLE 54
SARS-COV2 antibodies testing results

| | Volume | | | Patient Call |
|---|---|---|---|---|
| | Sample | | % | (cutoff >30% |
| Sample | Captured | OD450 | Inhibition | inhibition) |
| cPass ™ Neg Ctrl | N/A Kit | 1.821 | 0% | Neg |
| | Controls | | | |
| cPass ™ Pos Ctrl | N/A Kit | 0.846 | 54% | Pos |
| | Controls | | | |
| Neg SOR Pool | 1 mL | 2.061 | −13% | Neg |
| Mid SOR Pool | 1 mL | 2.078 | −14% | Neg |
| Mid SOR Pool 2x | 2 mL | 1.581 | 13% | Neg |
| Vol | | | | |
| High SOR Pool | 1 mL | 1.634 | 10% | Neg |
| High SOR Pool 2x | 2 mL | 1.086 | 40% | Pos |
| Volume | | | | |
| Patient 1 | 1 mL | 1.452 | 20% | Neg |

The Biomarker purification protocol increased the sensitivity of an existing GenScript® cPass™ assay.

### Example 23: Lyme Disease (Borrelia burgdorferi) Antibody Test

Lyme disease antibody test was performed with the process disclosed herein in serum samples. 2 weeks prior to collecting and testing Patient 1's serum, Patient A self-reported a tick bite and was diagnosed by clinical symptomology and treated with antibiotics. However, Patient 1 was false negative by the FDA approved ELISA and Western Blot 2-tier algorithm when tested.

The 60 μL serum sample was cleaned with 100 μL clean beads to remove sample interferences and improve test specificity. A pool of 3 different Borrelia burgdorferi antigen (OspC, DbpA, and VlsE) coated capture beads to increase the likelihood and sensitivity of detecting Lyme disease antibodies.

If 2 or 3 out of 3 of IgG, IgA, and/or IgM are positive the result is positive. This is to eliminate false positive that a patient may have cross-reactive IgA, IgG, or IgM antibodies against a different bacteria or pathogen but may be detected in Lyme disease serology tests as false positives if only one out of 3 of IgG, IgA, and/or IgM is positive.

The testing results are shown in Table 55. Patient 1's serum was positive since 2 out of 3 antibodies were positive, or IgG and IgM were both positive for Borrelia burgdorferi antibodies. This serum testing example demonstrates the sensitivity and ability of the process disclosed herein to detect low abundance biomarkers in serum samples.

TABLE 55
Lyme disease antibody testing result

| | | | | Pooled | Cutoff = 1.0 |
|---|---|---|---|---|---|
| Patient ID | OspC | DbpA | VlsE | Antigens | Patient Call |
| IgG Detection in Serum (550/580 em/ex) | | | | | |
| BioRad Neg Ctrl | 0.71 | 0.50 | 0.50 | 0.62 | Neg |
| BioRad Pos Ctrl | 1.57 | 3.22 | 2.80 | 5.12 | Pos |
| Negative Sera | 0.89 | 0.66 | 0.84 | 0.86 | Neg |
| Patient 1 | 0.94 | 1.03 | 1.14 | 1.33 | Pos |
| Positive Sera | 6.43 | 3.74 | 3.79 | 11.7 | Pos |
| Negative Sera | 0.85 | 0.09 | 0.60 | 0.79 | Neg |
| IgA Detection in Serum (588/621 em/ex) | | | | | |
| BioRad Neg Ctrl | N/A | N/A | N/A | N/A | N/A |
| BioRad Pos Ctrl | N/A | N/A | N/A | N/A | N/A |
| Negative Sera | 0.96 | 0.68 | 0.83 | 0.88 | Neg |
| Patient 1 | 1.12 | 0.76 | 0.74 | 0.94 | Neg |
| Positive Sera | 1.44 | 0.37 | 0.63 | 1.28 | Pos |
| Negative Sera | 0.84 | 0.17 | 0.46 | 0.77 | Neg |
| IgM Detection in Serum (495/530 em/ex) | | | | | |
| BioRad Neg Ctrl | 0.79 | 0.67 | 0.78 | 0.53 | Neg |
| BioRad Pos Ctrl | 7.74 | 0.81 | 1.40 | 4.93 | Pos |
| Negative Sera | 0.90 | 0.79 | 0.91 | 0.69 | Neg |
| Patient 1 | 1.28 | 1.12 | 1.28 | 1.15 | Pos |
| Positive Sera | 2.31 | 0.60 | 2.80 | 2.8 | Pos |
| Negative Sera | 0.91 | 0.43 | 0.89 | 0.84 | Neg |

N/A = Commercial Control not available.

Lyme disease antibody test was performed with the process disclosed herein in SOR samples. Patient 1 was diagnosed by clinical symptomology and tested positive by the Lyme Disease Antibody Test for Serum as shown above. Patient 2 did not know they had Lyme disease prior to the SOR testing but was subsequently confirmed positive by a commercially available FDA approved serum ELISA and WB 2-Tier Algorithm test. Patients 3-6 did not have any tick bites.

The SOR protocol was as follows.

Clean Step: 100 μL clean beads (1.6 μm, Low NSB, superparamagnetic) were added to 1,300 μL SOR and incubated for 30 min to remove sample interferences.

Capture Step: 100 μL Capture Beads (150 μg antigen coated Capture Beads comprising 50 μg DbpA beads, 50 μg OspC beads, and 50 μg VisE beads) were added to the 1,300 μL cleaned SOR and incubated for 30 min to capture total anti-Borrelia immunoglobulins.

Wash Step: the Capture Beads were washed 3 times with Wash Buffer to remove SOR sample matrix.

Conjugate Step: triplex fluorescent conjugate against hIgA, hIgG, and hIgM were added to the capture beads and incubated for 30 min.

Wash Step: the Capture Beads were washed 3 times with Wash Buffer to remove unbound or excess conjugate.

Purification/enrichment Step: the captured biomarkers were eluted, neutralized, and concentrated.

The testing results are shown in Table 56. Patient 1's SOR was positive since 2 out of 3 antibodies were positive, or IgA and IgM were both positive for Borrelia burgdorferi antibodies. Patient 2's SOR was positive since all 3 antibodies were positive, or IgA, IgG and IgM were all positive for Borrelia burgdorferi antibodies.

TABLE 56
Lyme disease antibody testing result

| Lyme Disease (Borrelia) Antibody Test for SOR | | | | Cutoff = 1.0 |
|---|---|---|---|---|
| Patient ID | IgG | IgA | IgM | Patient Call |
| BioRad Neg Ctrl | 0.78 | N/A | 0.88 | Neg |
| BioRad Pos Ctrl | 1.43 | N/A | 4.34 | Pos |
| Patient 1 | 0.61 | 1.92 | 1.34 | Pos |
| Patient 2 | 1.76 | 2.44 | 2.25 | Pos |
| Patient 3 | 0.79 | 0.67 | 0.54 | Neg |
| Patient 4 | 0.52 | 0.47 | 0.52 | Neg |
| Patient 5 | 0.75 | 0.76 | 0.75 | Neg |
| Patient 6 | 0.41 | 0.23 | 0.4 | Neg |

N/A = Commercial Control not available.

Patient 1 was tested positive for IgA and IgM in SOR while positive for IgG and IgM in serum. IgA is the primary immunoglobulin expressed in saliva and is the first line of defense against a respiratory pathogen or pathogen in the oral cavity.

Patient 2 was tested positive for all 3 immunoglobulin classes (hIgA, hIgG, and hIgM) with IgA being their strongest response detected. Patient did not know they had Lyme disease but was complaining of Lyme disease like symptoms for the past 6 months but thought it was related to a different health issue and therapeutic treatment. Patient 2's serum was subsequently tested by an FDA approved ELISA and Western Blot 2-tier algorithm and tested positive for Lyme disease. All other patients tested strong negative, as well as the negative control.

This SOR testing example demonstrates the sensitivity and ability of the process disclosed herein to detect immunoglobulins against Borrelia burgdorferi in SOR samples.

### Example 24: Celiac Disease Biomarker Test

100 μL clean beads designed to selectively target, bind, and remove endogenous interfering substances such as HAMA, RF, human anti-animal antibody (e.g., goat, rabbit and bovine IgG), free biotin, human anti-streptavidin, anti-HIS-Tag, and/or non-specific binding interferences were added to 60 μL serum sample and incubated for 30 min. After the removal of the clean beads, Capture Beads coated with biotinylated antigens were added to the cleaned and conditioned sample and incubated for 30 min. Capture beads were a 60 μg pool of 2 different tTG antigens (T051 open confirmation antigen and T067 closed confirmation antigen) sourced from Zedira in Germany that have been shown to significantly improve test sensitivity as compared to purified or recombinant closed confirmation htTG. T067 is human tissue transglutaminase, endotoxin free, recombinant produced in insect cells, and is in the closed confirmation and requires the addition of 10 mM Ca²⁺ to activate His6-rhTG2 to the open confirmation. T051 is open tTG, inhibited human tissue transglutaminase, stabilized in its open conformation, recombinantly produced in insect cells. Human tissue transglutaminase recombinantly produced in insect cells, conjugated with a specific inhibitor in order to stabilize the open conformation.

The Capture Beads were subsequently removed from the sample and washed. A conjugate specific to human IgG and IgA was added to the Capture Beads. The Capture Beads were washed to remove excess conjugate or any non-specific bound substances. After the washing, the conjugate (with the captured biomarker) was eluted and the eluate was transferred to a reading plate, neutralized, and read fluorometrically. The assay signals were directly proportional to the tTG-IgA and tTG-IgG autoantibody concentrations. IgA results are summarized in Table 57 and a comparison with the ELISA Comparator test is summarized in Table 58.

TABLE 57
IgA testing results for Celiac disease

| | | This disclosure | | | ELISA Comparator | |
|---|---|---|---|---|---|---|
| | | Criteria | | | | |
| | | CutOff > 1.0 | CutOff > 1.0 | CutOff > 1.0 | Pos ≥ 4 | Pos ≥ 4 |
| | | ID | | | | |
| | | T051 IgA | T067 IgA | 51 + 67 | | Enriched |
| | | RFU | RFU | RFU | IgA | IgA |
| Presumed | 40339-04212 | 0.9 | 0.8 | 0.9 | 0.6 | −0.1 |
| negative | 40339-04044 | 0.8 | 0.8 | 0.8 | 0.5 | 0.0 |
| | 404339-04916 | 0.9 | 0.8 | 0.9 | 1.7 | 0.0 |
| | 404339-04760 | 0.8 | 0.8 | 0.8 | 0.0 | −0.1 |
| | 40339-04442 | 0.8 | 0.8 | 0.8 | 0.5 | −0.1 |
| | 40227-11448 | 0.7 | 0.9 | 0.8 | 1.8 | 0.0 |
| Presumed | Celiac Pos - | 1.6 | 1.4 | 1.5 | 195.5 | 72.8 |
| positive | 209020 | | | | | |
| | Celiac Pos - | 1.0 | 1.1 | 1.1 | 2.7 | 7.4 |
| | 194724 | | | | | |
| | Celiac Pos - | 1.1 | 1.1 | 1.2 | 5.2 | 4.7 |
| | 218654 | | | | | |
| | Celiac Pos - | 1.1 | 1.0 | 1.1 | 6.2 | 10.5 |
| | 213528 | | | | | |
| | BioRad IgA++ | 1.0 | 1.3 | 1.2 | | |
| | BioRad IgA+ | 1.4 | 1.1 | 1.3 | | |
| | BioRad IgA− | 0.8 | 1.0 | 0.9 | | |

TABLE 58
IgA testing results without and with
cleaning/purification/enrichment
Not Cleaned, Purified and Enriched

| tTG-IgA | ELISA Comparator | ELISA Comparator |
|---|---|---|
| Celiac Test | Test Positive | Test Negative |
| Test Positive (this | 3 | 1 |
| disclosure) | | |
| Test Negative (this | 0 | 27 |
| disclosure) | | |

| Agreement | | 95% CI |
|---|---|---|
| Sensitivity | 100% | 31%-100% |
| Specificity | 96.4% | 80%-100% |

Cleaned, Purified and Enriched

| tTG-IgA | ELISA | ELISA |
|---|---|---|
| Celiac Test | Test Positive | Test Negative |
| Test Positive (this | 4 | 0 |
| disclosure) | | |
| Test Negative (this | 0 | 27 |
| disclosure) | | |

| Agreement | | 95% CI |
|---|---|---|
| Sensitivity | 100% | 40%-100% |
| Specificity | 100% | 84%-100% |

The method provided in the present disclosure has good agreement with the IgA ELISA Comparator test. Sample ID Celiac Pos—194724 was tested positive with the method provided in the present disclosure, while it was tested negative (false negative) with the IgA ELISA Comparator test when no cleaning/purification/enrichment was performed prior to the IgA ELI SA Comparator test. After cleaning/purification/enrichment was performed prior to the IgA ELISA Comparator test, the sample was tested positive. Cleaning/purification/enrichment of the test sample improved the test specificity to 10000.

IgG results are summarized in Table 59 and a comparison with the ELISA Comparator test is summarized in Table 60.

TABLE 59
IgG testing results for Celiac disease

| | | This disclosure | | | ELISA Comparator | |
|---|---|---|---|---|---|---|
| | | Criteria | | | | |
| | | CutOff > 1.0 | CutOff > 1.0 | CutOff > 1.0 | Pos ≥ 6 | Pos ≥ 6 |
| | | ID | | | | |
| | | T051 IgG | T067 | 51 + 67 | | Enriched |
| | | RFU | IgG RFU | RFU | IgG | IgG |
| Presumed | 40339-04212 | 0.8 | 0.7 | 0.8 | −1.7 | −2.2 |
| negative | 40339-04044 | 0.9 | 0.7 | 0.9 | −0.8 | −2.2 |
| | 404339-04916 | 0.8 | 0.7 | 0.8 | 5.7 | −1.8 |
| | 404339-04760 | 0.7 | 0.7 | 0.7 | −0.4 | −2.1 |
| | 40339-04442 | 0.6 | 0.9 | 0.8 | 12.0 | −2.1 |
| | 40227-11448 | 0.6 | 0.8 | 0.8 | −0.7 | −2.1 |
| | Celiac Pos - | 1.2 | 0.8 | 1.1 | 3.4 | −1.0 |
| | 209020 | | | | | |
| Presumed | Celiac Pos - | 0.9 | 1.1 | 1.0 | 8.8 | 26.2 |
| positive | 194724 | | | | | |
| | Celiac Pos - | 1.0 | 1.1 | 1.1 | 2.6 | −1.7 |
| | 218654 | | | | | |
| | Celiac Pos - | 0.8 | 0.8 | 0.9 | 4.6 | −1.7 |
| | 213528 | | | | | |
| | BioRad IgG++ | 2.0 | 2.0 | 2.1 | | |
| | BioRad IgG+ | 1.5 | 1.1 | 1.4 | | |
| | BioRad IgG− | 0.7 | 0.8 | 0.8 | | |

TABLE 60
IgG testing results without and with
cleaning/purification/enrichment
Not Cleaned, Purified and Enriched

| tTG-IgG | ELISA Comparator | ELISA Comparator |
|---|---|---|
| Celiac Test | Test Positive | Test Negative |
| Test Positive (this | 1 | 3 |
| disclosure) | | |
| Test Negative (this | 1 | 6 |
| disclosure) | | |

| Agreement | | 95% CI |
|---|---|---|
| Sensitivity | 50% | 27%-97% |
| Specificity | 75.0% | 36%-96% |

Cleaned, Purified and Enriched

| tTG-IgG | ELISA Comparator | ELISA Comparator |
|---|---|---|
| Celiac Test | Test Positive | Test Negative |
| Test Positive (this | 1 | 2 |
| disclosure) | | |
| Test Negative (this | 0 | 7 |
| disclosure) | | |

| Agreement | | 95% CI |
|---|---|---|
| Sensitivity | 100% | 55%-100% |
| Specificity | 77.8% | 40%-96% |

The method provided in the present disclosure has good agreement with the IgA ELISA Comparator test. Sample ID Celiac Pos—194724 was tested positive with the method provided in the present disclosure, while it was tested negative (false negative) with the IgA ELISA Comparator test when no cleaning/purification/enrichment was performed prior to the IgA ELISA test. After cleaning/purification/enrichment was performed prior to the IgA ELISA Comparator test, the sample was tested positive. Cleaning/purification/enrichment of the test sample improved the test specificity to 100%.

Negative sample ID 40339-04442 was tested positive (false positive) with the IgG ELISA test when no cleaning/purification/enrichment was performed prior to the IgG ELISA Comparator test. After cleaning/purification/enrichment was performed prior to the IgA ELISA Comparator test, the sample was tested negative. This demonstrates the clean beads and purification process (e.g., cleaning/purification/enrichment process) eliminated or mitigated an IgG immunoglobulin heterophilic or autoantibody interference mechanism in this sample causing a false positive IgG ELISA Comparator result.

The sensitivity of the IgG ELISA Comparator for sample ID Celiac Pos—194724 was greatly improved (increased from 8.8 to 26.2) after the cleaning/purification/enrichment process. Celiac positive commercially sourced serum sample (Sample ID Celiac Pos—213528) was tested negative for IgG by both the method provided in this disclosure and IgG ELISA Comparator test. Celiac positive commercially sourced serum samples (Sample ID Celiac Pos—209020 and 218654) were tested positive for IgG by the method provided in this disclosure but negative by the IgG ELISA Comparator test.

The capture beads were subsequently modified with 2 different open confirmation tTG antigens (antigens T051 and T246 sourced from Zedira in Germany) instead of the prior 2 antigens of an open and closed antigen (antigens T051 and T067 sourced from Zedira). The four Celiac positive commercially sourced serum samples were all tested positive by the method disclosed in this disclosure. This may be due to the closed confirmation tTG antigen (T067 from Zedira) may not capture autoantibodies in the samples directed against the open confirmation of tTG and not the closed confirmation.

31 serum samples (20 presumed negative and 11 presumed positive) were tested by the method provided in the present disclosure and the ELISA Comparator. The testing results are summarized in Table 61 and the agreement value is summarized in Table 62.

TABLE 61
IgA testing results for Celiac disease

| | | | | | ELISA |
|---|---|---|---|---|---|
| | | This disclosure | | | Comparator |
| | Criteria | CutOff > 1.0 | CutOff > 1.0 | CutOff > 1.0 | Pos ≥ 4 |
| | ID | T051 IgA | T067 IgA | T051 + T067 | IgA |
| Presumed | 40339-04226 | 0.85 | 0.73 | 0.83 | 0.5 |
| Negative | 40339-04212 | 0.84 | 0.73 | 0.83 | 0.6 |
| | 40339-04044 | 0.79 | 0.73 | 0.81 | 0.5 |
| | 40339-04195 | 0.82 | 0.66 | 0.78 | 0.3 |
| | 40339-04364 | 0.88 | 0.68 | 0.82 | 1.1 |
| | 40339-04848 | 0.77 | 0.74 | 0.80 | 0.5 |
| | 40227-11105 | 0.90 | 0.80 | 0.90 | 0.8 |
| | 40227-11243 | 0.81 | 0.87 | 0.89 | 1.2 |
| | 40227-11155 | 0.75 | 0.40 | 0.60 | 0.4 |
| | 404339-04916 | 0.89 | 0.78 | 0.88 | 1.7 |
| | 404339-04597 | 0.81 | 0.79 | 0.85 | 0.5 |
| | 404339-04671 | 0.93 | 0.76 | 0.89 | 1.4 |
| | 404339-04486 | 0.81 | 0.71 | 0.80 | 0.3 |
| | 404339-04760 | 0.74 | 0.74 | 0.79 | 0.0 |
| | 404339-04580 | 0.80 | 0.76 | 0.83 | 0.0 |
| | 40227-13164 | 0.79 | 0.84 | 0.86 | 0.2 |
| | 40339-04442 | 0.75 | 0.72 | 0.78 | 0.5 |
| | 40227-11228 | 0.87 | 0.89 | 0.94 | 0.1 |
| | 40227-11448 | 0.65 | 0.86 | 0.80 | 1.8 |
| | 40227-14645 | 0.77 | 0.85 | 0.86 | 0.4 |
| Presumed | Celiac Pos - | 0.43 | 0.85 | 0.69 | 2.6 |
| Positive | 215706 | | | | |
| | Celiac Pos - | 0.80 | 0.79 | 0.84 | 0.6 |
| | 209690 | | | | |
| | Celiac Pos - | 0.88 | 0.74 | 0.85 | 0.4 |
| | 200545 | | | | |
| | Celiac Pos - | 0.87 | 0.91 | 0.94 | 0.5 |
| | 220839 | | | | |
| | Celiac Pos - | 1.47 | 1.34 | 1.49 | 195.5 |
| | 209020 | | | | |
| | Celiac Pos - | 0.90 | 0.98 | 0.99 | 0.5 |
| | 185909 | | | | |
| | Celiac Pos - | 0.86 | 0.96 | 0.97 | 0.0 |
| | 199992 | | | | |
| | Celiac Pos - | 0.97 | 1.05 | 1.07 | 2.7 |
| | 194724 | | | | |
| | Celiac Pos - | 1.06 | 1.06 | 1.12 | 5.2 |
| | 218654 | | | | |
| | Celiac Pos - | 1.03 | 0.99 | 1.07 | 6.2 |
| | 213528 | | | | |
| | Celiac Pos - | 0.83 | 0.75 | 0.84 | 3.5 |
| | 205038 | | | | |
| | BioRad IgA++ | 0.90 | 1.21 | 1.13 | |
| | BioRad IgA+ | 1.35 | 1.06 | 1.27 | |
| | BioRad IgA− | 0.77 | 0.95 | 0.92 | |
| | BioRad IgG++ | 0.78 | 0.74 | 0.81 | |
| | BioRad IgG+ | 0.84 | 0.72 | 0.82 | |
| | BioRad IgG− | 0.73 | 0.68 | 0.75 | |

TABLE 62
IgA testing results without and with
cleaning/purification/enrichment

| | Not Cleaned, Purified and Enriched | |
|---|---|---|
| | ELISA Comparator | ELISA Comparator |
| IgA | Test Positive | Test Negative |
| Test Positive (this disclosure) | 3 | 1 |
| Test Negative (this disclosure) | 0 | 27 |

| | Agreement | 95% CI |
|---|---|---|
| Sensitivity | 100% | 31%-100% |
| Specificity | 96.4% | 80%-100% |

| | Cleaned, Purified and Enriched | |
|---|---|---|
| | ELISA Comparator | ELISA Comparator |
| IgA | Test Positive | Test Negative |
| Test Positive (this disclosure) | 4 | 0 |
| Test Negative (this disclosure) | 0 | 27 |

| | Agreement | 95% CI |
|---|---|---|
| Sensitivity | 100% | 40%-100% |
| Specificity | 100.0% | 84%-100% |

The method provided in the present disclosure has good agreement with the IgA ELISA Comparator test with a 100% sensitivity and 96.4% specificity when no cleaning/purification/enrichment was performed prior to the IgA ELISA test. Sample ID Celiac Pos—194724 was tested positive with the method provided in the present disclosure, while it was tested negative (false negative) with the IgA ELISA Comparator test when no cleaning/purification/enrichment was performed prior to the IgA ELISA test. After cleaning/purification/enrichment was performed prior to the IgA ELISA Comparator test, the sample was tested positive. Cleaning/purification/enrichment of the test sample improved the test specificity to 100%.

Celiac positive commercially sourced serum samples (Sample ID Celiac Pos—215706, 209690, 200545, 220839, 185909, 199992, and 205038) were tested negative for IgG by both the method provided in this disclosure and IgG ELISA Comparator test.

The capture beads were subsequently modified with 2 different open confirmation tTG antigens (antigens T051 and T246 sourced from Zedira in Germany) instead of the prior 2 antigens of an open and closed antigen (antigens T051 and T067 sourced from Zedira). The eleven Celiac positive commercially sourced serum samples were all tested positive by the method disclosed in this disclosure. This may be due to the closed confirmation tTG antigen (T067 from Zedira) may not capture autoantibodies in the samples directed against the open confirmation of tTG and not the closed confirmation.

This example demonstrates the accuracy, sensitivity, and specificity of Celiac Biomarkers Test provided in the present disclosure as compared against the FDA cleared tTG-IgA ELISA Comparator and tTG-IgG ELISA Comparator.

### Example 25: Alzheimer's Disease Biomarker Purification and Test

Saline oral rinse (SOR) samples were collected from healthy controls and moderate Alzheimer's patients using custom manufactured SOR collection kits from Sarstedt, Inc.

1,300 μL SOR samples were cleaned with 100 μL clean beads, followed by total Tau capture with 200 μg anti-total Tau (clone HT7) capture beads. After a 30 min incubation at 37° C. with mixing, the capture beads were washed 2 times with wash buffer.

Total Tau was eluted using 180 μL elution buffer, and 175 μL eluate was neutralized using 26 μL neutralization buffer to generate a 201 μL sample for testing. The SOR samples was transformed to matrix-free samples while purifying and enriching the β-AMYLOID (AB40 and AB42; Mab clone 3D6) and phosphorylated Tau (pTau181; Mab clone HT7).

The sample protein profiles were analyzed by SDS PAGE by running 25 μL sample through a 4-20% gradient Tris-Glycine gel. 100 μg of pTau181 was used as a positive control. The arrow indicates the pTau181 protein.

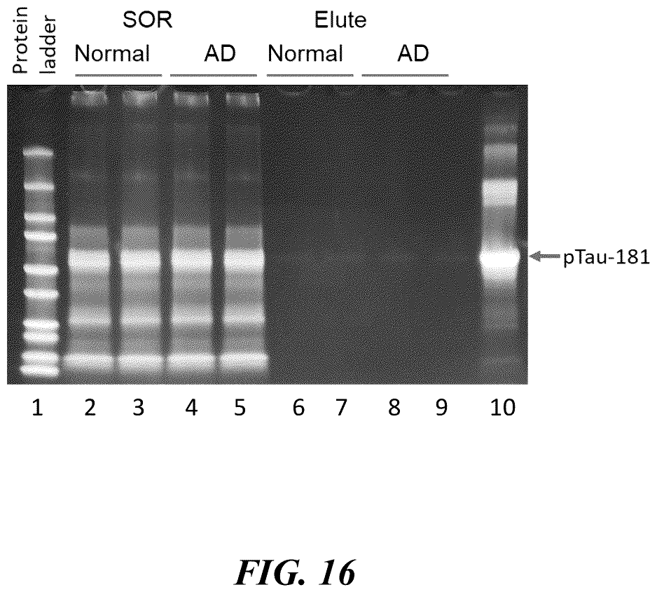

FIG. 16 shows the gel picture of SDS PAGE. There were 10 lanes (1-10) in the silver stained SDS Gel from left to right. The 1^{st} lane (lane 1) in the gel was the MW standards, where each band represents a different molecular weight from a large 250 KD protein (top of lane) to a small 10 KD protein (bottom of lane). Lane 10 was the SDS page profile of purified pTau181 lysate run as a sample. The molecular weight of pTau181 is approximately 50 KD.

Lanes 2 and 3 demonstrate the neat Normal (apparently healthy) SOR samples loaded on the Gel which were not processed by the Alzheimer's Biomarker Purification Kit. There were many bands in lanes 2 and 3; some of them were very dark and broad indicating a high protein concentration at those molecular weights. They represent different proteins and enzymes (e.g., amylase), mucin, secretory agglutinins, bacteria, and other constituents in the SOR matrix.

Lanes 4 and 5 demonstrate neat AD (Alzheimer's disease) SOR samples loaded on the Gel which were not processed by the Alzheimer's Biomarker Purification Kit. There were many bands in lanes 4 and 5; some of them were very dark and broad indicating a high protein concentration at those molecular weights. They represent different proteins and enzymes (e.g., amylase), mucin, secretory agglutinins, bacteria, and other constituents in the SOR matrix. There is no discernable difference between the 2 neat Normal SOR and 2 neat AD SOR sample lanes.

Lanes 6 and 7 demonstrate 2 different neat Normal SOR samples (the corresponding samples in lanes 2 and 3) which were purified and enriched by the Alzheimer's Biomarker Purification Kit into matrix-free buffer samples. As can be visually seen in lanes 6 and 7, the total Tau has been purified and enriched in the matrix free buffer as a single band in the Gel.

Lanes 8 and 9 demonstrate 2 different neat AD SOR samples (the corresponding samples in lanes 4 and 5) which were purified and enriched by the Alzheimer's Biomarker Purification Kit into a matrix-free buffer sample. As can be visually seen in lanes 8 and 9, the total Tau has been purified and enriched in the matrix free buffer as a single band in the Gel.

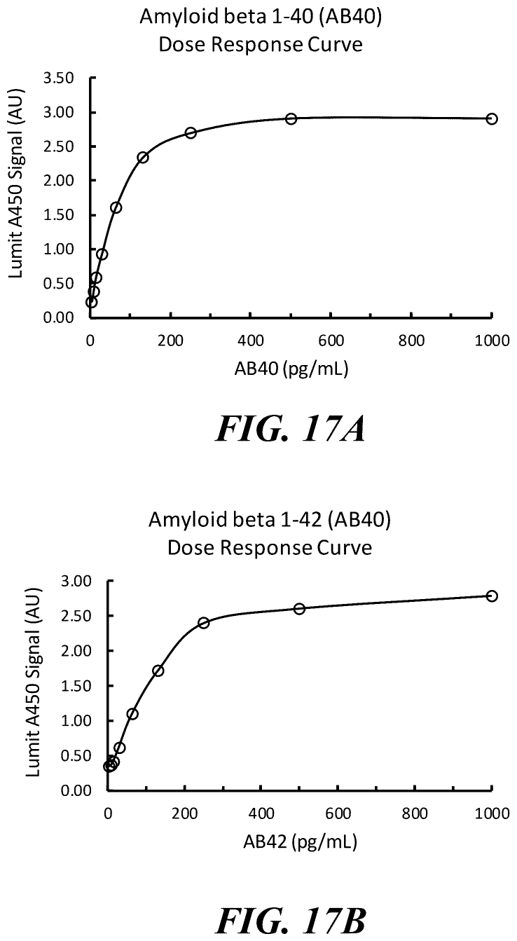

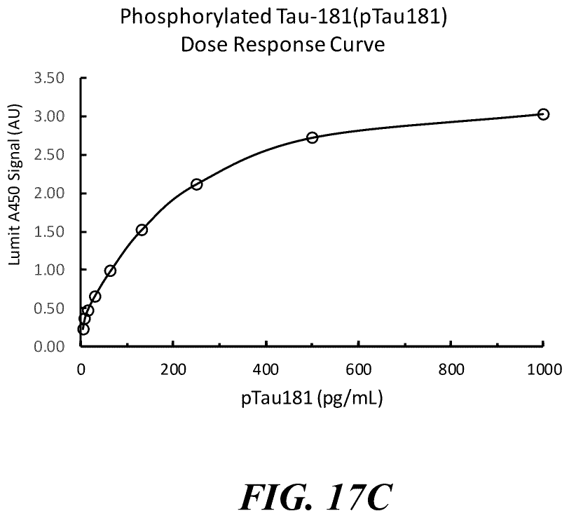

The SOR samples were spiked with amyloid beta 1-40 (AB40), amyloid beta 1-42 (AB42), and phosphorylated Tau181 (pTau181) at 12 different levels from 488 fg/mL to 1000 pg/mL in Sarstedt low binding resin polypropylene tubes. The samples were cleaned with clean beads, captured with capture beads, purified, and enriched for AB40, AB42, and pTau181 detections. The clean beads can target and multiplex remove endogenous heterophilic interference such as HAMA or RF interference, human anti-animal antibodies (e.g., anti-goat, anti-rabbit, anti-sheep, anti-mouse, and anti-bovine IgG), anti-streptavidin, anti-biotin, anti-polyethylene glycol (e.g., anti-PEG or anti-polyethylene oxide), anti-polyvinylpyrrolidone (e.g., anti-PVP), and non-specific binding (e.g., NSB). The clean beads can be coated with different antibody species (mouse, goat, rabbit, sheep, and bovine IgG), streptavidin, and polyethylene glycol (PEG). The capture beads were coated with anti-total Amyloid beta monoclonal antibody and anti-total Tau monoclonal antibodies. The samples were tested using INNOTEST ELISAs from Fujirebio for AB40, AB42, and pTau181 to generate dose response curves for each biomarker (A450 vs. dose). Table 63 shows the response for each biomarker. FIGS. 17A-17C show the dose response curves for AB40, AB42, and pTau181 respectively.

TABLE 63
Dose response for AB40, AB42, and pTau181

| Amyloid beat 1-40 | | | Amyloid beat 1-42 | | | Phosphorylated Tau-181 | | |
|---|---|---|---|---|---|---|---|---|
| | Con. | | | Con. | | | Con. | |
| AB40 | (pg/mL) | AU450 | AB42 | (pg/mL) | AU450 | pTau181 | (pg/mL) | AU450 |
| 1 | 1000 | 2.918 | 1 | 1000 | 2.790 | 1 | 1000 | 3.028 |
| 2 | 500 | 2.915 | 2 | 500 | 2.607 | 2 | 500 | 2.719 |
| 3 | 250 | 2.702 | 3 | 250 | 2.397 | 3 | 250 | 2.116 |
| 4 | 132 | 2.349 | 4 | 132 | 1.721 | 4 | 132 | 1.525 |
| 5 | 63.6 | 1.615 | 5 | 63.6 | 1.096 | 5 | 63.6 | 0.987 |
| 6 | 29.1 | 0.937 | 6 | 29.1 | 0.610 | 6 | 29.1 | 0.654 |
| 7 | 14.0 | 0.589 | 7 | 14.0 | 0.406 | 7 | 14.0 | 0.475 |
| 8 | 7.80 | 0.379 | 8 | 7.80 | 0.359 | 8 | 7.80 | 0.361 |
| 9 | 3.90 | 0.234 | 9 | 3.90 | 0.346 | 9 | 3.90 | 0.234 |
| 10 | 1.95 | 0.231 | 10 | 1.95 | 0.311 | 10 | 1.95 | 0.231 |
| 11 | 0.98 | 0.229 | 11 | 0.98 | 0.315 | 11 | 0.98 | 0.230 |
| 12 | 0.49 | 0.227 | 12 | 0.49 | 0.318 | 12 | 0.49 | 0.231 |
| 13 | 0 | 0.222 | 13 | 0 | 0.249 | 13 | 0 | 0.222 |

The process provided in the present disclosure increased the limit of detection of the INNOTEST AB40 ELISA by 8.0-fold, i.e., from 7.8 pg/mL to 0.98 pg/mL, increased the limit of detection of the INNOTEST AB42 ELISA by 16.2-fold, i.e., from 63 pg/mL to 3.9 pg/mL, and increased the limit of detection of the INNOTEST pTau181 ELISA by 6.4-fold, i.e., from 25 pg/mL to 3.9 pg/mL.

The Lumit™ immunoassay was used for the immunoassay procedure. The Lumit™ immunoassay was a homogeneous (no-wash) assay that detected a given analyte (e.g., AB40 peptide, AB42 peptide, or pTau181 peptide in test samples). The immunoassay did not require immobilization of detection antibodies to plate, beads, or other surfaces. The immunoassay procedure comprised adding the assay reagents to a test sample, waiting for about 90 minutes, adding detection reagent, and reading. The immunoassay was based on a luminescent structural complementation system consisting of two small peptide tags and a reagent-based polypeptide (LgT). Antibodies were chemically labeled with the peptide tags SmTrip9 and SmTrip10. In the presence of analyte(s), the labeled antibodies bound the analyte, bringing the peptide subunits into close proximity allowing the binding of LgT present in solution to reassemble into a functional luminescent enzyme that generated a luminescent signal in the presence of furimazine substrate.

The immunoassay background signal was determined by testing each antibody pair for AB40, AB42, and pTau181 with 10 μL Assay Buffer as the sample (no analytes). Antibody pairs include clones HT7 for total Tau, AT270 for pTau181, 3D6 for total amyloid beta, 2G3 for AB40, and 21F12 for AB42.

10 μL of neat SOR was tested by the AB40, AB42, and pTau181 immunoassays and no analyte signal (RLU) greater than background signal was detected. The immunoassay did not detect endogenous AB40, AB42, or pTau181 in neat SOR.

Neat SOR, and neat SOR spiked with 10 pg/mL or 100 pg/mL AB40, AB42, and pTau181 were processed by the Alzheimer's Biomarker Purification Kit to clean, capture, purify and enrich AB40, AB42, and pTau181. 10 μL of each the above processed sample was tested by the AB40, AB42, and pTau181 immunoassays. All 3 biomarkers were detected with significantly greater signal than the background signal, and assay signal increased with 10 pg/mL spikes and further increased with 100 pg/mL spikes. The signal response for each biomarker increased proportionally to the amount of biomarker spiked (AB40 signal increased from 323 RLU neat to 896 RLU with 10 pg/mL AB40 spike to 3,794 RLU with 100 pg/mL AB40 spike; AB42 signal increased from 621 RLU neat to 1,420 RLU with 10 pg/mL AB42 spike to 5,811 RLU with 100 pg/mL AB42 spike; pTau181 signal increased from 543 RLU neat to 1,034 RLU with 10 pg/mL pTau181 spike to 4,172 RLU with 100 pg/mL pTau181 spike). The signal (RLU) is summarized in Table 64.

TABLE 64
Signal for SOR samples for Alzheimer's biomarker detection

| | Signal (RLU) | | |
|---|---|---|---|
| Sample | AB40 | AB42 | pTau181 |
| Immunoassay Background | 219 | 481 | 377 |
| Signal | | | |
| SOR | 216 | 466 | 317 |
| (Neat Sample) | | | |
| SOR | 323 | 621 | 543 |
| (Purified and Enriched Sample) | | | |
| Spike 1 SOR (10 pg/mL) | 896 | 1,420 | 1,034 |
| (Purified and Enriched Sample) | | | |
| Spike 2 SOR (100 pg/mL) | 3,794 | 5,811 | 4,172 |
| (Purified and Enriched Sample) | | | |

Neat SOR and neat SOR spiked with 8 different levels of AB40, AB42, and pTau181 from 0 to 400 pg/mL were processed by the Alzheimer's Biomarker Purification Kit in triplicate (N=3) to remove interference, capture, purify and enrich AB40, AB42, and pTau181. 10 μL of each processed sample was tested by the AB40, AB42, and pTau181 immunoassays, and all 3 biomarkers were detected in the 6 pg/mL up to 400 pg/mL spikes with significantly greater signal as compared to the Neat Saliva and Buffer vehicle control spikes (0 pg/mL). The testing results are summarized in Table 65.

TABLE 65
Signal for SOR samples for Alzheimer's biomarker detection

AB40 Immunoassay Signal (RLU)

| AB40 Spiked | Average | Standard | |
|---|---|---|---|
| SOR (pg/mL) | (N = 3 reps) | deviation | % CV |
| 400 | 342,567 | 12,357 | 3.6% |
| 200 | 149,167 | 6,116 | 4.1% |
| 100 | 64,050 | 2,237 | 3.5% |
| 50 | 27,300 | 1,843 | 6.8% |
| 25 | 13,003 | 775 | 6.0% |
| 13 | 6,935 | 527 | 7.6% |
| 6 | 3,112 | 218 | 7.0% |
| 0 | 309 | 31 | 9.9% |
| Neat SOR | 331 | 66 | 20.0% |

AB42 Immunoassay Signal (RLU)

| AB42 Spiked | Average | Standard | |
|---|---|---|---|
| SOR (pg/mL) | (N = 3 reps) | deviation | % CV |
| 400 | 339,367 | 11,210 | 3.3% |
| 200 | 142,367 | 1,498 | 1.1% |
| 100 | 72,023 | 3,485 | 4.8% |
| 50 | 33,933 | 412 | 1.2% |
| 25 | 15,967 | 462 | 2.9% |
| 13 | 8,255 | 113 | 1.4% |
| 6 | 4,229 | 143 | 3.4% |
| 0 | 394 | 59 | 15.0% |
| Neat SOR | 271 | 17 | 6.5% |

pTau181 Immunoassay Signal (RLU)

| pTau181 Spiked | Average | Standard | |
|---|---|---|---|
| SOR (pg/mL) | (N = 3 reps) | deviation | % CV |
| 400 | 19,127 | 483 | 2.5% |
| 200 | 8,806 | 137 | 1.6% |
| 100 | 4,222 | 91 | 2.2% |
| 50 | 1,912 | 33 | 1.7% |
| 25 | 1,154 | 10 | 0.9% |
| 13 | 670 | 5 | 0.7% |
| 6 | 586 | 38 | 6.5% |
| 0 | 391 | 62 | 15.9% |
| Neat SOR | 296 | 12 | 4.2% |

The sample transformation and biomarker purification process with subsequent immunoassay detection demonstrated overall % CVs from 3.5% to 7.6% for AB40 (5.5% mean CV), 1.1% to 4.8% for AB42 (2.6% mean CV), and 0.7% to 6.5% for pTau181 (2.3% mean CV) for SOR spikes from 6 pg/mL up to 400 pg/mL.

Purified and enriched SOR samples (of healthy controls and moderate Alzheimer's patients) were tested using the Lumit immunoassay for AB40, AB42, and pTau181 biomarkers.

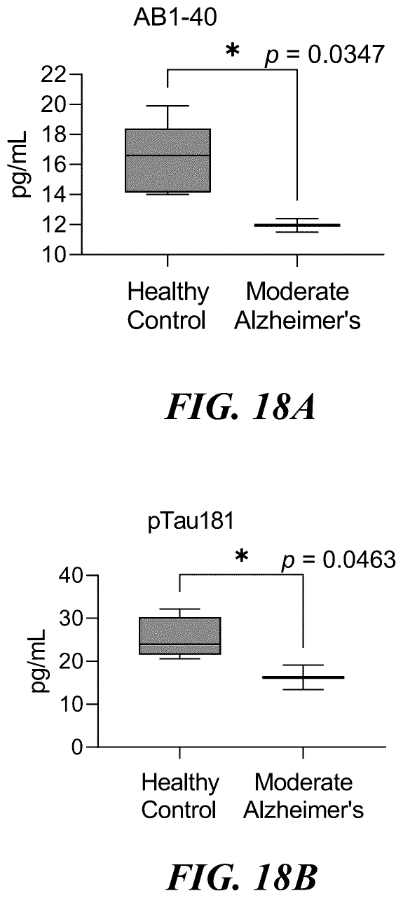

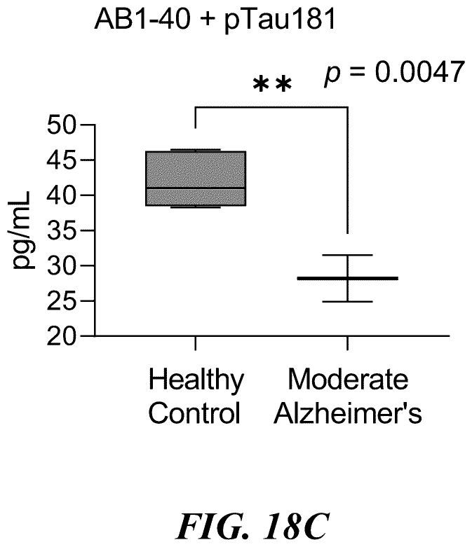

FIGS. 18A-18C show the levels of Amyloid beta 1-40 (AB40 or AB1-40, FIG. 18A), phosphorylated Tau (pTau181, FIG. 18B), and AB40+pTau181 (FIG. 18C) were lower in Moderate Alzheimer's patients' SOR samples (N=2) as compared to Healthy Controls' SOR samples (N=6).

This example demonstrates the sensitivity and specificity of the method provided herein in processing SOR samples for Alzheimer's biomarkers detection.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-119. (canceled)

120. An assay method comprising:
(a) contacting a sample comprising a biomarker with a biomarker capture particle, thereby capturing the biomarker by the biomarker capture particle to yield a captured biomarker; and
(b) binding the captured biomarker with a detection composition, wherein the detection composition has an interference removed by an interference capture composition.

121. The assay method of claim 120, wherein the interference capture composition comprises a capture moiety.

122. The assay method of claim 120, further comprising cleaning the detection composition by removing the interference from the detection composition with the interference capture composition.

123. The assay method of claim 122, wherein the cleaning of the detection composition reduces a background signal.

124. The assay method of claim 120, wherein the detection composition comprises a signal detection moiety.

125. The assay method of claim 120, further comprising, prior to (a), removing an interference from the sample and enriching the biomarker in the sample.

126. The assay method of claim 120, wherein the biomarker is an antigen or fragment thereof, and the detection composition comprises an antibody or fragment thereof that is specific to the antigen or fragment thereof.

127. The assay method of claim 120, wherein the biomarker is an antibody or fragment thereof, and the detection composition comprises an antigen or fragment thereof that is specific to the antibody or fragment thereof.

128. The assay method of claim 120, further comprising eluting the captured biomarker and detecting the detection composition bound by the captured biomarker.

129. The assay method of claim 128, wherein the detecting comprises immunoassay, homogeneous assay, agglutination based assay, affinity assay, immuno-PCR, sandwich assay, fluorescence based assay, chemiluminescence based assay, enzyme-linked immunoassay, mass spectrometry, electrochemiluminescence based assay, or molecular assay.

130. An assay method comprising:
(a) contacting a sample comprising a biomarker with a first agglutination particle comprising a first color and a second agglutination particle comprising a second color, wherein the first agglutination particle and the second agglutination particle aggregate upon binding the biomarker in the sample to form an aggregated agglutination particle; and
(b) detecting the aggregated agglutination particle.

131. The assay method of claim 130, wherein the sample comprises a plurality of biomarkers, wherein the plurality of biomarkers binds to a plurality of agglutination particles, thereby forming a network comprising the plurality of biomarkers and the plurality of agglutination particles.

132. The assay method of claim 131, wherein the network is visually detectable.

133. The assay method of claim 131, wherein the detecting comprises a visual detection, turbidimetric detection, UV-vis detection, infrared detection, light scattering detection, or microscopy detection.

134. The assay method of claim 131, wherein the biomarker binds to the first agglutination particle and the second agglutination particle.

135. An assay method comprising:
(a) contacting a sample comprising a biomarker with (i) a plurality of magnetic particles and (ii) a plurality of non-magnetic particles, wherein the plurality of magnetic particles are larger in size than the plurality of non-magnetic particles, wherein a magnetic particle of the plurality of magnetic particles and a non-magnetic particle of the plurality of non-magnetic particles form a complex upon binding the biomarker; and
(b) detecting the complex.

136. The assay method of claim 135, wherein the plurality of magnetic particles has a size from 200 nanometers (nm) to 3000 nm, and the plurality of non-magnetic particles has a size from 10 nm to 300 nm.

137. The assay method of claim 135, wherein the magnetic particle comprises a first capture moiety that binds the biomarker and the non-magnetic particle comprises a second capture moiety that binds the biomarker.

138. The assay method of claim 135, further comprising removing the complex from the sample.

139. The assay method of claim 138, wherein the plurality of non-magnetic particles comprises a color, and wherein the detecting the complex comprises detecting a color of the complex, a color change of the sample, or a color intensity change of the sample, or a combination thereof.
